SEQUENCE LISTING (186 sequences)

SEQ ID NO: 1 - protein, 150 aa, synthetic construct
  REGION 1..150: note = Synthetic peptide
  SITE 1: note = MISC_FEATURE - Optional N-terminal signal peptide
  REGION 83..85: note = MISC_FEATURE - sequon sequence
  SITE 150: note = MISC_FEATURE - Optional C-terminal purification tag
KYDGSKLRIGILHARGNAEIILELVLGALKRLQEFGVKRENIIIETVPGSFELPYGSKLFVEKQKRLGKPLDAIIPIGVLIRNETAHFDYIADSTTHQLMKLNFELGIPVIFGVLTTESDEQAEERAGTKAGNHGEDWGAAAVEMATKFN

SEQ ID NO: 2 - protein, 150 aa, synthetic construct
  REGION 1..150: note = Synthetic peptide
  SITE 1: note = MISC_FEATURE - Optional N-terminal signal peptide
  REGION 84..86: note = MISC_FEATURE - sequon sequence
  SITE 150: note = MISC_FEATURE - Optional C-terminal purification tag
KYDGSKLRIGILHARGNAEIILELVLGALKRLQEFGVKRENIIIETVPGSFELPYGSKLFVEKQKRLGKPLDAIIPIGVLIRGNDSHFDYIADSTTHQLMKLNFELGIPVIFGVLTTESDEQAEERAGTKAGNHGEDWGAAAVEMATKFN

SEQ ID NO: 3 - protein, 150 aa, synthetic construct
  REGION 1..150: note = Synthetic peptide
  SITE 1: note = MISC_FEATURE - Optional N-terminal signal peptide
  REGION 118..120: note = MISC_FEATURE - sequon sequence
  SITE 150: note = MISC_FEATURE - Optional C-terminal purification tag
KYDGSKLRIGILHARGNAEIILELVLGALKRLQEFGVKRENIIIETVPGSFELPYGSKLFVEKQKRLGKPLDAIIPIGVLIRGSTAHFDYIADSTTHQLMKLNFELGIPVIFGVLTTNSTEQAEERAGTKAGNHGEDWGAAAVEMATKFN

SEQ ID NO: 4 - protein, 150 aa, synthetic construct
  REGION 1..150: note = Synthetic peptide
  SITE 1: note = MISC_FEATURE - Optional N-terminal signal peptide
  REGION 120..122: note = MISC_FEATURE - sequon sequence
  SITE 150: note = MISC_FEATURE - Optional C-terminal purification tag
KYDGSKLRIGILHARGNAEIILELVLGALKRLQEFGVKRENIIIETVPGSFELPYGSKLFVEKQKRLGKPLDAIIPIGVLIRGSTAHFDYIADSTTHQLMKLNFELGIPVIFGVLTTESNESAEERAGTKAGNHGEDWGAAAVEMATKFN

SEQ ID NO: 5 - protein, 150 aa, synthetic construct
  REGION 1..150: note = Synthetic peptide
  SITE 1: note = MISC_FEATURE - Optional N-terminal signal peptide
  REGION 118..122: note = MISC_FEATURE - sequon sequence
  SITE 150: note = MISC_FEATURE - Optional C-terminal purification tag
KYDGSKLRIGILHARGNAEIILELVLGALKRLQEFGVKRENIIIETVPGSFELPYGSKLFVEKQKRLGKPLDAIIPIGVLIRGSTAHFDYIADSTTHQLMKLNFELGIPVIFGVLTTFSNESAEERAGTKAGNHGEDWGAAAVEMATKFN

SEQ ID NO: 6 - protein, 150 aa, synthetic construct
  REGION 1..150: note = Synthetic peptide
  SITE 1: note = MISC_FEATURE - Optional N-terminal signal peptide
  REGION 121..123: note = MISC_FEATURE - sequon sequence
  SITE 150: note = MISC_FEATURE - Optional C-terminal purification tag
KYDGSKLRIGILHARGNAEIILELVLGALKRLQEFGVKRENIIIETVPGSFELPYGSKLFVEKQKRLGKPLDAIIPIGVLIRGSTAHFDYIADSTTHQLMKLNFELGIPVIFGVLTTESDNESEERAGTKAGNHGEDWGAAAVEMATKFN

SEQ ID NO: 7 - protein, 150 aa, synthetic construct
  REGION 1..150: note = Synthetic peptide
  SITE 1: note = MISC_FEATURE - Optional N-terminal signal peptide
  REGION 119..123: note = MISC_FEATURE - sequon sequence
  SITE 150: note = MISC_FEATURE - Optional C-terminal purification tag
KYDGSKLRIGILHARGNAEIILELVLGALKRLQEFGVKRENIIIETVPGSFELPYGSKLFVEKQKRLGKPLDAIIPIGVLIRGSTAHFDYIADSTTHQLMKLNFELGIPVIFGVLTTEFENESEERAGTKAGNHGEDWGAAAVEMATKFN

SEQ ID NO: 8 - protein, 150 aa, synthetic construct
  REGION 1..150: note = Synthetic peptide
  SITE 1: note = MISC_FEATURE - Optional N-terminal signal peptide
  REGION 130..132: note = MISC_FEATURE - sequon sequence
  SITE 150: note = MISC_FEATURE - Optional C-terminal purification tag
KYDGSKLRIGILHARGNAEIILELVLGALKRLQEFGVKRENIIIETVPGSFELPYGSKLFVEKQKRLGKPLDAIIPIGVLIRGSTAHFDYIADSTTHQLMKLNFELGIPVIFGVLTTESDEQAEERAGTNASNHGEDWGAAAVEMATKFN

SEQ ID NO: 9 - protein, 150 aa, synthetic construct
  REGION 1..150: note = Synthetic peptide
  SITE 1: note = MISC_FEATURE - Optional N-terminal signal peptide
  REGION 131..133: note = MISC_FEATURE - sequon sequence
  SITE 150: note = MISC_FEATURE - Optional C-terminal purification tag
KYDGSKLRIGILHARGNAEIILELVLGALKRLQEFGVKRENIIIETVPGSFELPYGSKLFVEKQKRLGKPLDAIIPIGVLIRGSTAHFDYIADSTTHQLMKLNFELGIPVIFGVLTTESDEQAEERAGTKNGSHGEDWGAAAVEMATKFN

SEQ ID NO: 10 - protein, 119 aa, synthetic construct
  REGION 1..119: note = Synthetic peptide
  SITE 1: note = MISC_FEATURE - Optional N-terminal signal peptide
  REGION 43..45: note = MISC_FEATURE - sequon sequence
  SITE 119: note = MISC_FEATURE - Optional C-terminal purification tag
EEAELAYLLGELAYKLGEYRIAIRAYRIALKRNHTNAEAWYNLGNAYYKQGRYREAIEYYQKALELDPNNAEAWYNLGNAYYERGEYEEAIEYYRKALRLDPNNADAMQNLLNAKMREE

SEQ ID NO: 11 - protein, 119 aa, synthetic construct
  REGION 1..119: note = Synthetic peptide
  SITE 1: note = MISC_FEATURE - Optional N-terminal signal peptide
  REGION 31..35: note = MISC_FEATURE - sequon sequence
  SITE 119: note = MISC_FEATURE - Optional C-terminal purification tag
EEAELAYLLGELAYKLGEYRIAIRAYRIALFFNHTNAEAWYNLGNAYYKQGRYREAIEYYQKALELDPNNAEAWYNLGNAYYERGEYEEAIEYYRKALRLDPNNADAMQNLLNAKMREE

SEQ ID NO: 12 - protein, 119 aa, synthetic construct
  REGION 1..119: note = Synthetic peptide
  SITE 1: note = MISC_FEATURE - Optional N-terminal signal peptide
  REGION 34..36: note = MISC_FEATURE - sequon sequence
  SITE 119: note = MISC_FEATURE - Optional C-terminal purification tag
EEAELAYLLGELAYKLGEYRIAIRAYRIALKRDNLSAEAWYNLGNAYYKQGRYREAIEYYQKALELDPNNAEAWYNLGNAYYERGEYEEAIEYYRKALRLDPNNADAMQNLLNAKMREE

SEQ ID NO: 13 - protein, 119 aa, synthetic construct
  REGION 1..119: note = Synthetic peptide
  SITE 1: note = MISC_FEATURE - Optional N-terminal signal peptide
  REGION 32..36: note = MISC_FEATURE - sequon sequence
  SITE 119: note = MISC_FEATURE - Optional C-terminal purification tag
EEAELAYLLGELAYKLGEYRIAIRAYRIALKFDNLSAEAWYNLGNAYYKQGRYREAIEYYQKALELDPNNAEAWYNLGNAYYERGEYEEAIEYYRKALRLDPNNADAMQNLLNAKMREE

SEQ ID NO: 14 - protein, 119 aa, synthetic construct
  REGION 1..119: note = Synthetic peptide
  SITE 1: note = MISC_FEATURE - Optional N-terminal signal peptide
  REGION 35..37: note = MISC_FEATURE - sequon sequence
  SITE 119: note = MISC_FEATURE - Optional C-terminal purification tag
EEAELAYLLGELAYKLGEYRIAIRAYRIALKRDPNNSEAWYNLGNAYYKQGRYREAIEYYQKALELDPNNAEAWYNLGNAYYERGEYEEAIEYYRKALRLDPNNADAMQNLLNAKMREE

SEQ ID NO: 15 - protein, 119 aa, synthetic construct
  REGION 1..119: note = Synthetic peptide
  SITE 1: note = MISC_FEATURE - Optional N-terminal signal peptide
  REGION 33..37: note = MISC_FEATURE - sequon sequence
  SITE 119: note = MISC_FEATURE - Optional C-terminal purification tag
EEAELAYLLGELAYKLGEYRIAIRAYRIALKRWHNNSEAWYNLGNAYYKQGRYREAIEYYQKALELDPNNAEAWYNLGNAYYERGEYEEAIEYYRKALRLDPNNADAMQNLLNAKMREE

SEQ ID NO: 16 - protein, 119 aa, synthetic construct
  REGION 1..119: note = Synthetic peptide
  SITE 1: note = MISC_FEATURE - Optional N-terminal signal peptide
  REGION 58..60: note = MISC_FEATURE - sequon sequence
  SITE 119: note = MISC_FEATURE - Optional C-terminal purification tag
EEAELAYLLGELAYKLGEYRIAIRAYRIALKRDPNNAEAWYNLGNAYYKQGRYREAINYSQKALELDPNNAEAWYNLGNAYYERGEYEEAIEYYRKALRLDPNNADAMQNLLNAKMREE

SEQ ID NO: 17 - protein, 119 aa, synthetic construct
  REGION 1..119: note = Synthetic peptide
  SITE 1: note = MISC_FEATURE - Optional N-terminal signal peptide
  REGION 56..60: note = MISC_FEATURE - sequon sequence
  SITE 119: note = MISC_FEATURE - Optional C-terminal purification tag
EEAELAYLLGELAYKLGEYRIAIRAYRIALKRDPNNAEAWYNLGNAYYKQGRYREFINYSQKALELDPNNAEAWYNLGNAYYERGEYEEAIEYYRKALRLDPNNADAMQNLLNAKMREE

SEQ ID NO: 18 - protein, 119 aa, synthetic construct
  REGION 1..119: note = Synthetic peptide
  SITE 1: note = MISC_FEATURE - Optional N-terminal signal peptide
  REGION 61..63: note = MISC_FEATURE - sequon sequence
  SITE 119: note = MISC_FEATURE - Optional C-terminal purification tag
EEAELAYLLGELAYKLGEYRIAIRAYRIALKRDPNNAEAWYNLGNAYYKQGRYREAIEYYNISLELDPNNAEAWYNLGNAYYERGEYEEAIEYYRKALRLDPNNADAMQNLLNAKMREE

SEQ ID NO: 19 - protein, 119 aa, synthetic construct
  REGION 1..119: note = Synthetic peptide
  SITE 1: note = MISC_FEATURE - Optional N-terminal signal peptide
  REGION 60..64: note = MISC_FEATURE - sequon sequence
  SITE 119: note = MISC_FEATURE - Optional C-terminal purification tag
EEAELAYLLGELAYKLGEYRIAIRAYRIALKRDPNNAEAWYNLGNAYYKQGRYREAIEYFLNATELDPNNAEAWYNLGNAYYERGEYEEAIEYYRKALRLDPNNADAMQNLLNAKMREE

SEQ ID NO: 20 - protein, 119 aa, synthetic construct
  REGION 1..119: note = Synthetic peptide
  SITE 1: note = MISC_FEATURE - Optional N-terminal signal peptide
  REGION 65..67: note = MISC_FEATURE - sequon sequence
  SITE 119: note = MISC_FEATURE - Optional C-terminal purification tag
EEAELAYLLGELAYKLGEYRIAIRAYRIALKRDPNNAEAWYNLGNAYYKQGRYREAIEYYQKALNLSPNNAEAWYNLGNAYYERGEYEEAIEYYRKALRLDPNNADAMQNLLNAKMREE

SEQ ID NO: 21 - protein, 119 aa, synthetic construct
  REGION 1..119: note = Synthetic peptide
  SITE 1: note = MISC_FEATURE - Optional N-terminal signal peptide
  REGION 68..70: note = MISC_FEATURE - sequon sequence
  SITE 119: note = MISC_FEATURE - Optional C-terminal purification tag
EEAELAYLLGELAYKLGEYRIAIRAYRIALKRDPNNAEAWYNLGNAYYKQGRYREAIEYYQKALELDNLSAEAWYNLGNAYYERGEYEEAIEYYRKALRLDPNNADAMQNLLNAKMREE

SEQ ID NO: 22 - protein, 119 aa, synthetic construct
  REGION 1..119: note = Synthetic peptide
  SITE 1: note = MISC_FEATURE - Optional N-terminal signal peptide
  REGION 66..70: note = MISC_FEATURE - sequon sequence
  SITE 119: note = MISC_FEATURE - Optional C-terminal purification tag
EEAELAYLLGELAYKLGEYRIAIRAYRIALKRDPNNAEAWYNLGNAYYKQGRYREAIEYYQKALEFDNLSAEAWYNLGNAYYERGEYEEAIEYYRKALRLDPNNADAMQNLLNAKMREE

SEQ ID NO: 23 - protein, 119 aa, synthetic construct
  REGION 1..119: note = Synthetic peptide
  SITE 1: note = MISC_FEATURE - Optional N-terminal signal peptide
  REGION 89..91: note = MISC_FEATURE - sequon sequence
  SITE 119: note = MISC_FEATURE - Optional C-terminal purification tag
EEAELAYLLGELAYKLGEYRIAIRAYRIALKRDPNNAEAWYNLGNAYYKQGRYREAIEYYQKALELDPNNAEAWYNLGNAYYERGEYENATEYYRKALRLDPNNADAMQNLLNAKMREE

SEQ ID NO: 24 - protein, 119 aa, synthetic construct
  REGION 1..119: note = Synthetic peptide
  SITE 1: note = MISC_FEATURE - Optional N-terminal signal peptide
  REGION 94..98: note = MISC_FEATURE - sequon sequence
  SITE 119: note = MISC_FEATURE - Optional C-terminal purification tag
EEAELAYLLGELAYKLGEYRIAIRAYRIALKRDPNNAEAWYNLGNAYYKQGRYREAIEYYQKALELDPNNAEAWYNLGNAYYERGEYEEAIEYFLNASRLDPNNADAMQNLLNAKMREE

SEQ ID NO: 25 - protein, 119 aa, synthetic construct
  REGION 1..119: note = Synthetic peptide
  SITE 1: note = MISC_FEATURE - Optional N-terminal signal peptide
  REGION 100..102: note = MISC_FEATURE - sequon sequence
  SITE 119: note = MISC_FEATURE - Optional C-terminal purification tag
EEAELAYLLGELAYKLGEYRIAIRAYRIALKRDPNNAEAWYNLGNAYYKQGRYREAIEYYQKALELDPNNAEAWYNLGNAYYERGEYEEAIEYYRKALRNNSNNADAMQNLLNAKMREE

SEQ ID NO: 26 - protein, 200 aa, synthetic construct
  REGION 1..200: note = Synthetic peptide
  SITE 1: note = MISC_FEATURE - Optional N-terminal signal peptide
  REGION 43..45: note = MISC_FEATURE - sequon sequence
  SITE 200: note = MISC_FEATURE - Optional C-terminal purification tag
EELFKKHKIVAVLRANSVEEAIEKAVAVFAGGVHLIEITFTVNNSDTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGC

SEQ ID NO: 27 - protein, 200 aa, synthetic construct
  REGION 1..200: note = Synthetic peptide
  SITE 1: note = MISC_FEATURE - Optional N-terminal signal peptide
  REGION 41..45: note = MISC_FEATURE - sequon sequence
  SITE 200: note = MISC_FEATURE - Optional C-terminal purification tag
EELFKKHKIVAVLRANSVEEAIEKAVAVFAGGVHLIEITFWVNNSDTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGC

SEQ ID NO: 28 - protein, 200 aa, synthetic construct
  REGION 1..200: note = Synthetic peptide
  SITE 1: note = MISC_FEATURE - Optional N-terminal signal peptide
  REGION 44..46: note = MISC_FEATURE - sequon sequence
  SITE 200: note = MISC_FEATURE - Optional C-terminal purification tag
EELFKKHKIVAVLRANSVEEAIEKAVAVFAGGVHLIEITFTVPNASTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGC

SEQ ID NO: 29 - protein, 200 aa, synthetic construct
  REGION 1..200: note = Synthetic peptide
  SITE 1: note = MISC_FEATURE - Optional N-terminal signal peptide
  REGION 57..59: note = MISC_FEATURE - sequon sequence
  SITE 200: note = MISC_FEATURE - Optional C-terminal purification tag
EELFKKHKIVAVLRANSVEEAIEKAVAVFAGGVHLIEITFTVPDADTVIKALSVLNKSGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGC

SEQ ID NO: 30 - protein, 200 aa, synthetic construct
  REGION 1..200: note = Synthetic peptide
  SITE 1: note = MISC_FEATURE - Optional N-terminal signal peptide
  REGION 54..58: note = MISC_FEATURE - sequon sequence
  SITE 200: note = MISC_FEATURE - Optional C-terminal purification tag
EELFKKHKIVAVLRANSVEEAIEKAVAVFAGGVHLIEITFTVPDADTVIKALSYLNKSGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGC

SEQ ID NO: 31 - protein, 200 aa, synthetic construct
  REGION 1..200: note = Synthetic peptide
  SITE 1: note = MISC_FEATURE - Optional N-terminal signal peptide
  REGION 68..72: note = MISC_FEATURE - sequon sequence
  SITE 200: note = MISC_FEATURE - Optional C-terminal purification tag
EELFKKHKIVAVLRANSVEEAIEKAVAVFAGGVHLIEITFTVPDADTVIKALSVLKEKGAIIGAGTVFSNETCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGC

SEQ ID NO: 32 - protein, 200 aa, synthetic construct
  REGION 1..200: note = Synthetic peptide
  SITE 1: note = MISC_FEATURE - Optional N-terminal signal peptide
  REGION 69..73: note = MISC_FEATURE - sequon sequence
  SITE 200: note = MISC_FEATURE - Optional C-terminal purification tag
EELFKKHKIVAVLRANSVEEAIEKAVAVFAGGVHLIEITFTVPDADTVIKALSVLKEKGAIIGAGTVTYVNVTRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGC

SEQ ID NO: 33 - protein, 200 aa, synthetic construct
  REGION 1..200: note = Synthetic peptide
  SITE 1: note = MISC_FEATURE - Optional N-terminal signal peptide
  REGION 73..75: note = MISC_FEATURE - sequon sequence
  SITE 200: note = MISC_FEATURE - Optional C-terminal purification tag
EELFKKHKIVAVLRANSVEEAIEKAVAVFAGGVHLIEITFTVPDADTVIKALSVLKEKGAIIGAGTVTSVEQCNRSVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGC

SEQ ID NO: 34 - protein, 200 aa, synthetic construct
  REGION 1..200: note = Synthetic peptide
  SITE 1: note = MISC_FEATURE - Optional N-terminal signal peptide
  REGION 72..76: note = MISC_FEATURE - sequon sequence
  SITE 200: note = MISC_FEATURE - Optional C-terminal purification tag
EELFKKHKIVAVLRANSVEEAIEKAVAVFAGGVHLIEITFTVPDADTVIKALSVLKEKGAIIGAGTVTSVEYANRSVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGC

SEQ ID NO: 35 - protein, 200 aa, synthetic construct
  REGION 1..200: note = Synthetic peptide
  SITE 1: note = MISC_FEATURE - Optional N-terminal signal peptide
  REGION 78..80: note = MISC_FEATURE - sequon sequence
  SITE 200: note = MISC_FEATURE - Optional C-terminal purification tag
EELFKKHKIVAVLRANSVEEAIEKAVAVFAGGVHLIEITFTVPDADTVIKALSVLKEKGAIIGAGTVTSVEQCRKAVNASAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGC

SEQ ID NO: 36 - protein, 200 aa, synthetic construct
  REGION 1..200: note = Synthetic peptide
  SITE 1: note = MISC_FEATURE - Optional N-terminal signal peptide
  REGION 80..82: note = MISC_FEATURE - sequon sequence
  SITE 200: note = MISC_FEATURE - Optional C-terminal purification tag
EELFKKHKIVAVLRANSVEEAIEKAVAVFAGGVHLIEITFTVPDADTVIKALSVLKEKGAIIGAGTVTSVEQCRKAVESNASFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGC

SEQ ID NO: 37 - protein, 200 aa, synthetic construct
  REGION 1..200: note = Synthetic peptide
  SITE 1: note = MISC_FEATURE - Optional N-terminal signal peptide
  REGION 78..82: note = MISC_FEATURE - sequon sequence
  SITE 200: note = MISC_FEATURE - Optional C-terminal purification tag
EELFKKHKIVAVLRANSVEEAIEKAVAVFAGGVHLIEITFTVPDADTVIKALSVLKEKGAIIGAGTVTSVEQCRKAVWANASFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGC

SEQ ID NO: 38 - protein, 200 aa, synthetic construct
  REGION 1..200: note = Synthetic peptide
  SITE 1: note = MISC_FEATURE - Optional N-terminal signal peptide
  REGION 82..84: note = MISC_FEATURE - sequon sequence
  SITE 200: note = MISC_FEATURE - Optional C-terminal purification tag
EELFKKHKIVAVLRANSVEEAIEKAVAVFAGGVHLIEITFTVPDADTVIKALSVLKEKGAIIGAGTVTSVEQCRKAVESGANFTVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGC

SEQ ID NO: 39 - protein, 200 aa, synthetic construct
  REGION 1..200: note = Synthetic peptide
  SITE 1: note = MISC_FEATURE - Optional N-terminal signal peptide
  REGION 80..84: note = MISC_FEATURE - sequon sequence
  SITE 200: note = MISC_FEATURE - Optional C-terminal purification tag
EELFKKHKIVAVLRANSVEEAIEKAVAVFAGGVHLIEITFTVPDADTVIKALSVLKEKGAIIGAGTVTSVEQCRKAVESWANFTVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGC

SEQ ID NO: 40 - protein, 200 aa, synthetic construct
  REGION 1..200: note = Synthetic peptide
  SITE 1: note = MISC_FEATURE - Optional N-terminal signal peptide
  REGION 95..97: note = MISC_FEATURE - sequon sequence
  SITE 200: note = MISC_FEATURE - Optional C-terminal purification tag
EELFKKHKIVAVLRANSVEEAIEKAVAVFAGGVHLIEITFTVPDADTVIKALSVLKEKGAIIGAGTVTSVEQCRKAVESGAEFIVSPHLDEEISNFTKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGC

SEQ ID NO: 41 - protein, 200 aa, synthetic construct
  REGION 1..200: note = Synthetic peptide
  SITE 1: note = MISC_FEATURE - Optional N-terminal signal peptide
  REGION 98..100: note = MISC_FEATURE - sequon sequence
  SITE 200: note = MISC_FEATURE - Optional C-terminal purification tag
EELFKKHKIVAVLRANSVEEAIEKAVAVFAGGVHLIEITFTVPDADTVIKALSVLKEKGAIIGAGTVTSVEQCRKAVESGAEFIVSPHLDEEISQFCNESGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGC

SEQ ID NO: 42 - protein, 200 aa, synthetic construct
  REGION 1..200: note = Synthetic peptide
  SITE 1: note = MISC_FEATURE - Optional N-terminal signal peptide
  REGION 98..101: note = MISC_FEATURE - sequon sequence
  SITE 200: note = MISC_FEATURE - Optional C-terminal purification tag
EELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKNASVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGC

SEQ ID NO: 43 - protein, 200 aa, synthetic construct
  REGION 1..200: note = Synthetic peptide
  SITE 1: note = MISC_FEATURE - Optional N-terminal signal peptide
  REGION 101..103: note = MISC_FEATURE - sequon sequence
  SITE 200: note = MISC_FEATURE - Optional C-terminal purification tag
EELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKNVSYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGC

SEQ ID NO: 44 - protein, 200 aa, synthetic construct
  REGION 1..200: note = Synthetic peptide
  SITE 1: note = MISC_FEATURE - Optional N-terminal signal peptide
  REGION 120..122: note = MISC_FEATURE - sequon sequence
  SITE 200: note = MISC_FEATURE - Optional C-terminal purification tag
EELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKNGTTILKLFPGEVVGPQFVKAMKGPFPNVKFVPTGGVNLDNVCEWFKAGVLAVGVGSALVKGTPDEVREKAKAFVEKIRGC

SEQ ID NO: 45 - protein, 200 aa, synthetic construct
  REGION 1..200: note = Synthetic peptide
  SITE 1: note = MISC_FEATURE - Optional N-terminal signal peptide
  REGION 121..123: note = MISC_FEATURE - sequon sequence
  SITE 200: note = MISC_FEATURE - Optional C-terminal purification tag
EELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLNVTILKLFPGEVVGPQFVKAMKGPFPNVKFVPTGGVNLDNVCEWFKAGVLAVGVGSALVKGTPDEVREKAKAFVEKIRGC

SEQ ID NO: 46 - protein, 200 aa, synthetic construct
  REGION 1..200: note = Synthetic peptide
  SITE 1: note = MISC_FEATURE - Optional N-terminal signal peptide
  REGION 119..123: note = MISC_FEATURE - sequon sequence
  SITE 200: note = MISC_FEATURE - Optional C-terminal purification tag
EELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMWLNHTILKLFPGEVVGPQFVKAMKGPFPNVKFVPTGGVNLDNVCEWFKAGVLAVGVGSALVKGTPDEVREKAKAFVEKIRGC

SEQ ID NO: 47 - protein, 200 aa, synthetic construct
  REGION 1..200: note = Synthetic peptide
  SITE 1: note = MISC_FEATURE - Optional N-terminal signal peptide
  REGION 147..149: note = MISC_FEATURE - sequon sequence
  SITE 200: note = MISC_FEATURE - Optional C-terminal purification tag
EELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNTSFVPTGGVNLDNVCEWFKAGVLAVGVGSALVKGTPDEVREKAKAFVEKIRGC

SEQ ID NO: 48 - protein, 200 aa, synthetic construct
  REGION 1..200: note = Synthetic peptide
  SITE 1: note = MISC_FEATURE - Optional N-terminal signal peptide
  REGION 145..149: note = MISC_FEATURE - sequon sequence
  SITE 200: note = MISC_FEATURE - Optional C-terminal purification tag
EELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFHNTSFVPTGGVNLDNVCEWFKAGVLAVGVGSALVKGTPDEVREKAKAFVEKIRGC

SEQ ID NO: 49 - protein, 150 aa, synthetic construct
  REGION 1..150: note = Synthetic peptide
  SITE 1: note = MISC_FEATURE - Optional N-terminal signal peptide
  REGION 83..85: note = MISC_FEATURE - sequon sequence
  REGION 118..120: note = MISC_FEATURE - sequon sequence
  REGION 130..132: note = MISC_FEATURE - sequon sequence
  SITE 150: note = MISC_FEATURE - Optional C-terminal purification tag
KYDGSKLRIGILHARGNAEIILELVLGALKRLQEFGVKRENIIIETVPGSFELPYGSKLFVEKQKRLGKPLDAIIPIGVLIRNETAHFDYIADSTTHQLMKLNFELGIPVIFGVLTTNDTEQAEERAGTNATNHGEDWGAAAVEMATKFN

SEQ ID NO: 50 - protein, 150 aa, synthetic construct
  REGION 1..150: note = Synthetic peptide
  SITE 1: note = MISC_FEATURE - Optional N-terminal signal peptide
  REGION 84..86: note = MISC_FEATURE - sequon sequence
  REGION 120..122: note = MISC_FEATURE - sequon sequence
  REGION 131..133: note = MISC_FEATURE - sequon sequence
  SITE 150: note = MISC_FEATURE - Optional C-terminal purification tag
KYDGSKLRIGILHARGNAEIILELVLGALKRLQEFGVKRENIIIETVPGSFELPYGSKLFVEKQKRLGKPLDAIIPIGVLIRGNDTHFDYIADSTTHQLMKLNFELGIPVIFGVLTTESNETAEERAGTKNGTHGEDWGAAAVEMATKFN

SEQ ID NO: 51 - protein, 150 aa, synthetic construct
  REGION 1..150: note = Synthetic peptide
  SITE 1: note = MISC_FEATURE - Optional N-terminal signal peptide
  REGION 84..86: note = MISC_FEATURE - sequon sequence
  REGION 118..120: note = MISC_FEATURE - sequon sequence
  REGION 130..132: note = MISC_FEATURE - sequon sequence
  SITE 150: note = MISC_FEATURE - Optional C-terminal purification tag
KYDGSKLRIGILHARGNAEIILELVLGALKRLQEFGVKRENIIIETVPGSFELPYGSKLFVEKQKRLGKPLDAIIPIGVLIRGNDTHFDYIADSTTHQLMKLNFELGIPVIFGVLTTNDTEQAEERAGTNATNHGEDWGAAAVEMATKFN

SEQ ID NO: 52 - protein, 150 aa, synthetic construct
  REGION 1..150: note = Synthetic peptide
  SITE 1: note = MISC_FEATURE - Optional N-terminal signal peptide
  REGION 83..85: note = MISC_FEATURE - sequon sequence
  REGION 130..132: note = MISC_FEATURE - sequon sequence
  SITE 150: note = MISC_FEATURE - Optional C-terminal purification tag
KYDGSKLRIGILHARGNAEIILELVLGALKRLQEFGVKRENIIIETVPGSFELPYGSKLFVEKQKRLGKPLDAIIPIGVLIRNDTAHFDYIADSTTHQLMKLNFELGIPVIFGVLTTESDEQAEERAGTNATNHGEDWGAAAVEMATKFN

SEQ ID NO: 53 - protein, 150 aa, synthetic construct
  REGION 1..150: note = Synthetic peptide
  SITE 1: note = MISC_FEATURE - Optional N-terminal signal peptide
  REGION 84..86: note = MISC_FEATURE - sequon sequence
  REGION 131..133: note = MISC_FEATURE - sequon sequence
  SITE 150: note = MISC_FEATURE - Optional C-terminal purification tag
KYDGSKLRIGILHARGNAEIILELVLGALKRLQEFGVKRENIIIETVPGSFELPYGSKLFVEKQKRLGKPLDAIIPIGVLIRGNDTHFDYIADSTTHQLMKLNFELGIPVIFGVLTTESDEQAEERAGTKNGTHGEDWGAAAVEMATKFN

SEQ ID NO: 54 - protein, 150 aa, synthetic construct
  REGION 1..150: note = Synthetic peptide
  SITE 1: note = MISC_FEATURE - Optional N-terminal signal peptide
  REGION 84..86: note = MISC_FEATURE - sequon sequence
  REGION 118..120: note = MISC_FEATURE - sequon sequence
  SITE 150: note = MISC_FEATURE - Optional C-terminal purification tag
KYDGSKLRIGILHARGNAEIILELVLGALKRLQEFGVKRENIIIETVPGSFELPYGSKLFVEKQKRLGKPLDAIIPIGVLIRGNDTHFDYIADSTTHQLMKLNFELGIPVIFGVLTTNSTEQAEERAGTKAGNHGEDWGAAAVEMATKFN

SEQ ID NO: 55 - protein, 150 aa, synthetic construct
  REGION 1..150: note = Synthetic peptide
  SITE 1: note = MISC_FEATURE - Optional N-terminal signal peptide
  REGION 120..122: note = MISC_FEATURE - sequon sequence
  REGION 130..132: note = MISC_FEATURE - sequon sequence
  SITE 150: note = MISC_FEATURE - Optional C-terminal purification tag
KYDGSKLRIGILHARGNAEIILELVLGALKRLQEFGVKRENIIIETVPGSFELPYGSKLFVEKQKRLGKPLDAIIPIGVLIRGSTAHFDYIADSTTHQLMKLNFELGIPVIFGVLTTESNETAEERAGTNATNHGEDWGAAAVEMATKFN

SEQ ID NO: 56 - protein, 119 aa, synthetic construct
  REGION 1..119: note = Synthetic peptide
  SITE 1: note = MISC_FEATURE - Optional N-terminal signal peptide
  REGION 33..36: note = MISC_FEATURE - sequon sequence
  REGION 57..59: note = MISC_FEATURE - sequon sequence
  REGION 89..91: note = MISC_FEATURE - sequon sequence
  SITE 119: note = MISC_FEATURE - Optional C-terminal purification tag
EEAELAYLLGELAYKLGEYRIAIRAYRIALKRNHTNAEAWYNLGNAYYKQGRYREAIEYYQKALELNHTNAEAWYNLGNAYYERGEYENATEYYRKALRLDPNNADAMQNLLNAKMREE

SEQ ID NO: 57 - protein, 119 aa, synthetic construct
  REGION 1..119: note = Synthetic peptide
  SITE 1: note = MISC_FEATURE - Optional N-terminal signal peptide
  REGION 32..36: note = MISC_FEATURE - sequon sequence
  REGION 67..69: note = MISC_FEATURE - sequon sequence
  REGION 89..91: note = MISC_FEATURE - sequon sequence
  SITE 119: note = MISC_FEATURE - Optional C-terminal purification tag
EEAELAYLLGELAYKLGEYRIAIRAYRIALKYDNLTAEAWYNLGNAYYKQGRYREAIEYYQKALELNHTNAEAWYNLGNAYYERGEYENATEYYRKALRLDPNNADAMQNLLNAKMREE

SEQ ID NO: 58 - protein, 119 aa, synthetic construct
  REGION 1..119: note = Synthetic peptide
  SITE 1: note = MISC_FEATURE - Optional N-terminal signal peptide
  REGION 33..35: note = MISC_FEATURE - sequon sequence
  REGION 89..91: note = MISC_FEATURE - sequon sequence
  SITE 119: note = MISC_FEATURE - Optional C-terminal purification tag
EEAELAYLLGELAYKLGEYRIAIRAYRIALKRNHTNAEAWYNLGNAYYKQGRYREAIEYYQKALELDPNNAEAWYNLGNAYYERGEYENATEYYRKALRLDPNNADAMQNLLNAKMREE

SEQ ID NO: 59 - protein, 119 aa, synthetic construct
  REGION 1..119: note = Synthetic peptide
  SITE 1: note = MISC_FEATURE - Optional N-terminal signal peptide
  REGION 32..36: note = MISC_FEATURE - sequon sequence
  REGION 89..91: note = MISC_FEATURE - sequon sequence
  SITE 119: note = MISC_FEATURE - Optional C-terminal purification tag
EEAELAYLLGELAYKLGEYRIAIRAYRIALKYDNLTAEAWYNLGNAYYKQGRYREAIEYYQKALELDPNNAEAWYNLGNAYYERGEYENATEYYRKALRLDPNNADAMQNLLNAKMREE

SEQ ID NO: 60 - protein, 119 aa, synthetic construct
  REGION 1..119: note = Synthetic peptide
  SITE 1: note = MISC_FEATURE - Optional N-terminal signal peptide
  REGION 67..69: note = MISC_FEATURE - sequon sequence
  REGION 89..91: note = MISC_FEATURE - sequon sequence
  SITE 119: note = MISC_FEATURE - Optional C-terminal purification tag
EEAELAYLLGELAYKLGEYRIAIRAYRIALKRDPNNAEAWYNLGNAYYKQGRYREAIEYYQKALELNHTNAEAWYNLGNAYYERGEYENATEYYRKALRLDPNNADAMQNLLNAKMREE

SEQ ID NO: 61 - protein, 200 aa, synthetic construct
  REGION 1..200: note = Synthetic peptide
  SITE 1: note = MISC_FEATURE - Optional N-terminal signal peptide
  REGION 43..45: note = MISC_FEATURE - sequon sequence
  REGION 72..76: note = MISC_FEATURE - sequon sequence
  REGION 78..80: note = MISC_FEATURE - sequon sequence
  REGION 82..84: note = MISC_FEATURE - sequon sequence
  REGION 95..97: note = MISC_FEATURE - sequon sequence
  REGION 99..101: note = MISC_FEATURE - sequon sequence
  REGION 121..123: note = MISC_FEATURE - sequon sequence
  REGION 145..149: note = MISC_FEATURE - sequon sequence
  SITE 200: note = MISC_FEATURE - Optional C-terminal purification tag
EELFKKHKIVAVLRANSVEEAIEKAVAVFAGGVHLIEITFTVNNTDTVIKALSVLKEKGAIIGAGTVTSVEYANLTVNATANFTVSPHLDEEISNFTKNATVFYMPGVMTPTELVKAMKLNVTILKLFPGEVVGPQFVKAMKGPFHNATFVPTGGVNLDNVCEWFKAGVLAVGVGSALVKGTPDEVREKAKAFVEKIRGC

SEQ ID NO: 62 - protein, 200 aa, synthetic construct
  REGION 1..200: note = Synthetic peptide
  SITE 1: note = MISC_FEATURE - Optional N-terminal signal peptide
  REGION 44..46: note = MISC_FEATURE - sequon sequence
  REGION 72..76: note = MISC_FEATURE - sequon sequence
  REGION 78..80: note = MISC_FEATURE - sequon sequence
  REGION 82..84: note = MISC_FEATURE - sequon sequence
  REGION 95..97: note = MISC_FEATURE - sequon sequence
  REGION 101..103: note = MISC_FEATURE - sequon sequence
  REGION 119..123: note = MISC_FEATURE - sequon sequence
  REGION 145..149: note = MISC_FEATURE - sequon sequence
  SITE 200: note = MISC_FEATURE - Optional C-terminal purification tag
EELFKKHKIVAVLRANSVEEAIEKAVAVFAGGVHLIEITFTVPNATTVIKALSVLKEKGAIIGAGTVTSVEYANETVNATANFTVSPHLDEEISNFTKEKNVTYMPGVMTPTELVKAMWLNVTILKLFPGEVVGPQFVKAMKGPFHNATFVPTGGVNLDNVCEWFKAGVLAVGVGSALVKGTPDEVREKAKAFVEKIRGC

SEQ ID NO: 63 - protein, 200 aa, synthetic construct
  REGION 1..200: note = Synthetic peptide
  SITE 1: note = MISC_FEATURE - Optional N-terminal signal peptide
  REGION 68..72: note = MISC_FEATURE - sequon sequence
  REGION 78..80: note = MISC_FEATURE - sequon sequence
  REGION 82..84: note = MISC_FEATURE - sequon sequence
  REGION 95..97: note = MISC_FEATURE - sequon sequence
  REGION 101..103: note = MISC_FEATURE - sequon sequence
  SITE 200: note = MISC_FEATURE - Optional C-terminal purification tag
EELFKKHKIVAVLRANSVEEAIEKAVAVFAGGVHLIEITFTVPDADTVIKALSVLKEKGAIIGAGTVFSNDTCRKAVNATANFTVSPHLDEEISNFTKEKNVTYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGC

SEQ ID NO: 64 - protein, 200 aa, synthetic construct
  REGION 1..200: note = Synthetic peptide
  SITE 1: note = MISC_FEATURE - Optional N-terminal signal peptide
  REGION 69..71: note = MISC_FEATURE - sequon sequence
  REGION 78..80: note = MISC_FEATURE - sequon sequence
  REGION 82..84: note = MISC_FEATURE - sequon sequence
  REGION 95..97: note = MISC_FEATURE - sequon sequence
  REGION 101..103: note = MISC_FEATURE - sequon sequence
  SITE 200: note = MISC_FEATURE - Optional C-terminal purification tag
EELFKKHKIVAVLRANSVEEAIEKAVAVFAGGVHLIEITFTVPDADTVIKALSVLKEKGAIIGAGTVTYVNITRKAVNATANFTVSPHLDEEISNFTKEKNVTYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGC

SEQ ID NO: 65 - protein, 200 aa, synthetic construct
  REGION 1..200: note = Synthetic peptide
  SITE 1: note = MISC_FEATURE - Optional N-terminal signal peptide
  REGION 68..72: note = MISC_FEATURE - sequon sequence
  REGION 78..80: note = MISC_FEATURE - sequon sequence
  REGION 82..84: note = MISC_FEATURE - sequon sequence
  REGION 95..97: note = MISC_FEATURE - sequon sequence
  REGION 99..101: note = MISC_FEATURE - sequon sequence
  SITE 200: note = MISC_FEATURE - Optional C-terminal purification tag
EELFKKHKIVAVLRANSVEEAIEKAVAVFAGGVHLIEITFTVPDADTVIKALSVLKEKGAIIGAGTVFSNDTCRKAVNATANFTVSPHLDEEISNFTKNATVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGC

SEQ ID NO: 66 - protein, 200 aa, synthetic construct
  REGION 1..200: note = Synthetic peptide
  SITE 1: note = MISC_FEATURE - Optional N-terminal signal peptide
  REGION 68..72: note = MISC_FEATURE - sequon sequence
  REGION 78..80: note = MISC_FEATURE - sequon sequence
  REGION 82..84: note = MISC_FEATURE - sequon sequence
  REGION 95..97: note = MISC_FEATURE - sequon sequence
  REGION 99..101: note = MISC_FEATURE - sequon sequence
  SITE 200: note = MISC_FEATURE - Optional C-terminal purification tag
EELFKKHKIVAVLRANSVEEAIEKAVAVFAGGVHLIEITFTVPDADTVIKALSVLKEKGAIIGAGTVTYVNITRKAVNATANFTVSPHLDEEISNFTKNATVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGC

SEQ ID NO: 67 - protein, 200 aa, synthetic construct
  REGION 1..200: note = Synthetic peptide
  SITE 1: note = MISC_FEATURE - Optional N-terminal signal peptide
  REGION 44..46: note = MISC_FEATURE - sequon sequence
  REGION 72..76: note = MISC_FEATURE - sequon sequence
  REGION 95..97: note = MISC_FEATURE - sequon sequence
  REGION 145..149: note = MISC_FEATURE - sequon sequence
  SITE 200: note = MISC_FEATURE - Optional C-terminal purification tag
EELFKKHKIVAVLRANSVEEAIEKAVAVFAGGVHLIEITFTVPNATTVIKALSVLKEKGAIIGAGTVTSVEYANETVESGAEFIVSPHLDEEISNFTKEKGVFYMPGVMTPTELVKAMKLGHTILKLFPGEVVGPQFVKAMKGPFHNATFVPTGGVNLDNVCEWFKAGVLAVGVGSALVKGTPDEVREKAKAFVEKIRGC

SEQ ID NO: 68 - protein, 200 aa, synthetic construct
  REGION 1..200: note = Synthetic peptide
  SITE 1: note = MISC_FEATURE - Optional N-terminal signal peptide
  REGION 44..46: note = MISC_FEATURE - sequon sequence
  REGION 72..76: note = MISC_FEATURE - sequon sequence
  REGION 80..82: note = MISC_FEATURE - sequon sequence
  REGION 95..97: note = MISC_FEATURE - sequon sequence
  SITE 200: note = MISC_FEATURE - Optional C-terminal purification tag
EELFKKHKIVAVLRANSVEEAIEKAVAVFAGGVHLIEITFTVPNATTVIKALSVLKEKGAIIGAGTVTSVEYANETVESNATFIVSPHLDEEISNFTKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGC

SEQ ID NO: 69 - protein, 200 aa, synthetic construct
  REGION 1..200: note = Synthetic peptide
  SITE 1: note = MISC_FEATURE - Optional N-terminal signal peptide
  REGION 95..97: note = MISC_FEATURE - sequon sequence
  REGION 101..103: note = MISC_FEATURE - sequon sequence
  REGION 121..123: note = MISC_FEATURE - sequon sequence
  REGION 145..149: note = MISC_FEATURE - sequon sequence
  SITE 200: note = MISC_FEATURE - Optional C-terminal purification tag
EELFKKHKIVAVLRANSVEEAIEKAVAVFAGGVHLIEITFTVPDADTVIKALSVLKEKGAIIGAGTVTSVEQCRKAVESGAEFIVSPHLDEEISNFTKEKNVTYMPGVMTPTELVKAMKLNVTILKLFPGEVVGPQFVKAMKGPFHNATFVPTGGVNLDNVCEWFKAGVLAVGVGSALVKGTPDEVREKAKAFVEKIRGC

SEQ ID NO: 70 - protein, 200 aa, synthetic construct
  REGION 1..200: note = Synthetic peptide
  SITE 1: note = MISC_FEATURE - Optional N-terminal signal peptide
  REGION 68..72: note = MISC_FEATURE - sequon sequence
  REGION 80..82: note = MISC_FEATURE - sequon sequence
  REGION 95..97: note = MISC_FEATURE - sequon sequence
  REGION 101..103: note = MISC_FEATURE - sequon sequence
  SITE 200: note = MISC_FEATURE - Optional C-terminal purification tag
EELFKKHKIVAVLRANSVEEAIEKAVAVFAGGVHLIEITFTVPDADTVIKALSVLKEKGAIIGAGTVFSNETCRKAVESNATFIVSPHLDEEISNFTKEKNVTYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGC

SEQ ID NO: 71 - protein, 200 aa, synthetic construct
  REGION 1..200: note = Synthetic peptide
  SITE 1: note = MISC_FEATURE - Optional N-terminal signal peptide
  REGION 44..46: note = MISC_FEATURE - sequon sequence
  REGION 72..76: note = MISC_FEATURE - sequon sequence
  REGION 95..97: note = MISC_FEATURE - sequon sequence
  REGION 121..123: note = MISC_FEATURE - sequon sequence
  REGION 145..149: note = MISC_FEATURE - sequon sequence
  SITE 200: note = MISC_FEATURE - Optional C-terminal purification tag
EELFKKHKIVAVLRANSVEEAIEKAVAVFAGGVHLIEITFTVPNATTVIKALSVLKEKGAIIGAGTVTSVEYANETVESGAEFIVSPHLDEEISNFTKEKGVFYMPGVMTPTELVKAMKLNVTILKLFPGEVVGPQFVKAMKGPFHNATFVPTGGVNLDNVCEWFKAGVLAVGVGSALVKGTPDEVREKAKAFVEKIRGC

SEQ ID NO: 72 - protein, 200 aa, synthetic construct
  REGION 1..200: note = Synthetic peptide
  SITE 1: note = MISC_FEATURE - Optional N-terminal signal peptide
  REGION 44..46: note = MISC_FEATURE - sequon sequence
  REGION 72..76: note = MISC_FEATURE - sequon sequence
  REGION 95..97: note = MISC_FEATURE - sequon sequence
  REGION 119..123: note = MISC_FEATURE - sequon sequence
  REGION 145..149: note = MISC_FEATURE - sequon sequence
  SITE 200: note = MISC_FEATURE - Optional C-terminal purification tag
EELFKKHKIVAVLRANSVEEAIEKAVAVFAGGVHLIEITFTVPNATTVIKALSVLKEKGAIIGAGTVTSVEYANETVESGAEFIVSPHLDEEISNFTKEKGVFYMPGVMTPTELVKAMWLNHTILKLFPGEVVGPQFVKAMKGPFHNATFVPTGGVNLDNVCEWFKAGVLAVGVGSALVKGTPDEVREKAKAFVEKIRGC

SEQ ID NO: 73 - protein, 200 aa, synthetic construct
  REGION 1..200: note = Synthetic peptide
  SITE 1: note = MISC_FEATURE - Optional N-terminal signal peptide
  REGION 44..46: note = MISC_FEATURE - sequon sequence
  REGION 68..72: note = MISC_FEATURE - sequon sequence
  REGION 95..97: note = MISC_FEATURE - sequon sequence
  REGION 101..103: note = MISC_FEATURE - sequon sequence
  REGION 121..123: note = MISC_FEATURE - sequon sequence
  REGION 144..149: note = MISC_FEATURE - sequon sequence
  SITE 200: note = MISC_FEATURE - Optional C-terminal purification tag
EELFKKHKIVAVLRANSVEEAIEKAVAVFAGGVHLIEITFTVPNATTVIKALSVLKEKGAIIGAGTVFSNETCRKAVESGAEFIVSPHLDEEISNFTKEKNVTYMPGVMTPTELVKAMKLNVTILKLFPGEVVGPQFVKAMKGPFHNATFVPTGGVNLDNVCEWFKAGVLAVGVGSALVKGTPDEVREKAKAFVEKIRGC

SEQ ID NO: 74 - protein, 200 aa, synthetic construct
  REGION 1..200: note = Synthetic peptide
  SITE 1: note = MISC_FEATURE - Optional N-terminal signal peptide
  REGION 44..46: note = MISC_FEATURE - sequon sequence
  REGION 80..82: note = MISC_FEATURE - sequon sequence
  REGION 95..97: note = MISC_FEATURE - sequon sequence
  REGION 101..103: note = MISC_FEATURE - sequon sequence
  REGION 121..123: note = MISC_FEATURE - sequon sequence
  REGION 144..149: note = MISC_FEATURE - sequon sequence
  SITE 200: note = MISC_FEATURE - Optional C-terminal purification tag
EELFKKHKIVAVLRANSVEEAIEKAVAVFAGGVHLIEITFTVPNATTVIKALSVLKEKGAIIGAGTVTSVEQCRKAVESNATFIVSPHLDEEISNFTKEKNVTYMPGVMTPTELVKAMKLNVTILKLFPGEVVGPQFVKAMKGPFHNATFVPTGGVNLDNVCEWFKAGVLAVGVGSALVKGTPDEVREKAKAFVEKIRGC

SEQ ID NO: 75 - protein, 200 aa, synthetic construct
  REGION 1..200: note = Synthetic peptide
  SITE 1: note = MISC_FEATURE - Optional N-terminal signal peptide
  REGION 44..46: note = MISC_FEATURE - sequon sequence
  REGION 72..76: note = MISC_FEATURE - sequon sequence
  REGION 95..97: note = MISC_FEATURE - sequon sequence
  REGION 101..103: note = MISC_FEATURE - sequon sequence
  REGION 121..123: note = MISC_FEATURE - sequon sequence
  REGION 145..149: note = MISC_FEATURE - sequon sequence
  SITE 200: note = MISC_FEATURE - Optional C-terminal purification tag
EELFKKHKIVAVLRANSVEEAIEKAVAVFAGGVHLIEITFTVPNATTVIKALSVLKEKGAIIGAGTVTSVEYANETVESGAEFIVSPHLDEEISNFTKEKNVTYMPGVMTPTELVKAMKLNVTILKLFPGEVVGPQFVKAMKGPFHNATFVPTGGVNLDNVCEWFKAGVLAVGVGSALVKGTPDEVREKAKAFVEKIRGC

SEQ ID NO: 76 - protein, 200 aa, synthetic construct
  REGION 1..200: note = Synthetic peptide
  SITE 1: note = MISC_FEATURE - Optional N-terminal signal peptide
  REGION 44..46: note = MISC_FEATURE - sequon sequence
  REGION 68..72: note = MISC_FEATURE - sequon sequence
  REGION 80..82: note = MISC_FEATURE - sequon sequence
  REGION 95..97: note = MISC_FEATURE - sequon sequence
  REGION 101..103: note = MISC_FEATURE - sequon sequence
  REGION 121..123: note = MISC_FEATURE - sequon sequence
  REGION 145..149: note = MISC_FEATURE - sequon sequence
  SITE 200: note = MISC_FEATURE - Optional C-terminal purification tag
EELFKKHKIVAVLRANSVEEAIEKAVAVFAGGVHLIEITFTVPNATTVIKALSVLKEKGAIIGAGTVFSNETCRKAVESNATFIVSPHLDEEISNFTKEKNVTYMPGVMTPTELVKAMKLNVTILKLFPGEVVGPQFVKAMKGPFHNATFVPTGGVNLDNVCEWFKAGVLAVGVGSALVKGTPDEVREKAKAFVEKIRGC

SEQ ID NO: 77 - protein, 200 aa, synthetic construct
  REGION 1..200: note = Synthetic peptide
  SITE 1: note = MISC_FEATURE - Optional N-terminal signal peptide
  REGION 44..46: note = MISC_FEATURE - sequon sequence
  REGION 72..76: note = MISC_FEATURE - sequon sequence
  REGION 80..82: note = MISC_FEATURE - sequon sequence
  REGION 95..97: note = MISC_FEATURE - sequon sequence
  REGION 101..103: note = MISC_FEATURE - sequon sequence
  REGION 121..123: note = MISC_FEATURE - sequon sequence
  REGION 145..149: note = MISC_FEATURE - sequon sequence
  SITE 200: note = MISC_FEATURE - Optional C-terminal purification tag
EELFKKHKIVAVLRANSVEEAIEKAVAVFAGGVHLIEITFTVPNATTVIKALSVLKEKGAIIGAGTVTSVEYANETVESNATFIVSPHLDEEISNFTKEKNVTYMPGVMTPTELVKAMKLNVTILKLFPGEVVGPQFVKAMKGPFHNATFVPTGGVNLDNVCEWFKAGVLAVGVGSALVKGTPDEVREKAKAFVEKIRGC

SEQ ID NO: 78 - protein, 150 aa, synthetic construct
  REGION 1..150: note = Synthetic peptide
KYDGSKLRIGILHARGNAEIILELVLGALKRLQEFGVKRENIIIETVPGSFELPYGSKLFVEKQKRLGKPLDAIIPIGVLIRGSTAHFDYIADSTTHQLMKLNFELGIPVIFGVLTTESDEQAEERAGTKAGNHGEDWGAAAVEMATKFN

SEQ ID NO: 79 - protein, 119 aa, synthetic construct
  REGION 1..119: note = Synthetic peptide
EEAELAYLLGELAYKLGEYRIAIRAYRIALKRDPNNAEAWYNLGNAYYKQGRYREAIEYYQKALELDPNNAEAWYNLGNAYYERGEYEEAIEYYRKALRLDPNNADAMQNLLNAKMREE

SEQ ID NO: 80 - protein, 205 aa, synthetic construct
  REGION 1..205: note = Synthetic peptide
MKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTE

SEQ ID NO: 81 - protein, 5 aa, synthetic construct
  REGION 1..5: note = Synthetic peptide
FSNES

SEQ ID NO: 82 - protein, 5 aa, synthetic construct
  REGION 1..5: note = Synthetic peptide
FENES

SEQ ID NO: 83 - protein, 5 aa, synthetic construct
  REGION 1..5: note = Synthetic peptide
FFNHT

SEQ ID NO: 84 - protein, 5 aa, synthetic construct
  REGION 1..5: note = Synthetic peptide
FDNLS

SEQ ID NO: 85 - protein, 5 aa, synthetic construct
  REGION 1..5: note = Synthetic peptide
WHNNS

SEQ ID NO: 86 - protein, 5 aa, synthetic construct
  REGION 1..5: note = Synthetic peptide
FINYS

SEQ ID NO: 87 - protein, 5 aa, synthetic construct
  REGION 1..5: note = Synthetic peptide
FLNAT

SEQ ID NO: 88 - protein, 5 aa, synthetic construct
  REGION 1..5: note = Synthetic peptide
FLNAS

SEQ ID NO: 89 - protein, 5 aa, synthetic construct
  REGION 1..5: note = Synthetic peptide
WVNNS

SEQ ID NO: 90 - protein, 5 aa, synthetic construct
  REGION 1..5: note = Synthetic peptide
YLNKS

SEQ ID NO: 91 - protein, 5 aa, synthetic construct
  REGION 1..5: note = Synthetic peptide
FSNET

SEQ ID NO: 92 - protein, 5 aa, synthetic construct
  REGION 1..5: note = Synthetic peptide
YVNVT

SEQ ID NO: 93 - protein, 5 aa, synthetic construct
  REGION 1..5: note = Synthetic peptide
YANRS

SEQ ID NO: 94 - protein, 5 aa, synthetic construct
  REGION 1..5: note = Synthetic peptide
WANAS

SEQ ID NO: 95 - protein, 5 aa, synthetic construct
  REGION 1..5: note = Synthetic peptide
WANFT

SEQ ID NO: 96 - protein, 5 aa, synthetic construct
  REGION 1..5: note = Synthetic peptide
WLNHT

SEQ ID NO: 97 - protein, 30 aa, synthetic construct
  REGION 1..30: note = Synthetic peptide
MDSKGSSQKGSRLLLLLVVSNLLLPQGVLA

SEQ ID NO: 98 - protein, 18 aa, synthetic construct
  REGION 1..18: note = Synthetic peptide
LEEQKLISEEDLHHHHHH

SEQ ID NO: 99 - protein, 204 aa, synthetic construct
  REGION 1..204: note = Synthetic peptide
RFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKST

SEQ ID NO: 100 - protein, 208 aa, synthetic construct
  REGION 1..208: note = Synthetic peptide
ETGTRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKST

SEQ ID NO: 101 - protein, 1198 aa, synthetic construct
  REGION 1..1198: note = Synthetic peptide
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPSGAGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIK

SEQ ID NO: 102 - protein, 1202 aa, synthetic construct
  REGION 1..1202: note = Synthetic peptide
  REGION 1..4: note = MISC_FEATURE - Optional residues
ETGTQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPSGAGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIK

SEQ ID NO: 103 - protein, 1230 aa, synthetic construct
  REGION 1..1230: note = Synthetic peptide
  REGION 1..32: note = MISC_FEATURE - Optional residues
MGILPSPGMPALLSLVSLLSVLLMGCVAETGTQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPSGAGSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIK

SEQ ID NO: 104 - protein, 1208 aa, synthetic construct
  REGION 1..1208: note = Synthetic peptide
  REGION 1..15: note = MISC_FEATURE - Optional residues
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQ

SEQ ID NO: 105 - protein, 1160 aa, synthetic construct
  REGION 1..1160: note = Synthetic peptide
  REGION 1..15: note = MISC_FEATURE - Optional residues
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHT

SEQ ID NO: 106 - protein, 1195 aa, synthetic construct
  REGION 1..1195: note = Synthetic peptide
  REGION 1..2: note = MISC_FEATURE - Optional residues
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQ

SEQ ID NO: 107 - protein, 1147 aa, synthetic construct
  REGION 1..1147: note = Synthetic peptide
  REGION 1..2: note = MISC_FEATURE - Optional residues
QCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHT

SEQ ID NO: 108 - protein, 1193 aa, synthetic construct
  REGION 1..1193: note = Synthetic peptide
VNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQ

SEQ ID NO: 109 - protein, 1145 aa, synthetic construct
  REGION 1..1145: note = Synthetic peptide
VNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHT

SEQ ID NO: 110 - protein, 1199 aa, synthetic construct
  REGION 1..1199: note = Synthetic peptide
ETCTQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQ

SEQ ID NO: 111 - protein, 1151 aa, synthetic construct
  REGION 1..1151: note = Synthetic peptide
ETCTQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHT

SEQ ID NO: 112 - protein, 869 aa, synthetic construct
  REGION 1..869: note = Synthetic peptide
MRVKGIKKNYQHWWRGGIMLLGMLMICSSAEKLWVTVYYGVPVWKEATTTLFCASDAKAQNPEMHNIWATHACVPTDPNPQEVILKNLTEEFNMWKNNMVEQMHEDIISLWDQSLKPCVKLTPLCVTLNCTNAESLNCTATNGTNNCSASTKPMEEMKNCSFNITTSVQDKKQQEYALFYKLDIIPIDNNENDLNNTNYTSYRLISCNTSVITQACPKITFEPIPIHYCAPAGFAILKCKDKRFNGTGPCKNVSTVQCTHGIRPVVSTQLLLNGSLAEEGVVLRSENFTDNAKNIIVQLKDPVNITCTRPNNNTRKSITIGPGRAFYATGQVIGDIRKAHCDLNGTEWDNALKQIVEELRKQYGNNITIFNSSSGGDPEIVMHSFNCGGEFFYCNTAQLFNSTWLFNSTWNSTERLGNDTERTNDTITLPCKIKQVINMWQTVGKAMYAPPIRGLIRCSSNITGLILTRDGSGNTTGNETFRPGGGNMKDNWRSELYKYKVVKIEPLGVAPTRAKRRVVQREKRAAGLGALFLGFLGMAGSTMGAASLTLTVQARQLLSGIVQQQNNLLRAIEAQQHLLQLTVWGIKQLQARVLAVERYLRDQQLLGIWGCSGKLICTTTVPWNASWSNKSLDNIWENMTWMQWEKEIDNYTDVIYKLLEESQNQQEKNEQELLELDKWASLWNWFDITRWLWYIKIFIMIVGGLVGLRIVFAVLSIVNRVRQGYSPLSFQTLFPAPRGPDRPEGTEEGGGERGRDSSDRSAHGFLALIWGDLWSLCLFSYRRLRDLLLIAARIVELLGRRGWEVLKYWWSLLQYWSQELKKSAVSLLNATAIAVAEGTDRIIEIVQRAGRAIIHIPRRIRQGAERALL

SEQ ID NO: 113 - protein, 492 aa, synthetic construct
  REGION 1..492: note = Synthetic peptide
LWVTVYYGVPVWKEATTTLFCASDAKAQNPEMHNIWATHACVPTDPNPQEVILKNLTEEFNMWKNNMVEQMHEDIISLWDQSLKPCVKLTPLCVTLNCTNAESLNCTATNGTNNCSASTKPMEEMKNCSFNITTSVQDKKQQEYALFYKLDIIPIDNNENDLNNTNYTSYRLISCNTSVITQACPKITFEPIPIHYCAPAGFAILKCKDKRFNGTGPCKNVSTVQCTHGIRPVVSTQLLLNGSLAEEGVVLRSENFTDNAKNIIVQLKDPVNITCTRPNNNTRKSITIGPGRAFYATGQVIGDIRKAHCDLNGTEWDNALKQIVEELRKQYGNNITIFNSSSGGDPEIVMHSFNCGGEFFYCNTAQLFNSTWLFNSTWNSTERLGNDTERTNDTITLPCKIKQVINMWQTVGKAMYAPPIRGLIRCSSNITGLILTRDGSGNTTGNETFRPGGGNMKDNWRSELYKYKVVKIEPLGVAPTRAKRRVVQREKR

SEQ ID NO: 114 - protein, 191 aa, synthetic construct
  REGION 1..191: note = Synthetic peptide
MGAASLTLTVQARQLLSGIVQQQNNLLRAIEAQQHLLQLTVWGIKQLQARVLAVERYLRDQQLLGIWGCSGKLICTTTVPWNASWSNKSLDNIWENMTWMQWEKEIDNYTDVIYKLLEESQNQQEKNEQELLELDKWASLWNWFDITRWLWYIKIFIMIVGGLVGLRIVFAVLSIVNRVRQGYSPLSFQTL

SEQ ID NO: 115 - protein, 22 aa, synthetic construct
  REGION 1..22: note = Synthetic peptide
ELDKWASLWNWFDITRWLWYIK

SEQ ID NO: 116 - protein, 574 aa, synthetic construct
  REGION 1..574: note = Synthetic peptide
MELPILKTNAITTILAAVTLCFASSQNITEEFYQSTCSAVSKGYLSALRTGWYTSVITIELSNIKENKCNGTDAKVKLIKQELDKYKNAVTELQLLMQSTPAANNRARRELPRFMNYTLNNTKNNNVTLSKKRKRRFLGFLLGVGSAIASGIAVSKVLHLEGEVNKIKNALLSTNKAVVSLSNGVSVLTSKVLDLKNYIDKQLLPIVNKQSCSISNIETVIEFQQKNNRLLEITREFSVNAGVTTPVSTYMLTNSELLSLINDMPITNDQKKLMSNNVQIVRQQSYSIMSIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNIDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLHNVNVGKSTTNIMITTIIIVIIVILLLLIAVGLFLYCKARSTPVTLSKDQLSGINNIAFSN

SEQ ID NO: 117 - protein, 566 aa, synthetic construct
  REGION 1..566: note = Synthetic peptide
MKAILVVLLYTFATANADTLCIGYHANNSTDTVDTVLEKNVTVTHSVNLLEDKHNGKLCKLRGVAPLHLGKCNIAGWILGNPECESLSTASSWSYIVETPSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADAYVFVGSSRYSKKFKPEIAIRPKVRDQEGRMNYYWTLVEPGDKITFEATGNLVVPRYAFAMERNAGSGIIISDTPVHDCNTTCQTPKGAINTSLPFQNIHPITIGKCPKYVKSTKLRLATGLRNIPSIQSRGLFGAIAGFIEGGWTGMVDGWYGYHHQNEQGSGYAADLKSTQNAIDEITNKVNSVIEKMNTQFTAVGKEFNHLEKRIENLNKKVDDGFLDIWTYNAELLVLLENERTLDYHDSNVKNLYEKVRSQLKNNAKEIGNGCFEFYHKCDNTCMESVKNGTYDYPKYSEEAKLNREEIDGVKLESTRIYQILAIYSTVASSLVLVVSLGAISFWMCSNGSLQCRICI

SEQ ID NO: 118 - protein, 584 aa, synthetic construct
  REGION 1..584: note = Synthetic peptide
MKAIIVLLMVVTSNADRICTGITSSNSPHVVKTATQGEVNVTGVIPLTTTPTKSYFANLKGTKTRGKLCPDCLNCTDLDVALGRPMCVGTTPSAKASILHEVRPVTSGCFPIMHDRTKIRQLANLLRGYENIRLSTQNVIDAEKAPGGPYRLGTSGSCPNATSKSGFFATMAWAVPKDNNKNATNPLTVEVPYICAEGEDQITVWGFHSDNKTQMKNLYGDSNPQKFTSSANGVTTHYVSQIGGFPDQTEDGGLPQSGRIVVDYMMQKPGKTGTIVYQRGVLLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEFSLPTFDSLNITAASLNDDGLDNHTILLYYSTAASSLAVTLMLAIFIVYMVSRDNVSCSICL

SEQ ID NO: 119 - protein, 907 aa, synthetic construct
  REGION 1..907: note = Synthetic peptide
MEAALLVCQYTIQSLIHLTGEDPGFFNVEIPEFPFYPTCNVCTADVNVTINFDVGGKKHQLDLDFGQLTPHTKAVYQPRGAFGGSENATNLFLLELLGAGELALTMRSKKLPINVTTGEEQQVSLESVDVYFQDVFGTMWCHHAEMQNPVYLIPETVPYIKWDNCNSTNITAVVRAQGLDVTLPLSLPTSAQDSNFSVKTEMLGNEIDIECIMEDGEISQVLPGDNKFNITCSGYESHVPSGGILTSTSPVATPIPGTGYAYSLRLTPRPVSRFLGNNSILYVFYSGNGPKASGGDYCIQSNIVFSDEIPASQDMPTNTTDITYVGDNATYSVPMVTSEDANSPNVTVTAFWAWPNNTETDFKCKWTLTSGTPSGCENISGAFASNRTFDITVSGLGTAPKTLIITRTATNATTTTHKVIFSKAPESTTTSPTLNTTGFADPNTTTGLPSSTHVPTNLTAPASTGPTVSTADVTSPTPAGTTSGASPVTPSPSPWDNGTESKAPDMTSSTSPVTTPTPNATSPTPAVTTPTPNATSPTPAVTTPTPNATSPTLGKTSPTSAVTTPTPNATSPTLGKTSPTSAVTTPTPNATSPTLGKTSPTSAVTTPTPNATGPTVGETSPQANATNHTLGGTSPTPVVTSQPKNATSAVTTGQHNITSSSTSSMSLRPSSNPETLSPSTSDNSTSHMPLLTSAHPTGGENITQVTPASISTHHVSTSSPAPRPGTTSQASGPGNSSTSTKPGEVNVTKGTPPQNATSPQAPSGQKTAVPTVTSTGGKANSTTGGKHTTGHGARTSTEPTTDYGGDSTTPRPRYNATTYLPPSTSSKLRPRWTFTSPPVTTAQATVPVPPTSQPRFSNLSMLVLQWASLAVLTLLLLLVMADCAFRRNLSTSHTYTTPPYDDAETYV

SEQ ID NO: 120 - protein, 906 aa, synthetic construct
  REGION 1..906: note = Synthetic peptide
MESRIWCLVVCVNLCIVCLGAAVSSSSTSHATSSTHNGSHTSRTTSAQTRSVYSQHVTSSEAVSHRANETIYNTTLKYGDVVGVNTTKYPYRVCSMAQGTDLIRFERNIICTSMKPINEDLDEGIMVVYKRNIVAHTFKVRVYQKVLTFRRSYAYIYTTYLLGSNTEYVAPPMWEIHHINKFAQCYSSYSRVIGGTVFVAYHRDSYENKTMQLIPDDYSNTHSTRYVTVKDQWHSRGSTWLYRETCNLNCMLTITTARSKYPYHFFATSTGDVVYISPFYNGTNRNASYFGENADKFFIFPNYTIVSDFGRPNAAPETHRLVAFLERADSVISWDIQDEKNVTCQLTFWEASERTIRSEAEDSYHFSSAKMTATFLSKKQEVNMSDSALDCVRDEAINKLQQIFNTSYNQTYEKYGNVSVFETSGGLVVFWQGIKQKSLVELERLANRSSLNITHRTRRSTSDNNTTHLSSMESVHNLVYAQLQFTYDTLRGYINRALAQIAEAWCVDQRRTLEVFKELSKINPSAILSAIYNKPIAARFMGDVLGLASCVTINQTSVKVLRDMNVKESPGRCYSRPVVIFNFANSSYVQYGQLGEDNEILLGNHRTEECQLPSLKIFIAGNSAYEYVDYLFKRMIDLSSISTVDSMIALDIDPLENTDFRVLELYSQKELRSSNVFDLEEIMREFNSYKQRVKYVEDKVVDPLPPYLKGLDDLMSGLGAAGKAVGVAIGAVGGAVASVVEGVATFLKNPFGAFTIILVAIAVVIITYLIYTRQRRLCTQPLQNLFPYLVSADGTTVTSGSTKDTSLQAPPSYEESVYNSGRKGPGPPSSDASTAAPPYTNEQAYQMLLALARLDAEQRAQQNGTDSLDGQTGTQDKGQKPNLLDRLRHRKNGYRHLKDSDEEENV

SEQ ID NO: 121 - protein, 171 aa, synthetic construct
  REGION 1..171: note = Synthetic peptide
MSPKDLTPFLTTLWLLLGHSRVPRVRAEECCEFINVNHPPERCYDFKMCNRFTVALRCPDGEVCYSPEKTAEIRGIVTTMTHSLTRQVVHNKLTSCNYNPLYLEADGRIRCGKVNDKAQYLLGAAGSVPYRWINLEYDKITRIVGLDQYLESVKKHKRLDVCRAKMGYMLQ

SEQ ID NO: 122 - protein, 214 aa, synthetic construct
  REGION 1..214: note = Synthetic peptide
MLRLLLRHHFHCLLLCAVWATPCLASPWSTLTANQNPSPPWSKLTYSKPHDAATFYCPFLYPSPPRSPLQFSGFQRVSTGPECRNETLYLLYNREGQTLVERSSTWVKKVIWYLSGRNQTILQRMPRTASKPSDGNVQISVEDAKIFGAHMVPKQTKLLRFVVNDGTRYQMCVMKLESWAHVFRDYSVSFQVRLTFTEANNQTYTFCTHPNLIV

SEQ ID NO: 123 - protein, 129 aa, synthetic construct
  REGION 1..129: note = Synthetic peptide
MRLCRVWLSVCLCAVVLGQCQRETAEKNDYYRVPHYWDACSRALPDQTRYKYVEQLVDLTLNYHYDASHGLDNFDVLKRINVTEVSLLISDFRRQNRRGGTNKRTTFNAAGSLAPHARSLEFSVRLFAN

SEQ ID NO: 124 - protein, 743 aa, synthetic construct
  REGION 1..743: note = Synthetic peptide
MRPGLPPYLTVFTVYLLSHLPSQRYGADAASEALDPHAFHLLLNTYGRPIRFLRENTTQCTYNSSLRNSTVVRENAISFNFFQSYNQYYVFHMPRCLFAGPLAEQFLNQVDLTETLERYQQRLNTYALVSKDLASYRSFSQQLKAQDSLGQQPTTVPPPIDLSIPHVWMPPQTTPHDWKGSHTTSGLHRPHFNQTCILFDGHDLLFSTVTPCLHQGFYLMDELRYVKITLTEDFFVVTVSIDDDTPMLLIFGHLPRVLFKAPYQRDNFILRQTEKHELLVLVKKAQLNRHSYLKDSDFLDAALDFNYLDLSALLRNSFHRYAVDVLKSGRCQMLDRRTVEMAFAYALALFAAARQEEAGTEISIPRALDRQAALLQIQEFMITCLSQTPPRTTLLLYPTAVDLAKRALWTPDQITDITSLVRLVYILSKQNQQHLIPQWALRQIADFALQLHKTHLASFLSAFARQELYLMGSLVHSMLVHTTERREIFIVETGLCSLAELSHFTQLLAHPHHEYLSDLYTPCSSSGRRDHSLERLTRLFPDATVPATVPAALSILSTMQPSTLETFPDLFCLPLGESFSALTVSEHVSYVVTNQYLIKGISYPVSTTVVGQSLIITQTDSQTKCELTRNMHTTHSITAALNISLENCAFCQSALLEYDDTQGVINIMYMHDSDDVLFALDPYNEVVVSSPRTHYLMLLKNGTVLEVTDVVVDATDSRLLMMSVYALSAIIGIYLLYRMLKTC

SEQ ID NO: 125 - protein, 278 aa, synthetic construct
  REGION 1..278: note = Synthetic peptide
MCRRPDCGFSFSPGPVVLLWCCLLLPIVSSVAVSVAPTAAEKVPAECPELTRRCLLGEVFQGDKYESWLRPLVNVTRRDGPLSQLIRYRPVTPEAANSVLLDDAFLDTLALLYNNPDQLRALLTLLSSDTAPRWMTVMRGYSECGDGSPAVYTCVDDLCRGYDLTRLSYGRSIFTEHVLGFELVPPSLFNVVVAIRNEATRTNRAVRLPVSTAAAPEGITLFYGLYNAVKEFCLRHQLDPPLLRHLDKYYAGLPPELKQTRVNLPAHSRYGPQAVDAR

SEQ ID NO: 126 - protein, 272 aa, synthetic construct
  REGION 1..272: note = Synthetic peptide
MKKYLLGIGLILALIACKQNVSSLDEKNSVSVDVPGGMKVLVSKEKNKDGKYDLMATVDNVDLKGTSDKNNGSGILEGVKADKSKVKLTVADDLSKTTLEVLKEDGTVVSRKVTSKDKSTTEAKFNEKGELSEKTMTRANGTTLEYSQMTNEDNAAKAVETLKNGIKFEGNLASGKTAVEIKEGTVTLKREIDKNGKVTVSLNDTASGSKKTASWQESTSTLTISANSKKTKDLVFLTNGTITVQNYDSAGTKLEGSAAEIKKLDELKNALR

SEQ ID NO: 127 - protein, 269 aa, synthetic construct
  REGION 1..269: note = Synthetic peptide
MRCTRAIRQTARTGWLTWLAILAVTAPVTSPAWADDPPATVYRYDSRPPEDVFQNGFTAWGNNDNVLDHLTGRSCQVGSSNSAFVSTSSSRRYTEVYLEHRMQEAVEAERAGRGTGHFIGYIYEVRADNNFYGAASSYFEYVDTYGDNAGRILAGALATYQSEYLAHRRIPPENIRRVTRVYHNGITGETTTTEYSNARYVSQQTRANPNPYTSRRSVASIVGTLVRMAPVIGACMARQAESSEAMAAWSERAGEAMVLVYYESIAYSF

SEQ ID NO: 128 - protein, 495 aa, synthetic construct
  REGION 1..495: note = Synthetic peptide
MRCVGIGNRDFVEGLSGATWVDVVLEHGSCVTTMAKDKPTLDIELLKTEVTNPAVLRKLCIEAKISNTTTDSRCPTQGEATLVEEQDTNFVCRRTFVDRGWGNGCGLFGKGSLITCAKFKCVTKLEGKIVQYENLKYSVIVTVHTGDQHQVGNETTEHGTTATITPQAPTSEIQLTDYGALTLDCSPRTGLDFNEMVLLTMEKKSWLVHKQWFLDLPLPWTSGASTSQETWNRQDLLVTFKTAHAKKQEVVVLGSQEGAMHTALTGATEIQTSGTTTIFAGHLKCRLKMDKLTLKGMSYVMCTGSFKLEKEVAETQHGTVLVQVKYEGTDAPCKIPFSSQDEKGVTQNGRLITANPIVTDKEKPVNIEAEPPFGESYIVVGAGEKALKLSWFKKGSSIGKMFEATARGARRMAILGDTAWDFGSIGGVFTSVGKLIHQIFGTAYGVLFSGVSWTMKIGIGILLTWLGLNSRSTSLSMTCIAVGMVTLYLGVMVQA

SEQ ID NO: 129 - protein, 1255 aa, synthetic construct
  REGION 1..1255: note = Synthetic peptide
MFIFLLFLTLTSGSDLDRCTTFDDVQAPNYTQHTSSMRGVYYPDEIFRSDTLYLTQDLFLPFYSNVTGFHTINHTFGNPVIPFKDGIYFAATEKSNVVRGWVFGSTMNNKSQSVIIINNSTNVVIRACNFELCDNPFFAVSKPMGTQTHTMIFDNAFNCTFEYISDAFSLDVSEKSGNFKHLREFVFKNKDGFLYVYKGYQPIDVVRDLPSGFNTLKPIFKLPLGINITNFRAILTAFSPAQDIWGTSAAAYFVGYLKPTTFMLKYDENGTITDAVDCSQNPLAELKCSVKSFEIDKGIYQTSNFRVVPSGDVVRFPNITNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPKLSTDLIKNQCVNFNFNGLTGTGVLTPSSKRFQPFQQFGRDVSDFTDSVRDPKTSEILDISPCSFGGVSVITPGTNASSEVAVLYQDVNCTDVSTAIHADQLTPAWRIYSTGNNVFQTQAGCLIGAEHVDTSYECDIPIGAGICASYHTVSLLRSTSQKSIVAYTMSLGADSSIAYSNNTIAIPTNFSISITTEVMPVSMAKTSVDCNMYICGDSTECANLLLQYGSFCTQLNRALSGIAAEQDRNTREVFAQVKQMYKTPTLKYFGGFNFSQILPDPLKPTKRSFIEDLLFNKVTLADAGFMKQYGECLGDINARDLICAQKFNGLTVLPPLLTDDMIAAYTAALVSGTATAGWTFGAGAALQIPFAMQMAYRFNGIGVTQNVLYENQKQIANQFNKAISQIQESLTTTSTALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQAAPHGVVFLHVTYVPSQERNFTTAPAICHEGKAYFPREGVFVFNGTSWFITQRNFFSPQIITTDNTFVSGNCDVVIGIINNTVYDPLQPELDSFKEELDKYFKNHTSPDVDLGDISGINASVVNIQKEIDRLNEVAKNLNESLIDLQELGKYEQYIKWPWYVWLGFIAGLIAIVMVTILLCCMTSCCSCLKGACSCGSCCKFDEDDSEPVLKGVKLHYT

SEQ ID NO: 130 - protein, 1353 aa, synthetic construct
  REGION 1..1353: note = Synthetic peptide
MIHSVFLLMFLLTPTESYVDVGPDSIKSACIEVDIQQTFFDKTWPRPIDVSKADGIIYPQGRTYSNITITYQGLFPYQGDHGDMYVYSAGHATGTTPQKLFVANYSQDVKQFANGFVVRIGAAANSTGTVIISPSTSATIRKIYPAFMLGSSVGNFSDGKMGRFFNHTLVLLPDGCGTLLRAFYCILEPRSGNHCPAGNSYTSFATYHTPATDCSDGNYNRNASLNSFKEYFNLRNCTFMYTYNITEDEILEWFGITQTAQGVHLFSSRYVDLYGGNMFQFATLPVYDTIKYYSIIPHSIRSIQSDRKAWAAFYVYKLQPLTFLLDFSVDGYIRRAIDCGFNDLSQLHCSYESFDVESGVYSVSSFEAKPSGSVVEQAEGVECDFSPLLSGTPPQVYNFKRLVFTNCNYNLTKLLSLFSVNDFTCSQISPAAIASNCYSSLILDYFSYPLSMKSDLSVSSAGPISQFNYKQSFSNPTCLILATVPHNLTTITKPLKYSYINKCSRLLSDDRTEVPQLVNANQYSPCVSIVPSTVWEDGDYYRKQLSPLEGGGWLVASGSTVAMTEQLQMGFGITVQYGTDTNSVCPKLEFANDTKIASQLGNCVEYSLYGVSGRGVFQNCTAVGVRQQRFVYDAYQNLVGYYSDDGNYYCLRACVSVPVSVIYDKETKTHATLFGSVACEHISSTMSQYSRSTRSMLKRRDSTYGPLQTPVGCVLGLVNSSLFVEDCKLPLGQSLCALPDTPSTLTPRSVRSVPGEMRLASIAFNHPIQVDQLNSSYFKLSIPTNFSFGVTQEYIQTTIQKVTVDCKQYVCNGFQKCEQLLREYGQFCSKINQALHGANLRQDDSVRNLFASVKSSQSSPIIPGFGGDFNLTLLEPVSISTGSRSARSAIEDLLFDKVTIADPGYMQGYDDCMQQGPASARDLICAQYVAGYKVLPPLMDVNMEAAYTSSLLGSIAGVGWTAGLSSFAAIPFAQSIFYRLNGVGITQQVLSENQKLIANKFNQALGAMQTGFTTTNEAFHKVQDAVNNNAQALSKLASELSNTFGAISASIGDIIQRLDVLEQDAQIDRLINGRLTTLNAFVAQQLVRSESAALSAQLAKDKVNECVKAQSKRSGFCGQGTHIVSFVVNAPNGLYFMHVGYYPSNHIEVVSAYGLCDAANPTNCIAPVNGYFIKTNNTRIVDEWSYTGSSFYAPEPITSLNTKYVAPQVTYQNISTNLPPPLLGNSTGIDFQDELDEFFKNVSTSIPNFGSLTQINTTLLDLTYEMLSLQQVVKALNESYIDLKELGNYTYYNKWPWYIWLGFIAGLVALALCVFFILCCTGCGTNCMGKLKCNRCCDRYEEYDLEPHKVHVH

SEQ ID NO: 131 - protein, 676 aa, synthetic construct
  REGION 1..676: note = Synthetic peptide
MGVTGILQLPRDRFKRTSFFLWVIILFQRTFSIPLGVIHNSTLQVSDVDKLVCRDKLSSTNQLRSVGLNLEGNGVATDVPSATKRWGFRSGVPPKVVNYEAGEWAENCYNLEIKKPDGSECLPAAPDGIRGFPRCRYVHKVSGTGPCAGDFAFHKEGAFFLYDRLASTVIYRGTTFAEGVVAFLILPQAKKDFFSSHPLREPVNATEDPSSGYYSTTIRYQATGFGTNETEYLFEVDNLTYVQLESRFTPQFLLQLNETIYTSGKRSNTTGKLIWKVNPEIDTTIGEWAFWETKKNLTRKIRSEELSFTVVSNGAKNISGQSPARTSSDPGTNTTTEDHKIMASENSSAMVQVHSQGREAAVSHLTTLATISTSPQSLTTKPGPDNSTHNTPVYKLDISEATQVEQHHRRTDNDSTASDTPSATTAAGPPKAENTNTSKSTDFLDPATTTSPQNHSETAGNNNTHHQDTGEESASSGKLGLITNTIAGVAGLITGGRRTRREAIVNAQPKCNPNLHYWTTQDEGAAIGLAWIPYFGPAAEGIYIEGLMHNQDGLICGLRQLANETTQALQLFLRATTELRTFSILNRKAIDFLLQRWGGTCHILGPDCCIEPHDWTKNITDKIDQIIHDFVDKTLPDQGDNDNWWTGWRQWIPAGIGVTGVIIAVIALFCICKFVF

SEQ ID NO: 132 - protein, 681 aa, synthetic construct
  REGION 1..681: note = Synthetic peptide
MKTTCFLISLILIQGTKNLPILEIASNNQPQNVDSVCSGTLQKTEDVHLMGFTLSGQKVADSPLEASKRWAFRTGVPPKNVEYTEGEEAKTCYNISVTDPSGKSLLLDPPTNIRDYPKCKTIHHIQGQNPHAQGIALHLWGAFFLYDRIASTTMYRGKVFTEGNIAAMIVNKTVHKMIFSRQGQGYRHMNLTSTNKYWTSSNGTQTNDTGCFGALQEYNSTKNQTCAPSKIPPPLPTARPEIKLTSTPTDATKLNTTDPSSDDEDLATSGSGSGEREPHTTSDAVTKQGLSSTMPPTPSPQPSTPQQGGNNTNHSQDAVTELDKNNTTAQPSMPPHNTTTISTNNTSKHNFSTLSAPLQNTTNDNTQSTITENEQTSAPSITTLPPTGNPTTAKSTSSKKGPATTAPNTTNEHFTSPPPTPSSTAQHLVYFRRKRSILWREGDMFPFLDGLINAPIDFDPVPNTKTIFDESSSSGASAEEDQHASPNISLTLSYFPNINENTAYSGENENDCDAELRIWSVQEDDLAAGLSWIPFFGPGIEGLYTAVLIKNQNNLVCRLRRLANQTAKSLELLLRVTTEERTFSLINRHAIDFLLTRWGGTCKVLGPDCCIGIEDLSKNISEQIDQIKKDEQKEGTGWGLGGKWWTSDWGVLTNLGILLLLSIAVLIALSCICRIFTKYIG

SEQ ID NO: 133 - protein, 630 aa, synthetic construct
  REGION 1..630: note = Synthetic peptide
LRNVYDMKIECPHTVSFGENSVIGYVELPPVPLADTAQMVPESSCNMDNHQSLNTITKYTQVSWRGKADQSQSSQNSFETVSTEVDLKGTCVLKHKMVEESYRSRKSVTCYDLSCNSTYCKPTLYMIVPIHACNMMKSCLIALGPYRVQVVYERSYCMTGVLIEGKCFVPDQSVVSIIKHGIFDIASVHIVCFFVAVKGNTYKIFEQVKKSFESTCNDTENKVQGYYICIVGGNSAPIYVPTLDDFRSMEAFTGIFRSPHGEDHDLAGEEIASYSIVGPANAKVPHSASSDTLSLIAYSGIPSYSSLSILTSSTEAKHVFSPGLFPKLNHTNCDKSAIPLIWTGMIDLPGYYEAVHPCTVFCVLSGPGASCEAFSEGGIFNITSPMCLVSKQNRFRLTEQQVNFVCQRVDMDIVVYCNGQRKVILTKTLVIGQCIYTITSLFSLLPGVAHSIAVELCVPGFHGWATAALLVTFCFGWVLIPAITFIILTVLKFIANIFHTSNQENRLKSVLRKIKEEFEKTKGSMVCDVCKYECETYKELKAHGVSCPQSQCPYCFTHCEPTEAAFQAHYKVCQVTHRFRDDLKKTVTPQNFTPGCYRTLNLFRYKSRCYIFTMWIFLLVLESILWAASA

SEQ ID NO: 134 - protein, 487 aa, synthetic construct
  REGION 1..487: note = Synthetic peptide
SETPLTPVWNDNAHGVGSVPMHTDLELDFSLTSSSKYTYRRKLTNPLEEAQSIDLHIEIEEQTIGVDVHALGHWFDGRLNLKTSFHCYGACTKYEYPWHTAKCHYERDYQYETSWGCNPSDCPGVGTGCTACGLYLDQLKPVGSAYKIITIRYSRRVCVQFGEENLCKIIDMNDCFVSRHVKVCIIGTVSKFSQGDTLLFFGPLEGGGLIFKHWCTSTCQFGDPGDIMSPRDKGFLCPEFPGSFRKKCNFATTPICEYDGNMVSGYKKVMATIDSFQSFNTSTMHFTDERIEWKDPDGMLRDHINILVTKDIDFDNLGENPCKIGLQTSSIEGAWGSGVGFTLTCLVSLTECPTFLTSIKACDKAICYGAESVTLTRGQNTVKVSGKGGHSGSTFRCCHGEDCSQIGLHAAAPHLDKVNGISEIENSKVYDDGAPQCGIKCWFVKSGEWISGIFSGNWIVLIVLCVFLLFSLVLLSILCPVRKHKKS

SEQ ID NO: 135 - protein, 226 aa, synthetic construct
  REGION 1..226: note = Synthetic peptide
MENITSGFLGPLLVLQAGFFLLTKILTIPQSLNSWWTSLSFLGGNTVCLGQNSQSPTSNHSPTSCPPTCPGYRWMCLRRFIIFLFILLLCLIFLLVLLDYQGMLPVCPLIPGSSTTSTGPCRTCKTPAQGTSMYPSCCCTKPSDGNCTCIPIPSSWAFGKFLWEWASARFSWLSLIVPFVQWFVGLSPTVWLSVIWMMWYWGPSLYSILSPFLPLLPIFFCLWVYI

SEQ ID NO: 136 - protein, 617 aa, synthetic construct
  REGION 1..617: note = Synthetic peptide
MSPQRDRINAFYKDNPHPKGSRIVINREHLMIDRPYVLLAVLFVMFLSLIGLLAIAGIRLHRAAIYTAEIHKSLSTNLDVTNSIEHQVKDVLTPLFKIIGDEVGLRTPQRFTDLVKFISDKIKFLNPDREYDFRDLTWCINPPERIKLDYDQYCADVAAEELMNALVNSTLLETRTTNQFLAVSKGNCSGPTTIRGQFSNMSLSLLDLYLGRGYNVSSIVTMTSQGMYGGTYLVEKPNLSSKRSELSQLSMYRVFEVGVIRNPGLGAPVFHMTNYLEQPVSNDLSNCMVALGELKLAALCHGEDSITIPYQGSGKGVSFQLVKLGVWKSPTDMQSWVPLSTDDPVIDRLYLSSHRGVIADNQAKWAVPTTRTDDKLRMETCFQQACKGKIQALCENPEWAPLKDNRIPSYGVLSVDLSLTVELKIKIASGFGPLITHGSGMDLYKSNHNNVYWLTIPPMKNLALGVINTLEWIPRFKVSPYLFNVPIKEAGEDCHAPTYLPAEVDGDVKLSSNLVILPGQDLQYVLATYDTSRVEHAVVYYVYSPSRSFSYFYPFRLPIKGVPIELQVECFTWDQKLWCRHFCVLADSESGGHITHSGMEGMGVSCTVTREDGTNRR

SEQ ID NO: 137 - protein, 550 aa, synthetic construct
  REGION 1..550: note = Synthetic peptide
MGLKVNVSAIFMAVLLTLQTPTGQIHWGNLSKIGVVGIGSASYKVMTRSSHQSLVIKLMPNITLLNNCTRVEIAEYRRLLRTVLEPIRDALNAMTQNIRPVQSVASSRRHKRFAGVVLAGAALGVATAAQITAGIALHQSMLNSQAIDNLRASLETTNQAIEAIRQAGQEMILAVQGVQDYINNELIPSMNQLSCDLIGQKLGLKLLRYYTEILSLFGPSLRDPISAEISIQALSYALGGDINKVLEKLGYSGGDLLGILESRGIKARITHVDTESYFIVLSIAYPTLSEIKGVIVHRLEGVSYNIGSQEWYTTVPKYVATQGYLISNFDESSCTFMPEGTVCSQNALYPMSPLLQECLRGSTKSCARTLVSGSFGNRFILSQGNLIANCASILCKCYTTGTIINQDPDKILTYIAADHCPVVEVNGVTIQVGSRRYPDAVYLHRIDLGPPISLERLDVGTNLGNAIAKLEDAKELLESSDQILRSMKGLSSTSIVYILIAVCLGGLIGIPALICCCRGRCNKKGEQVGMSRPGLKPDLTGTSKSYVRSL

SEQ ID NO: 138 - protein, 500 aa, synthetic construct
  REGION 1..500: note = Synthetic peptide
IRCIGVSNRDFVEGMSGGTWVDVVLEHGGCVTVMAQDKPTVDIELVTTTVSNMAEVRSYCYEASISDMASDSRCPTQGEAYLDKQSDTQYVCKRTLVDRGWGNGCGLFGKGSLVTCAKFTCSKKMTGKSIQPENLEYRIMLSVHGSQHSGMIGYETDEDRAKVEVTPNSPRAEATLGGFGSLGLDCEPRTGLDFSDLYYLTMNNKHWLVHKEWFHDIPLPWHAGADTGTPHWNNKEALVEFKDAHAKRQTVVVLGSQEGAVHTALAGALEAEMDGAKGRLFSGHLKCRLKMDKLRLKGVSYSLCTAAFTFTKVPAETLHGTVTVEVQYAGTDGPCKIPVQMAVDMQTLTPVGRLITANPVITESTENSKMMLELDPPFGDSYIVIGVGDKKITHHWHRSGSTIGKAFEATVRGAKRMAVLGDTAWDFGSVGGVFNSLGKGIHQIFGAAFKSLFGGMSWFSQILIGTLLVWLGLNTKNGSISLTCLALGGVMIFLSTAVSA

SEQ ID NO: 139 - protein, 378 aa, synthetic construct
  REGION 1..378: note = Synthetic peptide
MKNFILLAVSSILLVDLFPTHCGHNVDLSKAINLNGVNFNNVDASSLGAAHVGQSASRGRGLGENPDDEEGDAKKKKDGKKAEPKNPRENKLKQPGDRADGQPAGDRADGQPAGDRADGQPAGDRAAGQPAGDRADGQPAGDRADGQPAGDRADGQPAGDRADGQPAGDRAAGQPAGDRAAGQPAGDRADGQPAGDRAAGQPAGDRADGQPAGDRAAGQPAGDRADGQPAGDRAAGQPAGDRAAGQPAGDRAAGQPAGDRAAGQPAGNGAGGQAAGGNAGGGQGQNNEGANAPNEKSVKEYLDKVRATVGTEWTPCSVTCGVGVRVRRRVNAANKKPEDLTLNDLETDVCTMDKCAGIFNVVSNSLGLVILLVLALFN

SEQ ID NO: 140 - protein, 546 aa, synthetic construct
  REGION 1..546: note = Synthetic peptide
MVVILDKRCYCNLLILILMISECSVGILHYEKLSKIGLVKGVTRKYKIKSNPLTKDIVIKMIPNVSNMSQCTGSVMENYKTRLNGILTPIKGALEIYKNNTHDLVGDVRLAGVIMAGVAIGIATAAQITAGVALYEAMKNADNINKLKSSIESTNEAVVKLQETAEKTVYVLTALQDYINTNLVPTIDKISCKQTELSLDLALSKYLSDLLFVFGPNLQDPVSNSMTIQAISQAFGGNYETLLRTLGYATEDFDDLLESDSITGQIIYVDLSSYYIIVRVYFPILTEIQQAYIQELLPVSFNNDNSEWISIVPNFILVRNTLISNIEIGFCLITKRSVICNQDYATPMTNNMRECLTGSTEKCPRELVVSSHVPRFALSNGVLFANCISVTCQCQTTGRAISQSGEQTLLMIDNTTCPTAVLGNVIISLGKYLGSVNYNSEGIAIGPPVFTDKVDISSQISSMNQSLQQSKDYIKEAQRLLDTVNPSLISMLSMIILYVLSIASLCIGLITFISFIIVEKKRNTYSRLEDRRVRPTSSGDLYYIGT

SEQ ID NO: 141 - protein, 602 aa, synthetic construct
  REGION 1..602: note = Synthetic peptide
MPAENKKVRFENTTSDKGKIPSKVIKSYYGTMDIKKINEGLLDSKILSAFNTVIALLGSIVIIVMNIMIIQNYTRSTDNQAVIKDALQGIQQQIKGLADKIGTEIGPKVSLIDTSSTITIPANIGLLGSKISQSTASINENVNEKCKFTLPPLKIHECNISCPNPLPFREYRPQTEGVSNLVGLPNNICLQKTSNQILKPKLISYTLPVVGQSGTCITDPLLAMDEGYFAYSHLERIGSCSRGVSKQRIIGVGEVLDRGDEVPSLFMTNVWTPPNPNTVYHCSAVYNNEFYYVLCAVSTVGDPILNSTYWSGSLMMTRLAVKPKSNGGGYNQHQLALRSIEKGRYDKVMPYGPSGIKQGDTLYFPAVGFLVRTEFKYNDSNCPITKCQYSKPENCRLSMGIRPNSHYILRSGLLKYNLSDGENPKVVFIEISDQRLSIGSPSKIYDSLGQPVFYQASFSWDTMIKFGDVLTVNPLVVNWRNNTVISRPGQSQCPRFNTCPEICWEGVYNDAFLIDRINWISAGVFLDSNQTAENPVFTVFKDNEILYRAQLASEDTNAQKTITNCFLLKNKIWCISLVEIYDTGDNVIRPKLFAVKIPEQCT

SEQ ID NO: 142 - protein, 775 aa, synthetic construct
  REGION 1..775: note = Synthetic peptide
MASLIYRQLLTNSYSVDLHDEIEQIGSEKTQNVTINPSPFAQTRYAPVNWGHGEINDSTTVEPILDGPYQPTTFTPPNDYWILINSNTNGVVYESTNNSDFWTAVVAIEPHVNPVDRQYTIFGESKQFNVSNDSNKWKFLEMFRSSSQNEFYNRRTLTSDTRFVGILKYGGRVWTFHGETPRATTDSSSTANLNNISITIHSEFYIIPRSQESKCNEYINNGLPPIQNTRNVVPLPLSSRSIQYKRAQVNEDIIVSKTSLWKEMQYNRDIIIRFKFGNSIVKMGGLGYKWSEISYKAANYQYNYLRDGEQVTAHTTCSVNGVNNFSYNGGSLPTDFGISRYEVIKENSYVYVDYWDDSKAFRNMVYVRSLAANLNSVKCTGGSYNFSIPVGAWPVMNGGAVSLHFAGVTLSTQFTDFVSLNSLRFRFSLTVDEPPFSILRTRTVNLYGLPAANPNNGNEYYEISGRFSLIYLVPTNDDYQTPIMNSVTVRQDLERQLTDLREEFNSLSQEIAMAQLIDLALLPLDMFSMFSGIKSTIDLTKSMATSVMKKFRKSKLATSISEMTNSLSDAASSASRNVSIRSNLSAISNWTNVSNDVSNVTNSLNDISTQTSTISKKFRLKEMITQTEGMSFDDISAAVLKTKIDMSTQIGKNTLPDIVTEASEKFIPKRSYRILKDDEVMEINTEGKFFAYKINTFDEVPFDVNKFAELVTDSPVISAIIDFKTLKNLNDNYGITRTEALNLIKSNPNMLRNFINQNNPIIRNRIEQLILQCKL

SEQ ID NO: 143 - protein, 230 aa, synthetic construct
  REGION 1..230: note = Synthetic peptide
MASLIYRQLLTNSYSVDLHDEIEQIGSEKTQNVTINPSPFAQTRYAPVNWGHGEINDSTTVEPILDGPYQPTTFTPPNDYWILINSNTNGVVYESTNNSDFWTAVVAIEPHVNPVDRQYTIFGESKQFNVSNDSNKWKFLEMFRSSSQNEFYNRRTLTSDTRFVGILKYGGRVWTFHGETPRATTDSSSTANLNNISITIHSEFYIIPRSQESKCNEYINNGLPPIQNTR

SEQ ID NO: 144 - protein, 539 aa, synthetic construct
  REGION 1..539: note = Synthetic peptide
MSWKVVIIFSLLITPQHGLKESYLEESCSTITEGYLSVLRTGWYTNVFTLEVGDVENLTCSDGPSLIKTELDLTKSALRELKTVSADQLAREEQIENPRQSRFVLGAIALGVATAAAVTAGVAIAKTIRLESEVTAIKNALKTTNEAVSTLGNGVRVLATAVRELKDFVSKNLTRAINKNKCDIDDLKMAVSFSQFNRRFLNVVRQFSDNAGITPAISLDLMTDAELARAVSNMPTSAGQIKLMLENRAMVRRKGFGILIGVYGSSVIYMVQLPIFGVIDTPCWIVKAAPSCSGKKGNYACLLREDQGWYCQNAGSTVYYPNEKDCETRGDHVFCDTAAGINVAEQSKECNINISTTNYPCKVSTGRHPISMVALSPLGALVACYKGVSCSIGSNRVGIIKQLNKGCSYITNQDADTVTIDNTVYQLSKVEGEQHVIKGRPVSSSFDPIKFPEDQFNVALDQVFENIENSQALVDQSNRILSSAEKGNTGFIIVIILIAVLGSSMILVSIFIIIKKTKKPTGAPPELSGVTNNGFIPHS

SEQ ID NO: 145 - protein, 219 aa, synthetic construct
  REGION 1..219: note = Synthetic peptide
MEVKVENIRAIDMLKARVKNRVARSKCFKNASLILIGITTLSIALNIYLIINYTIQKTSSESEHHTSSPPTESNKEASTISTDNPDINPNSQHPTQQSTENPTLNPAASVSPSETEPASTPDTTNRLSSVDRSTAQPSESRTKTKPTVHTRNNPSTASSTQSPPRATTKAIRRATTFRMSSTGKRPTTTSVQSDSSTTTQNHEETGSANPQASVSTMQN

SEQ ID NO: 146 - protein, 555 aa, synthetic construct
  REGION 1..555: note = Synthetic peptide
MQKSEILFLIYSSLLLSSSLCQIPVDKLSNVGVIINEGKLLKIAGSYESRYIVLSLVPSIDLEDGCGTTQIIQYKNLLNRLLIPLKDALDLQESLITITNDTTVTNDNPQSRFFGAVIGTIALGVATAAQITAGIALAEAREARKDIALIKDSIIKTHNSVELIQRGIGEQIIALKTLQDFVNNEIRPAIGELRCETTALKLGIKLTQHYSELATAFSSNLGTIGEKSLTLQALSSLYSANITEILSTIKKDKSDIYDIIYTEQVKGTVIDVDLEKYMVTLLVKIPILSEIPGVLIYRASSISYNIEGEEWHVAIPNYIINKASSLGGADVTNCIESRLAYICPRDPTQLIPDNQQKCILGDVSKCPVTKVINNLVPKFAFINGGVVANCIASTCTCGTNRIPVNQDRSRGVTFLTYTNCGLIGINGIELYANKRGRDTTWGNQIIKVGPAVSIRPVDISLNLASATNFLEESKIELMKAKAIISAVGGWHNTESTQIIIIIIVCILIIIICGILYYLYRVRRLLVMINSTHNSPVNTYTLESRMRNPYIGNNSN

SEQ ID NO: 147 - protein, 571 aa, synthetic construct
  REGION 1..571: note = Synthetic peptide
MEDYSNLSLKSIPKRTCRIIFRTATILGICTLIVLCSSILHEIIHLDVSSGLMDSDDSQQGIIQPIIESLKSLIALANQILYNVAIIIPLKIDSIETVIFSALKDMHTGSMSNTNCTPGNLLLHDAAYINGINKFLVLKSYNGTPKYGPLLNIPSFIPSATSPNGCTRIPSFSLIKTHWCYTHNVMLGDCLDFTTSNQYLAMGIIQQSAAAFPIFRTMKTIYLSDGINRKSCSVTAIPGGCVLYCYVATRSEKEDYATTDLAELRLAFYYYNDTFIERVISLPNTTGQWATINPAVGSGIYHLGFILFPVYGGLISGTPSYNKQSSRYFIPKHPNITCAGNSSEQAAAARSSYVIRYHSNRLIQSAVLICPLSDMHTARCNLVMFNNSQVMMGAEGRLYVIDNNLYYYQRSSSWWSASLFYRINTDFSKGIPPIIEAQWVPSYQVPRPGVMPCNATSFCPANCITGVYADVWPLNDPEPTSQNALNPNYRFAGAFLRNESNRTNPTFYTASASALLNTTGFNNTNHKAAYTSSTCFKNTGTQKIYCLIIIEMGSSLLGEFQIIPFLRELIP

SEQ ID NO: 148 - protein, 217 aa, synthetic construct
  REGION 1..217: note = Synthetic peptide
MNKLYSLFLFLFIQLSIKYNNAKVTVDTVCKRGFLIQMSGHLECKCENDLVLVNEETCEEKVLKCDEKTVNKPCGDFSKCIKIDGNPVSYACKCNLGYDMVNNVCIPNECKNVTCGNGKCILDTSNPVKTGVCSCNIGKVPNVQDQNKCSKDGETKCSLKCLKENETCKAVDGIYKCDCKDGFIIDNESSICTAFSAYNILNLSIMFILFSVCFFIM

SEQ ID NO: 149 - protein, 274 aa, synthetic construct
  REGION 1..274: note = Synthetic peptide
MNRTAFCCLSLTTALILTACSSGGGGVAADIGAGLADALTAPLDHKDKGLQSLTLDQSVRKNEKLKLAAQGAEKTYGNGDSLNTGKLKNDKVSRFDFIRQIEVDGQLITLESGEFQVYKQSHSALTAFQTEQIQDSEHSGKMVAKRQFRIGDIAGEHTSFDKLPEGGRATYRGTAFGSDDAGGKLTYTIDFAAKQGNGKIEHLKSPELNVDLAAADIKPDGKRHAVISGSVLYNQAEKGSYSLGIFGGKAQEVAGSAEVKTVNGIRHIGLAAKQ

SEQ ID NO: 150 - protein, 370 aa, synthetic construct
  REGION 1..370: note = Synthetic peptide
MQTAARRSFDYDMPLIQTPTSACQIRQAWAKVADTPDRETAGRLKDEIKALLKETNAVLVAHYYVDPLIQDLALETGGCVGDSLEMARFGAEHEAGTLVVAGVRFMGESAKILCPEKTVLMPDLEAECSLDLGCPEEAFSAFCDQHPDRTVVVYANTSAAVKARADWVVTSSVALEIVSYLKSRGEKLIWGPDRHLGDYIRRETGADMLLWQGSCIVHNEFKGQELAALKAEHPDAVVLVHPESPQSVIELGDVVGSTSKLLKAAVSRPEKKFIVATDLGILHEMQKQAPDKQFIAAPTAGNGGSCKSCAFCPWMAMNSLGGIKYALTSGHNEILLDRKLGEAAKLPLQRMLDFAAGLKRGDVFNGMGPA

SEQ ID NO: 151 - protein, 492 aa, synthetic construct
  REGION 1..492: note = Synthetic peptide
MFKRSVIAMACIFALSACGGGGGGSPDVKSADTLSKPAAPVVSEKETEAKEDAPQAGSQGQGAPSAQGGQDMAAVSEENTGNGGAAATDKPKNEDEGAQNDMPQNAADTDSLTPNHTPASNMPAGNMENQAPDAGESEQPANQPDMANTADGMQGDDPSAGGENAGNTAAQGTNQAENNQTAGSQNPASSTNPSATNSGGDFGRTNVGNSVVIDGPSQNITLTHCKGDSCSGNNFLDEEVQLKSEFEKLSDADKISNYKKDGKNDGKNDKFVGLVADSVQMKGINQYIIFYKPKPTSFARFRRSARSRRSLPAEMPLIPVNQADTLIVDGEAVSLTGHSGNIFAPEGNYRYLTYGAEKLPGGSYALRVQGEPSKGEMLAGTAVYNGEVLHFHTENGRPSPSRGRFAAKVDFGSKSVDGIIDSGDGLHMGTQKFKAAIDGNGFKGTWTENGGGDVSGKFYGPAGEEVAGKYSYRPTDAEKGGFGVFAGKKEQD

SEQ ID NO: 152 - protein, 157 aa, synthetic construct
  REGION 1..157: note = Synthetic peptide
  SITE 1: note = MISC_FEATURE - Optional residue
MNQHSHKDYETVRIAVVRARWHAEIVDACVSAFEAAMADIGGDRFAVDVFDVPGAYEIPLHARTLAETGRYGAVLGTAFVVNGGIYRHEFVASAVIDGMMNVQLSTGVPVLSAVLTPHRYRDSDAHTLLFLALFAVKGMEAARACVEILAAREKIAA

SEQ ID NO: 153 - protein, 167 aa, synthetic construct
  REGION 1..167: note = Synthetic peptide
  SITE 1: note = MISC_FEATURE - Optional residue
  REGION 158..167: note = MISC_FEATURE - Optional residue
MNQHSHKDHETVRIAVVRARWHAEIVDACVSAFEAAMRDIGGDRFAVDVFDVPGAYEIPLHARTLAETGRYGAVLGTAFVVNGGIYRHEFVASAVINGMMNVQLNTGVPVLSAVLTPHNYDKSKAHTLLFLALFAVKGMEAARACVEILAAREKIAAGSLEHHHHHH

SEQ ID NO: 154 - protein, 163 aa, synthetic construct
  REGION 1..163: note = Synthetic peptide
  REGION 156..163: note = MISC_FEATURE - Optional residues
MGKYDGSKLRIGILHARWNAEIILALVLGALKRLQEFGVKRENIIIETVPGSFELPYGSKLFVEKQKRLGKPLDAIIPIGVLIKGSTMHFEYICDSTTHQLMKLNFELGIPVIFGVLTCLTDEQAEARAGLIEGKMHNHGEDWGAAAVEMATKFNLEHHHHHH

SEQ ID NO: 155 - protein, 171 aa, synthetic construct
  REGION 1..171: note = Synthetic peptide
  REGION 157..171: note = MISC_FEATURE - Optional residues
MGKYDGSKLRIGILHARGNAEIILALVLGALKRLQEFGVKRENIIIETVPGSFELPYGSKLFVEKQKRLGKPLDAIIPIGVLIRGSTPHFDYIADSTTHQLMKLNFELGIPVIFGVITADTCEQAEARAGLIEGKMHNHGEDWGAAAVEMATKFNGGWELQLEGSHHHHHH

SEQ ID NO: 156 - protein, 171 aa, synthetic construct
  REGION 1..171: note = Synthetic peptide
  REGION 157..171: note = MISC_FEATURE - Optional residues
MGKYDGSKLRIGILHARGNAEIILALVLGALKRLQEFGVKRENIIIETVPGSFELPYGSKLFVEKQKRLGKPLDAIIPIGVLIRGCTPHFDYIADSTTHQLMKLNFELGIPVIFGVITADTCEQAEARAGLIEGKMHNHGEDWGAAAVEMATKFNGGWELQLEGSHHHHHH

SEQ ID NO: 157 - protein, 230 aa, synthetic construct
  REGION 1..230: note = Synthetic peptide
  REGION 2..11: note = MISC_FEATURE - Optional residues
MGSHHHHHHGSDEQAEERAGTKAGNHGEDWGAAAVEMATKFNGSGGSGKYDGSKLRIGILHARGNAEIILELVLGALKRLQEFGVKRENIIIETVPGSFELPYGSKLFVEKQKRLGKPLDAIIPIGVLIRGSTAHFDYIADSTTHQLMKLNFELGIPVIFGVLTTESGGSVPGAGVPGVGVPGVGVPGAGVPGVGVPGAGVPGVGVPGAGVPGVGVPGVGVPGAGVPGVG

SEQ ID NO: 158 - protein, 230 aa, synthetic construct
  REGION 1..230: note = Synthetic peptide
  REGION 2..11: note = MISC_FEATURE - Optional residues
MGSHHHHHHGSDEQAEERAGTKAGNHGEDWGAAAVEMATKFNGSGGSGKYDGSKLRIGILHARGNAEIILELVLGALKRLQEFGVKRENIIIETVPGSFELPYGSKLFVEKQKRLGKPLDAIIPIGVLIRGSTAHFDYIADSTTHQLMKLNFELGIPVIFGVLTTESGGSVPGAGVPGAGVPGAGVPGAGVPGAGVPGAGVPGAGVPGAGVPGAGVPGAGVPGAGVPGAG

SEQ ID NO: 159 - protein, 233 aa, synthetic construct
  REGION 1..233: note = Synthetic peptide
  REGION 2..11: note = MISC_FEATURE - Optional residues
MGSHHHHHHGSDEQAEERAGTKAGNHGEDWGAAAVEMATKFNGSGGSGKYDGSKLRIGILHARGNAEIILELVLGALKRLQEFGVKRENIIIETVPGSFELPYGSKLFVEKQKRLGKPLDAIIPIGVLIRGSTAHFDYIADSTTHQLMKLNFELGIPVIFGVLTTESGGSASPAAPAPASPAAPAPSAPAAASPAAPAPASPAAPAPSAPAAASPAAPAPASPAAPAPSAPAA

SEQ ID NO: 160 - protein, 198 aa, synthetic construct
  REGION 1..198: note = Synthetic peptide
  REGION 181..198: note = MISC_FEATURE - Optional residues
MDSKGSSQKGSRLLLLLVVSNLLLPQGVLAKYDGSKLRIGILHARGNAEIILELVLGALKRLQEFGVKRENIIIETVPGSFELPYGSKLFVEKQKRLGKPLDAIIPIGVLIRGSTAHFDYIADSTTHQLMKLNFELGIPVIFGVLTTESDEQAEERAGTKAGNHGEDWGAAAVEMATKFNLEEQKLISEEDLHHHHHH

SEQ ID NO: 161 - protein, 198 aa, synthetic construct
  REGION 1..198: note = Synthetic peptide
  REGION 181..198: note = MISC_FEATURE - Optional residues
MDSKGSSQKGSRLLLLLVVSNLLLPQGVLAKYDGSKLRIGILHARGNAEIILELVLGALKRLQEFGVKRENIIIETVPGSFELPYGSKLFVEKQKRLGKPLDAIIPIGVLIRGNDTHFDYIADSTTHQLMKLNFELGIPVIFGVLTTNSTEQAEERAGTKAGNHGEDWGAAAVEMATKFNLEEQKLISEEDLHHHHHH

SEQ ID NO: 162 - protein, 136 aa, synthetic construct
  REGION 1..136: note = Synthetic peptide
  REGION 121..136: note = MISC_FEATURE - Optional residues
MEEAELAYLLGELAYKLGEYRIAIRAYRIALKRDPNNAEAWYNLGNAYYKQGRYREAIEYYQKALELDPNNAEAWYNLGNAYYERGEYEEAIEYYRKALRLDPNNADAMQNLLNAKMREEGGWELQGSLEHHHHHH

SEQ ID NO: 163 - protein, 167 aa, synthetic construct
  REGION 1..167: note = Synthetic peptide
  REGION 150..167: note = MISC_FEATURE - Optional residues
MDSKGSSQKGSRLLLLLVVSNLLLPQGVLAEEAELAYLLGELAYKLGEYRIAIRAYRIALKYDNLTAEAWYNLGNAYYKQGRYREAIEYYQKALELDPNNAEAWYNLGNAYYERGEYENATEYYRKALRLDPNNADAMQNLLNAKMREELEEQKLISEEDLHHHHHH

SEQ ID NO: 164 - protein, 221 aa, synthetic construct
  REGION 1..221: note = Synthetic peptide
  REGION 204..221: note = MISC_FEATURE - Optional residues
MKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCLEEQKLISEEDLHHHHHH

SEQ ID NO: 165 - protein, 248 aa, synthetic construct
  REGION 1..248: note = Synthetic peptide
  REGION 231..248: note = MISC_FEATURE - Optional residues
MDSKGSSQKGSRLLLLLVVSNLLLPQGVLAEELFKKHKIVAVLRANSVEEAIEKAVAVFAGGVHLIEITFTVPNATTVIKALSVLKEKGAIIGAGTVTSVEYANETVESGAEFIVSPHLDEEISNFTKEKGVFYMPGVMTPTELVKAMKLGHTILKLFPGEVVGPQFVKAMKGPFHNATFVPTGGVNLDNVCEWFKAGVLAVGVGSALVKGTPDEVREKAKAFVEKIRGCLEEQKLISEEDLHHHHHH

SEQ ID NO: 166 - protein, 167 aa, synthetic construct; identical to SEQ ID NO: 153
  REGION 1..167: note = Synthetic peptide
  REGION 158..167: note = MISC_FEATURE - Optional residues

SEQ ID NO: 167 - protein, 167 aa, synthetic construct
  REGION 1..167: note = Synthetic peptide
  REGION 158..167: note = MISC_FEATURE - Optional residues
MNQHSHKDYETVRIAVVRARWHADIVDQCVSAFEAEMADIGGDRFAVDVFDVPGAYEIPLHARTLAETGRYGAVLGTAFVVNGGIYRHEFVASAVIDGMMNVQLSTGVPVLSAVLTPHNYHDSAEHHRFFFEHFTVKGKEAARACVEILAAREKIAAGSLEHHHHHH

SEQ ID NO: 168 - protein, 653 aa, synthetic construct
  REGION 1..653: note = Synthetic peptide
  REGION 642..653: note = MISC_FEATURE - Optional residues
MKAILVVLLYTFTTANADTLCIGYHANNSTDTVDTVLEKNVTVTHSVNLLEDKHNGKLCKLRGVAPLHLGKCNIAGWILGNPECESLSTASSWSYIVETSNSDNGTCFPGDFINYEELREQLSSVSSFERFEIFPKTSSWPNHDSNKGVTAACPHAGAKSFYKNLIWLVKKGNSYPKLNQSYINDKGKEVLVLWGIHHPSTTADQQSLYQNADAYVFVGTSRYSKKFKPEIATRPKVRDQEGRMNYYWTLVEPGDKITFEATGNLVVPRYAFTMERNAGSGIIISDTPVHDCNTTCQTPEGAINTSLPFQNIHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVSAEEAELAYLLGELAYKLGEYRIAIRAYRIALKRDPNNAEAWYNLGNAYYKQGRYREAIEYYQKALELDPNNAEAWYNLGNAYYERGEYEEAIEYYRKALRLDPNNADAMQNLLNAKMREEGGWELQHHHHHH

SEQ ID NO: 169 - protein, 782 aa, synthetic construct
  REGION 1..782: note = Synthetic peptide
  REGION 765..782: note = MISC_FEATURE - Optional residues
MDSKGSSQKGSRLLLLLVVSNLLLPQGVLADTLCIGYHANNSTDTVDTVLEKNVTVTHSVNLLEDKHNGKLCKLRGVAPLHLGKCNIAGWILGNPECESLSTASSWSYIVETSNSDNGTCFPGDFINYEELREQLSSVSSFERFEIFPKTSSWPNHDSNKGVTAACPHAGAKSFYKNLIWLVKKGNSYPKLNQSYINDKGKEVLVLWGIHHPSTTADQQSLYQNADAYVFVGTSRYSKKFKPEIATRPKVRDQEGRMNYYWTLVEPGDKITFEATGNLVVPRYAFTMERNAGSGIIISDTPVHDCNTTCQTPEGAINTSLPFQNIHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVSAGSGGSGGSGGSGGSEKAAKAEEAARKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTELEEQKLISEEDLHHHHHH

SEQ ID NO: 170 - protein, 586 aa, synthetic construct
  REGION 1..586: note = Synthetic peptide
  REGION 534..586: note = MISC_FEATURE - Optional residues
MDSKGSSQKGSRLLLLLVVSNLLLPQGVLADTLCIGYHANNSTDTVDTVLEKNVTVTHSVNLLEDKHNGKLCKLRGVAPLHLGKCNIAGWILGNPECESLSTASSWSYIVETSNSDNGTCFPGDFINYEELREQLSSVSSFERFEIFPKTSSWPNHDSNKGVTAACPHAGAKSFYKNLIWLVKKGNSYPKLNQSYINDKGKEVLVLWGIHHPSTTADQQSLYQNADAYVFVGTSRYSKKFKPEIATRPKVRDQEGRMNYYWTLVEPGDKITFEATGNLVVPRYAFTMERNAGSGIIISDTPVHDCNTTCQTPEGAINTSLPFQNIHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVGSGYIPEAPRDGQAYVRKDGEWVLLSTFLGSGLNDIFEAQKIEWHEGHHHHHH

SEQ ID NO: 171 - protein, 653 aa, synthetic construct
  REGION 1..653: note = Synthetic peptide
  REGION 642..653: note = MISC_FEATURE - Optional residues
MKAILVVLLYTFTTANADTLCIGYHANNSTDTVDTVLEKNVTVTHSVNLLEDKHNGKLCKLRGVAPLHLGKCNIAGWILGNPECESLSTASSWSYIVETSNSDNGTCFPGDFINYEELREQLSSVSSFERFEIFPKTSSWPNHDSNKGVTAACPHAGAKSFYKNLIWLVKKGNSYPKLNQSYINDKGKEVLVLWGIHHPSTTADQQSLYQNADAYVFVGTSRYSKKFKPEIATRPKVRDQEGRMNYYWTLVEPGDKITFEATGNLVVPRYAFTMERNAGSGIIISDTPVHDCNTTCQTPEGAINTSLPFQNIHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRNQLKNNAKEIGNGCFEFYHKCDNTCMESVKNGTYDYPKYSEEAKLNREKIDGVSAEEAELAYLLGELAYKLGEYRIAIRAYRIALKRDPNNAEAWYNLGNAYYKQGDYDEAIEYYQKALELDPNNAEAWYNLGNAYYKQGDYDEAIEYYQKALELDPNNAEAKQNLGNAKQKQGGGWELQHHHHHH

SEQ ID NO: 172 - protein, 478 aa, synthetic construct
  REGION 1..478: note = Synthetic peptide
  REGION 469..478: note = MISC_FEATURE - Optional residues
MGILPSPGMPALLSLVSLLSVLLMGCVAETGTRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTGGSGGSGSGGSGGSGSEKAAKAEEAARKMEELFKKHKIVAVLRANSVEEAIEKAVAVFAGGVHLIEITFTVPDADTVIKALSVLKEKGAIIGAGTVTSVEQARKAVESGAEFIVSPHLDEEISQFAKEKGVFYMPGVMTPTELVKAMKLGHTILKLFPGEVVGPQFVKAMKGPFPNVKFVPTGGVNLDNVAEWFKAGVLAVGVGSALVKGTPDEVREKAKAFVEKIRGATEGGSHHHHHHHH

SEQ ID NO: 173 - protein, 1288 aa, synthetic construct
  REGION 1..1288: note = Synthetic peptide
  REGION 1209..1288: note = MISC_FEATURE - Optional residues
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPGSASSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQGSGYIPEAPRDGQAYVRKDGEWVLLSTFLGRSLEVLFQGPGHHHHHHHHSAWSHPQFEKGGGSGGGGSGGSAWSHPQFEK

SEQ ID NO: 174 - protein, 748 aa, synthetic construct
  REGION 1..748: note = Synthetic peptide
  REGION 741..748: note = MISC_FEATURE - Optional residues
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLGSGGSGSGSGGSEKAAKAEEAARKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTELEHHHHHH

SEQ ID NO: 175 - protein, 616 aa, synthetic construct
  REGION 1..616: note = Synthetic peptide
  REGION 517..616: note = MISC_FEATURE - Optional residues
MELLILKANAITTILTAVTFCFASGQNITEEFYQSTCSAVSKGYLSALRTGWYTSVITIELSNIKENKCNGTDAKVKLIKQELDKYKNAVTELQLLMQSTPATNNRARRELPRFMNYTLNNAKKTNVTLSKKRKRRFLGFLLGVGSAIASGVAVCKVLHLEGEVNKIKSALLSTNKAVVSLSNGVSVLTFKVLDLKNYIDKQLLPILNKQSCSISNIETVIEFQQKNNRLLEITREFSVNAGVTTPVSTYMLTNSELLSLINDMPITNDQKKLMSNNVQIVRQQSYSIMCIIKEEVLAYVVQLPLYGVIDTPCWKLHTSPLCTTNTKEGSNICLTRTDRGWYCDNAGSVSFFPQAETCKVQSNRVFCDTMNSLTLPSEVNLCNVDIFNPKYDCKIMTSKTDVSSSVITSLGAIVSCYGKTKCTASNKNRGIIKTFSNGCDYVSNKGVDTVSVGNTLYYVNKQEGKSLYVKGEPIINFYDPLVFPSDEFDASISQVNEKINQSLAFIRKSDELLSAIGGYIPEAPRDGQAYVRKDGEWVLLSTFLENLYFQSSAWSHPQFEKGGGSGGGSGGSAWSHPQFEKGSGSGSGLNDIFEAQKIEWHEGSGSGSHHHHHHHH

SEQ ID NO: 176 - protein, 869 aa, synthetic construct
  REGION 1..869: note = Synthetic peptide
ADKLWVTVYYGVPVWKDACTTLFCASDAKAYDTEKRNVWATHCCVPTDPNPQEVVLENVTENFNMWKNDMVEQMHEDIISLWDQSLKPCVKLTPLCVTLNCTDYVGNATNASTTNATGGIGGTVERGEIKNCSFNITTSLRDKVQKEYALFYKLDIVPIDNDNTNNTYRLINCNTTVIKQACPKVSFEPIPIHYCAPAGFAILKCNDKKFNGTGPCTNVSTVQCTHGIRPVVSTQLLLNGSLAEKEVIIRSQNFTNNAKVIIVQLNESVVINCTRPNNNTVKSIHIAPGQWFYYTGAIIGDIRQAHCNISRVKWNNTLKQIATKLREQFKNKTIAFNQSSGGDPEIVMHSFNCGGEFFYCNTTQLFNSTWNDTEVSNYTDITHITLPCRIKQIINMWQRVGQAMYAPPIRGQIRCSSNITGLLLTRDGGSNENKTSETETFRPAGGDMRDNWRSELYKYKVVKIEPLGVAPTRCKRRVVQRRRRRRAVGAIGAVSLGFLGAAGSTMGAASMTLTVQARQLLSGIVQQQNNCLRAPECQQHMLKDTHWGIKQLQARVLAVEHYLRDQQLLGIWGCSGKLICCTAVPWNNTWSNRSLDMIWNNMTWIEWEREIDNYTGLIYNLLEESQNQQEKNEQELLELDGSGGSGGSGGSGGSEKAAKAEEAARKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTE

SEQ ID NO: 177 - protein, 839 aa, synthetic construct
  REGION 1..839: note = Synthetic peptide
AENLWVTVYYGVPVWKDAETTLFCASDAKAYDTEKRNVWATHCCVPTDPNPQEIVLENVTENFNMWKNNMVEQMHTDIISLWDQSLKPCVKLTPLCVTLNCTDVNATNNTTNNEEIKNCSFNITTELRDKKKKVYALFYKLDVVPIDDNNSYRLINCNTSAITQACPKVSFEPIPIHYCAPAGFAILKCNDKKFNGTGPCKNVSTVQCTHGIKPVVSTQLLLNGSLAEEEIIIRSENITNNAKTIIVQLNESVEINCTRPNNNTRKSIRIGPGQWFYATGDIIGDIRQAHCNISRTKWNKTLQQVAKKLREHFNKTIIFNPSSGGDLEITTHSFNCGGEFFYCNTSELFNSTWNGTNNTITLPCRIKQIINMWQRVGQAMYAPPIEGKIRCTSNITGLLLTRDGGNNNTETFRPGGGDMRDNWRSELYKYKVVKIEPLGVAPTRCKRRVVERRRRRRAVGIGAVFLGFLGAAGSTMGAASMTLTVQARNLLSGIVQQQSNLLRAPECQQHLLQLTVWGIKQLQARVLAVERYLKDQQLLGIWGCSGKLICCTNVPWNSSWSNKSQDEIWDNMTWMEWDKEINNYTDIIYSLIEESQNQQEKNEQELLALDGSGGSGGSGGSGGSEKAAKAEEAARKMEELFKKHKIVAVLRANSVEEAIEKAVAVFAGGVHLIEITFTVPDADTVIKALSVLKEKGAIIGAGTVTSVEQCRKAVESGAEFIVSPHLDEEISQFCKEKGVFYMPGVMTPTELVKAMKLGHTILKLFPGEVVGPQFVKAMKGPFPNVKFVPTGGVNLDNVCEWFKAGVLAVGVGSALVKGTPDEVREKAKAFVEKIRGCTE

SEQ ID NO: 178 - protein, 656 aa, synthetic construct
  REGION 1..656: note = Synthetic peptide
  REGION 641..656: note = MISC_FEATURE - Optional residues
ADKLWVTVYYGVPVWKDACTTLFCASDAKAYDTEKRNVWATHCCVPTDPNPQEVVLENVTENFNMWKNDMVEQMHEDIISLWDQSLKPCVKLTPLCVTLNCTDYVGNATNASTTNATGGIGGTVERGEIKNCSFNITTSLRDKVQKEYALFYKLDIVPIDNDNTNNTYRLINCNTTVIKQACPKVSFEPIPIHYCAPAGFAILKCNDKKFNGTGPCTNVSTVQCTHGIRPVVSTQLLLNGSLAEKEVIIRSQNFTNNAKVIIVQLNESVVINCTRPNNNTVKSIHIAPGQWFYYTGAIIGDIRQAHCNISRVKWNNTLKQIATKLREQFKNKTIAFNQSSGGDPEIVMHSFNCGGEFFYCNTTQLFNSTWNDTEVSNYTDITHITLPCRIKQIINMWQRVGQAMYAPPIRGQIRCSSNITGLLLTRDGGSNENKTSETETFRPAGGDMRDNWRSELYKYKVVKIEPLGVAPTRCKRRVVQRRRRRRAVGAIGAVSLGFLGAAGSTMGAASMTLTVQARQLLSGIVQQQNNCLRAPECQQHMLKDTHWGIKQLQARVLAVEHYLRDQQLLGIWGCSGKLICCTAVPWNNTWSNRSLDMIWNNMTWIEWEREIDNYTGLIYNLLEESQNQQEKNEQELLELDGSGSGGSGHHHHHHHH

SEQ ID NO: 179 - protein, 626 aa, synthetic construct
  REGION 1..626: note = Synthetic peptide
  REGION 611..626: note = MISC_FEATURE - Optional residues
AENLWVTVYYGVPVWKDAETTLFCASDAKAYDTEKRNVWATHCCVPTDPNPQEIVLENVTENFNMWKNNMVEQMHTDIISLWDQSLKPCVKLTPLCVTLNCTDVNATNNTTNNEEIKNCSFNITTELRDKKKKVYALFYKLDVVPIDDNNSYRLINCNTSAITQACPKVSFEPIPIHYCAPAGFAILKCNDKKFNGTGPCKNVSTVQCTHGIKPVVSTQLLLNGSLAEEEIIIRSENITNNAKTIIVQLNESVEINCTRPNNNTRKSIRIGPGQWFYATGDIIGDIRQAHCNISRTKWNKTLQQVAKKLREHFNKTIIFNPSSGGDLEITTHSFNCGGEFFYCNTSELFNSTWNGTNNTITLPCRIKQIINMWQRVGQAMYAPPIEGKIRCTSNITGLLLTRDGGNNNTETFRPGGGDMRDNWRSELYKYKVVKIEPLGVAPTRCKRRVVERRRRRRAVGIGAVFLGFLGAAGSTMGAASMTLTVQARNLLSGIVQQQSNLLRAPECQQHLLQLTVWGIKQLQARVLAVERYLKDQQLLGIWGCSGKLICCTNVPWNSSWSNKSQDEIWDNMTWMEWDKEINNYTDIIYSLIEESQNQQEKNEQELLALDGSGSGGSGHHHHHHHH

SEQ ID NO: 180 - protein, 15 aa, synthetic construct
  REGION 1..15: note = Synthetic peptide
GSGGSGSGSGGSGSG

SEQ ID NO: 181 - protein, 8 aa, synthetic construct
  REGION 1..8: note = Synthetic peptide
GGSGGSGS

SEQ ID NO: 182 - protein, 8 aa, synthetic construct
  REGION 1..8: note = Synthetic peptide
GSGGSGSG

SEQ ID NO: 183 - protein, 4 aa, synthetic construct
  REGION 1..4: note = Synthetic peptide
AGGA

SEQ ID NO: 184 - protein, 5 aa, synthetic construct
  REGION 1..5: note = Synthetic peptide
AGGAM

SEQ ID NO: 185 - protein, 4 aa, synthetic construct
  REGION 1..4: note = Synthetic peptide
GSGS

SEQ ID NO: 186 - protein, 4 aa, synthetic construct
  REGION 1..4: note = Synthetic peptide
ETGT

(19) United States
(12) Patent Application Publication
(10) Pub. No.: US 2024/0042018 A1
(43) Pub. Date: Feb. 8, 2024

(54) Glycoengineered protein nanoparticles and uses thereof

(71) Applicant: University of Washington, Seattle, WA (US)

(72) Inventors: John Cavin KRAFT II, Seattle, WA (US); Isaac SAPPINGTON, Seattle, WA (US); Neil P. KING, Seattle, WA (US)

(21) Appl. No.: 18/360,190

(22) Filed: Jul. 27, 2023

Related U.S. Application Data
(60) Provisional application No. 63/369,843, filed on Jul. 29, 2022.

(51) Int. Cl. A61K 39/385; C07K 14/00; A61K 39/145

(52) U.S. Cl. CPC A61K 39/385; C07K 14/00; A61K 39/145; C07K 2319/735; C07K 2319/40; A61K 2039/6031

(57) ABSTRACT

Polypeptides including the amino acid sequence of SEQ ID NO:78-80, substituted with one or more sequon, are provided, as are fusion proteins and nanoparticles formed from such polypeptides, and methods for their use.

### CROSS REFERENCE

This application claims priority to U.S. Provisional Patent Application Ser. No. 63/369,843 filed Jul. 29, 2022, incorporated by reference herein in its entirety.

### FEDERAL FUNDING STATEMENT

This invention was made with government support under Grant No. HDTRA1-18-1-0001, awarded by the Defense Threat Reduction Agency (DTRA). The government has certain rights in the invention.

### REFERENCE TO SEQUENCE LISTING

A computer readable form of the Sequence Listing is filed with this application by electronic submission and is incorporated into this application by reference in its entirety. The Sequence Listing is contained in the file created on Jul. 27, 2023, having the file name “22-1043-US_Sequence-Listing.xml” and is 432,387 bytes in size.

### BACKGROUND

Protein nanoparticle scaffolds are increasingly used in next-generation vaccine designs and several have established records of clinical safety and efficacy. Yet the rules for how immune responses specific to nanoparticle scaffolds affect the immunogenicity of displayed antigens have not been established.

### SUMMARY

In a first aspect, the disclosure provides polypeptides comprising the amino acid sequence of SEQ ID NO:78-80, substituted with one or more sequon, wherein the N-terminal residue may be present or may be absent. In various embodiments, each sequon may independently consist of the amino acid sequence selected from the group consisting of NET, NDS, NST, FSNES (SEQ ID NO:81), NES, FENES (SEQ ID NO:82), NAS, NGS, NHT, FFNHT (SEQ ID NO:83), NLS, FDNLS (SEQ ID NO:84), NNS, WHNNS (SEQ ID NO:85), NYS, FINYS (SEQ ID NO:86), NIS, FLNAT (SEQ ID NO:87), NAT, FLNAS (SEQ ID NO:88), WVNNS (SEQ ID NO:89), NKS, YLNKS (SEQ ID NO:90), FSNET (SEQ ID NO:91), YVNVT (SEQ ID NO:92), NRS, YANRS (SEQ ID NO:93), WANAS (SEQ ID NO:94), NFT, WANFT (SEQ ID NO:95), NVS, NGT, NVT, WLNHT (SEQ ID NO:96), and NTS.

In certain embodiments, the polypeptide comprises the amino acid sequence selected from the group consisting of SEQ ID NO:1-3, 5, 8-10, 13, 23, 26-28, 31-32, 34-38, 40, 42-46, 48-55, 59-60, and 67-74, wherein:
(a) each sequon may independently be substituted with any other sequon;
(b) X1 may be present or absent, and when present comprises a signal peptide; and
(c) X2 may be present or absent, and when present comprises a purification tag.

In some embodiments X1 is absent. In other embodiments, X1 is present. When present, X1 may be any signal peptide as appropriate for an intended use. In embodiment, X1 may comprise or consist of the amino acid sequence MDSKGSSQKGSRLLLLLVVSNLLLPQGVLA (SEQ ID NO:97). In one embodiment, X3 is absent. In another embodiment, X3 may be present and comprises a purification tag. In one embodiment, X3 may comprise or consist of the amino acid sequence LEEQKLISEEDLHIIHIHH (SEQ ID NO:98).

In some embodiments, X1 and X3 are both absent. In other embodiments, X1 is present and X3 is absent. In further embodiments, X1 and X3 are both present.

In one embodiment, the polypeptide comprises the amino acid sequence selection from the group consisting of SEQ ID NO: 1-3, 5, 8-10, 13, 23, 26-28, 31-32, 34-38, 40, 42-46, 48-55, 59-60, and 67-74. In a further embodiment, the polypeptide comprises the amino acid sequence selection from the group consisting of SEQ ID NO: 49-55, 59-60, and 67-74. In one embodiment, the polypeptide comprises the amino acid sequence selection from the group consisting of SEQ ID NO: 55, 59, 67, and 73.

In another embodiment, the disclosure provides fusion proteins, comprising
(a) the polypeptide of any embodiment disclosed herein; and
(b) a functional domain linked to the polypeptide, either directly or via an optional amino acid linker. The functional domain may be N-terminal or C-terminal to the polypeptide. In one embodiment, the functional domain is N-terminal to the polypeptide. In one embodiment, the polypeptide domain and the functional domain are linked via an amino acid linker, which may be of any suitable length or amino acid composition. In other embodiments, the polypeptide domain and the functional domain are linked without an intervening amino acid linker.

In one embodiment, the functional domain comprises a polypeptide antigen. In some embodiments, the antigen comprises a bacterial antigen, a viral antigen, a fungal antigen, or a cancer antigen. In other embodiments, the antigen comprises a SARS-CoV-2 antigen or a variant or homolog thereof.

In other embodiments, the antigen comprises an antigen from an infectious agent listed in Table 5, or comprises and antigen listed in Table 6 or an antigenic fragment or mutated version thereof.

In various embodiments of the fusion proteins of the disclosure, the polypeptide comprises the amino acid sequence selection from the group consisting of SEQ ID NO: 10, 13, 23, 26-28, 31-32, 34-38, 40, 42-46, 48, and 59-60, and 67-74; or the polypeptide comprises the amino acid sequence selection from the group consisting of SEQ ID NO: 59-60 and 67-74; or the polypeptide comprises the amino acid sequence selection from the group consisting of SEQ ID NO: 59, 67, and 73.

The disclosure also provides nanoparticles, comprising:
(a) a plurality of first assemblies, each first assembly comprising a plurality of identical first proteins comprising the amino acid sequence selected from the group consisting of SEQ ID NO: 10, 13, 23, and 59-60; and,
(b) a plurality of second assemblies, each second assembly comprising a plurality of second proteins comprising the amino acid sequence selected from the group consisting of SEQ ID NO: 1-3, 5, 8-9, and 49-55;
wherein the plurality of first assemblies non-covalently interact with the plurality of second assemblies to form the nanoparticle.

In one embodiment, each first assembly comprises a plurality of identical first proteins comprising an amino acid sequence selected from the group consisting of SEQ ID NO: 10, 13, and 59-60. In another embodiment, each first assembly comprises a plurality of identical first proteins comprising an amino acid sequence selected from the group consisting of SEQ ID NO: 59-60.

In one embodiment, each second assembly comprising a plurality of second proteins comprising an amino acid sequence selected from the group consisting of SEQ ID NO: 1-3, 5, 8-9, 26-28, 31-32, 34-38, 40, 42-46, 48-55, and 67-74. In another embodiment, each second assembly comprising a plurality of second proteins comprising an amino acid sequence selected from the group consisting of SEQ ID NO: 49-55, and 67-74. In a further embodiment, each second assembly comprising a plurality of second proteins comprising an amino acid sequence selected from the group consisting of SEQ ID NO: 55, 67, and 73.

In another embodiment, the disclosure provides nanoparticles, comprising:
(a) a plurality of first assemblies, each first assembly comprising a plurality of identical first proteins comprising the amino acid sequence selected of SEQ ID NO:152 or 153; and,
(b) a plurality of second assemblies, each second assembly comprising a plurality of second proteins comprising an amino acid sequence selected from the group consisting of SEQ ID NO: 26-48 and 61-77;
wherein the plurality of first assemblies non-covalently interact with the plurality of second assemblies to form the nanoparticle.

In various embodiments, the plurality of second proteins comprise an amino acid sequence selected from the group consisting of SEQ ID NO: 26-28, 31-32, 34-38, 40, 42-46, 48, and 67-77; or the plurality of second proteins comprise an amino acid sequence selected from the group consisting of SEQ ID NO: 67-74; or the plurality of second proteins comprise an amino acid sequence selected from the group consisting of SEQ ID NO: 67 and 73.

In one embodiment of all nanoparticles of the disclosure, some (at least 1%, 5%, 10%, 20%, 25%, 30%, 40%, 50%, 60%, 70%, 75%, 80%, 85%, 90%, 95%) of the second proteins comprise a fusion protein of any embodiment or combination of embodiments herein. In one embodiment, all of the second proteins comprise a fusion protein. In one embodiment, the fusion protein comprises an antigen according to any embodiment disclosed herein, and the nanoparticle displays the antigen(s) on an exterior of the nanoparticle. In some embodiments, each second protein of the nanostructure bears an antigen as a genetic fusion; these nanoparticles display antigen at full (100%) valiancy. In other embodiments, the nanoparticles of the disclosure comprise one or more second proteins bearing antigens as genetic fusions as well as one or more second proteins that do not bear antigens as genetic fusions; these nanoparticles display the antigens at partial valency. In other embodiments, the nanoparticles of the disclosure comprise two or more distinct second proteins bearing different antigens as genetic fusions.

In various embodiments, the nanoparticles are between about 20 nanometers (nm) to about 40 nm in diameter, with interior lumens between about 15 nm to about 32 nm across and pore sizes in the protein shells between about 1 nm to about 14 nm in their longest dimensions.

In another aspect the disclosure provides nucleic acids encoding the polypeptide or fusion protein of any embodiment or combination of embodiments of the disclosure. In a further aspect, the disclosure provides expression vectors comprising the nucleic acid of any aspect of the disclosure operatively linked to a suitable control sequence. In another aspect, the disclosure provides host cells that comprise the polypeptide, fusion protein, nanoparticle, nucleic acid or expression vector (i.e.: episomal or chromosomally integrated) disclosed herein.

In a further aspect, the disclosure provides a composition comprising a plurality of the nucleic acids, expression vectors, proteins, fusion proteins, and/or nanoparticles of the disclosure. In one embodiment, the composition comprises a pharmaceutical composition or an immunogenic composition (such as a vaccine) comprising an effective amount of the nanoparticle of any embodiment or combination of embodiments of the disclosure that incorporates an antigen; and a pharmaceutically acceptable carrier.

In another aspect, the disclosure provides methods for generating an immune response to an antigen in a subject, comprising administering to the subject an effective amount of the immunogenic composition of any embodiment or combination of embodiments of the disclosure to generate the immune response. In a further aspect, the disclosure provides methods for treating or preventing an infection in a subject, comprising administering to the subject an effective amount of the immunogenic composition of any embodiment or combination of embodiments of the disclosure that comprises an antigen, or antigenic fragment thereof, from the infectious agent to be treated or prevented, thereby treating or preventing infection in the subject.

Exemplary antigens and infectious agents are disclosed herein.

### DESCRIPTION OF FIGURES

FIG. 1. Design and Characterization of HA-I53_dn5 Nanoparticle Immunogens with a Glycosylated, PEGylated, or PASylated Scaffold. Structural models of the glycosylated pentameric I53_dn5A_2gly (I53_dn5A, glycans at PNGS 84-NDT-86 and 118-NST-120) (A), PEGylated pentameric I53_dn5A_2C2kPEG (2 kDa PEG at Cys84 and Cys120) (E), PASylated pentameric I53_dn5A_PAS (63 amino acid C-terminal “PAS” polypeptide) (I) and trimeric HA-I53_dn5B (HA, glycans, and I53_dn5B) components. Upon mixing in vitro, 20 trimeric and 12 pentameric components spontaneously assemble to form nanoparticle immunogens with icosahedral symmetry. Each nanoparticle displays 20 HA trimers and is approximately 50 nm in diameter. SEC purification of the HA-I53_dn5_Agly (B), HA-I53_dn5_2C2kPEG (F), and HA-I53_dn5_PAS (J) nanoparticle immunogens after in vitro assembly using a Superose™ 6 Increase 10/300 GL column. The nanoparticle immunogen elutes at the void volume of the column (bar). Residual, unassembled trimeric HA-I53_dn5B component elutes around 16.5 mL. The diameter and polydispersity index (PDI) of SEC-purified nanoparticles measured by DLS is reported at the top of the SEC chromatogram; DLS plots are shown in FIG. 5J. Reducing SDS-PAGE of SEC-purified HA-I53_dn5_Agly (without and with enzymatic cleavage of glycans by ˜35 kDa PNGase F) (C), HA-I53_dn5_2C2kPEG (G), and HA-I53_dn5_PAS (K) nanoparticle immunogens and residual, unassembled trimeric HA-I53_dn5B and pentameric I53_dn5A_PAS components. Representative electron micrographs of negatively-stained HA-I53_dn5_Agly (D), HA-I53_dn5_2C2kPEG (H), and HA-I53_dn5_PAS (L) nanoparticles. Scale bars, 100 nm.

FIG. 2. Glycosylating, PEGylating, or PASylating the Nanoparticle Scaffold of HA-I53_dn5 Immunogens does not Enhance Anti-HA Antibody Responses. (A-D) Post-2^{nd} boost (week 10) anti-H1 MI15 hemagglutinin (A), anti-I53_dn5A pentamer (B), anti-I53_dn5B trimer (C), and anti-I53_dn5 nanoparticle (D) serum IgG binding titers in BALB/c mice, measured by enzyme linked immunosorbent assay (ELISA) and plotted as the area under the curve (AUC) for each serum dilution series. Each symbol represents an individual animal and the geometric mean AUC and the geometric mean standard deviation from each group is indicated by the bar and error bar, respectively (N=5 mice/group). The inset depicts the study timeline and the blood collection time point that each data panel represents. (E) Post-2nd boost (week 10) anti-I53_dn5 nanoparticle and anti-H1 MI15 hemagglutinin serum IgG levels (mg/mL) elicited by HA-I53_dn5 and HA-I53_dn5_2C2kPEG nanoparticle immunogens in BALB/c mice, measured by ELISA. (F-H) Number of I53_dn5A pentamer⁺ (F), I53_dn5B trimer⁺ (G), and H1 MI15 hemagglutinin⁺ (H) lymph node GC precursors and B cells (CD38^{+/-}GL7⁺) detected for each immunization group in BALB/c mice. N=6 across two experiments for each group. (I) Post-prime (week 2), post-1^{st} boost (week 6), and post-2^{nd} boost (week 10) anti-H1 MI15 hemagglutinin geometric mean Ab avidity index. The mouse immunization study was repeated twice, and representative data are shown. The dashed line represents levels for the HA-I53_dn5 immunogen for comparison, and the dotted line represents the lower limit of detection of the assay. Mouse immunization studies were repeated twice, and representative data are shown. P values between groups were determined by Brown-Forsythe and Welch one-way ANOVA test, with Dunnett's T3 multiple comparisons test. *p<0.05; **p<0.01; ***p<0.001; ****p<0.0001.

FIG. 3. Only HIV-1 Env is Subdominant to the Nanoparticle Scaffold in a Series of Different Nanoparticle Immunogens that all Use the Same I53-50 Scaffold. (A) Schematic representation of the series of nanoparticle immunogens used in this study that all use the same I53-50 scaffold, highlighting the structural differences in the displayed antigen for each immunogen. (B) Table listing the nanoparticle and non-assembling control immunogens and schematic depicting the study timeline and blood collection time points that each data panel represents. (C and D) Antigen-specific (C) and I53-50 scaffold-specific (D) serum IgG binding titers in BALB/c mice immunized with the immunogens listed in the table in panel B, measured by ELISA and plotted as the area under the curve (AUC) for each serum dilution series. Antigen-specific IgG titers were measured by Ni-NTA-capture ELISA for more accurate comparison among immunogen groups. Each symbol represents an individual animal and the geometric mean AUC from each group is indicated by the bar (N=10 mice/group). The dashed line in panel D represents levels for the ConM-I53-50 immunogen for comparison. (E) Ratio of the antigen-specific (C) to I53-50 scaffold-specific (D) binding antibody AUC titers. The dashed line indicates a ratio of 1. (F) Spearman's correlations between post-2^{nd} boost (week 10) anti-antigen and anti-I53-50 scaffold serum IgG titers (AUC) for all immunogens on the same plot. Shaded areas represent 95% confidence intervals. Each symbol represents a mouse (N=10 per immunogen). P values between groups were determined by Brown-Forsythe and Welch one-way ANOVA test, with Dunnett's T3 multiple comparisons test. ns, non-significant; *p<0.05; **p<0.01; ***p<0.001; ****p<0.0001.

FIG. 4. Design of Glycosylated I53_dn5 Nanoparticle Scaffolds, Related to FIG. 1. (A and B) Rosetta™ total_energy vs. backbone (Ca) root mean square deviation (RMSD, A) for design models of glycosylated I53_dn5A pentamers (A) and I53_dn5B trimers (B). Dotted lines indicate filter cut-offs for selection of designs to experimentally test for protein expression and glycosylation. (C and D) Reducing western blots of concentrated cell supernatants for single PNGS variants (C) and combination PNGS variants (D) for glycosylated I53_dn5A pentamer and I53_dn5B trimer designs, detected using a mouse anti-myc tag primary mAb and a horse anti-mouse HRP-coupled secondary mAb. Numbers indicate the amino acid residue where an Asn was inserted. enh0, typical (non-enhanced) N-linked sequon; enh1, enhanced N-linked sequon (Huang et al., 2017; Murray et al., 2015). Glycosylated I53_dn5A and I53_dn5B variants carried forward for nanoparticle immunogen assembly and in vivo testing are indicated. L, molecular weight ladder.

FIG. 5. Characterization of Glycosylated, PEGylated, and PASylated I53_dn5 Nanoparticle Scaffolds, Related to FIG. 1. (A, C, and E) SEC purification of the I53_dn5 scaffold masked with glycans (A), PEG (C), or unstructured polypeptides (E) after in vitro assembly using a Superose™ 6 Increase 10/300 GL column. The nanoparticles elute at 9-15 mL and residual, unassembled components elute at larger volumes. In addition to the peak shifts being consistent with the molecular weight of the masking agent, in most cases modest effects on the in vitro assembly efficiency were also observed. (B, D, and F) Reducing SDS-PAGE of SEC-purified I53_dn5 scaffold masked with glycans (B), PEG (D), or unstructured polypeptides (F) and residual, unassembled components. The presence of more unassembled components in the 18.5 mL peak for I53_dn5_Bgly compared to I53_dn5_Agly indicates that the I53_dn5B_2gly component has the lower nanoparticle assembly efficiency (A and B). Similarly, 5 kDa PEG, XTEN, and PAS polypeptides all have larger amounts of unassembled components in the 15-20 mL elution volumes compared to the smaller 1 and 2 kDa PEG and ELP polypeptide, indicating that these larger masking agents impeded nanoparticle assembly efficiency the most (C-F). From the SDS-PAGE presented in panel (D), we estimate PEG conjugation efficiency was >90% in all cases. (G) SEC purification of PEGylated HA-I53_dn5 nanoparticle immunogens after in vitro assembly using a Superose™ 6 Increase 10/300 GL column. The nanoparticle immunogen elutes at the void volume. Residual, unassembled components elute around 15-18 mL. Note the declining in vitro assembly efficiency as the PEG molecular weight increases, suggesting larger PEG sterically hinders nanoparticle assembly when HA is fused to the I53_dn5B trimer. (H) Reducing SDS-PAGE of SEC-purified PEGylated HA-I53_dn5 nanoparticle immunogens and residual, unassembled components. Only excess HA-I53_dn5B trimer was detected in the residual, unassembled component peak for both HA-I53_dn5_1C1kPEG and HA-I53_dn5_1C2kPEG immunogens, confirming complete nanoparticle assembly. However, both HA-I53_dn5B trimer and I53_dn5A_1C5kPEG pentamer were present in the 15.5 mL unassembled component peak for HA-I53_dn5_1C5kPEG immunogen, indicating that 5 kDa PEG on the I53_dn5A pentamer impeded efficient nanoparticle assembly. (I) Reducing SDS-PAGE of I53_dn5A_D120C and I53_dn5A_S84C_D120C pentamers coupled to 1, 2, or 5 kDa PEG. Note the larger molecular weight shifts when PEG is coupled to I53_dn5A_S84C_D120C compared to I53_dn5A_D120C due to the presence of two unpaired cysteines (10 vs. 5 cysteines per pentamer, respectively). We estimate PEG conjugation efficiency was >90% in all cases. (J) Dynamic light scattering of SEC-purified nanoparticle immunogens, including unmodified I53_dn5.

FIG. 6. Masking the I53_dn5 Scaffold Reduces Scaffold-specific Antibody Responses when no Glycoprotein Antigen is Present, but Scaffold Masking does not Enhance Antigen-specific Responses when I53_dn5 and I53-50 Scaffolds Display a Glycoprotein Antigen, Related to FIG. 2. (A-C) Post-2^{nd} boost (week 10) anti-I53_dn5A pentamer (A), anti-I53_dn5B trimer (B), and anti-I53_dn5 nanoparticle (C) serum IgG binding titers in BALB/c mice, measured by ELISA and plotted as the area under the curve (AUC) for each serum dilution series. Each symbol represents an individual animal and the geometric mean AUC and the geometric mean standard deviation from each group is indicated by the bar and error bar, respectively (N=5 mice/group). The dashed line represents levels for the unmodified I53_dn5 nanoparticle for comparison. The inset depicts the study timeline and the blood collection time point that each data panel represents. P values between groups were determined by Brown-Forsythe and Welch one-way ANOVA test, with Dunnett's T3 multiple comparisons test. *p<0.05; **p<0.01; ***p<0.001; ****p<0.0001. (D-K) Post-1^{st} boost (week 6) (D-G) and post-2^{nd} boost (week 10) (H-K) anti-H1MI15 influenza hemagglutinin (D and H), anti-I53_dn5A pentamer (E and I), anti-I53_dn5B trimer (F and J), and anti-I53_dn5 nanoparticle (G and K) serum IgG ELISA curves in BALB/c mice. Each symbol represents the geometric mean absorbance at 450 nm+/− geometric mean SD (N=5 mice/group). (L-O) Post-1^{st} boost (week 6) (L and N) and post-2^{nd} boost (week 10) (M and O) anti-DS-Cav1 RSV F protein (L and M) and anti-I53-50 nanoparticle (N and O) IgG ELISA curves in BALB/c mice. Each symbol represents the geometric mean absorbance at 450 nm+/− geometric mean SD (N=5 mice/group). P values between the 405 nm absorption values for I53-50 and RSV F-I53-50 at the indicated serum dilutions were determined by unpaired t tests. *p<0.05; **p<0.01; ***p<0.001; ****p<0.0001. (P) Representative gating strategy for evaluating I53_dn5A-, I53_dn5B-, and HA-specific B cells, germinal center (GC) precursors and B cells (CD38^{+/-}-GL7⁺), and B cell isotypes. Top row, gating strategy for measuring numbers of live, non-doublet B cells. Bottom row, representative data from a mouse immunized with HA-I53_dn5 formulated with AddaVax. HA⁺CD38^{+/-}-GL7⁺ cells that did not bind decoys were counted as antigen-specific GC precursors and B cells. GC precursors and B cells were further analyzed to characterize B cell receptor isotypes.

FIG. 7. SEC Purification and SDS-PAGE of I53-50-based Nanoparticle Immunogens, Related to FIG. 3. (A and B) SEC chromatograms from purification of the RSV F-I53-50, RBD-I53-50, HA-I53-50, ConM-I53-50, and AMC009-I53-50 nanoparticle immunogens after in vitro assembly using a HiLoad 26/600 Superdexm 200 pg column for RBD-I53-50 and a Superose™ 6 Increase 10/300 GL column for the other nanoparticle immunogens (A), and SDS-PAGE of these nanoparticle immunogens after SEC purification (B).

### DETAILED DESCRIPTION

All references cited are herein incorporated by reference in their entirety. Within this application, unless otherwise stated, the techniques utilized may be found in any of several well-known references such as: Molecular Cloning: A Laboratory Manual (Sambrook, et al., 1989, Cold Spring Harbor Laboratory Press), Gene Expression Technology (Methods in Enzymology, Vol. 185, edited by D. Goeddel, 1991. Academic Press, San Diego, CA), “Guide to Protein Purification” in Methods in Enzymology (M. P. Deutshcer, ed., (1990) Academic Press, Inc.); PCR Protocols: A Guide to Methods and Applications (Innis, et al. 1990. Academic Press, San Diego, CA), Culture of Animal Cells: A Manual of Basic Technique, 2^{nd} Ed. (R. I. Freshney. 1987. Liss, Inc. New York, NY), Gene Transfer and Expression Protocols, pp. 109-128, ed. E. J. Murray, The Humana Press Inc., Clifton, N.J.).

As used herein, the singular forms “a”, “an” and “the” include plural referents unless the context clearly dictates otherwise.

Any N-terminal methionine residues are optional, and may be present in the claimed polypeptides, or may be absent/deleted.

As used herein, the amino acid residues are abbreviated as follows: alanine (Ala; A), asparagine (Asn; N), aspartic acid (Asp; D), arginine (Arg; R), cysteine (Cys; C), glutamic acid (Glu; E), glutamine (Gln; Q), glycine (Gly; G), histidine (His; H), isoleucine (Ile; I), leucine (Leu; L), lysine (Lys; K), methionine (Met; M), phenylalanine (Phe; F), proline (Pro; P), serine (Ser; S), threonine (Thr; T), tryptophan (Trp; W), tyrosine (Tyr; Y), and valine (Val; V).

All embodiments of any aspect of the disclosure can be used in combination, unless the context clearly dictates otherwise.

Unless the context clearly requires otherwise, throughout the description and the claims, the words ‘comprise’, ‘comprising’, and the like are to be construed in an inclusive sense as opposed to an exclusive or exhaustive sense; that is to say, in the sense of “including, but not limited to”. Words using the singular or plural number also include the plural and singular number, respectively. Additionally, the words “herein,” “above,” and “below” and words of similar import, when used in this application, shall refer to this application as a whole and not to any particular portions of the application.

The description of embodiments of the disclosure is not intended to be exhaustive or to limit the disclosure to the precise form disclosed. While the specific embodiments of, and examples for, the disclosure are described herein for illustrative purposes, various equivalent modifications are possible within the scope of the disclosure, as those skilled in the relevant art will recognize.

In a first aspect, the disclosure provides polypeptides comprising the amino acid sequence of I53_dn5A (SEQ ID NO: 78), I53_dn5B (SEQ ID NO:79), or I53-50A (SEQ ID NO:80), substituted with one or more sequon, wherein the N-terminal residue may be present or may be absent. As used herein, a sequon is a sequence of consecutive amino acids that can serve as the attachment site to a polysaccharide.

The polypeptides of the disclosure have the ability to self-assemble in pairs to form nanoparticles that can be used, for example, to display antigens on the exterior surface of the nanoparticle. The nanoparticles so formed include symmetrically repeated, non-covalent polypeptide-polypeptide interfaces that orient a first assembly and a second assembly into a nanoparticle. The attachment of glycans to the polypeptides via the sequons, and nanoparticles comprising the glycosylated polypeptides, helps mimic the natural presentation of sugars on glycoproteins, optimize the pharmacokinetics and biologic activity of protein nanoparticles, and dissect the importance of different protein-carbohydrate combinations for the various applications that protein nanoparticles may be used for (e.g., as vaccine scaffolds and for drug delivery). For example, protein nanoparticle immunogens bearing high-mannose N-linked glycans can traffic more efficiently to draining lymph nodes and B cell follicles in vivo, resulting in enhanced germinal center formation and antibody responses against the displayed antigen or nanoparticle immunogen.

The sequences of SEQ ID NO:78-80 are shown in Table 1. The polypeptides of the disclosure include one or more sequons that replace (“substitute”) amino acid residues in the reference sequence.

TABLE 1

| | |
|---|---|
| I53_dn5A | KYDGSKLRIGILHARGNAEIILELVLGALKRLQE |
| (SEQ ID NO: 78) | FGVKRENIIIETVPGSFELPYGSKLFVEKQKRLG |
| | KPLDAIIPIGVLIRGSTAHFDYIADSTTHQLMKL |
| | NFELGIPVIFGVLTTESDEQAEERAGTKAGNHGE |
| | DWGAAAVEMATKFN |
| I53-dn5 B | EEAELAYLLGELAYKLGEYRIAIRAYRIALKRDP |
| (SEQ ID NO 79:) | NNAEAWYNLGNAYYKQGRYREAIEYYQKALELDP |
| | NNAEAWYNLGNAYYERGEYEEAIEYYRKALRLDP |
| | NNADAMQNLLNAKMREE |
| I53-50A | MKMEELFKKHKIVAVLRANSVEEAIEKAVAVFAG |
| SEQ ID NO: 80 | GVHLIEITFTVPDADTVIKALSVLKEKGAIIGAG |
| | TVTSVEQCRKAVESGAEFIVSPHLDEEISQFCKE |
| | KGVFYMPGVMTPTELVKAMKLGHTILKLFPGEVV |
| | GPQFVKAMKGPFPNVKFVPTGGVNLDNVCEWFKA |
| | GVLAVGVGSALVKGTPDEVREKAKAFVEKIRGCT |
| | E |

In various embodiments, each sequon may independently consist of the amino acid sequence selected from the group consisting of NET, NDS, NST, FSNES (SEQ ID NO:81), NES, FENES (SEQ ID NO:82), NAS, NGS, NHT, FFNHT (SEQ ID NO:83), NLS, FDNLS (SEQ ID NO:84), NNS, WHNNS (SEQ ID NO:85), NYS, FINYS (SEQ ID NO:86), NIS, FLNAT (SEQ ID NO:87), NAT, FLNAS (SEQ ID NO:88), WVNNS (SEQ ID NO:89), NKS, YLNKS (SEQ ID NO:90), FSNET (SEQ ID NO:91), YVNVT (SEQ ID NO:92), NRS, YANRS (SEQ ID NO:93), WANAS (SEQ ID NO:94), NFT, WANFT (SEQ ID NO:95), NVS, NGT, NVT, WLNHT (SEQ ID NO:96), and NTS.

The polypeptide may be substituted with a single sequon or multiple sequons. If substituted with multiple sequons, each sequon may be the same or may be different.

In certain embodiments, the polypeptide comprises the amino acid sequence selected from the group consisting of SEQ ID NO: 1-3, 5, 8-10, 13, 23, 26-28, 31-32, 34-38, 40, 42-46, 48-55, 59-60, and 67-74, wherein:
(a) each sequon may independently be substituted with any other sequon;
(b) X1 may be present or absent, and when present comprises a signal peptide; and
(c) X2 may be present or absent, and when present comprises a purification tag.

The amino acid sequence of these exemplified polypeptides are provided in Table 2, with the sequons underlined.

TABLE 2
Sequence ID 1 (I53_dn5A_1gly) N-linked glycan sequons are underlined
X1-
KYDGSKLRIGILHARGNAEIILELVLGALKRLQEFGVKRENIIIETVPGSFELPYGSKLFVEKQKRLGK
PLDAIIPIGVLIRNETAHFDYIADSTTHQLMKLNFELGIPVIFGVLTTESDEQAEERAGTKAGNHGEDW
GAAAVEMATKFN-(X2) (SEQ ID NO: 1)
Sequence ID 2 (I53_dn5A_1gly) N-linked glycan sequons are underlined
(X1)-
KYDGSKLRIGILHARGNAEIILELVLGALKRLQEFGVKRENIIIETVPGSFELPYGSKLFVEKQKRLGK
PLDAIIPIGVLIRGNDSHFDYIADSTTHQLMKLNFELGIPVIFGVLTTESDEQAEERAGTKAGNHGEDW
GAAAVEMATKFN-(X2) (SEQ ID NO: 2)
Sequence ID 3 (I53_dn5A_1gly) N-linked glycan sequons are underlined
(X1)-
KYDGSKLRIGILHARGNAEIILELVLGALKRLQEFGVKRENIIIETVPGSFELPYGSKLFVEKQKRLGK
PLDAIIPIGVLIRGSTAHFDYIADSTTHQLMKLNFELGIPVIFGVLTTNSTEQAEERAGTKAGNHGEDW
GAAAVEMATKFN-(X2) (SEQ ID NO: 3)
Sequence ID 4 (I53_dn5A_1gly) N-linked glycan sequons are underlined
(X1)-
KYDGSKLRIGILHARGNAEIILELVLGALKRLQEFGVKRENIIIETVPGSFELPYGSKLFVEKQKRLGK
PLDAIIPIGVLIRGSTAHFDYIADSTTHQLMKLNFELGIPVIFGVLTTESNESAEERAGTKAGNHGEDW
GAAAVEMATKFN-(X2) (SEQ ID NO: 4)
Sequence ID 5 (I53_dn5A_1gly) N-linked glycan sequons are underlined
(X1)-
KYDGSKLRIGILHARGNAEIILELVLGALKRLQEFGVKRENIIIETVPGSFELPYGSKLFVEKQKRLGK
PLDAIIPIGVLIRGSTAHFDYIADSTTHQLMKLNFELGIPVIFGVLTTFSNESAEERAGTKAGNHGEDW
GAAAVEMATKFN-(X2) (SEQ ID NO: 5)
Sequence ID 6 (I53_dn5A_1gly) N-linked glycan sequons are underlined
(X1)-
KYDGSKLRIGILHARGNAEIILELVLGALKRLQEFGVKRENIIIETVPGSFELPYGSKLFVEKQKRLGK
PLDAIIPIGVLIRGSTAHFDYIADSTTHQLMKLNFELGIPVIFGVLTTESDNESEERAGTKAGNHGEDW
GAAAVEMATKFN-(X2) (SEQ ID NO: 6)
Sequence ID 7 (I53_dn5A_1gly) N-linked glycan sequons are underlined
(X1)-
KYDGSKLRIGILHARGNAEIILELVLGALKRLQEFGVKRENIIIETVPGSFELPYGSKLFVEKQKRLGK
PLDAIIPIGVLIRGSTAHFDYIADSTTHQLMKLNFELGIPVIFGVLTTEFENESEERAGTKAGNHGEDW
GAAAVEMATKFN-(X2) (SEQ ID NO: 7)
Sequence ID 8 (I53_dn5A_1gly) N-linked glycan sequons are underlined
(X1)-
KYDGSKLRIGILHARGNAEIILELVLGALKRLQEFGVKRENIIIETVPGSFELPYGSKLFVEKQKRLGK
PLDAIIPIGVLIRGSTAHFDYIADSTTHQLMKLNFELGIPVIFGVLTTESDEQAEERAGTNASNHGEDW
GAAAVEMATKFN-(X2) (SEQ ID NO: 8)
Sequence ID 9 (I53_dn5A_1gly) N-linked glycan sequons are underlined
(X1)-
KYDGSKLRIGILHARGNAEIILELVLGALKRLQEFGVKRENIIIETVPGSFELPYGSKLFVEKQKRLGK
PLDAIIPIGVLIRGSTAHFDYIADSTTHQLMKLNFELGIPVIFGVLTTESDEQAEERAGTKNGSHGEDW
GAAAVEMATKFN-(X2) (SEQ ID NO: 9)
Sequence ID 10 (I53_dn5B_1gly) N-linked glycan sequons are underlined
(X1)-
EEAELAYLLGELAYKLGEYRIAIRAYRIALKRNHTNAEAWYNLGNAYYKQGRYREAIEYYQKALELDPN
NAEAWYNLGNAYYERGEYEEAIEYYRKALRLDPNNADAMQNLLNAKMREE-(X2) (SEQ ID NO: 10)
Sequence ID 11 (I53_dn5B_1gly) N-linked glycan sequons are underlined
(X1)-
EEAELAYLLGELAYKLGEYRIAIRAYRIALFFNHTNAEAWYNLGNAYYKQGRYREAIEYYQKALELDPN
NAEAWYNLGNAYYERGEYEEAIEYYRKALRLDPNNADAMQNLLNAKMREE-(X2) (SEQ ID NO: 11)
Sequence ID 12 (I53_dn5B_1gly) N-linked glycan sequons are underlined
(X1)-
EEAELAYLLGELAYKLGEYRIAIRAYRIALKRDNLSAEAWYNLGNAYYKQGRYREAIEYYQKALELDPN
NAEAWYNLGNAYYERGEYEEAIEYYRKALRLDPNNADAMQNLLNAKMREE-(X2) (SEQ ID NO: 12)
Sequence ID 13 (I53_dn5B_1gly) N-linked glycan sequons are underlined
(X1)-
EEAELAYLLGELAYKLGEYRIAIRAYRIALKFDNLSAEAWYNLGNAYYKQGRYREAIEYYQKALELDPN
NAEAWYNLGNAYYERGEYEEAIEYYRKALRLDPNNADAMQNLLNAKMREE-(X2) (SEQ ID NO: 13)
Sequence ID 14 (I53_dn5B_1gly) N-linked glycan sequons are underlined
(X1)-
EEAELAYLLGELAYKLGEYRIAIRAYRIALKRDPNNSEAWYNLGNAYYKQGRYREAIEYYQKALELDPN
NAEAWYNLGNAYYERGEYEEAIEYYRKALRLDPNNADAMQNLLNAKMREE-(X2) (SEQ ID NO: 14)
Sequence ID 15 (I53_dn5B_1gly) N-linked glycan sequons are underlined
(X1)-
EEAELAYLLGELAYKLGEYRIAIRAYRIALKRWHNNSEAWYNLGNAYYKQGRYREAIEYYQKALELDPN
NAEAWYNLGNAYYERGEYEEAIEYYRKALRLDPNNADAMQNLLNAKMREE-(X2) (SEQ ID NO: 15)
Sequence ID 16 (I53_dn5B_1gly) N-linked glycan sequons are underlined
(X1)-
EEAELAYLLGELAYKLGEYRIAIRAYRIALKRDPNNAEAWYNLGNAYYKQGRYREAINYSQKALELDPN
NAEAWYNLGNAYYERGEYEEAIEYYRKALRLDPNNADAMQNLLNAKMREE-(X2) (SEQ ID NO: 16)
Sequence ID 17 (I53_dn5B_1gly) N-linked glycan sequons are underlined
(X1)-
EEAELAYLLGELAYKLGEYRIAIRAYRIALKRDPNNAEAWYNLGNAYYKQGRYREFINYSQKALELDPN
NAEAWYNLGNAYYERGEYEEAIEYYRKALRLDPNNADAMQNLLNAKMREE-(X2) (SEQ ID NO: 17)
Sequence ID 18 (I53_dn5B_1gly) N-linked glycan sequons are underlined
(X1)-
EEAELAYLLGELAYKLGEYRIAIRAYRIALKRDPNNAEAWYNLGNAYYKQGRYREAIEYYNISLELDPN
NAEAWYNLGNAYYERGEYEEAIEYYRKALRLDPNNADAMQNLLNAKMREE-(X2) (SEQ ID NO: 18)
Sequence ID 19 (I53_dn5B_1gly) N-linked glycan sequons are underlined
(X1)-
EEAELAYLLGELAYKLGEYRIAIRAYRIALKRDPNNAEAWYNLGNAYYKQGRYREAIEYFLNATELDPN
NAEAWYNLGNAYYERGEYEEAIEYYRKALRLDPNNADAMQNLLNAKMREE-(X2) (SEQ ID NO: 19)
Sequence ID 20 (I53_dn5B_1gly) N-linked glycan sequons are underlined
(X1)-
EEAELAYLLGELAYKLGEYRIAIRAYRIALKRDPNNAEAWYNLGNAYYKQGRYREAIEYYQKALNLSPN
NAEAWYNLGNAYYERGEYEEAIEYYRKALRLDPNNADAMQNLLNAKMREE-(X2) (SEQ ID NO: 20)
Sequence ID 21 (I53_dn5B_1gly) N-linked glycan sequons are underlined
(X1)-
EEAELAYLLGELAYKLGEYRIAIRAYRIALKRDPNNAEAWYNLGNAYYKQGRYREAIEYYQKALELDNL
SAEAWYNLGNAYYERGEYEEAIEYYRKALRLDPNNADAMQNLLNAKMREE-(X2) (SEQ ID NO: 21)
Sequence ID 22 (I53_dn5B_1gly) N-linked glycan sequons are underlined
(X1)-
EEAELAYLLGELAYKLGEYRIAIRAYRIALKRDPNNAEAWYNLGNAYYKQGRYREAIEYYQKALEFDNL
SAEAWYNLGNAYYERGEYEEAIEYYRKALRLDPNNADAMQNLLNAKMREE-(X2) (SEQ ID NO: 22)
Sequence ID 23 (I53_dn5B_1gly) N-linked glycan sequons are underlined
(X1)-
EEAELAYLLGELAYKLGEYRIAIRAYRIALKRDPNNAEAWYNLGNAYYKQGRYREAIEYYQKALELDPN
NAEAWYNLGNAYYERGEYENATEYYRKALRLDPNNADAMQNLLNAKMREE-(X2) (SEQ ID NO: 23)
Sequence ID 24 (I53_dn5B_1gly) N-linked glycan sequons are underlined
(X1)-
EEAELAYLLGELAYKLGEYRIAIRAYRIALKRDPNNAEAWYNLGNAYYKQGRYREAIEYYQKALELDPN
NAEAWYNLGNAYYERGEYEEAIEYFLNASRLDPNNADAMQNLLNAKMREE-(X2) (SEQ ID NO: 24)
Sequence ID 25 (I53_dn5B_1gly) N-linked glycan sequons are underlined
(X1)-
EEAELAYLLGELAYKLGEYRIAIRAYRIALKRDPNNAEAWYNLGNAYYKQGRYREAIEYYQKALELDPN
NAEAWYNLGNAYYERGEYEEAIEYYRKALRNNSNNADAMQNLLNAKMREE-(X2) (SEQ ID NO: 25)
Sequence ID 26 (I53-50A_1gly) N-linked glycan sequons are underlined
(X1)-
EELFKKHKIVAVLRANSVEEAIEKAVAVFAGGVHLIEITFTVNNSDTVIKALSVLKEKGAIIGAGTVTS
VEQCRKAVESGAEFIVSPHLDEEISQFCKEKGVFYMPGVMTPTELVKAMKLGHTILKLFPGEVVGPQFV
KAMKGPFPNVKFVPTGGVNLDNVCEWFKAGVLAVGVGSALVKGTPDEVREKAKAFVEKIRGC-(X2)
(SEQ ID NO: 26)
Sequence ID 27 (I53-50A_1gly) N-linked glycan sequons are underlined
(X1)-
EELFKKHKIVAVLRANSVEEAIEKAVAVFAGGVHLIEITFWVNNSDTVIKALSVLKEKGAIIGAGTVTS
VEQCRKAVESGAEFIVSPHLDEEISQFCKEKGVFYMPGVMTPTELVKAMKLGHTILKLFPGEVVGPQFV
KAMKGPFPNVKFVPTGGVNLDNVCEWFKAGVLAVGVGSALVKGTPDEVREKAKAFVEKIRGC-(X2)
(SEQ ID NO: 27)
Sequence ID 28 (I53-50A_1gly) N-linked glycan sequons are underlined
(X1)-
EELFKKHKIVAVLRANSVEEAIEKAVAVFAGGVHLIEITFTVPNASTVIKALSVLKEKGAIIGAGTVTS
VEQCRKAVESGAEFIVSPHLDEEISQFCKEKGVFYMPGVMTPTELVKAMKLGHTILKLFPGEVVGPQFV
KAMKGPFPNVKFVPTGGVNLDNVCEWFKAGVLAVGVGSALVKGTPDEVREKAKAFVEKIRGC-(X2)
(SEQ ID NO: 28)
Sequence ID 29 (I53-50A_1gly) N-linked glycan sequons are underlined
(X1)-
EELFKKHKIVAVLRANSVEEAIEKAVAVFAGGVHLIEITFTVPDADTVIKALSVLNKSGAIIGAGTVTS
VEQCRKAVESGAEFIVSPHLDEEISQFCKEKGVFYMPGVMTPTELVKAMKLGHTILKLFPGEVVGPQFV
KAMKGPFPNVKFVPTGGVNLDNVCEWFKAGVLAVGVGSALVKGTPDEVREKAKAFVEKIRGC-(X2)
(SEQ ID NO: 29)
Sequence ID 30 (I53-50A_1gly) N-linked glycan sequons are underlined
(X1)-
EELFKKHKIVAVLRANSVEEAIEKAVAVFAGGVHLIEITFTVPDADTVIKALSYLNKSGAIIGAGTVTS
VEQCRKAVESGAEFIVSPHLDEEISQFCKEKGVFYMPGVMTPTELVKAMKLGHTILKLFPGEVVGPQFV
KAMKGPFPNVKFVPTGGVNLDNVCEWFKAGVLAVGVGSALVKGTPDEVREKAKAFVEKIRGC-(X2)
(SEQ ID NO: 30)
Sequence ID 31 (I53-50A_1gly) N-linked glycan sequons are underlined
(X1)-
EELFKKHKIVAVLRANSVEEAIEKAVAVFAGGVHLIEITFTVPDADTVIKALSVLKEKGAIIGAGTVFS
NETCRKAVESGAEFIVSPHLDEEISQFCKEKGVFYMPGVMTPTELVKAMKLGHTILKLFPGEVVGPQFV
KAMKGPFPNVKFVPTGGVNLDNVCEWFKAGVLAVGVGSALVKGTPDEVREKAKAFVEKIRGC-(X2)
(SEQ ID NO: 31)
Sequence ID 32 (I53-50A_1gly) N-linked glycan sequons are underlined
(X1)-
EELFKKHKIVAVLRANSVEEAIEKAVAVFAGGVHLIEITFTVPDADTVIKALSVLKEKGAIIGAGTVTY
VNVTRKAVESGAEFIVSPHLDEEISQFCKEKGVFYMPGVMTPTELVKAMKLGHTILKLFPGEVVGPQFV
KAMKGPFPNVKFVPTGGVNLDNVCEWFKAGVLAVGVGSALVKGTPDEVREKAKAFVEKIRGC-(X2)
(SEQ ID NO: 32)
Sequence ID 33 (I53-50A_1gly) N-linked glycan sequons are underlined
(X1)-
EELFKKHKIVAVLRANSVEEAIEKAVAVFAGGVHLIEITFTVPDADTVIKALSVLKEKGAIIGAGTVTS
VEQCNRSVESGAEFIVSPHLDEEISQFCKEKGVFYMPGVMTPTELVKAMKLGHTILKLFPGEVVGPQFV
KAMKGPFPNVKFVPTGGVNLDNVCEWFKAGVLAVGVGSALVKGTPDEVREKAKAFVEKIRGC-(X2)
(SEQ ID NO: 33)
Sequence ID 34 (I53-50A_1gly) N-linked glycan sequons are underlined
(X1)-
EELFKKHKIVAVLRANSVEEAIEKAVAVFAGGVHLIEITFTVPDADTVIKALSVLKEKGAIIGAGTVTS
VEYANRSVESGAEFIVSPHLDEEISQFCKEKGVFYMPGVMTPTELVKAMKLGHTILKLFPGEVVGPQFV
KAMKGPFPNVKFVPTGGVNLDNVCEWFKAGVLAVGVGSALVKGTPDEVREKAKAFVEKIRGC-(X2)
(SEQ ID NO: 34)
Sequence ID 35 (I53-50A_1gly) N-linked glycan sequons are underlined
(X1)-
EELFKKHKIVAVLRANSVEEAIEKAVAVFAGGVHLIEITFTVPDADTVIKALSVLKEKGAIIGAGTVTS
VEQCRKAVNASAEFIVSPHLDEEISQFCKEKGVFYMPGVMTPTELVKAMKLGHTILKLFPGEVVGPQFV
KAMKGPFPNVKFVPTGGVNLDNVCEWFKAGVLAVGVGSALVKGTPDEVREKAKAFVEKIRGC-(X2)
(SEQ ID NO: 35)
Sequence ID 36 (I53-50A_1gly) N-linked glycan sequons are underlined
(X1)-
EELFKKHKIVAVLRANSVEEAIEKAVAVFAGGVHLIEITFTVPDADTVIKALSVLKEKGAIIGAGTVTS
VEQCRKAVESNASFIVSPHLDEEISQFCKEKGVFYMPGVMTPTELVKAMKLGHTILKLFPGEVVGPQFV
KAMKGPFPNVKFVPTGGVNLDNVCEWFKAGVLAVGVGSALVKGTPDEVREKAKAFVEKIRGC-(X2)
(SEQ ID NO: 36)
Sequence ID 37 (I53-50A_1gly) N-linked glycan sequons are underlined
(X1)-
EELFKKHKIVAVLRANSVEEAIEKAVAVFAGGVHLIEITFTVPDADTVIKALSVLKEKGAIIGAGTVTS
VEQCRKAVWANASFIVSPHLDEEISQFCKEKGVFYMPGVMTPTELVKAMKLGHTILKLFPGEVVGPQFV
KAMKGPFPNVKFVPTGGVNLDNVCEWFKAGVLAVGVGSALVKGTPDEVREKAKAFVEKIRGC-(X2)
(SEQ ID NO: 37)
Sequence ID 38 (I53-50A_1gly) N-linked glycan sequons are underlined
(X1)-
EELFKKHKIVAVLRANSVEEAIEKAVAVFAGGVHLIEITFTVPDADTVIKALSVLKEKGAIIGAGTVTS
VEQCRKAVESGANFTVSPHLDEEISQFCKEKGVFYMPGVMTPTELVKAMKLGHTILKLFPGEVVGPQFV
KAMKGPFPNVKFVPTGGVNLDNVCEWFKAGVLAVGVGSALVKGTPDEVREKAKAFVEKIRGC-(X2)
(SEQ ID NO: 38)
Sequence ID 39 (I53-50A_1gly) N-linked glycan sequons are underlined
(X1)-
EELFKKHKIVAVLRANSVEEAIEKAVAVFAGGVHLIEITFTVPDADTVIKALSVLKEKGAIIGAGTVTS
VEQCRKAVESWANFTVSPHLDEEISQFCKEKGVFYMPGVMTPTELVKAMKLGHTILKLFPGEVVGPQFV
KAMKGPFPNVKFVPTGGVNLDNVCEWFKAGVLAVGVGSALVKGTPDEVREKAKAFVEKIRGC-(X2)
(SEQ ID NO: 39)
Sequence ID 40 (I53-50A_1gly) N-linked glycan sequons are underlined
(X1)-
EELFKKHKIVAVLRANSVEEAIEKAVAVFAGGVHLIEITFTVPDADTVIKALSVLKEKGAIIGAGTVTS
VEQCRKAVESGAEFIVSPHLDEEISNFTKEKGVFYMPGVMTPTELVKAMKLGHTILKLFPGEVVGPQFV
KAMKGPFPNVKFVPTGGVNLDNVCEWFKAGVLAVGVGSALVKGTPDEVREKAKAFVEKIRGC-(X2)
(SEQ ID NO: 40)
Sequence ID 41 (I53-50A_1gly) N-linked glycan sequons are underlined
(X1)-
EELFKKHKIVAVLRANSVEEAIEKAVAVFAGGVHLIEITFTVPDADTVIKALSVLKEKGAIIGAGTVTS
VEQCRKAVESGAEFIVSPHLDEEISQFCNESGVFYMPGVMTPTELVKAMKLGHTILKLFPGEVVGPQFV
KAMKGPFPNVKFVPTGGVNLDNVCEWFKAGVLAVGVGSALVKGTPDEVREKAKAFVEKIRGC-(X2)
(SEQ ID NO: 41)
Sequence ID 42 (I53-50A_1gly) N-linked glycan sequons are underlined
(X1)-
EELFKKHKIVAVLRANSVEEAIEKAVAVFAGGVHLIEITFTVPDADTVIKALSVLKEKGAIIGAGTVTS
VEQCRKAVESGAEFIVSPHLDEEISQFCKNASVFYMPGVMTPTELVKAMKLGHTILKLFPGEVVGPQFV
KAMKGPFPNVKFVPTGGVNLDNVCEWFKAGVLAVGVGSALVKGTPDEVREKAKAFVEKIRGC-(X2)
(SEQ ID NO: 42)
Sequence ID 43 (I53-50A_1gly) N-linked glycan sequons are underlined
(X1)-
EELFKKHKIVAVLRANSVEEAIEKAVAVFAGGVHLIEITFTVPDADTVIKALSVLKEKGAIIGAGTVTS
VEQCRKAVESGAEFIVSPHLDEEISQFCKEKNVSYMPGVMTPTELVKAMKLGHTILKLFPGEVVGPQFV
KAMKGPFPNVKFVPTGGVNLDNVCEWFKAGVLAVGVGSALVKGTPDEVREKAKAFVEKIRGC-(X2)
(SEQ ID NO: 43)
Sequence ID 44 (I53-50A_1gly) N-linked glycan sequons are underlined
(X1)-
EELFKKHKIVAVLRANSVEEAIEKAVAVFAGGVHLIEITFTVPDADTVIKALSVLKEKGAIIGAGTVTS
VEQCRKAVESGAEFIVSPHLDEEISQFCKEKGVFYMPGVMTPTELVKAMKNGTTILKLFPGEVVGPQFV
KAMKGPFPNVKFVPTGGVNLDNVCEWFKAGVLAVGVGSALVKGTPDEVREKAKAFVEKIRGC-(X2)
(SEQ ID NO: 44)
Sequence ID 45 (I53-50A_1gly) N-linked glycan sequons are underlined
(X1)-
EELFKKHKIVAVLRANSVEEAIEKAVAVFAGGVHLIEITFTVPDADTVIKALSVLKEKGAIIGAGTVTS
VEQCRKAVESGAEFIVSPHLDEEISQFCKEKGVFYMPGVMTPTELVKAMKLNVTILKLFPGEVVGPQFV
KAMKGPFPNVKFVPTGGVNLDNVCEWFKAGVLAVGVGSALVKGTPDEVREKAKAFVEKIRGC-(X2)
(SEQ ID NO: 45)
Sequence ID 46 (I53-50A_1gly) N-linked glycan sequons are underlined
(X1)-
EELFKKHKIVAVLRANSVEEAIEKAVAVFAGGVHLIEITFTVPDADTVIKALSVLKEKGAIIGAGTVTS
VEQCRKAVESGAEFIVSPHLDEEISQFCKEKGVFYMPGVMTPTELVKAMWLNHTILKLFPGEVVGPQFV
KAMKGPFPNVKFVPTGGVNLDNVCEWFKAGVLAVGVGSALVKGTPDEVREKAKAFVEKIRGC-(X2)
(SEQ ID NO: 46)
Sequence ID 47 (I53-50A_1gly) N-linked glycan sequons are underlined
(X1)-
EELFKKHKIVAVLRANSVEEAIEKAVAVFAGGVHLIEITFTVPDADTVIKALSVLKEKGAIIGAGTVTS
VEQCRKAVESGAEFIVSPHLDEEISQFCKEKGVFYMPGVMTPTELVKAMKLGHTILKLFPGEVVGPQFV
KAMKGPFPNTSFVPTGGVNLDNVCEWFKAGVLAVGVGSALVKGTPDEVREKAKAFVEKIRGC-(X2)
(SEQ ID NO: 47)
Sequence ID 48 (I53-50A_1gly) N-linked glycan sequons are underlined
(X1)-
EELFKKHKIVAVLRANSVEEAIEKAVAVFAGGVHLIEITFTVPDADTVIKALSVLKEKGAIIGAGTVTS
VEQCRKAVESGAEFIVSPHLDEEISQFCKEKGVFYMPGVMTPTELVKAMKLGHTILKLFPGEVVGPQFV
KAMKGPFHNTSFVPTGGVNLDNVCEWFKAGVLAVGVGSALVKGTPDEVREKAKAFVEKIRGC-(X2)
(SEQ ID NO: 48)
Sequence ID 49 (I53_dn5A_3gly) N-linked glycan sequons are underlined
(X1)-
KYDGSKLRIGILHARGNAEIILELVLGALKRLQEFGVKRENIIIETVPGSFELPYGSKLFVEKQKRLGK
PLDAIIPIGVLIRNETAHFDYIADSTTHQLMKLNFELGIPVIFGVLTTNDTEQAEERAGTNATNHGEDW
GAAAVEMATKFN-(X2) (SEQ ID NO: 49)
Sequence ID 50 (I53_dn5A_3gly) N-linked glycan sequons are underlined
(X1)-
KYDGSKLRIGILHARGNAEIILELVLGALKRLQEFGVKRENIIIETVPGSFELPYGSKLFVEKQKRLGK
PLDAIIPIGVLIRGNDTHEDYIADSTTHQLMKLNFELGIPVIFGVLTTESNETAEERAGTKNGTHGEDW
GAAAVEMATKFN-(X2) (SEQ ID NO: 50)
Sequence ID 51 (I53_dn5A_3gly) N-linked glycan sequons are underlined
(X1)-
KYDGSKLRIGILHARGNAEIILELVLGALKRLQEFGVKRENIIIETVPGSFELPYGSKLFVEKQKRLGK
PLDAIIPIGVLIRGNDTHEDYIADSTTHQLMKLNFELGIPVIFGVLTTNDTEQAEERAGTNATNHGEDW
GAAAVEMATKFN-(X2) (SEQ ID NO: 51)
Sequence ID 52 (I53_dn5A_2gly) N-linked glycan sequons are underlined
(X1)-
KYDGSKLRIGILHARGNAEIILELVLGALKRLQEFGVKRENIIIETVPGSFELPYGSKLFVEKQKRLGK
PLDAIIPIGVLIRNDTAHFDYIADSTTHQLMKLNFELGIPVIFGVLTTESDEQAEERAGTNATNHGEDW
GAAAVEMATKFN-(X2) (SEQ ID NO: 52)
Sequence ID 53 (I53_dn5A_2gly) N-linked glycan sequons are underlined
(X1)-
KYDGSKLRIGILHARGNAEIILELVLGALKRLQEFGVKRENIIIETVPGSFELPYGSKLFVEKQKRLGK
PLDAIIPIGVLIRGNDTHEDYIADSTTHQLMKLNFELGIPVIFGVLTTESDEQAEERAGTKNGTHGEDW
GAAAVEMATKFN-(X2) (SEQ ID NO: 53)
Sequence ID 54 (I53_dn5A_2gly) N-linked glycan sequons are underlined
(X1)-
KYDGSKLRIGILHARGNAEIILELVLGALKRLQEFGVKRENIIIETVPGSFELPYGSKLFVEKQKRLGK
PLDAIIPIGVLIRGNDTHEDYIADSTTHQLMKLNFELGIPVIFGVLTTNSTEQAEERAGTKAGNHGEDW
GAAAVEMATKFN-(X2) (SEQ ID NO: 54)
Sequence ID 55 (I53_dn5A_2gly) N-linked glycan sequons are underlined
(X1)-
KYDGSKLRIGILHARGNAEIILELVLGALKRLQEFGVKRENIIIETVPGSFELPYGSKLFVEKQKRLGK
PLDAIIPIGVLIRGSTAHFDYIADSTTHQLMKLNFELGIPVIFGVLTTESNETAEERAGTNATNHGEDW
GAAAVEMATKFN-(X2) (SEQ ID NO: 55)
Sequence ID 56 (I53_dn5B_3gly) N-linked glycan sequons are underlined
(X1)-
EEAELAYLLGELAYKLGEYRIAIRAYRIALKRNHTNAEAWYNLGNAYYKQGRYREAIEYYQKALELNHT
NAEAWYNLGNAYYERGEYENATEYYRKALRLDPNNADAMQNLLNAKMREE-(X2) (SEQ ID NO: 56)
Sequence ID 57 (I53_dn5B_3gly) N-linked glycan sequons are underlined
(X1)-
EEAELAYLLGELAYKLGEYRIAIRAYRIALKYDNLTAEAWYNLGNAYYKQGRYREAIEYYQKALELNHT
NAEAWYNLGNAYYERGEYENATEYYRKALRLDPNNADAMQNLLNAKMREE-(X2) (SEQ ID NO: 57)
Sequence ID 58 (I53_dn5B_2gly) N-linked glycan sequons are underlined
(X1)-
EEAELAYLLGELAYKLGEYRIAIRAYRIALKRNHTNAEAWYNLGNAYYKQGRYREAIEYYQKALELDPN
NAEAWYNLGNAYYERGEYENATEYYRKALRLDPNNADAMQNLLNAKMREE-(X2) (SEQ ID NO: 58)
Sequence ID 59 (I53_dn5B_2gly) N-linked glycan sequons are underlined
(X1)-
EEAELAYLLGELAYKLGEYRIAIRAYRIALKYDNLTAEAWYNLGNAYYKQGRYREAIEYYQKALELDPN
NAEAWYNLGNAYYERGEYENATEYYRKALRLDPNNADAMQNLLNAKMREE-(X2) (SEQ ID NO: 59)
Sequence ID 60 (I53_dn5B_2gly) N-linked glycan sequons are underlined
(X1)-
EEAELAYLLGELAYKLGEYRIAIRAYRIALKRDPNNAEAWYNLGNAYYKQGRYREAIEYYQKALELNHT
NAEAWYNLGNAYYERGEYENATEYYRKALRLDPNNADAMQNLLNAKMREE-(X2) (SEQ ID NO: 60)
Sequence ID 61 (I53-50A_8gly) N-linked glycan sequons are underlined
(X1)-
EELFKKHKIVAVLRANSVEEAIEKAVAVFAGGVHLIEITFTVNNTDTVIKALSVLKEKGAIIGAGTVTS
VEYANLTVNATANFTVSPHLDEEISNFTKNATVFYMPGVMTPTELVKAMKLNVTILKLFPGEVVGPQFV
KAMKGPFHNATFVPTGGVNLDNVCEWFKAGVLAVGVGSALVKGTPDEVREKAKAFVEKIRGC-(X2)
(SEQ ID NO: 61)
Sequence ID 62 (I53-50A_8gly) N-linked glycan sequons are underlined
(X1)-
EELFKKHKIVAVLRANSVEEAIEKAVAVFAGGVHLIEITFTVPNATTVIKALSVLKEKGAIIGAGTVTS
VEYANETVNATANFTVSPHLDEEISNFTKEKNVTYMPGVMTPTELVKAMWLNVTILKLFPGEVVGPQFV
KAMKGPFHNATFVPTGGVNLDNVCEWFKAGVLAVGVGSALVKGTPDEVREKAKAFVEKIRGC-(X2)
(SEQ ID NO: 62)
Sequence ID 63 (I53-50A_5gly) N-linked glycan sequons are underlined
(X1)-
EELFKKHKIVAVLRANSVEEAIEKAVAVFAGGVHLIEITFTVPDADTVIKALSVLKEKGAIIGAGTVFS
NDTCRKAVNATANFTVSPHLDEEISNFTKEKNVTYMPGVMTPTELVKAMKLGHTILKLFPGEVVGPQFV
KAMKGPFPNVKFVPTGGVNLDNVCEWFKAGVLAVGVGSALVKGTPDEVREKAKAFVEKIRGC-(X2)
(SEQ ID NO: 63)
Sequence ID 64 (I53-50A_5gly) N-linked glycan sequons are underlined
(X1)-
EELFKKHKIVAVLRANSVEEAIEKAVAVFAGGVHLIEITFTVPDADTVIKALSVLKEKGAIIGAGTVTY
VNITRKAVNATANFTVSPHLDEEISNFTKEKNVTYMPGVMTPTELVKAMKLGHTILKLFPGEVVGPQFV
KAMKGPFPNVKFVPTGGVNLDNVCEWFKAGVLAVGVGSALVKGTPDEVREKAKAFVEKIRGC-(X2)
(SEQ ID NO: 64)
Sequence ID 65 (I53-50A_5gly) N-linked glycan sequons are underlined
(X1)-
EELFKKHKIVAVLRANSVEEAIEKAVAVFAGGVHLIEITFTVPDADTVIKALSVLKEKGAIIGAGTVFS
NDTCRKAVNATANFTVSPHLDEEISNFTKNATVFYMPGVMTPTELVKAMKLGHTILKLFPGEVVGPQFV
KAMKGPFPNVKFVPTGGVNLDNVCEWFKAGVLAVGVGSALVKGTPDEVREKAKAFVEKIRGC-(X2)
(SEQ ID NO: 65)
Sequence ID 66 (I53-50A_5gly) N-linked glycan sequons are underlined
(X1)-
EELFKKHKIVAVLRANSVEEAIEKAVAVFAGGVHLIEITFTVPDADTVIKALSVLKEKGAIIGAGTVTY
VNITRKAVNATANFTVSPHLDEEISNFTKNATVFYMPGVMTPTELVKAMKLGHTILKLFPGEVVGPQFV
KAMKGPFPNVKFVPTGGVNLDNVCEWFKAGVLAVGVGSALVKGTPDEVREKAKAFVEKIRGC-(X2)
(SEQ ID NO: 66)
Sequence ID 67 (I53-50A_4gly) N-linked glycan sequons are underlined
(X1)-
EELFKKHKIVAVLRANSVEEAIEKAVAVFAGGVHLIEITFTVPNATTVIKALSVLKEKGAIIGAGTVTS
VEYANETVESGAEFIVSPHLDEEISNFTKEKGVFYMPGVMTPTELVKAMKLGHTILKLFPGEVVGPQFV
KAMKGPFHNATFVPTGGVNLDNVCEWFKAGVLAVGVGSALVKGTPDEVREKAKAFVEKIRGC-(X2)
(SEQ ID NO: 67)
Sequence ID 68 (I53-50A_4gly) N-linked glycan sequons are underlined
(X1)-
EELFKKHKIVAVLRANSVEEAIEKAVAVFAGGVHLIEITFTVPNATTVIKALSVLKEKGAIIGAGTVTS
VEYANETVESNATFIVSPHLDEEISNFTKEKGVFYMPGVMTPTELVKAMKLGHTILKLFPGEVVGPQFV
KAMKGPFPNVKFVPTGGVNLDNVCEWFKAGVLAVGVGSALVKGTPDEVREKAKAFVEKIRGC-(X2)
(SEQ ID NO: 68)
Sequence ID 69 (I53-50A_4gly) N-linked glycan sequons are underlined
(X1)-
EELFKKHKIVAVLRANSVEEAIEKAVAVFAGGVHLIEITFTVPDADTVIKALSVLKEKGAIIGAGTVTS
VEQCRKAVESGAEFIVSPHLDEEISNFTKEKNVTYMPGVMTPTELVKAMKLNVTILKLFPGEVVGPQFV
KAMKGPFHNATFVPTGGVNLDNVCEWFKAGVLAVGVGSALVKGTPDEVREKAKAFVEKIRGC-(X2)
(SEQ ID NO: 69)
Sequence ID 70 (I53-50A_4gly) N-linked glycan sequons are underlined
(X1)-
EELFKKHKIVAVLRANSVEEAIEKAVAVFAGGVHLIEITFTVPDADTVIKALSVLKEKGAIIGAGTVFS
NETCRKAVESNATFIVSPHLDEEISNFTKEKNVTYMPGVMTPTELVKAMKLGHTILKLFPGEVVGPQFV
KAMKGPFPNVKFVPTGGVNLDNVCEWFKAGVLAVGVGSALVKGTPDEVREKAKAFVEKIRGC-(X2)
(SEQ ID NO: 70)
Sequence ID 71 (I53-50A_5gly) N-linked glycan sequons are underlined
(X1)-
EELFKKHKIVAVLRANSVEEAIEKAVAVFAGGVHLIEITFTVPNATTVIKALSVLKEKGAIIGAGTVTS
VEYANETVESGAEFIVSPHLDEEISNFTKEKGVFYMPGVMTPTELVKAMKLNVTILKLFPGEVVGPQFV
KAMKGPFHNATFVPTGGVNLDNVCEWFKAGVLAVGVGSALVKGTPDEVREKAKAFVEKIRGC-(X2)
(SEQ ID NO: 71)
Sequence ID 72 (I53-50A_5gly) N-linked glycan sequons are underlined
(X1)-
EELFKKHKIVAVLRANSVEEAIEKAVAVFAGGVHLIEITFTVPNATTVIKALSVLKEKGAIIGAGTVTS
VEYANETVESGAEFIVSPHLDEEISNFTKEKGVFYMPGVMTPTELVKAMWLNHTILKLFPGEVVGPQFV
KAMKGPFHNATFVPTGGVNLDNVCEWFKAGVLAVGVGSALVKGTPDEVREKAKAFVEKIRGC-(X2)
(SEQ ID NO: 72)
Sequence ID 73 (I53-50A_6gly) N-linked glycan sequons are underlined
(X1)-
EELFKKHKIVAVLRANSVEEAIEKAVAVFAGGVHLIEITFTVPNATTVIKALSVLKEKGAIIGAGTVFS
NETCRKAVESGAEFIVSPHLDEEISNFTKEKNVTYMPGVMTPTELVKAMKLNVTILKLFPGEVVGPQFV
KAMKGPFHNATFVPTGGVNLDNVCEWFKAGVLAVGVGSALVKGTPDEVREKAKAFVEKIRGC-(X2)
(SEQ ID NO: 73)
Sequence ID 74 (I53-50A_6gly) N-linked glycan sequons are underlined
(X1)-
EELFKKHKIVAVLRANSVEEAIEKAVAVFAGGVHLIEITFTVPNATTVIKALSVLKEKGAIIGAGTVTS
VEQCRKAVESNATFIVSPHLDEEISNFTKEKNVTYMPGVMTPTELVKAMKLNVTILKLFPGEVVGPQFV
KAMKGPFHNATFVPTGGVNLDNVCEWFKAGVLAVGVGSALVKGTPDEVREKAKAFVEKIRGC-(X2)
(SEQ ID NO: 74)
Sequence ID 75 (I53-50A_6gly) N-linked glycan sequons are underlined
(X1)-
EELFKKHKIVAVLRANSVEEAIEKAVAVFAGGVHLIEITFTVPNATTVIKALSVLKEKGAIIGAGTVTS
VEYANETVESGAEFIVSPHLDEEISNFTKEKNVTYMPGVMTPTELVKAMKLNVTILKLFPGEVVGPQFV
KAMKGPFHNATFVPTGGVNLDNVCEWFKAGVLAVGVGSALVKGTPDEVREKAKAFVEKIRGC-(X2)
(SEQ ID NO: 75)
Sequence ID 76 (I53-50A_7gly) N-linked glycan sequons are underlined
(X1)-
EELFKKHKIVAVLRANSVEEAIEKAVAVFAGGVHLIEITFTVPNATTVIKALSVLKEKGAIIGAGTVFS
NETCRKAVESNATFIVSPHLDEEISNFTKEKNVTYMPGVMTPTELVKAMKLNVTILKLFPGEVVGPQFV
KAMKGPFHNATFVPTGGVNLDNVCEWFKAGVLAVGVGSALVKGTPDEVREKAKAFVEKIRGC-(X2)
(SEQ ID NO: 76)
Sequence ID 77 (I53-50A_7gly) N-linked glycan sequons are underlined
(X1)-
EELFKKHKIVAVLRANSVEEAIEKAVAVFAGGVHLIEITFTVPNATTVIKALSVLKEKGAIIGAGTVTS
VEYANETVESNATFIVSPHLDEEISNFTKEKNVTYMPGVMTPTELVKAMKLNVTILKLFPGEVVGPQFV
KAMKGPFHNATFVPTGGVNLDNVCEWFKAGVLAVGVGSALVKGTPDEVREKAKAFVEKIRGC-(X2)
(SEQ ID NO: 77)

In some embodiments X1 is absent. In other embodiments, X1 is present. This domain is a secretion signal peptide that can be used by mammalian cells to secrete the protein out of the cell; and are not needed when making the protein in bacteria such as E. coli. When present, X1 may be any signal peptide as appropriate for an intended use. In one non-limiting embodiment, X1 may comprise or consist of the amino acid sequence MDSKGSSQKGSRLLLLLVVSNLLLPQGVLA (SEQ ID NO:97).

In one embodiment, X3 is absent. In another embodiment, X3 may be present and comprises a purification tag. When present, X3 may be any purification tag as appropriate for an intended use. In one non-limiting embodiment, X3 may comprise or consist of the amino acid sequence LEEQKLISEEDLHIHHHHH (SEQ ID NO:98).

In some embodiments, X1 and X3 are both absent. In other embodiments, X1 is present and X3 is absent. In further embodiments, X1 and X3 are both present.

Table 3 presents data on expression, glycosylation, and nanoparticle assembly competency of these exemplary polypeptides. Listed are the sequon locations (first, single sequon inserts (SEQ IDs 1-48), and then combinations of sequon inserts (SEQ IDs 49-77)), expression, glycosylation, and nanoparticle assembly competency for each sequence.

Experimentally, first, sequences with a single sequon insert were validated for expression and glycosylation. Next, a limited set of those sequences with single sequon inserts that both expressed and glycosylated were combined into sequences that contained multiple sequon inserts.

Table 3 lists sequences and experimental outcomes of all possible locations in I53-50A, I53_dn5A, and I53_dn5B that can be glycosylated for de novo glycan display, either as single sequon inserts or as combinations of sequon inserts on a single protein chain. For I53-50A, it also discloses other glycan combinations that successfully assembled into nanoparticles that are not exemplified in the examples. Designs according to SEQ ID NO: 1-3, 5, 8-10, 13, 23, 26-28, 31-32, 34-38, 40, 42-46, 48-55, 59-60, and 67-74 showed high levels of both expression and glycosylation.

TABLE 3
Amino acid sequence IDs of glycosylated components of I53_dn5 and I53-50
self-assembling protein nanoparticles
“−” means no expression, glycosylation, or nanoparticle assembly, while
“+” means successful expression, glycosylation, or nanoparticle assembly,
with “+++” meaning best nanoparticle assembly.

| | | | | | |
|---|---|---|---|---|---|
| SEQ | Parent | | | | Nanoparticle |
| ID # | Protein | Sequon Location(s) | Expression | Glycosylation | Assembly |

Single sequon insertions

| | | | | | |
|---|---|---|---|---|---|
| 1 | I53_dn5A | 83-NET-85 | + | + | n/a |
| 2 | I53_dn5A | 84-NDS-86 | + | + | n/a |
| 3 | I53_dn5A | 118-NST-120 | + | + | n/a |
| 4 | I53_dn5A | 120-NES-122 | + | − | n/a |
| 5 | I53_dn5A | 118-FSNES-122 | + | + | n/a |
| 6 | I53_dn5A | 121-NES-123 | + | − | n/a |
| 7 | I53_dn5A | 119-FENES-123 | + | − | n/a |
| 8 | I53_dn5A | 130-NAS-132 | + | + | n/a |
| 9 | I53_dn5A | 131-NGS-133 | + | + | n/a |
| 10 | I53_dn5B | 33-NHT-35 | + | + | n/a |
| 11 | I53_dn5B | 31-FFNHT-35 | − | − | n/a |
| 12 | I53_dn5B | 34-NLS-36 | + | − | n/a |
| 13 | I53_dn5B | 32-FDNLS-36 | + | + | n/a |
| 14 | I53_dn5B | 35-NNS-37 | + | − | n/a |
| 15 | I53_dn5B | 33-WHNNS-37 | − | − | n/a |
| 16 | I53_dn5B | 58-NYS-60 | + | − | n/a |
| 17 | I53_dn5B | 56-FINYS-60 | + | − | n/a |
| 18 | I53_dn5B | 61-NIS-63 | − | − | n/a |
| 19 | I53_dn5B | 60-FLNAT-64 | − | − | n/a |
| 20 | I53_dn5B | 65-NLS-67 | + | − | n/a |
| 21 | I53_dn5B | 68-NLS-70 | + | − | n/a |
| 22 | I53_dn5B | 66-FDNLS-70 | + | − | n/a |
| 23 | I53_dn5B | 89-NAT-91 | + | + | n/a |
| 24 | I53_dn5B | 94-FLNAS-98 | − | − | n/a |
| 25 | I53_dn5B | 100-NNS-102 | − | − | n/a |
| 26 | I53-50A | 44-NNS-46 | + | + | n/a |
| 27 | I53-50A | 42-WVNNS-46 | + | + | n/a |
| 28 | I53-50A | 45-NAS-47 | + | + | n/a |
| 29 | I53-50A | 57-NKS-59 | + | − | n/a |
| 30 | I53-50A | 55-YLNKS-59 | − | − | n/a |
| 31 | I53-50A | 69-FSNET-73 | + | + | n/a |
| 32 | I53-50A | 70-YVNVT-74 | + | + | n/a |
| 33 | I53-50A | 75-NRS-77 | − | − | n/a |
| 34 | I53-50A | 73-YANRS-77 | + | + | n/a |
| 35 | I53-50A | 79-NAS-81 | + | + | n/a |
| 36 | I53-50A | 81-NAS-83 | + | + | n/a |
| 37 | I53-50A | 79-WANAS-83 | + | + | n/a |
| 38 | I53-50A | 83-NFT-85 | + | + | n/a |
| 39 | I53-50A | 81-WANFT-85 | + | − | n/a |
| 40 | I53-50A | 96-NFT-98 | + | + | n/a |
| 41 | I53-50A | 99-NES-101 | − | − | n/a |
| 42 | I53-50A | 100-NAS-102 | + | + | n/a |
| 43 | I53-50A | 102-NVS-104 | + | + | n/a |
| 44 | I53-50A | 121-NGT-123 | + | + | n/a |
| 45 | I53-50A | 122-NVT-124 | + | + | n/a |
| 46 | I53-50A | 120-WLNHT-124 | + | + | n/a |
| 47 | I53-50A | 148-NTS-150 | + | − | n/a |
| 48 | I53-50A | 146-FHNTS-150 | + | + | n/a |

Combination sequon inserts

| | | | | | |
|---|---|---|---|---|---|
| 49 | I53_dn5A | 83-NET-85; 118-NDT-120; | + | + | + |
| | | 130-NAT-132 | | | |
| 50 | I53_dn5A | 84-NDT-86; 120-NET-122; | + | + | + |
| | | 131-NGT-133 | | | |
| 51 | I53_dn5A | 84-NDT-86; 118-NDT-120; | + | + | + |
| | | 130-NAT-132 | | | |
| 52 | I53_dn5A | 83-NDT-85; 130-NAT-132 | + | + | + |
| 53 | I53_dn5A | 84-NDT-86; 131-NGT-133 | + | + | + |
| 54 | I53_dn5A | 84-NDT-86; 118-NST-120 | + | + | + |
| 55 | I53_dn5A | 120-NET-122; 130-NAT-132 | + | + | ++++ |
| 56 | I53_dn5B | 33-NHT-35; 67-NHT-69; 89- | − | − | |
| | | NAT-91 | | | |
| 57 | I53_dn5B | 32-YDNLT-36; 67-NHT-69; | − | − | n/a |
| | | 89-NAT-91 | | | |
| 58 | I53_dn5B | 33-NHT-35; 89-NAT-91 | − | − | n/a |
| 59 | I53_dn5B | 32-YDNLT-36; 89-NAT-91 | + | + | +++ |
| 60 | I53_dn5B | 67-NHT-69; 89-NAT-91 | + | + | + |
| 61 | I53-50A | 44-NNT-46; 73-YANLT-77; | − | − | n/a |
| | | 79-NAT-81; 83-NFT-85; 96- | | | |
| | | NFT-98; 100-NAT-102; 122- | | | |
| | | NVT-124; 146-FHNAT-150 | | | |
| 62 | I53-50A | 45-NAT-47; 73-YANET-77; | − | − | n/a |
| | | 79-NAT-81; 83-NFT-85; 96- | | | |
| | | NFT-98; 102-NVT-104; 120- | | | |
| | | WLNVT-124; 146-FHNAT-150 | | | |
| 63 | I53-50A | 69-FSNDT-73; 79-NAT-81; | − | − | n/a |
| | | 83-NFT-85; 96-NFT-98; 102- | | | |
| | | NVT-104 | | | |
| 64 | I53-50A | 70-YVNIT-74; 79-NAT-81; 83- | − | − | n/a |
| | | NFT-85; 96-NFT-98; 102- | | | |
| | | NVT-104 | | | |
| 65 | I53-50A | 69-FSNDT-73; 79-NAT-81; | − | − | n/a |
| | | 83-NFT-85; 96-NFT-98; 100- | | | |
| | | NAT-102 | | | |
| 66 | I53-50A | 70-YVNIT-74; 79-NAT-81; 83- | − | − | n/a |
| | | NFT-85; 96-NFT-98; 100- | | | |
| | | NAT-102 | | | |
| 67 | I53-50A | 45-NAT-47; 73-YANET-77; | + | + | +++ |
| | | 96-NFT-98; 146-FHNAT-150 | | | |
| 68 | I53-50A | 45-NAT-47; 73-YANET-77; | + | + | + |
| | | 81-NAT-83; 96-NFT-98 | | | |
| 69 | I53-50A | 96-NFT-98; 102-NVT-104; | + | + | + |
| | | 122-NVT-124; 146-FHNAT-150 | | | |
| 70 | I53-50A | 69-FSNET-73; 81-NAT-83; | + | + | + |
| | | 96-NFT-98; 102-NVT-104 | | | |
| 71 | I53-50A | 45-NAT-47; 73-YANET-77; | + | + | + |
| | | 96-NFT-98; 122-NVT-124; | | | |
| | | 146-FHNAT-150 | | | |
| 72 | I53-50A | 78-NAT-80; 73-YANET-77; | + | + | + |
| | | 96-NFT-98; 120-WLNHT-124; | | | |
| | | 146-FHNAT-150 | | | |
| 73 | I53-50A | 45-NAT-47; 69-FSNET-73; | + | + | +++ |
| | | 96-NFT-98; 102-NVT-104; | | | |
| | | 122-NVT-124; 146-FHNAT-150 | | | |
| 74 | I53-50A | 45-NAT-47; 81-NAT-83; 96- | + | + | + |
| | | NFT-98; 102-NVT-104; 122- | | | |
| | | NVT-124; 146-FHNAT-150 | | | |
| 75 | I53-50A | 45-NAT-47; 73-YANET-77; | + | + | − |
| | | 96-NFT-98; 102-NVT-104; | | | |
| | | 122-NVT-124; 146-FHNAT-150 | | | |
| 76 | I53-50A | 45-NAT-47; 69-FSNET-73; | + | + | − |
| | | 81-NAT-83; 96-NFT-98; 102- | | | |
| | | NVT-104; 122-NVT-124; 146- | | | |
| | | FHNAT-150 | | | |
| 77 | I53-50A | 45-NAT-47; 73-YANET-773; | + | + | − |
| | | 81-NAT-83; 96-NFT-98; 102- | | | |
| | | NVT-104; 122-NVT-124; 146- | | | |
| | | FHNAT-150 | | | |

In one embodiment, the polypeptide comprises the amino acid sequence selection from the group consisting of SEQ ID NO: 1-3, 5, 8-10, 13, 23, 26-28, 31-32, 34-38, 40, 42-46, 48-55, 59-60, and 67-74. In a further embodiment, the polypeptide comprises the amino acid sequence selection from the group consisting of SEQ ID NO: 49-55, 59-60, and 67-74. In one embodiment, the polypeptide comprises the amino acid sequence selection from the group consisting of SEQ ID NO: 55, 59, 67, and 73.

In another embodiment, the disclosure provides fusion proteins, comprising
(a) the polypeptide of any embodiment disclosed herein; and
(b) a functional domain linked to the polypeptide, either directly or via an optional amino acid linker.

The functional domain may be any polypeptide domain of interest to be displayed on a nanoparticle comprising the fusion proteins of the disclosure. The functional domain may be N-terminal or C-terminal to the polypeptide. In one embodiment, the functional domain is N-terminal to the polypeptide. In one embodiment, the polypeptide domain and the functional domain are linked via an amino acid linker, which may be of any suitable length or amino acid composition.

Any suitable linker can be used; there is no amino acid sequence requirement to serve as an appropriate linker. In some embodiments, the linker may comprise a Gly-Ser linker (i.e.: a linker consisting of glycine and serine residues) of any suitable length. In various embodiments, the Gly-Ser linker may be 3, 4, 5, 6, 7, 8, 9, 10, 11, 12, 13, 14, 15, 16, 17, 18, 19, 20, or more amino acids in length. In various embodiments, the Gly-Ser linker may comprise or consist of the amino acid sequence of GSGGSGSGSGGSGSG (SEQ ID NO:180), GGSGGSGS (SEQ ID NO:181), GSGGSGSG (SEQ ID NO:182), AGGA (SEQ ID NO:183), G, AGGAM (SEQ ID NO:184), GS, or GSGS (SEQ ID NO:185).

In other embodiments, the polypeptide domain and the functional domain are linked without an intervening amino acid linker.

In one embodiment, the functional domain comprises a polypeptide antigen. Nanoparticles comprising such fusion proteins are useful, for example, to generate an immune response in a subject in need thereof. Any polypeptide antigen may be used as deemed appropriate for an intended use. In some embodiments, the antigen comprises a bacterial antigen, a viral antigen, a fungal antigen, or a cancer antigen.

In other embodiments, the antigen comprises a SARS-CoV-2 antigen or a variant or homolog thereof. In one embodiment, the SARS-CoV-2 antigen or a variant or homolog thereof comprises an amino acid sequence having at least 75%, 80%, 85%, 90%, 91%, 92%, 93%, 94%, 95%, 96%, 97%, 98%, 99%, or 100% amino acid sequence identity to a Spike (S) protein extracellular domain (ECD) amino acid sequence, an S1 subunit amino acid sequence, an S2 subunit amino acid sequence, an S1 receptor binding domain (RBD) amino acid sequence, and/or an N-terminal domain (NTD) amino acid sequence, from SARS-CoV-2. In further embodiments, the SARS-CoV-2 antigen or a variant or homolog thereof is at least 75%, 80%, 85%, 90%, 91%, 92%, 93%, 94%, 95%, 96%, 97%, 98%, 99%, or 100% amino acid sequence identity to the amino acid sequence selected from the group consisting of SEQ ID NO:99-111. These sequences are shown in Table 4.

TABLE 4
Exemplary SARS-COV-2 antigen sequences

RFPNITNLCPFGEVFNATRFASVYAWNRKRISNCVADYSVLYNSASFSTFKCYGVSPTKLNDLCFTNVY
ADSFVIRGDEVRQIAPGQTGKIADYNYKLPDDFTGCVIAWNSNNLDSKVGGNYNYLYRLFRKSNLKPFE
RDISTEIYQAGSTPCNGVEGFNCYFPLQSYGFQPTNGVGYQPYRVVVLSFELLHAPATVCGPKKST
(RBD)SEQ ID NO: 99
ETGTRFPNITNLCPFGEVFNATRFASVYAWNRKRISNCVADYSVLYNSASFSTFKCYGVSPTKLNDLCF
TNVYADSFVIRGDEVRQIAPGQTGKIADYNYKLPDDFTGCVIAWNSNNLDSKVGGNYNYLYRLFRKSNL
KPFERDISTEIYQAGSTPCNGVEGFNCYFPLQSYGFQPTNGVGYQPYRVVVLSFELLHAPATVCGPKKS
T (RBD)SEQ ID NO: 100
QCVNLTTRTQLPPAYTNSFTRGVYYPDKVFRSSVLHSTQDLFLPFFSNVTWFHAIHVSGTNGTKRFDNP
VLPFNDGVYFASTEKSNIIRGWIFGTTLDSKTQSLLIVNNATNVVIKVCEFQFCNDPFLGVYYHKNNKS
WMESEFRVYSSANNCTFEYVSQPFLMDLEGKQGNFKNLREFVFKNIDGYFKIYSKHTPINLVRDLPQGF
SALEPLVDLPIGINITRFQTLLALHRSYLTPGDSSSGWTAGAAAYYVGYLQPRTFLLKYNENGTITDAV
DCALDPLSETKCTLKSFTVEKGIYQTSNFRVQPTESIVRFPNITNLCPFGEVFNATRFASVYAWNRKRI
SNCVADYSVLYNSASFSTFKCYGVSPTKLNDLCFTNVYADSFVIRGDEVRQIAPGQTGKIADYNYKLPD
DFTGCVIAWNSNNLDSKVGGNYNYLYRLFRKSNLKPFERDISTEIYQAGSTPCNGVEGFNCYFPLQSYG
FQPTNGVGYQPYRVVVLSFELLHAPATVCGPKKSTNLVKNKCVNFNFNGLTGTGVLTESNKKFLPFQQF
GRDIADTTDAVRDPQTLEILDITPCSFGGVSVITPGTNTSNQVAVLYQDVNCTEVPVAIHADQLTPTWR
VYSTGSNVFQTRAGCLIGAEHVNNSYECDIPIGAGICASYQTQTNSPSGAGSVASQSIIAYTMSLGAEN
SVAYSNNSIAIPTNFTISVTTEILPVSMTKTSVDCTMYICGDSTECSNLLLQYGSFCTQLNRALTGIAV
EQDKNTQEVFAQVKQIYKTPPIKDFGGFNFSQILPDPSKPSKRSFIEDLLFNKVTLADAGFIKQYGDCL
GDIAARDLICAQKFNGLTVLPPLLTDEMIAQYTSALLAGTITSGWTFGAGAALQIPFAMQMAYRFNGIG
VTQNVLYENQKLIANQFNSAIGKIQDSLSSTASALGKLQDVVNQNAQALNTLVKQLSSNFGAISSVIND
ILSRLDPPEAEVQIDRLITGRLQSLQTYVTQQLIRAAEIRASANLAATKMSECVLGQSKRVDFCGKGYH
LMSFPQSAPHGVVFLHVTYVPAQEKNFTTAPAICHDGKAHFPREGVFVSNGTHWFVTQRNFYEPQIITT
DNTFVSGNCDVVIGIVNNTVYDPLQPELDSFKEELDKYFKNHTSPDVDLGDISGINASVVNIQKEIDRL
NEVAKNLNESLIDLQELGKYEQYIK (Spike (S) protein extracellular domain (ECD))
SEQ ID NO: 101
(ETGT)QCVNLTTRTQLPPAYTNSFTRGVYYPDKVFRSSVLHSTQDLFLPFFSNVTWFHAIHVSGTNGT
KRFDNPVLPFNDGVYFASTEKSNIIRGWIFGTTLDSKTQSLLIVNNATNVVIKVCEFQFCNDPFLGVYY
HKNNKSWMESEFRVYSSANNCTFEYVSQPFLMDLEGKQGNFKNLREFVFKNIDGYFKIYSKHTPINLVR
DLPQGFSALEPLVDLPIGINITRFQTLLALHRSYLTPGDSSSGWTAGAAAYYVGYLQPRTFLLKYNENG
TITDAVDCALDPLSETKCTLKSFTVEKGIYQTSNFRVQPTESIVRFPNITNLCPFGEVFNATRFASVYA
WNRKRISNCVADYSVLYNSASFSTFKCYGVSPTKLNDLCFTNVYADSFVIRGDEVRQIAPGQTGKIADY
NYKLPDDFTGCVIAWNSNNLDSKVGGNYNYLYRLFRKSNLKPFERDISTEIYQAGSTPCNGVEGFNCYF
PLQSYGFQPTNGVGYQPYRVVVLSFELLHAPATVCGPKKSTNLVKNKCVNFNFNGLTGTGVLTESNKKF
LPFQQFGRDIADTTDAVRDPQTLEILDITPCSFGGVSVITPGTNTSNQVAVLYQDVNCTEVPVAIHADQ
LTPTWRVYSTGSNVFQTRAGCLIGAEHVNNSYECDIPIGAGICASYQTQTNSPSGAGSVASQSIIAYTM
SLGAENSVAYSNNSIAIPTNFTISVTTEILPVSMTKTSVDCTMYICGDSTECSNLLLQYGSFCTQLNRA
LTGIAVEQDKNTQEVFAQVKQIYKTPPIKDFGGFNFSQILPDPSKPSKRSFIEDLLFNKVTLADAGFIK
QYGDCLGDIAARDLICAQKFNGLTVLPPLLTDEMIAQYTSALLAGTITSGWTFGAGAALQIPFAMQMAY
RFNGIGVTQNVLYENQKLIANQFNSAIGKIQDSLSSTASALGKLQDVVNQNAQALNTLVKQLSSNFGAI
SSVLNDILSRLDPPEAEVQIDRLITGRLQSLQTYVTQQLIRAAEIRASANLAATKMSECVLGQSKRVDF
CGKGYHLMSFPQSAPHGVVFLHVTYVPAQEKNFTTAPAICHDGKAHFPREGVFVSNGTHWFVTQRNFYE
PQIITTDNTFVSGNCDVVIGIVNNTVYDPLQPELDSFKEELDKYFKNHTSPDVDLGDISGINASVVNIQ
KEIDRLNEVAKNLNESLIDLQELGKYEQYIK (Spike (S) protein extracellular domain
(ECD), including N-terminal linker related to signal peptide in parentheses,
which may be present or absent) SEQ ID NO: 102
(MGILPSPGMPALLSLVSLLSVLLMGCVAETGT)QCVNLTTRTQLPPAYTNSFTRGVYYPDKVFRSSVL
HSTQDLFLPFFSNVTWFHAIHVSGTNGTKRFDNPVLPFNDGVYFASTEKSNIIRGWIFGTTLDSKTQSL
LIVNNATNVVIKVCEFQFCNDPFLGVYYHKNNKSWMESEFRVYSSANNCTFEYVSQPFLMDLEGKQGNF
KNLREFVFKNIDGYFKIYSKHTPINLVRDLPQGFSALEPLVDLPIGINITRFQTLLALHRSYLTPGDSS
SGWTAGAAAYYVGYLQPRTFLLKYNENGTITDAVDCALDPLSETKCTLKSFTVEKGIYQTSNFRVQPTE
SIVRFPNITNLCPFGEVFNATRFASVYAWNRKRISNCVADYSVLYNSASFSTFKCYGVSPTKLNDLCFT
NVYADSFVIRGDEVRQIAPGQTGKIADYNYKLPDDFTGCVIAWNSNNLDSKVGGNYNYLYRLFRKSNLK
PFERDISTEIYQAGSTPCNGVEGFNCYFPLQSYGFQPTNGVGYQPYRVVVLSFELLHAPATVCGPKKST
NLVKNKCVNFNFNGLTGTGVLTESNKKFLPFQQFGRDIADTTDAVRDPQTLEILDITPCSFGGVSVITP
GTNTSNQVAVLYQDVNCTEVPVAIHADQLTPTWRVYSTGSNVFQTRAGCLIGAEHVNNSYECDIPIGAG
ICASYQTQTNSPSGAGSVASQSIIAYTMSLGAENSVAYSNNSIAIPTNFTISVTTEILPVSMTKTSVDC
TMYICGDSTECSNLLLQYGSFCTQLNRALTGIAVEQDKNTQEVFAQVKQIYKTPPIKDFGGENESQILP
DPSKPSKRSFIEDLLFNKVTLADAGFIKQYGDCLGDIAARDLICAQKFNGLTVLPPLLTDEMIAQYTSA
LLAGTITSGWTFGAGAALQIPFAMQMAYRFNGIGVTQNVLYENQKLIANQFNSAIGKIQDSLSSTASAL
GKLQDVVNQNAQALNTLVKQLSSNFGAISSVLNDILSRLDPPEAEVQIDRLITGRLQSLQTYVTQQLIR
AAEIRASANLAATKMSECVLGQSKRVDFCGKGYHLMSFPQSAPHGVVFLHVTYVPAQEKNFTTAPAICH
DGKAHFPREGVFVSNGTHWFVTQRNFYEPQIITTDNTFVSGNCDVVIGIVNNTVYDPLQPELDSFKEEL
DKYFKNHTSPDVDLGDISGINASVVNIQKEIDRLNEVAKNLNESLIDLQELGKYEQYIK (SEQ ID
NO: 103) mu phosphatase signal peptide, and the ETGT (SEQ ID NO: 186) is
left over as a remnant after signal peptide cleavage
(MFVFLVLLPLVSSQC)VNLTTRTQLPPAYTNSFTRGVYYPDKVFRSSVLHSTQDLFLPFFSNVTWFHAIHVSGTN
GTKRFDNPVLPFNDGVYFASTEKSNIIRGWIFGTTLDSKTQSLLIVNNATNVVIKVCEFQFCNDPFLGVYYHKNNK
SWMESEFRVYSSANNCTFEYVSQPFLMDLEGKQGNFKNLREFVFKNIDGYFKIYSKHTPINLVRDLPQGFSALEPL
VDLPIGINITRFQTLLALHRSYLTPGDSSSGWTAGAAAYYVGYLQPRTFLLKYNENGTITDAVDCALDPLSETKCT
LKSFTVEKGIYQTSNFRVQPTESIVRFPNITNLCPFGEVFNATRFASVYAWNRKRISNCVADYSVLYNSASFSTFK
CYGVSPTKLNDLCFTNVYADSFVIRGDEVRQIAPGQTGKIADYNYKLPDDFTGCVIAWNSNNLDSKVGGNYNYLYR
LFRKSNLKPFERDISTEIYQAGSTPCNGVEGFNCYFPLQSYGFQPTNGVGYQPYRVVVLSFELLHAPATVCGPKKS
TNLVKNKCVNFNFNGLTGTGVLTESNKKFLPFQQFGRDIADTTDAVRDPQTLEILDITPCSFGGVSVITPGTNTSN
QVAVLYQDVNCTEVPVAIHADQLTPTWRVYSTGSNVFQTRAGCLIGAEHVNNSYECDIPIGAGICASYQTQTNSPG
SASSVASQSIIAYTMSLGAENSVAYSNNSIAIPTNFTISVTTEILPVSMTKTSVDCTMYICGDSTECSNLLLQYGS
FCTQLNRALTGIAVEQDKNTQEVFAQVKQIYKTPPIKDFGGFNFSQILPDPSKPSKRSPIEDLLFNKVTLADAGFI
KQYGDCLGDIAARDLICAQKFNGLTVLPPLLTDEMIAQYTSALLAGTITSGWTFGAGPALQIPFPMQMAYRFNGIG
VTQNVLYENQKLIANQFNSAIGKIQDSLSSTPSALGKLQDVVNQNAQALNTLVKQLSSNFGAISSVLNDILSRLDP
PEAEVQIDRLITGRLQSLQTYVTQQLIRAAEIRASANLAATKMSECVLGQSKRVDFCGKGYHLMSFPQSAPHGVVF
LHVTYVPAQEKNFTTAPAICHDGKAHFPREGVFVSNGTHWFVTQRNFYEPQIITTDNTFVSGNCDVVIGIVNNTVY
DPLQPELDSFKEELDKYFKNHTSPDVDLGDISGINASVVNIQKEIDRLNEVAKNLNESLIDLQELGKYEQ
(SEQ ID NO: 104)
(MFVFLVLLPLVSSQC)VNLTTRTQLPPAYTNSFTRGVYYPDKVFRSSVLHSTQDLFLPFFSNVTWFHAIHVSGTN
GTKRFDNPVLPFNDGVYFASTEKSNIIRGWIFGTTLDSKTQSLLIVNNATNVVIKVCEFQFCNDPFLGVYYHKNNK
SWMESEFRVYSSANNCTFEYVSQPFLMDLEGKQGNFKNLREFVFKNIDGYFKIYSKHTPINLVRDLPQGFSALEPL
VDLPIGINITRFQTLLALHRSYLTPGDSSSGWTAGAAAYYVGYLQPRTFLLKYNENGTITDAVDCALDPLSETKCT
LKSFTVEKGIYQTSNFRVQPTESIVRFPNITNLCPFGEVFNATRFASVYAWNRKRISNCVADYSVLYNSASFSTFK
CYGVSPTKLNDLCFTNVYADSFVIRGDEVRQIAPGQTGKIADYNYKLPDDFTGCVIAWNSNNLDSKVGGNYNYLYR
LFRKSNLKPFERDISTEIYQAGSTPCNGVEGFNCYFPLQSYGFQPTNGVGYQPYRVVVLSFELLHAPATVCGPKKS
TNLVKNKCVNFNFNGLTGTGVLTESNKKFLPFQQFGRDIADTTDAVRDPQTLEILDITPCSFGGVSVITPGTNTSN
QVAVLYQDVNCTEVPVAIHADQLTPTWRVYSTGSNVFQTRAGCLIGAEHVNNSYECDIPIGAGICASYQTQTNSPG
SASSVASQSIIAYTMSLGAENSVAYSNNSIAIPTNFTISVTTEILPVSMTKTSVDCTMYICGDSTECSNLLLQYGS
FCTQLNRALTGIAVEQDKNTQEVFAQVKQIYKTPPIKDFGGFNFSQILPDPSKPSKRSPIEDLLENKVTLADAGFI
KQYGDCLGDIAARDLICAQKFNGLTVLPPLLTDEMIAQYTSALLAGTITSGWTFGAGPALQIPFPMQMAYRFNGIG
VTQNVLYENQKLIANQFNSAIGKIQDSLSSTPSALGKLQDVVNQNAQALNTLVKQLSSNFGAISSVLNDILSRLDP
PEAEVQIDRLITGRLQSLQTYVTQQLIRAAEIRASANLAATKMSECVLGQSKRVDFCGKGYHLMSFPQSAPHGVVF
LHVTYVPAQEKNFTTAPAICHDGKAHFPREGVFVSNGTHWFVTQRNFYEPQIITTDNTFVSGNCDVVIGIVNNTVY
DPLQPELDSFKEELDKYFKNHT (SEQ ID NO: 105)
(QC)VNLTTRTQLPPAYTNSFTRGVYYPDKVFRSSVLHSTQDLFLPFFSNVTWFHAIHVSGTNGTKRFDNPVLPFN
DGVYFASTEKSNIIRGWIFGTTLDSKTQSLLIVNNATNVVIKVCEFQFCNDPFLGVYYHKNNKSWMESEFRVYSSA
NNCTFEYVSQPFLMDLEGKQGNFKNLREFVFKNIDGYFKIYSKHTPINLVRDLPQGFSALEPLVDLPIGINITRFQ
TLLALHRSYLTPGDSSSGWTAGAAAYYVGYLQPRTFLLKYNENGTITDAVDCALDPLSETKCTLKSFTVEKGIYQT
SNFRVQPTESIVRFPNITNLCPFGEVFNATRFASVYAWNRKRISNCVADYSVLYNSASFSTFKCYGVSPTKLNDLC
FTNVYADSFVIRGDEVRQIAPGQTGKIADYNYKLPDDFTGCVIAWNSNNLDSKVGGNYNYLYRLFRKSNLKPFERD
ISTEIYQAGSTPCNGVEGFNCYFPLQSYGFQPTNGVGYQPYRVVVLSFELLHAPATVCGPKKSTNLVKNKCVNFNF
NGLTGTGVLTESNKKFLPFQQFGRDIADTTDAVRDPQTLEILDITPCSFGGVSVITPGTNTSNQVAVLYQDVNCTE
VPVAIHADQLTPTWRVYSTGSNVFQTRAGCLIGAEHVNNSYECDIPIGAGICASYQTQTNSPGSASSVASQSIIAY
TMSLGAENSVAYSNNSIAIPTNFTISVTTEILPVSMTKTSVDCTMYICGDSTECSNLLLQYGSFCTQLNRALTGIA
VEQDKNTQEVFAQVKQIYKTPPIKDFGGFNFSQILPDPSKPSKRSPIEDLLENKVTLADAGFIKQYGDCLGDIAAR
DLICAQKFNGLTVLPPLLTDEMIAQYTSALLAGTITSGWTFGAGPALQIPFPMQMAYRFNGIGVTQNVLYENQKLI
ANQFNSAIGKIQDSLSSTPSALGKLQDVVNQNAQALNTLVKQLSSNFGAISSVLNDILSRLDPPEAEVQIDRLITG
RLQSLQTYVTQQLIRAAEIRASANLAATKMSECVLGQSKRVDFCGKGYHLMSFPQSAPHGVVFLHVTYVPAQEKNF
TTAPAICHDGKAHFPREGVFVSNGTHWFVTQRNFYEPQIITTDNTFVSGNCDVVIGIVNNTVYDPLQPELDSFKEE
LDKYFKNHTSPDVDLGDISGINASVVNIQKEIDRLNEVAKNLNESLIDLQELGKYEQ (SEQ ID NO: 106)
(QC)VNLTTRTQLPPAYTNSFTRGVYYPDKVFRSSVLHSTQDLFLPFFSNVTWFHAIHVSGTNGTKRFDNPVLPFN
DGVYFASTEKSNIIRGWIFGTTLDSKTQSLLIVNNATNVVIKVCEFQFCNDPFLGVYYHKNNKSWMESEFRVYSSA
NNCTFEYVSQPFLMDLEGKQGNFKNLREFVFKNIDGYFKIYSKHTPINLVRDLPQGFSALEPLVDLPIGINITRFQ
TLLALHRSYLTPGDSSSGWTAGAAAYYVGYLQPRTFLLKYNENGTITDAVDCALDPLSETKCTLKSFTVEKGIYQT
SNFRVQPTESIVRFPNITNLCPFGEVFNATRFASVYAWNRKRISNCVADYSVLYNSASFSTFKCYGVSPTKLNDLC
FTNVYADSFVIRGDEVRQIAPGQTGKIADYNYKLPDDFTGCVIAWNSNNLDSKVGGNYNYLYRLFRKSNLKPFERD
ISTEIYQAGSTPCNGVEGFNCYFPLQSYGFQPTNGVGYQPYRVVVLSFELLHAPATVCGPKKSTNLVKNKCVNFNF
NGLTGTGVLTESNKKFLPFQQFGRDIADTTDAVRDPQTLEILDITPCSFGGVSVITPGTNTSNQVAVLYQDVNCTE
VPVAIHADQLTPTWRVYSTGSNVFQTRAGCLIGAEHVNNSYECDIPIGAGICASYQTQTNSPGSASSVASQSIIAY
TMSLGAENSVAYSNNSIAIPTNFTISVTTEILPVSMTKTSVDCTMYICGDSTECSNLLLQYGSFCTQLNRALTGIA
VEQDKNTQEVFAQVKQIYKTPPIKDFGGFNFSQILPDPSKPSKRSPIEDLLFNKVTLADAGFIKQYGDCLGDIAAR
DLICAQKFNGLTVLPPLLTDEMIAQYTSALLAGTITSGWTFGAGPALQIPFPMQMAYRFNGIGVTQNVLYENQKLI
ANQFNSAIGKIQDSLSSTPSALGKLQDVVNQNAQALNTLVKQLSSNFGAISSVLNDILSRLDPPEAEVQIDRLITG
RLQSLQTYVTQQLIRAAEIRASANLAATKMSECVLGQSKRVDFCGKGYHLMSFPQSAPHGVVFLHVTYVPAQEKNF
TTAPAICHDGKAHFPREGVFVSNGTHWFVTQRNFYEPQIITTDNTFVSGNCDVVIGIVNNTVYDPLQPELDSFKEE
LDKYFKNHT (SEQ ID NO: 107)
VNLTTRTQLPPAYTNSFTRGVYYPDKVFRSSVLHSTQDLFLPFFSNVTWFHAIHVSGTNGTKRFDNPVLPFNDGVY
FASTEKSNIIRGWIFGTTLDSKTQSLLIVNNATNVVIKVCEFQFCNDPFLGVYYHKNNKSWMESEFRVYSSANNCT
FEYVSQPFLMDLEGKQGNFKNLREFVFKNIDGYFKIYSKHTPINLVRDLPQGFSALEPLVDLPIGINITRFQTLLA
LHRSYLTPGDSSSGWTAGAAAYYVGYLQPRTFLLKYNENGTITDAVDCALDPLSETKCTLKSFTVEKGIYQTSNFR
VQPTESIVRFPNITNLCPFGEVFNATRFASVYAWNRKRISNCVADYSVLYNSASFSTFKCYGVSPTKLNDLCFTNV
YADSFVIRGDEVRQIAPGQTGKIADYNYKLPDDFTGCVIAWNSNNLDSKVGGNYNYLYRLFRKSNLKPFERDISTE
IYQAGSTPCNGVEGFNCYFPLQSYGFQPTNGVGYQPYRVVVLSFELLHAPATVCGPKKSTNLVKNKCVNFNFNGLT
GTGVLTESNKKFLPFQQFGRDIADTTDAVRDPQTLEILDITPCSFGGVSVITPGTNTSNQVAVLYQDVNCTEVPVA
IHADQLTPTWRVYSTGSNVFQTRAGCLIGAEHVNNSYECDIPIGAGICASYQTQTNSPGSASSVASQSIIAYTMSL
GAENSVAYSNNSIAIPTNFTISVTTEILPVSMTKTSVDCTMYICGDSTECSNLLLQYGSFCTQLNRALTGIAVEQD
KNTQEVFAQVKQIYKTPPIKDFGGFNFSQILPDPSKPSKRSPIEDLLFNKVTLADAGFIKQYGDCLGDIAARDLIC
AQKFNGLTVLPPLLTDEMIAQYTSALLAGTITSGWTFGAGPALQIPFPMQMAYRFNGIGVTQNVLYENQKLIANQF
NSAIGKIQDSLSSTPSALGKLQDVVNQNAQALNTLVKQLSSNFGAISSVLNDILSRLDPPEAEVQIDRLITGRLQS
LQTYVTQQLIRAAEIRASANLAATKMSECVLGQSKRVDFCGKGYHLMSFPQSAPHGVVFLHVTYVPAQEKNFTTAP
AICHDGKAHFPREGVFVSNGTHWFVTQRNFYEPQIITTDNTFVSGNCDVVIGIVNNTVYDPLQPELDSFKEELDKY
FKNHTSPDVDLGDISGINASVVNIQKEIDRLNEVAKNLNESLIDLQELGKYEQ (SEQ ID NO: 108)
VNLTTRTQLPPAYTNSFTRGVYYPDKVFRSSVLHSTQDLFLPFFSNVTWFHAIHVSGTNGTKRFDNPVLPFNDGVY
FASTEKSNIIRGWIFGTTLDSKTQSLLIVNNATNVVIKVCEFQFCNDPFLGVYYHKNNKSWMESEFRVYSSANNCT
FEYVSQPFLMDLEGKQGNFKNLREFVFKNIDGYFKIYSKHTPINLVRDLPQGFSALEPLVDLPIGINITRFQTLLA
LHRSYLTPGDSSSGWTAGAAAYYVGYLQPRTFLLKYNENGTITDAVDCALDPLSETKCTLKSFTVEKGIYQTSNFR
VQPTESIVRFPNITNLCPFGEVFNATRFASVYAWNRKRISNCVADYSVLYNSASFSTFKCYGVSPTKLNDLCFTNV
YADSFVIRGDEVRQIAPGQTGKIADYNYKLPDDFTGCVIAWNSNNLDSKVGGNYNYLYRLFRKSNLKPFERDISTE
IYQAGSTPCNGVEGFNCYFPLQSYGFQPTNGVGYQPYRVVVLSFELLHAPATVCGPKKSTNLVKNKCVNFNFNGLT
GTGVLTESNKKFLPFQQFGRDIADTTDAVRDPQTLEILDITPCSFGGVSVITPGTNTSNQVAVLYQDVNCTEVPVA
IHADQLTPTWRVYSTGSNVFQTRAGCLIGAEHVNNSYECDIPIGAGICASYQTQTNSPGSASSVASQSIIAYTMSL
GAENSVAYSNNSIAIPTNFTISVTTEILPVSMTKTSVDCTMYICGDSTECSNLLLQYGSFCTQLNRALTGIAVEQD
KNTQEVFAQVKQIYKTPPIKDFGGFNFSQILPDPSKPSKRSPIEDLLFNKVTLADAGFIKQYGDCLGDIAARDLIC
AQKFNGLTVLPPLLTDEMIAQYTSALLAGTITSGWTFGAGPALQIPFPMQMAYRFNGIGVTQNVLYENQKLIANQF
NSAIGKIQDSLSSTPSALGKLQDVVNQNAQALNTLVKQLSSNFGAISSVLNDILSRLDPPEAEVQIDRLITGRLQS
LQTYVTQQLIRAAEIRASANLAATKMSECVLGQSKRVDFCGKGYHLMSFPQSAPHGVVFLHVTYVPAQEKNFTTAP
AICHDGKAHFPREGVFVSNGTHWFVTQRNFYEPQIITTDNTFVSGNCDVVIGIVNNTVYDPLQPELDSFKEELDKY
FKNHT (SEQ ID NO: 109)
ETCTQCVNLTTRTQLPPAYTNSFTRGVYYPDKVFRSSVLHSTQDLFLPFFSNVTWFHAIHVSGTNGTKRFDNPVLP
FNDGVYFASTEKSNIIRGWIFGTTLDSKTQSLLIVNNATNVVIKVCEFQFCNDPFLGVYYHKNNKSWMESEFRVYS
SANNCTFEYVSQPFLMDLEGKQGNFKNLREFVFKNIDGYFKIYSKHTPINLVRDLPQGFSALEPLVDLPIGINITR
FQTLLALHRSYLTPGDSSSGWTAGAAAYYVGYLQPRTFLLKYNENGTITDAVDCALDPLSETKCTLKSFTVEKGIY
QTSNFRVQPTESIVRFPNITNLCPFGEVFNATRFASVYAWNRKRISNCVADYSVLYNSASFSTFKCYGVSPTKLND
LCFTNVYADSFVIRGDEVRQIAPGQTGKIADYNYKLPDDFTGCVIAWNSNNLDSKVGGNYNYLYRLFRKSNLKPFE
RDISTEIYQAGSTPCNGVEGFNCYFPLQSYGFQPTNGVGYQPYRVVVLSFELLHAPATVCGPKKSTNLVKNKCVNF
NFNGLTGTGVLTESNKKFLPFQQFGRDIADTTDAVRDPQTLEILDITPCSFGGVSVITPGTNTSNQVAVLYQDVNC
TEVPVAIHADQLTPTWRVYSTGSNVFQTRAGCLIGAEHVNNSYECDIPIGAGICASYQTQTNSPGSASSVASQSII
AYTMSLGAENSVAYSNNSIAIPTNFTISVTTEILPVSMTKTSVDCTMYICGDSTECSNLLLQYGSFCTQLNRALTG
IAVEQDKNTQEVFAQVKQIYKTPPIKDFGGFNFSQILPDPSKPSKRSPIEDLLENKVTLADAGFIKQYGDCLGDIA
ARDLICAQKFNGLTVLPPLLTDEMIAQYTSALLAGTITSGWTFGAGPALQIPFPMQMAYRFNGIGVTQNVLYENQK
LIANQFNSAIGKIQDSLSSTPSALGKLQDVVNQNAQALNTLVKQLSSNFGAISSVLNDILSRLDPPEAEVQIDRLI
TGRLQSLQTYVTQQLIRAAEIRASANLAATKMSECVLGQSKRVDFCGKGYHLMSFPQSAPHGVVFLHVTYVPAQEK
NFTTAPAICHDGKAHFPREGVFVSNGTHWFVTQRNFYEPQIITTDNTFVSGNCDVVIGIVNNTVYDPLQPELDSFK
EELDKYFKNHTSPDVDLGDISGINASVVNIQKEIDRLNEVAKNLNESLIDLQELGKYEQ (SEQ ID NO: 110)
ETCTQCVNLTTRTQLPPAYTNSFTRGVYYPDKVFRSSVLHSTQDLFLPFFSNVTWFHAIHVSGTNGTKRFDNPVLP
FNDGVYFASTEKSNIIRGWIFGTTLDSKTQSLLIVNNATNVVIKVCEFQFCNDPFLGVYYHKNNKSWMESEFRVYS
SANNCTFEYVSQPFLMDLEGKQGNFKNLREFVFKNIDGYFKIYSKHTPINLVRDLPQGFSALEPLVDLPIGINITR
FQTLLALHRSYLTPGDSSSGWTAGAAAYYVGYLQPRTFLLKYNENGTITDAVDCALDPLSETKCTLKSFTVEKGIY
QTSNFRVQPTESIVRFPNITNLCPFGEVFNATRFASVYAWNRKRISNCVADYSVLYNSASFSTFKCYGVSPTKIND
LCFTNVYADSFVIRGDEVRQIAPGQTGKIADYNYKLPDDFTGCVIAWNSNNLDSKVGGNYNYLYRLFRKSNLKPFE
RDISTEIYQAGSTPCNGVEGFNCYFPLQSYGFQPTNGVGYQPYRVVVLSFELLHAPATVCGPKKSTNLVKNKCVNF
NFNGLTGTGVLTESNKKFLPFQQFGRDIADTTDAVRDPQTLEILDITPCSFGGVSVITPGTNTSNQVAVLYQDVNC
TEVPVAIHADQLTPTWRVYSTGSNVFQTRAGCLIGAEHVNNSYECDIPIGAGICASYQTQTNSPGSASSVASQSII
AYTMSLGAENSVAYSNNSIAIPTNFTISVTTEILPVSMTKTSVDCTMYICGDSTECSNLLLQYGSFCTQLNRALTG
IAVEQDKNTQEVFAQVKQIYKTPPIKDFGGFNFSQILPDPSKPSKRSPIEDLLFNKVTLADAGFIKQYGDCLGDIA
ARDLICAQKFNGLTVLPPLLTDEMIAQYTSALLAGTITSGWTFGAGPALQIPFPMQMAYRFNGIGVTQNVLYENQK
LIANQFNSAIGKIQDSLSSTPSALGKLQDVVNQNAQALNTLVKQLSSNFGAISSVLNDILSRLDPPEAEVQIDRLI
TGRLQSLQTYVTQQLIRAAEIRASANLAATKMSECVLGQSKRVDFCGKGYHLMSFPQSAPHGVVFLHVTYVPAQEK
NFTTAPAICHDGKAHFPREGVFVSNGTHWFVTQRNFYEPQIITTDNTFVSGNCDVVIGIVNNTVYDPLQPELDSFK
EELDKYFKNHT (SEQ ID NO: 111)

In another embodiment, the SARS-CoV-2 antigen or a variant or homolog thereof is at least 75%, 80%, 85%, 90%, 91%, 92%, 93%, 94%, 95%, 96%, 97%, 98%, 99%, or 100% identical to the amino acid sequence of SEQ ID NO:99, and comprise mutations at 1, 2, 3, 4, 5, 6, 7, or all 8 positions relative to SEQ ID NO:99 selected from the group consisting of K90N, K90T, G119S, Y126F, T151I, E157K, E157A, S167P, N174Y, and L125R, including but not limited to mutations comprising one of the following naturally occurring mutations or combinations of mutations:
N174Y (UK variant);
K90N/E157K/N174Y (South African variant);
K90N or T/E157K/N174Y (Brazil variant); or
L125R (LA variant).

The amino acid residue numbering of these naturally occurring variants is based on their position within SEQ ID NO:99, while they are generally described based on their residue number in the Spike protein (i.e.: K417 in spike=K90 in RBD; G446 in spike=G119 in RBD; L452 in spike=L125 in RBD; Y453 in spike=Y126 in RBD; T478 in spike=T151 in RBD; E484 in spike=E157 in RBD; S494 in spike=S167 in RBD; N501 in spike=N174 in RBD).

In another embodiment, the SARS-CoV-2 antigen or a variant or homolog thereof is at least 75%, 80%, 85%, 90%, 91%, 92%, 93%, 94%, 95%, 96%, 97%, 98%, 99%, or 100% identical to the amino acid sequence of SEQ ID NO:104, and wherein the antigen comprises mutations at 1, 2, 3, 4, 5, 6, 7, or all 8 positions relative to SEQ ID NO:104 selected from the group consisting of L18F, T20N, P26S, deletion of residues 69-70, D80A, D138Y, R190S, D215G, K417N, K417T, G446S, L452R, Y453F, T4781, E484K, S494P, N501Y, A570D, D614G, H655Y, P681H, A701V, T716L including but not limited to mutations comprising one of the following naturally occurring mutations or combinations of mutations:
N501Y, optionally further including 1, 2, 3, 4, or 5 of deletion of one or both of residues 69-70, A570D, D614G, P681H, and/or T716L (UK variant);
K417N/E484K/N501Y, optionally further including 1, 2, 3, 4, or 5 of L18F, D80A, D215G, D614G, and/or A701V (South African variant);
K417N or T/E484K/N501Y, optionally further including 1, 2, 3, 4, or 5 of L18F, T20N, P26S, D138Y, R190S, D614G, and/or H655Y (Brazil variant); or
L452R (LA variant).

In other embodiments, the antigen comprises an antigen from an infectious agent listed in Table 5, or comprises and antigen listed in Table 6 or an antigenic fragment or mutated version thereof.

In various embodiments of the fusion proteins of the disclosure, the polypeptide comprises the amino acid sequence selection from the group consisting of SEQ ID NO: 10, 13, 23, 26-28, 31-32, 34-38, 40, 42-46, 48, and 59-60, and 67-74; or the polypeptide comprises the amino acid sequence selection from the group consisting of SEQ ID NO: 59-60 and 67-74; or the polypeptide comprises the amino acid sequence selection from the group consisting of SEQ ID NO: 59, 67, and 73.

TABLE 5
Exemplary infectious agents and antigens

| Infectious | | |
|---|---|---|
| Agent | Antigens | Citation |
| HIV | gp160, gp140, gp21, | Sok, D., Le, K. M., Vadnais, M., Saye-Francisco, K. L., Jardine, J. G., Torres, |
| | MPER | J. L., et al. (2017). Rapid elicitation of broadly neutralizing antibodies to HIV |
| | | by immunization in cows. Nature, 548(7665), 108-111. |
| RSV | F protein (prefusion) | US20160046675A1, US 2016/0031972 A1, US 2017/0182151 A1, |
| | | WO 2010/149745 A1, WO 2012/158613 A1, WO 2013/139916 A1, |
| | | WO 2014/079842 A1, WO 2014/174018 A1, WO 2014/202570 A1, |
| | | WO 2015/013551 A1, WO 2017/040387 A2, WO2017172890A1 |
| Influenza | HA - Influenza A and B | Nabel et al. Induction of unnatural immunity: prospects for a broadly |
| | | protective universal influenza vaccine. Nat Med. 2010 December; 16(12): 1389-91. |
| EBV | glycoprotein 350/220 | Kanekiyo et al. Rational Design of an Epstein-Barr Virus Vaccine Targeting |
| | (gp350) | the Receptor-Binding Site. Cell. 2015 Aug. 27; 162(5): 1090-100. |
| CMV | gB; UL128, UL130, | Ciferri et al. Structural and biochemical studies of HCMV gH/gL/gO and |
| | UL131A, gH (UL75) | Pentamer reveal mutually exclusive cell entry complexes. Proc. Natl. Acad. |
| | and gL (UL115) | Sci. U.S.A. 112, 1767-1772 (2015). |
| | | Chandramouli et al. Structure of HCMV glycoprotein B in the postfusion |
| | | conformation bound to a neutralizing human |
| | | antibody. Nat Commun. 2015 Sep. 14; 6: 8176. |
| | | Chandramouli et al. Structural basis for potent antibody-mediated |
| | | neutralization of human cytomegalovirus Sci. Immunol. 2, eaan1457 (2017). |
| Lyme | Outer Surface Protein A | Ma et al. Safety, efficacy, and immunogenicity of a recombinant Osp subunit |
| | (OspA) | canine Lyme disease vaccine. Volume 14, Issue 14, October 1996, Pages |
| | | 1366-1374 |
| Pertussis | Pertussis toxin (PT) | Seubert et al. Genetically detoxified pertussis toxin (PT-9K/129G): |
| | | implications for immunization and vaccines. Expert Rev Vaccines. 2014 |
| | | October; 13(10): 1191-204. doi: 10.1586/14760584.2014.942641. Epub 2014 Sep. 3. |
| Dengue | E protein | Modis, Y., Ogata, S., Clements, D. & Harrison, S. C. (2003) Proc. Natl. Acad. |
| | | Sci. USA 100, 6986-6991. pmid: 12759475 |
| SARS | Spike (S) glycoprotein | Structure of SARS coronavirus spike receptor-binding domain complexed with |
| | | receptor. Science. 2005 Sep. 16; 309(5742): 1864-8; WO2006068663A2 |
| MERS | Spike (S) glycoprotein | Immunogenicity and structures of a rationally designed prefusion MERS-CoV |
| | | spike antigen. PNAS 2017 August, 114 (35) E7348-E7357. |
| | | doi.org/10.1073/pnas.1707304114 |
| Ebola | EBOV GP or sGP [GP₁ | Structures of Ebola virus GP and sGP in complex with therapeutic antibodies. |
| | and GP₂ subunits | Nat Microbiol. 2016 Aug. 8; 1(9): 16128. doi: 10.1038/nmicrobiol.2016.128. |
| Marberg | Marberg GP or sGP | Hashiguchi et al. Structural basis for Marburg virus neutralization by a cross- |
| | | reactive human antibody. Cell. 2015 Feb. 26; 160(5): 904-912. |
| Hantaan virus | Gn and Gc envelope | Hantavirus Gn and Gc Envelope Glycoproteins: Key Structural Units for Virus |
| | glycoproteins | Cell Entry and Virus Assembly. Viruses. 2014 April; 6(4): 1801-1822. |
| Hepatitis B | HepB surface antigen | Raldao et al. Virus-like particles in vaccine development. Expert Rev |
| | (HBs) | Vaccines. 2010 October; 9(10): 1149-76. |
| Measles | H and F proteins | Lobanova et al. The recombinant globular head domain of the measles virus |
| | | hemagglutinin protein as a subunit vaccine against measles. Vaccine. 2012 Apr. |
| | | 26; 30(20): 3061-7. |
| Nipah virus | G and F protein | Satterfield et al. Status of vaccine research and development of vaccines for |
| | | Nipah virus. Vaccine. 34(26): 2971-2975 (2016). |
| Rotatvirus | VP4 and VP8 | O'Ryan et al. Parenteral protein-based rotavirus vaccine. Lancet Infectious |
| | | Disease. 17(8): 786-787 (2017). |
| Human | G and F proteins | Aertes et al. Adjuvant effect of the human metapneumovirus (HMPV) matrix |
| Metapneumovirus | | protein in HMPV subunit vaccines. J Gen Virol. 2015 April; 96(Pt 4): 767-74; |
| | | US 20180008697 A1. |
| Parainfluenza | HN and F proteins | Morein et al. Protein subunit vaccines of parainfluenza type 3 virus: |
| virus | | immunogenic effect in lambs and mice. J Gen Virol. 1983 July; 64 (Pt 7): 1557- |
| | | 69. |
| Zika | Zika envelope domain | Recurrent Potent Human Neutralizing Antibodies to |
| | III (ZEDIII) | Zika Virus in Brazil and Mexico. Cell. 2017 May 4; 169(4): 597-609.e11. doi: |
| | | 10.1016/j.cell.2017.04.024. |
| Malaria | Pfs25, circumsporozoite | Lee et al. Assessment of Pfs25 expressed from multiple soluble expression |
| | protein (CSP) | platforms for use as transmission-blocking vaccine candidates. Malar J. 2016; |
| | | 15: 405. |
| | | Plassmeyer et al. Structure of the |
| | | Plasmodium falciparum circumsporozoite protein, a leading malaria vaccine |
| | | candidate. J Biol Chem. 2009 Sep. 25; 284(39): 26951-63. |
| MenB | fHbp, NadA and NHBA | Davide et al. The new multicomponent vaccine against meningococcal |
| | | serogroup B, 4CMenB: immunological, functional and structural |
| | | characterization of the antigens. Vaccine. 2012 May 30; 30(0 2): B87-B97. |
| MenA, C, W- | oligosaccharide | Tontini et al. Comparison of CRM197, diphtheria toxoid and tetanus toxoid as |
| 135, and Y | | protein carriers for meningococcal glycoconjugate vaccines. Vaccine. 2013 |
| | | Oct. 1; 31(42): 4827-33. |

TABLE 6
Exemplary Antigen Sequences

| Antigen | Amino Acid Sequence (UniProt) |
|---|---|
| Human | >tr\|A0A1C9TBY8\|A0A1C9TBY8_9HIV1 Envelope glycoprotein gp160 |
| immunodeficiency | OS = Human immunodeficiency virus 1 GN = env PE = 3 SV = 1 |
| virus 1 | MRVKGIKKNYQHWWRGGIMLLGMLMICSSAEKLWVTVYYGVPVWKEATTTLFCASDAKAQN |
| (HIV-1) | PEMHNIWATHACVPTDPNPQEVILKNLTEEFNMWKNNMVEQMHEDIISLWDQSLKPCVKLT |
| gp160 | PLCVTLNCTNAESLNCTATNGTNNCSASTKPMEEMKNCSFNITTSVQDKKQQEYALFYKLD |
| | IIPIDNNENDLNNTNYTSYRLISCNTSVITQACPKITFEPIPIHYCAPAGFAILKCKDKRF |
| | NGTGPCKNVSTVQCTHGIRPVVSTQLLINGSLAEEGVVLRSENFTDNAKNIIVQLKDPVNI |
| | TCTRPNNNTRKSITIGPGRAFYATGQVIGDIRKAHCDLNGTEWDNALKQIVEELRKQYGNN |
| | ITIFNSSSGGDPEIVMHSFNCGGEFFYCNTAQLENSTWLFNSTWNSTERLGNDTERTNDTI |
| | TLPCKIKQVINMWQTVGKAMYAPPIRGLIRCSSNITGLILTRDGSGNTTGNETFRPGGGNM |
| | KDNWRSELYKYKVVKIEPLGVAPTRAKRRVVQREKRAAGLGALFLGFLGMAGSTMGAASLT |
| | LTVQARQLLSGIVQQQNNLLRAIEAQQHLLQLTVWGIKQLQARVLAVERYLRDQQLLGIWG |
| | CSGKLICTTTVPWNASWSNKSLDNIWENMTWMQWFKEIDNYTDVIYKLLEESQNQQEKNEQ |
| | ELLELDKWASLWNWFDITRWLWYIKIFIMIVGGLVGLRIVFAVLSIVNRVRQGYSPLSFQT |
| | LFPAPRGPDRPEGTEEGGGERGRDSSDRSAHGFLALIWGDLWSLCLFSYRRLRDLLLIAAR |
| | IVELLGRRGWEVLKYWWSLLQYWSQELKKSAVSLLNATAIAVAEGTDRIIEIVQRAGRAII |
| | HIPRRIRQGAERALL (SEQ ID NO: 112) |
| Human | gp120>tr\|A0A1C9TBY8\|33-524 |
| immunodeficiency | LWVTVYYGVPVWKEATTTLFCASDAKAQNPEMHNIWATHACVPTDPNPQEVILKNLTEEFN |
| virus 1 | MWKNNMVEQMHEDIISLWDQSLKPCVKLTPLCVTLNCTNAESLNCTATNGTNNCSASTKPM |
| (HIV-1) | EEMKNCSFNITTSVQDKKQQEYALFYKLDIIPIDNNENDLNNTNYTSYRLISCNTSVITQA |
| | CPKITFEPIPIHYCAPAGFAILKCKDKRFNGTGPCKNVSTVQCTHGIRPVVSTQLLINGSL |
| gp120 | AEEGVVLRSENFTDNAKNIIVQLKDPVNITCTRPNNNTRKSITIGPGRAFYATGQVIGDIR |
| | KAHCDLNGTEWDNALKQIVEELRKQYGNNITIFNSSSGGDPEIVMHSFNCGGEFFYCNTAQ |
| | LFNSTWLFNSTWNSTERLGNDTERTNDTITLPCKIKQVINMWQTVGKAMYAPPIRGLIRCS |
| | SNITGLILTRDGSGNTTGNETFRPGGGNMKDNWRSELYKYKVVKIEPLGVAPTRAKRRVVQ |
| | REKR (SEQ ID NO: 113) |
| Human | gp41>tr\|A0A1C9TBY8\|543-733 |
| immunodeficiency | MGAASLTLTVQARQLLSGIVQQQNNLLRAIEAQQHLLQLTVWGIKQLQARVLAVERYLRDQ |
| virus 1 | QLLGIWGCSGKLICTTTVPWNASWSNKSLDNIWENMTWMQWFKEIDNYTDVIYKLLEESQN |
| (HIV-1) | QQEKNEQELLELDKWASLWNWFDITRWLWYIKIFIMIVGGLVGLRIVFAVLSIVNRVRQGY |
| gp41 | SPLSFQTL (SEQ ID NO: 114) |
| Human | >tr\|A0A1C9TBY8\|675-696 |
| immunodeficiency | ELDKWASLWNWFDITRWLWYIK (SEQ ID NO: 115) |
| virus 1 | |
| (HIV-1) | |
| MPER | |
| Respiratory | >tr\|X4Y973\|X4Y973_9MONO Fusion glycoprotein F0 OS = Respiratory |
| syncytial | syncytial virus type A GN = F PE = 3 SV = 1 |
| virus (RSV) | MELPILKTNAITTILAAVTLCFASSQNITEEFYQSTCSAVSKGYLSALRTGWYTSVITIEL |
| type A | SNIKFNKCNGTDAKVKLIKQELDKYKNAVTELQLLMQSTPAANNRARRELPREMNYTLNNT |
| F protein | KNNNVTLSKKRKRRFLGFLLGVGSAIASGIAVSKVLHLEGEVNKIKNALLSTNKAVVSLSN |
| | GVSVLTSKVLDLKNYIDKQLLPIVNKQSCSISNIETVIEFQQKNNRLLEITREFSVNAGVT |
| | TPVSTYMLTNSELLSLINDMPITNDQKKLMSNNVQIVRQQSYSIMSIIKEEVLAYVVQLPL |
| | YGVIDTPCWKLHTSPLCTTNTKEGSNICLTRTDRGWYCDNAGSVSFFPQAETCKVQSNRVF |
| | CDTMNSLTLPSEVNLCNIDIFNPKYDCKIMTSKTDVSSSVITSLGAIVSCYGKTKCTASNK |
| | NRGIIKTFSNGCDYVSNKGVDTVSVGNTLYYVNKQEGKSLYVKGEPIINFYDPLVFPSDEF |
| | DASISQVNEKINQSLAFIRKSDELLHNVNVGKSTTNIMITTIIIVIIVILLLLIAVGLFLY |
| | CKARSTPVTLSKDQLSGINNIAFSN (SEQ ID NO: 116) |
| Influenza A | >tr\|C3W5X2\|C3W5X2_9INFA Hemagglutinin OS = Influenza A virus |
| virus | (A/California/07/2009 (H1N1) ) GN = HA PE = 1 SV = 1 |
| HA | MKAILVVLLYTFATANADTLCIGYHANNSTDTVDTVLEKNVTVTHSVNLLEDKHNGKLCKL |
| | RGVAPLHLGKCNIAGWILGNPECESLSTASSWSYIVETPSSDNGTCYPGDFIDYEELREQL |
| | SSVSSFERFEIFPKTSSWPNHDSNKGVTAACPHAGAKSFYKNLIWLVKKGNSYPKLSKSYI |
| | NDKGKEVLVLWGIHHPSTSADQQSLYQNADAYVFVGSSRYSKKFKPEIAIRPKVRDQEGRM |
| | NYYWTLVEPGDKITFEATGNLVVPRYAFAMERNAGSGIIISDTPVHDCNTTCQTPKGAINT |
| | SLPFQNIHPITIGKCPKYVKSTKLRLATGLRNIPSIQSRGLFGAIAGFIEGGWTGMVDGWY |
| | GYHHQNEQGSGYAADLKSTQNAIDEITNKVNSVIEKMNTQFTAVGKEFNHLEKRIENLNKK |
| | VDDGFLDIWTYNAELLVLLENERTLDYHDSNVKNLYEKVRSQLKNNAKEIGNGCFEFYHKC |
| | DNTCMESVKNGTYDYPKYSEEAKLNREEIDGVKLESTRIYQILAIYSTVASSLVLVVSLGA |
| | ISFWMCSNGSLQCRICI (SEQ ID NO: 117) |
| Influenza B | >tr\|A0A140EM53\|A0A140EM53_9INFB Hemagglutinin OS = Influenza B |
| virus | virus (B/Victoria/809/2012) GN = HA PE = 3 SV = 1 |
| HA | MKAIIVLLMVVTSNADRICTGITSSNSPHVVKTATQGEVNVTGVIPLTTTPTKSYFANLKG |
| | TKTRGKLCPDCLNCTDLDVALGRPMCVGTTPSAKASILHEVRPVTSGCFPIMHDRTKIRQL |
| | ANLLRGYENIRLSTQNVIDAEKAPGGPYRLGTSGSCPNATSKSGFFATMAWAVPKDNNKNA |
| | TNPLTVEVPYICAEGEDQITVWGFHSDNKTQMKNLYGDSNPQKFTSSANGVTTHYVSQIGG |
| | FPDQTEDGGLPQSGRIVVDYMMQKPGKTGTIVYQRGVLLPQKVWCASGRSKVIKGSLPLIG |
| | EADCLHEKYGGLNKSKPYYTGEHAKAIGNCPIWVKTPLKLANGTKYRPPAKLLKERGFFGA |
| | IAGFLEGGWEGMIAGWHGYTSHGAHGVAVAADLKSTQEAINKITKNLNSLSELEVKNLQRL |
| | SGAMDELHNEILELDEKVDDLRADTISSQIELAVLLSNEGIINSEDEHLLALERKLKKMLG |
| | PSAVDIGNGCFETKHKCNQTCLDRIAAGTFNAGEFSLPTFDSLNITAASLNDDGLDNHTIL |
| | LYYSTAASSLAVTLMLAIFIVYMVSRDNVSCSICL (SEQ ID NO: 118) |
| Epstein-Barr | >sp\|P03200\|GP350_EBVB9 Envelope glycoprotein GP350 |
| virus (EBV) | OS = Epstein-Barr virus (strain B95-8) GN = BLLF1 PE = 1 SV = 1 |
| glycoprotein | MEAALLVCQYTIQSLIHLTGEDPGFFNVEIPEFPFYPTCNVCTADVNVTINFDVGGKKHQL |
| 350/220 | DLDFGQLTPHTKAVYQPRGAFGGSENATNLFLLELLGAGELALTMRSKKLPINVTTGEEQQ |
| (gp350) | VSLESVDVYFQDVFGTMWCHHAEMQNPVYLIPETVPYIKWDNCNSTNITAVVRAQGLDVTL |
| | PLSLPTSAQDSNFSVKTEMLGNEIDIECIMEDGEISQVLPGDNKFNITCSGYESHVPSGGI |
| | LTSTSPVATPIPGTGYAYSLRLTPRPVSRFLGNNSILYVFYSGNGPKASGGDYCIQSNIVF |
| | SDEIPASQDMPTNTTDITYVGDNATYSVPMVTSEDANSPNVTVTAFWAWPNNTETDFKCKW |
| | TLTSGTPSGCENISGAFASNRTFDITVSGLGTAPKTLIITRTATNATTTTHKVIFSKAPES |
| | TTTSPTLNTTGFADPNTTTGLPSSTHVPTNLTAPASTGPTVSTADVTSPTPAGTTSGASPV |
| | TPSPSPWDNGTESKAPDMTSSTSPVTTPTPNATSPTPAVTTPTPNATSPTPAVTTPTPNAT |
| | SPTLGKTSPTSAVTTPTPNATSPTLGKTSPTSAVTTPTPNATSPTLGKTSPTSAVTTPTPN |
| | ATGPTVGETSPQANATNHTLGGTSPTPVVTSQPKNATSAVTTGQHNITSSSTSSMSLRPSS |
| | NPETLSPSTSDNSTSHMPLLTSAHPTGGENITQVTPASISTHHVSTSSPAPRPGTTSQASG |
| | PGNSSTSTKPGEVNVTKGTPPQNATSPQAPSGQKTAVPTVTSTGGKANSTTGGKHTTGHGA |
| | RTSTEPTTDYGGDSTTPRPRYNATTYLPPSTSSKLRPRWTFTSPPVTTAQATVPVPPTSQP |
| | RFSNLSMLVLQWASLAVLTLLLLLVMADCAFRRNLSTSHTYTTPPYDDAETYV (SEQ ID |
| | NO: 119) |
| Human | >sp\|P06473\|GB_HCMVA Envelope glycoprotein B OS = Human |
| cytomegalovirus | cytomegalovirus (strain AD169) GN = gB PE = 1 SV = 1 |
| gB | MESRIWCLVVCVNLCIVCLGAAVSSSSTSHATSSTHNGSHTSRTTSAQTRSVYSQHVTSSE |
| | AVSHRANETIYNTTLKYGDVVGVNTTKYPYRVCSMAQGTDLIRFERNIICTSMKPINEDLD |
| | EGIMVVYKRNIVAHTFKVRVYQKVLTFRRSYAYIYTTYLLGSNTEYVAPPMWEIHHINKFA |
| | QCYSSYSRVIGGTVFVAYHRDSYENKTMQLIPDDYSNTHSTRYVTVKDQWHSRGSTWLYRE |
| | TCNLNCMLTITTARSKYPYHFFATSTGDVVYISPFYNGTNRNASYFGENADKFFIFPNYTI |
| | VSDFGRPNAAPETHRLVAFLERADSVISWDIQDEKNVTCQLTFWEASERTIRSEAEDSYHF |
| | SSAKMTATFLSKKQEVNMSDSALDCVRDEAINKLQQIFNTSYNQTYEKYGNVSVFETSGGL |
| | VVFWQGIKQKSLVELERLANRSSLNITHRTRRSTSDNNTTHLSSMESVHNLVYAQLQFTYD |
| | TLRGYINRALAQIAEAWCVDQRRTLEVFKELSKINPSAILSAIYNKPIAARFMGDVLGLAS |
| | CVTINQTSVKVLRDMNVKESPGRCYSRPVVIENFANSSYVQYGQLGEDNEILLGNHRTEEC |
| | QLPSLKIFIAGNSAYEYVDYLFKRMIDLSSISTVDSMIALDIDPLENTDFRVLELYSQKEL |
| | RSSNVFDLEEIMREFNSYKQRVKYVEDKVVDPLPPYLKGLDDLMSGLGAAGKAVGVAIGAV |
| | GGAVASVVEGVATFLKNPFGAFTIILVAIAVVIITYLIYTRQRRLCTQPLQNLFPYLVSAD |
| | GTTVTSGSTKDTSLQAPPSYEESVYNSGRKGPGPPSSDASTAAPPYTNEQAYQMLLALARL |
| | DAEQRAQQNGTDSLDGQTGTQDKGQKPNLLDRLRHRKNGYRHLKDSDEEENV (SEQ ID |
| | NO: 120) |
| Human | >sp\|P16837\|UL128_HCMVA Uncharacterized protein UL128 OS = Human |
| cytomegalovirus | cytomegalovirus (strain AD169) GN = UL128 PE = 1 SV = 2 |
| UL128 | MSPKDLTPFLTTLWLLLGHSRVPRVRAEECCEFINVNHPPERCYDFKMCNRFTVALRCPDG |
| | EVCYSPEKTAEIRGIVTTMTHSLTRQVVHNKLTSCNYNPLYLEADGRIRCGKVNDKAQYLL |
| | GAAGSVPYRWINLEYDKITRIVGLDQYLESVKKHKRLDVCRAKMGYMLQ (SEQ ID |
| | NO: 121) |
| Human | >sp\|F5HCP3\|UL130_HCMVM Envelope glycoprotein UL130 OS = Human |
| cytomegalovirus | cytomegalovirus (strain Merlin) GN = UL130 PE = 1 SV = 1 |
| UL130 | MLRLLLRHHFHCLLLCAVWATPCLASPWSTLTANQNPSPPWSKLTYSKPHDAATFYCPFLY |
| | PSPPRSPLQFSGFQRVSTGPECRNETLYLLYNREGQTLVERSSTWVKKVIWYLSGRNQTIL |
| | QRMPRTASKPSDGNVQISVEDAKIFGAHMVPKQTKLLRFVVNDGTRYQMCVMKLESWAHVF |
| | RDYSVSFQVRLTFTEANNQTYTFCTHPNLIV (SEQ ID NO: 122) |
| Human | >sp\|F5HET4\|U131A_HCMVM Protein UL131A OS = Human |
| cytomegalovirus | cytomegalovirus (strain Merlin) GN = UL131A PE = 1 SV = 1 |
| UL131A | MRLCRVWLSVCLCAVVLGQCQRETAEKNDYYRVPHYWDACSRALPDQTRYKYVEQLVDLTL |
| | NYHYDASHGLDNFDVLKRINVTEVSLLISDFRRQNRRGGTNKRTTFNAAGSLAPHARSLEF |
| | SVRLFAN (SEQ ID NO: 123) |
| Human | >sp\|P12824\|GH_HCMVA Envelope glycoprotein H OS = Human |
| cytomegalovirus | cytomegalovirus (strain AD169) GN = gH PE = 1 SV = 1 |
| gH (UL75) | MRPGLPPYLTVFTVYLLSHLPSQRYGADAASEALDPHAFHLLLNTYGRPIRFLRENTTQCT |
| | YNSSLRNSTVVRENAISFNFFQSYNQYYVFHMPRCLFAGPLAEQFLNQVDLTETLERYQQR |
| | LNTYALVSKDLASYRSFSQQLKAQDSLGQQPTTVPPPIDLSIPHVWMPPQTTPHDWKGSHT |
| | TSGLHRPHFNQTCILFDGHDLLFSTVTPCLHQGFYLMDELRYVKITLTEDFFVVTVSIDDD |
| | TPMLLIFGHLPRVLFKAPYQRDNFILRQTEKHELLVLVKKAQLNRHSYLKDSDFLDAALDF |
| | NYLDLSALLRNSFHRYAVDVLKSGRCQMLDRRTVEMAFAYALALFAAARQEEAGTEISIPR |
| | ALDRQAALLQIQEFMITCLSQTPPRTTLLLYPTAVDLAKRALWTPDQITDITSLVRLVYIL |
| | SKQNQQHLIPQWALRQIADFALQLHKTHLASFLSAFARQELYLMGSLVHSMLVHTTERREI |
| | FIVETGLCSLAELSHFTQLLAHPHHEYLSDLYTPCSSSGRRDHSLERLTRLFPDATVPATV |
| | PAALSILSTMQPSTLETFPDLFCLPLGESFSALTVSEHVSYVVTNQYLIKGISYPVSTTVV |
| | GQSLIITQTDSQTKCELTRNMHTTHSITAALNISLENCAFCQSALLEYDDTQGVINIMYMH |
| | DSDDVLFALDPYNEVVVSSPRTHYLMLLKNGTVLEVTDVVVDATDSRLLMMSVYALSAIIG |
| | IYLLYRMLKTC (SEQ ID NO: 124) |
| Human | >sp\|P16832\|GL_HCMVA Envelope glycoprotein L OS = Human |
| cytomegalovirus | cytomegalovirus (strain AD169) GN = gL PE = 1 SV = 2 |
| gL (UL115) | MCRRPDCGFSFSPGPVVLLWCCLLLPIVSSVAVSVAPTAAEKVPAECPELTRRCLLGEVFQ |
| | GDKYESWLRPLVNVTRRDGPLSQLIRYRPVTPEAANSVLLDDAFLDTLALLYNNPDQLRAL |
| | LTLLSSDTAPRWMTVMRGYSECGDGSPAVYTCVDDLCRGYDLTRLSYGRSIFTEHVLGFEL |
| | VPPSLFNVVVAIRNEATRTNRAVRLPVSTAAAPEGITLFYGLYNAVKEFCLRHQLDPPLLR |
| | HLDKYYAGLPPELKQTRVNLPAHSRYGPQAVDAR (SEQ ID NO: 125) |
| Lyme | >sp\|Q04968\|OSPA7_BORBG Outer surface protein A OS = Borreliella |
| Outer Surface | burgdorferi GN = ospA PE = 3 SV = 1 |
| Protein A | MKKYLLGIGLILALIACKQNVSSLDEKNSVSVDVPGGMKVLVSKEKNKDGKYDLMATVDNV |
| (OspA) | DLKGTSDKNNGSGILEGVKADKSKVKLTVADDLSKTTLEVLKEDGTVVSRKVTSKDKSTTE |
| | AKFNEKGELSEKTMTRANGTTLEYSQMTNEDNAAKAVETLKNGIKFEGNLASGKTAVEIKE |
| | GTVTLKREIDKNGKVTVSLNDTASGSKKTASWQESTSTLTISANSKKTKDLVELTNGTITV |
| | QNYDSAGTKLEGSAAEIKKLDELKNALR (SEQ ID NO: 126) |
| Bordetella | >sp\|P04977\|TOX1_BORPE Pertussis toxin subunit 1 OS = Bordetella |
| pertussis | pertussis (strain Tohama I/ATCC BAA-589/NCTC 13251) |
| Pertussis | GN = ptxA PE = 1 SV = 1 |
| toxin (PT) | MRCTRAIRQTARTGWLTWLAILAVTAPVTSPAWADDPPATVYRYDSRPPEDVFQNGFTAWG |
| subunits 1-5 | NNDNVLDHLTGRSCQVGSSNSAFVSTSSSRRYTEVYLEHRMQEAVEAERAGRGTGHFIGYI |
| | YEVRADNNFYGAASSYFEYVDTYGDNAGRILAGALATYQSEYLAHRRIPPFNIRRVTRVYH |
| | NGITGETTTTEYSNARYVSQQTRANPNPYTSRRSVASIVGTLVRMAPVIGACMARQAESSE |
| | AMAAWSERAGEAMVLVYYESIAYSF (SEQ ID NO: 127) |
| Dengue virus | >sp\|P17763\|281-775 |
| Envelope | MRCVGIGNRDFVEGLSGATWVDVVLEHGSCVTTMAKDKPTLDIELLKTEVTNPAVLRKLCI |
| protein E | EAKISNTTTDSRCPTQGEATLVEEQDTNFVCRRTFVDRGWGNGCGLFGKGSLITCAKFKCV |
| | TKLEGKIVQYENLKYSVIVTVHTGDQHQVGNETTEHGTTATITPQAPTSEIQLTDYGALTL |
| | DCSPRTGLDFNEMVLLTMEKKSWLVHKQWFLDLPLPWTSGASTSQETWNRQDLLVTFKTAH |
| | AKKQEVVVLGSQEGAMHTALTGATEIQTSGTTTIFAGHLKCRLKMDKLTLKGMSYVMCTGS |
| | FKLEKEVAETQHGTVLVQVKYEGTDAPCKIPFSSQDEKGVTQNGRLITANPIVTDKEKPVN |
| | IEAEPPFGESYIVVGAGEKALKLSWFKKGSSIGKMFEATARGARRMAILGDTAWDFGSIGG |
| | VFTSVGKLIHQIFGTAYGVLFSGVSWTMKIGIGILLTWLGLNSRSTSLSMTCIAVGMVTLY |
| | LGVMVQA (SEQ ID NO: 128) |
| Human SARS | >sp\|P59594\|SPIKE_CVHSA Spike glycoprotein OS = Human SARS |
| coronavirus | coronavirus GN = SPE = 1 SV = 1 |
| (SARS) | MFIFLLFLTLTSGSDLDRCTTFDDVQAPNYTQHTSSMRGVYYPDEIFRSDTLYLTQDLFLP |
| Spike (S) | FYSNVTGFHTINHTFGNPVIPFKDGIYFAATEKSNVVRGWVFGSTMNNKSQSVIIINNSTN |
| glycoprotein | VVIRACNFELCDNPFFAVSKPMGTQTHTMIFDNAFNCTFEYISDAFSLDVSEKSGNFKHLR |
| | EFVFKNKDGFLYVYKGYQPIDVVRDLPSGFNTLKPIFKLPLGINITNFRAILTAFSPAQDI |
| | WGTSAAAYFVGYLKPTTFMLKYDFNGTITDAVDCSQNPLAELKCSVKSFEIDKGIYQTSNF |
| | RVVPSGDVVRFPNITNLCPFGEVFNATKFPSVYAWERKKISNCVADYSVLYNSTFFSTFKC |
| | YGVSATKLNDLCFSNVYADSFVVKGDDVRQIAPGQTGVIADYNYKLPDDEMGCVLAWNTRN |
| | IDATSTGNYNYKYRYLRHGKLRPFERDISNVPFSPDGKPCTPPALNCYWPLNDYGFYTTTG |
| | IGYQPYRVVVLSFELLNAPATVCGPKLSTDLIKNQCVNFNFNGLTGTGVLTPSSKRFQPFQ |
| | QFGRDVSDFTDSVRDPKTSEILDISPCSFGGVSVITPGTNASSEVAVLYQDVNCTDVSTAI |
| | HADQLTPAWRIYSTGNNVFQTQAGCLIGAEHVDTSYECDIPIGAGICASYHTVSLLRSTSQ |
| | KSIVAYTMSLGADSSIAYSNNTIAIPTNFSISITTEVMPVSMAKTSVDCNMYICGDSTECA |
| | NLLLQYGSFCTQLNRALSGIAAEQDRNTREVFAQVKQMYKTPTLKYFGGFNFSQILPDPLK |
| | PTKRSFIEDLLFNKVTLADAGFMKQYGECLGDINARDLICAQKFNGLTVLPPLLTDDMIAA |
| | YTAALVSGTATAGWTFGAGAALQIPFAMQMAYRFNGIGVTQNVLYENQKQIANQFNKAISQ |
| | IQESLTTTSTALGKLQDVVNQNAQALNTLVKQLSSNFGAISSVLNDILSRLDKVEAEVQID |
| | RLITGRLQSLQTYVTQQLIRAAEIRASANLAATKMSECVLGQSKRVDFCGKGYHLMSFPQA |
| | APHGVVFLHVTYVPSQERNFTTAPAICHEGKAYFPREGVFVFNGTSWFITQRNFFSPQIIT |
| | TDNTFVSGNCDVVIGIINNTVYDPLQPELDSFKEELDKYFKNHTSPDVDLGDISGINASVV |
| | NIQKEIDRLNEVAKNLNESLIDLQELGKYEQYIKWPWYVWLGFIAGLIAIVMVTILLCCMT |
| | SCCSCLKGACSCGSCCKFDEDDSEPVLKGVKLHYT (SEQ ID NO: 129) |
| Middle East | >tr\|R9UCW7\|R9UCW7_9BETC Spike glycoprotein OS = Middle East |
| respiratory | respiratory syndrome-related coronavirus PE = 4 SV = 1 |
| syndrome- | MIHSVFLLMFLLTPTESYVDVGPDSIKSACIEVDIQQTFFDKTWPRPIDVSKADGIIYPQG |
| related | RTYSNITITYQGLFPYQGDHGDMYVYSAGHATGTTPQKLFVANYSQDVKQFANGFVVRIGA |
| coronavirus | AANSTGTVIISPSTSATIRKIYPAFMLGSSVGNFSDGKMGRFFNHTLVLLPDGCGTLLRAF |
| (MERS) | YCILEPRSGNHCPAGNSYTSFATYHTPATDCSDGNYNRNASLNSFKEYFNLRNCTFMYTYN |
| Spike (S) | ITEDEILEWFGITQTAQGVHLFSSRYVDLYGGNMFQFATLPVYDTIKYYSIIPHSIRSIQS |
| glycoprotein | DRKAWAAFYVYKLQPLTFLLDFSVDGYIRRAIDCGFNDLSQLHCSYESFDVESGVYSVSSF |
| | EAKPSGSVVEQAEGVECDFSPLLSGTPPQVYNFKRLVFTNCNYNLTKLLSLFSVNDFTCSQ |
| | ISPAAIASNCYSSLILDYFSYPLSMKSDLSVSSAGPISQFNYKQSFSNPTCLILATVPHNL |
| | TTITKPLKYSYINKCSRLLSDDRTEVPQLVNANQYSPCVSIVPSTVWEDGDYYRKQLSPLE |
| | GGGWLVASGSTVAMTEQLQMGFGITVQYGTDTNSVCPKLEFANDTKIASQLGNCVEYSLYG |
| | VSGRGVFQNCTAVGVRQQRFVYDAYQNLVGYYSDDGNYYCLRACVSVPVSVIYDKETKTHA |
| | TLFGSVACEHISSTMSQYSRSTRSMLKRRDSTYGPLQTPVGCVLGLVNSSLFVEDCKLPLG |
| | QSLCALPDTPSTLTPRSVRSVPGEMRLASIAFNHPIQVDQLNSSYFKLSIPTNFSFGVTQE |
| | YIQTTIQKVTVDCKQYVCNGFQKCEQLLREYGQFCSKINQALHGANLRQDDSVRNLFASVK |
| | SSQSSPIIPGFGGDFNLILLEPVSISTGSRSARSAIEDLLEDKVTIADPGYMQGYDDCMQQ |
| | GPASARDLICAQYVAGYKVLPPLMDVNMEAAYTSSLLGSIAGVGWTAGLSSFAAIPFAQSI |
| | FYRLNGVGITQQVLSENQKLIANKFNQALGAMQTGFTTTNEAFHKVQDAVNNNAQALSKLA |
| | SELSNTFGAISASIGDIIQRLDVLEQDAQIDRLINGRLTTLNAFVAQQLVRSESAALSAQL |
| | AKDKVNECVKAQSKRSGFCGQGTHIVSFVVNAPNGLYFMHVGYYPSNHIEVVSAYGLCDAA |
| | NPTNCIAPVNGYFIKTNNTRIVDEWSYTGSSFYAPEPITSLNTKYVAPQVTYQNISTNLPP |
| | PLLGNSTGIDFQDFLDEFFKNVSTSIPNFGSLTQINTTLLDLTYEMLSLQQVVKALNESYI |
| | DLKELGNYTYYNKWPWYIWLGFIAGLVALALCVFFILCCTGCGTNCMGKLKCNRCCDRYEE |
| | YDLEPHKVHVH (SEQ ID NO: 130) |
| Zaire | >sp\|Q05320\|VGP_EBOZM Envelope glycoprotein OS = Zaire |
| ebolavirus | ebolavirus (strain Mayinga-76) GN = GPPE = 1 SV = 1 |
| GP | MGVTGILQLPRDRFKRTSFFLWVIILFQRTFSIPLGVIHNSTLQVSDVDKLVCRDKLSSTN |
| | QLRSVGLNLEGNGVATDVPSATKRWGFRSGVPPKVVNYEAGEWAENCYNLEIKKPDGSECL |
| | PAAPDGIRGFPRCRYVHKVSGTGPCAGDFAFHKEGAFFLYDRLASTVIYRGTTFAEGVVAF |
| | LILPQAKKDFFSSHPLREPVNATEDPSSGYYSTTIRYQATGFGTNETEYLFEVDNLTYVQL |
| | ESRFTPQFLLQLNETIYTSGKRSNTTGKLIWKVNPEIDTTIGEWAFWETKKNLTRKIRSEE |
| | LSFTVVSNGAKNISGQSPARTSSDPGTNTTTEDHKIMASENSSAMVQVHSQGREAAVSHLT |
| | TLATISTSPQSLTTKPGPDNSTHNTPVYKLDISEATQVEQHHRRTDNDSTASDTPSATTAA |
| | GPPKAENTNTSKSTDFLDPATTTSPQNHSETAGNNNTHHQDTGEESASSGKLGLITNTIAG |
| | VAGLITGGRRTRREAIVNAQPKCNPNLHYWTTQDEGAAIGLAWIPYFGPAAEGIYIEGLMH |
| | NQDGLICGLRQLANETTQALQLFLRATTELRTFSILNRKAIDFLLQRWGGTCHILGPDCCI |
| | EPHDWTKNITDKIDQIIHDFVDKTLPDQGDNDNWWTGWRQWIPAGIGVTGVIIAVIALFCI |
| | CKFVF (SEQ ID NO: 131) |
| Marburg virus | >sp\|P35253\|VGP_MABVM Envelope glycoprotein OS = Lake Victoria |
| GP | marburgvirus (strain Musoke-80) GN = GPPE = 1 SV = 1 |
| | MKTTCFLISLILIQGTKNLPILEIASNNQPQNVDSVCSGTLQKTEDVHLMGFTLSGQKVAD |
| | SPLEASKRWAFRTGVPPKNVEYTEGEEAKTCYNISVTDPSGKSLLLDPPTNIRDYPKCKTI |
| | HHIQGQNPHAQGIALHLWGAFFLYDRIASTTMYRGKVFTEGNIAAMIVNKTVHKMIFSRQG |
| | QGYRHMNLTSTNKYWTSSNGTQTNDTGCFGALQEYNSTKNQTCAPSKIPPPLPTARPEIKL |
| | TSTPTDATKLNTTDPSSDDEDLATSGSGSGEREPHTTSDAVTKQGLSSTMPPTPSPQPSTP |
| | QQGGNNTNHSQDAVTELDKNNTTAQPSMPPHNTTTISTNNTSKHNFSTLSAPLQNTTNDNT |
| | QSTITENEQTSAPSITTLPPTGNPTTAKSTSSKKGPATTAPNTTNEHFTSPPPTPSSTAQH |
| | LVYFRRKRSILWREGDMFPFLDGLINAPIDFDPVPNTKTIFDESSSSGASAEEDQHASPNI |
| | SLTLSYFPNINENTAYSGENENDCDAELRIWSVQEDDLAAGLSWIPFFGPGIEGLYTAVLI |
| | KNQNNLVCRLRRLANQTAKSLELLLRVTTEERTFSLINRHAIDFLLTRWGGTCKVLGPDCC |
| | IGIEDLSKNISEQIDQIKKDEQKEGTGWGLGGKWWTSDWGVLTNLGILLLLSIAVLIALSC |
| | ICRIFTKYIG (SEQ ID NO: 132) |
| Hanta virus | >sp\|P08668\|19-648 |
| Gn envelope | LRNVYDMKIECPHTVSFGENSVIGYVELPPVPLADTAQMVPESSCNMDNHQSLNTITKYTQ |
| glycoprotein | VSWRGKADQSQSSQNSFETVSTEVDLKGTCVLKHKMVEESYRSRKSVTCYDLSCNSTYCKP |
| | TLYMIVPIHACNMMKSCLIALGPYRVQVVYERSYCMTGVLIEGKCFVPDQSVVSIIKHGIF |
| | DIASVHIVCFFVAVKGNTYKIFEQVKKSFESTCNDTENKVQGYYICIVGGNSAPIYVPTLD |
| | DFRSMEAFTGIFRSPHGEDHDLAGEEIASYSIVGPANAKVPHSASSDTLSLIAYSGIPSYS |
| | SLSILTSSTEAKHVFSPGLFPKLNHTNCDKSAIPLIWTGMIDLPGYYEAVHPCTVFCVLSG |
| | PGASCEAFSEGGIFNITSPMCLVSKQNRFRLTEQQVNFVCQRVDMDIVVYCNGQRKVILTK |
| | TLVIGQCIYTITSLFSLLPGVAHSIAVELCVPGFHGWATAALLVTFCFGWVLIPAITFIIL |
| | TVLKFIANIFHTSNQENRLKSVLRKIKEEFEKTKGSMVCDVCKYECETYKELKAHGVSCPQ |
| | SQCPYCFTHCFPTEAAFQAHYKVCQVTHRFRDDLKKTVTPQNFTPGCYRTLNLFRYKSRCY |
| | IFTMWIFLLVLESILWAASA (SEQ ID NO: 133) |
| Hanta virus | >sp\|P08668\|649-1135 |
| Gc envelope | SETPLTPVWNDNAHGVGSVPMHTDLELDFSLTSSSKYTYRRKLTNPLEEAQSIDLHIEIEE |
| glycoprotein | QTIGVDVHALGHWFDGRLNLKTSFHCYGACTKYEYPWHTAKCHYERDYQYETSWGCNPSDC |
| | PGVGTGCTACGLYLDQLKPVGSAYKIITIRYSRRVCVQFGEENLCKIIDMNDCFVSRHVKV |
| | CIIGTVSKFSQGDTLLFFGPLEGGGLIFKHWCTSTCQFGDPGDIMSPRDKGFLCPEFPGSF |
| | RKKCNFATTPICEYDGNMVSGYKKVMATIDSFQSFNTSTMHFTDFRIEWKDPDGMLRDHIN |
| | ILVTKDIDFDNLGENPCKIGLQTSSIEGAWGSGVGFTLTCLVSLTECPTFLTSIKACDKAI |
| | CYGAESVTLTRGQNTVKVSGKGGHSGSTFRCCHGEDCSQIGLHAAAPHLDKVNGISEIENS |
| | KVYDDGAPQCGIKCWFVKSGEWISGIFSGNWIVLIVLCVFLLFSLVLLSILCPVRKHKKS |
| | (SEQ ID NO: 134) |
| Hepatitis B | >tr\|Q9DIX1\|Q9DIX1_HBV Surface antigen HBsAg OS = Hepatitis B |
| HepB surface | virus GN = SPE = 4 SV = 1 |
| antigen (HBs) | MENITSGFLGPLLVLQAGFFLLTKILTIPQSLNSWWTSLSFLGGNTVCLGQNSQSPTSNHS |
| | PTSCPPTCPGYRWMCLRRFIIFLFILLLCLIFLLVLLDYQGMLPVCPLIPGSSTTSTGPCR |
| | TCKTPAQGTSMYPSCCCTKPSDGNCTCIPIPSSWAFGKFLWEWASARFSWLSLIVPFVQWF |
| | VGLSPTVWLSVIWMMWYWGPSLYSILSPFLPLLPIFFCLWVYI(SEQ ID NO: 135) |
| Measles | >sp\|P08362\|HEMA_MEASE Hemagglutinin glycoprotein OS = Measles |
| H protein | virus (strain Edmonston) GN = H PE = 1 SV = 1 |
| | MSPQRDRINAFYKDNPHPKGSRIVINREHLMIDRPYVLLAVLFVMFLSLIGLLAIAGIRLH |
| | RAAIYTAEIHKSLSTNLDVTNSIEHQVKDVLTPLFKIIGDEVGLRTPQRFTDLVKFISDKI |
| | KFLNPDREYDFRDLTWCINPPERIKLDYDQYCADVAAEELMNALVNSTLLETRTTNQFLAV |
| | SKGNCSGPTTIRGQFSNMSLSLLDLYLGRGYNVSSIVTMTSQGMYGGTYLVEKPNLSSKRS |
| | ELSQLSMYRVFEVGVIRNPGLGAPVFHMTNYLEQPVSNDLSNCMVALGELKLAALCHGEDS |
| | ITIPYQGSGKGVSFQLVKLGVWKSPTDMQSWVPLSTDDPVIDRLYLSSHRGVIADNQAKWA |
| | VPTTRTDDKLRMETCFQQACKGKIQALCENPEWAPLKDNRIPSYGVLSVDLSLTVELKIKI |
| | ASGFGPLITHGSGMDLYKSNHNNVYWLTIPPMKNLALGVINTLEWIPRFKVSPYLFNVPIK |
| | EAGEDCHAPTYLPAEVDGDVKLSSNLVILPGQDLQYVLATYDTSRVEHAVVYYVYSPSRSF |
| | SYFYPFRLPIKGVPIELQVECFTWDQKLWCRHFCVLADSESGGHITHSGMEGMGVSCTVTR |
| | EDGTNRR (SEQ ID NO: 136) |
| Measles | >sp\|P69353\|FUS_MEASE Fusion glycoprotein FO OS = Measles virus |
| F protein | (strain Edmonston) GN = F PE = 3 SV = 1 |
| | MGLKVNVSAIFMAVLLTLQTPTGQIHWGNLSKIGVVGIGSASYKVMTRSSHQSLVIKLMPN |
| | ITLLNNCTRVEIAEYRRLLRTVLEPIRDALNAMTQNIRPVQSVASSRRHKRFAGVVLAGAA |
| | LGVATAAQITAGIALHQSMLNSQAIDNLRASLETTNQAIEAIRQAGQEMILAVQGVQDYIN |
| | NELIPSMNQLSCDLIGQKLGLKLLRYYTEILSLFGPSLRDPISAEISIQALSYALGGDINK |
| | VLEKLGYSGGDLLGILESRGIKARITHVDTESYFIVLSIAYPTLSEIKGVIVHRLEGVSYN |
| | IGSQEWYTTVPKYVATQGYLISNFDESSCTFMPEGTVCSQNALYPMSPLLQECLRGSTKSC |
| | ARTLVSGSFGNRFILSQGNLIANCASILCKCYTTGTIINQDPDKILTYIAADHCPVVEVNG |
| | VTIQVGSRRYPDAVYLHRIDLGPPISLERLDVGTNLGNAIAKLEDAKELLESSDQILRSMK |
| | GLSSTSIVYILIAVCLGGLIGIPALICCCRGRCNKKGEQVGMSRPGLKPDLTGTSKSYVRS |
| | L (SEQ ID NO: 137) |
| Zika | >sp\|Q32ZE1\|291-790 |
| Zika envelope | IRCIGVSNRDFVEGMSGGTWVDVVLEHGGCVTVMAQDKPTVDIELVTTTVSNMAEVRSYCY |
| domain III | EASISDMASDSRCPTQGEAYLDKQSDTQYVCKRTLVDRGWGNGCGLFGKGSLVTCAKFTCS |
| (ZEDIII) | KKMTGKSIQPFNLEYRIMLSVHGSQHSGMIGYETDEDRAKVEVTPNSPRAEATLGGFGSLG |
| | LDCFPRTGLDFSDLYYLTMNNKHWLVHKEWFHDIPLPWHAGADTGTPHWNNKEALVEFKDA |
| | HAKRQTVVVLGSQEGAVHTALAGALEAEMDGAKGRLFSGHLKCRLKMDKLRLKGVSYSLCT |
| | AAFTFTKVPAETLHGTVTVEVQYAGTDGPCKIPVQMAVDMQTLTPVGRLITANPVITESTE |
| | NSKMMLELDPPFGDSYIVIGVGDKKITHHWHRSGSTIGKAFEATVRGAKRMAVLGDTAWDF |
| | GSVGGVFNSLGKGIHQIFGAAFKSLFGGMSWFSQILIGTLLVWLGLNTKNGSISLTCLALG |
| | GVMIFLSTAVSA (SEQ ID NO: 138) |
| Malaria | >sp\|P08677\|CSP_PLAVB Circumsporozoite protein OS = Plasmodium |
| circumsporozoite | vivax (strain Belem) PE = 3 SV = 2 |
| protein | MKNFILLAVSSILLVDLFPTHCGHNVDLSKAINLNGVNENNVDASSLGAAHVGQSASRGRG |
| (CSP) | LGENPDDEEGDAKKKKDGKKAEPKNPRENKLKQPGDRADGQPAGDRADGQPAGDRADGQPA |
| | GDRAAGQPAGDRADGQPAGDRADGQPAGDRADGQPAGDRADGQPAGDRAAGQPAGDRAAGQ |
| | PAGDRADGQPAGDRAAGQPAGDRADGQPAGDRAAGQPAGDRADGQPAGDRAAGQPAGDRAA |
| | GQPAGDRAAGQPAGDRAAGQPAGNGAGGQAAGGNAGGGQGQNNEGANAPNEKSVKEYLDKV |
| | RATVGTEWTPCSVTCGVGVRVRRRVNAANKKPEDLTLNDLETDVCTMDKCAGIFNVVSNSL |
| | GLVILLVLALEN (SEQ ID NO: 139) |
| Nipah virus | >sp\|Q9IH63\|FUS_NIPAV Fusion glycoprotein FO OS-Nipah virus |
| F protein | GN = F PE = 1 SV = 1 |
| | MVVILDKRCYCNLLILILMISECSVGILHYEKLSKIGLVKGVTRKYKIKSNPLTKDIVIKM |
| | IPNVSNMSQCTGSVMENYKTRLNGILTPIKGALEIYKNNTHDLVGDVRLAGVIMAGVAIGI |
| | ATAAQITAGVALYEAMKNADNINKLKSSIESTNEAVVKLQETAEKTVYVLTALQDYINTNL |
| | VPTIDKISCKQTELSLDLALSKYLSDLLFVFGPNLQDPVSNSMTIQAISQAFGGNYETLLR |
| | TLGYATEDFDDLLESDSITGQIIYVDLSSYYIIVRVYFPILTEIQQAYIQELLPVSENNDN |
| | SEWISIVPNFILVRNTLISNIEIGFCLITKRSVICNQDYATPMTNNMRECLTGSTEKCPRE |
| | LVVSSHVPRFALSNGVLFANCISVTCQCQTTGRAISQSGEQTLLMIDNTTCPTAVLGNVII |
| | SLGKYLGSVNYNSEGIAIGPPVFTDKVDISSQISSMNQSLQQSKDYIKEAQRLLDTVNPSL |
| | ISMLSMIILYVLSIASLCIGLITFISFIIVEKKRNTYSRLEDRRVRPTSSGDLYYIGT |
| | (SEQ ID NO: 140) |
| Nipah virus | >sp\|Q9IH62\|GLYCP_NIPAV Glycoprotein G OS = Nipah virus GN = G |
| G protein | PE = 1 SV = 1 |
| | MPAENKKVRFENTTSDKGKIPSKVIKSYYGTMDIKKINEGLLDSKILSAFNTVIALLGSIV |
| | IIVMNIMIIQNYTRSTDNQAVIKDALQGIQQQIKGLADKIGTEIGPKVSLIDTSSTITIPA |
| | NIGLLGSKISQSTASINENVNEKCKFTLPPLKIHECNISCPNPLPFREYRPQTEGVSNLVG |
| | LPNNICLQKTSNQILKPKLISYTLPVVGQSGTCITDPLLAMDEGYFAYSHLERIGSCSRGV |
| | SKQRIIGVGEVLDRGDEVPSLFMTNVWTPPNPNTVYHCSAVYNNEFYYVLCAVSTVGDPIL |
| | NSTYWSGSLMMTRLAVKPKSNGGGYNQHQLALRSIEKGRYDKVMPYGPSGIKQGDTLYFPA |
| | VGFLVRTEFKYNDSNCPITKCQYSKPFNCRLSMGIRPNSHYILRSGLLKYNLSDGENPKVV |
| | FIEISDQRLSIGSPSKIYDSLGQPVFYQASFSWDTMIKFGDVLTVNPLVVNWRNNTVISRP |
| | GQSQCPRFNTCPEICWEGVYNDAFLIDRINWISAGVELDSNQTAENPVFTVFKDNEILYRA |
| | QLASEDTNAQKTITNCFLLKNKIWCISLVEIYDTGDNVIRPKLFAVKIPEQCT (SEQ ID |
| | NO: 141) |
| Rotavirus | >sp\|P11193\|VP4_ROTHW Outer capsid protein VP4 OS = Rotavirus A |
| VP4 protein | (strain RVA/Human/United States/Wa/1974/G1P1A[8]) PE = 1 SV = 3 |
| | MASLIYRQLLTNSYSVDLHDEIEQIGSEKTQNVTINPSPFAQTRYAPVNWGHGEINDSTTV |
| | EPILDGPYQPTTFTPPNDYWILINSNTNGVVYESTNNSDFWTAVVAIEPHVNPVDRQYTIF |
| | GESKQFNVSNDSNKWKFLEMFRSSSQNEFYNRRTLTSDTRFVGILKYGGRVWTFHGETPRA |
| | TTDSSSTANLNNISITIHSEFYIIPRSQESKCNEYINNGLPPIQNTRNVVPLPLSSRSIQY |
| | KRAQVNEDIIVSKTSLWKEMQYNRDIIIRFKFGNSIVKMGGLGYKWSEISYKAANYQYNYL |
| | RDGEQVTAHTTCSVNGVNNFSYNGGSLPTDFGISRYEVIKFNSYVYVDYWDDSKAFRNMVY |
| | VRSLAANLNSVKCTGGSYNFSIPVGAWPVMNGGAVSLHFAGVTLSTQFTDFVSLNSLRFRE |
| | SLTVDEPPFSILRTRTVNLYGLPAANPNNGNEYYEISGRFSLIYLVPTNDDYQTPIMNSVT |
| | VRQDLERQLTDLREEFNSLSQEIAMAQLIDLALLPLDMFSMFSGIKSTIDLTKSMATSVMK |
| | KFRKSKLATSISEMTNSLSDAASSASRNVSIRSNLSAISNWTNVSNDVSNVINSLNDISTQ |
| | TSTISKKERLKEMITQTEGMSFDDISAAVLKTKIDMSTQIGKNTLPDIVTEASEKFIPKRS |
| | YRILKDDEVMEINTEGKFFAYKINTFDEVPFDVNKFAELVTDSPVISAIIDFKTLKNLNDN |
| | YGITRTEALNLIKSNPNMLRNFINQNNPIIRNRIEQLILQCKL (SEQ ID NO: 142) |
| Rotavirus | >sp\|P11193\|1-230 |
| VP8 protein | MASLIYRQLLTNSYSVDLHDEIEQIGSEKTQNVTINPSPFAQTRYAPVNWGHGEINDSTTV |
| | EPILDGPYQPTTFTPPNDYWILINSNTNGVVYESTNNSDFWTAVVAIEPHVNPVDRQYTIF |
| | GESKQFNVSNDSNKWKFLEMFRSSSQNEFYNRRTLTSDTRFVGILKYGGRVWTFHGETPRA |
| | TTDSSSTANLNNISITIHSEFYIIPRSQESKCNEYINNGLPPIQNTR (SEQ ID |
| | NO: 143) |
| Human | >sp\|Q6WB98\|FUS_HMPVC Fusion glycoprotein FO OS = Human |
| metapneumovirus | metapneumovirus (strain CAN97-83) GN = F PE = 1 SV = 1 |
| (hMPV) | MSWKVVIIFSLLITPQHGLKESYLEESCSTITEGYLSVLRTGWYTNVFTLEVGDVENLTCS |
| F protein | DGPSLIKTELDLTKSALRELKTVSADQLAREEQIENPRQSRFVLGAIALGVATAAAVTAGV |
| | AIAKTIRLESEVTAIKNALKTTNEAVSTLGNGVRVLATAVRELKDFVSKNLTRAINKNKCD |
| | IDDLKMAVSFSQFNRRFLNVVRQFSDNAGITPAISLDLMTDAELARAVSNMPTSAGQIKLM |
| | LENRAMVRRKGFGILIGVYGSSVIYMVQLPIFGVIDTPCWIVKAAPSCSGKKGNYACLLRE |
| | DQGWYCQNAGSTVYYPNEKDCETRGDHVFCDTAAGINVAEQSKECNINISTTNYPCKVSTG |
| | RHPISMVALSPLGALVACYKGVSCSIGSNRVGIIKQLNKGCSYITNQDADTVTIDNTVYQL |
| | SKVEGEQHVIKGRPVSSSFDPIKFPEDQFNVALDQVFENIENSQALVDQSNRILSSAEKGN |
| | TGFIIVIILIAVLGSSMILVSIFIIIKKTKKPTGAPPELSGVTNNGFIPHS(SEQ ID |
| | NO: 144) |
| Human | >sp\|Q6WB94\|VGLG_HMPVC Major surface glycoprotein G OS = Human |
| metapneumovirus | metapneumovirus (strain CAN97-83) GN = G PE = 1 SV = 1 |
| (hMPV) | MEVKVENIRAIDMLKARVKNRVARSKCFKNASLILIGITTLSIALNIYLIINYTIQKTSSE |
| G protein | SEHHTSSPPTESNKEASTISTDNPDINPNSQHPTQQSTENPTLNPAASVSPSETEPASTPD |
| | TTNRLSSVDRSTAQPSESRTKTKPTVHTRNNPSTASSTQSPPRATTKAIRRATTERMSSTG |
| | KRPTTTSVQSDSSTTTQNHEETGSANPQASVSTMQN (SEQ ID NO: 145) |
| Human | >sp\|P12605\|FUS_PI1HC Fusion glycoprotein FO OS = Human |
| parainfluenza | parainfluenza 1 virus (strain C39) GN = F PE = 2 SV = 1 |
| virus (PV) | MQKSEILFLIYSSLLLSSSLCQIPVDKLSNVGVIINEGKLLKIAGSYESRYIVLSLVPSID |
| F protein | LEDGCGTTQIIQYKNLLNRLLIPLKDALDLQESLITITNDTTVTNDNPQSRFFGAVIGTIA |
| | LGVATAAQITAGIALAEAREARKDIALIKDSIIKTHNSVELIQRGIGEQIIALKTLQDEVN |
| | NEIRPAIGELRCETTALKLGIKLTQHYSELATAFSSNLGTIGEKSLTLQALSSLYSANITE |
| | ILSTIKKDKSDIYDIIYTEQVKGTVIDVDLEKYMVTLLVKIPILSEIPGVLIYRASSISYN |
| | IEGEEWHVAIPNYIINKASSLGGADVINCIESRLAYICPRDPTQLIPDNQQKCILGDVSKC |
| | PVTKVINNLVPKFAFINGGVVANCIASTCTCGTNRIPVNQDRSRGVTFLTYTNCGLIGING |
| | IELYANKRGRDTTWGNQIIKVGPAVSIRPVDISLNLASATNFLEESKIELMKAKAIISAVG |
| | GWHNTESTQIIIIIIVCILIIIICGILYYLYRVRRLLVMINSTHNSPVNTYTLESRMRNPY |
| | IGNNSN (SEQ ID NO: 146) |
| Human | >sp\|P25466\|HN_PI2HT Hemagglutinin-neuraminidase OS = Human |
| parainfluenza | parainfluenza 2 virus (strain Toshiba) GN = HN PE = 2 SV = 1 |
| virus | MEDYSNLSLKSIPKRTCRIIFRTATILGICTLIVLCSSILHEIIHLDVSSGLMDSDDSQQG |
| HN protein | IIQPIIESLKSLIALANQILYNVAIIIPLKIDSIETVIFSALKDMHTGSMSNTNCTPGNLL |
| | LHDAAYINGINKFLVLKSYNGTPKYGPLLNIPSFIPSATSPNGCTRIPSFSLIKTHWCYTH |
| | NVMLGDCLDFTTSNQYLAMGIIQQSAAAFPIFRTMKTIYLSDGINRKSCSVTAIPGGCVLY |
| | CYVATRSEKEDYATTDLAELRLAFYYYNDTFIERVISLPNTTGQWATINPAVGSGIYHLGF |
| | ILFPVYGGLISGTPSYNKQSSRYFIPKHPNITCAGNSSEQAAAARSSYVIRYHSNRLIQSA |
| | VLICPLSDMHTARCNLVMENNSQVMMGAEGRLYVIDNNLYYYQRSSSWWSASLFYRINTDE |
| | SKGIPPIIEAQWVPSYQVPRPGVMPCNATSFCPANCITGVYADVWPLNDPEPTSQNALNPN |
| | YRFAGAFLRNESNRTNPTFYTASASALLNTTGFNNTNHKAAYTSSTCFKNTGTQKIYCLII |
| | IEMGSSLLGEFQIIPFLRELIP (SEQ ID NO: 147) |
| Malaria | >sp\|P13829\|OS25_PLAFO 25 kDa ookinete surface antigen |
| Pfs25 surface | OS = Plasmodium falciparum (isolate NF54) PE = 1 SV = 1 |
| antigen | MNKLYSLFLFLFIQLSIKYNNAKVTVDTVCKRGFLIQMSGHLECKCENDLVLVNEETCEEK |
| | VLKCDEKTVNKPCGDFSKCIKIDGNPVSYACKCNLGYDMVNNVCIPNECKNVTCGNGKCIL |
| | DTSNPVKTGVCSCNIGKVPNVQDQNKCSKDGETKCSLKCLKFNETCKAVDGIYKCDCKDGF |
| | IIDNESSICTAFSAYNILNLSIMFILESVCFFIM (SEQ ID NO: 148) |
| serogroup B | >tr\|Q6QCC2\|Q6QCC2_NEIME Factor H-binding protein OS = Neisseria |
| Neisseria | meningitidis OX = 487 GN = gna1870 PE = 1 SV = 1 |
| meningitidis | MNRTAFCCLSLTTALILTACSSGGGGVAADIGAGLADALTAPLDHKDKGLQSLTLDQSVRK |
| (MenB) | NEKLKLAAQGAEKTYGNGDSLNTGKLKNDKVSRFDFIRQIEVDGQLITLESGEFQVYKQSH |
| fHbp | SALTAFQTEQIQDSEHSGKMVAKRQFRIGDIAGEHTSFDKLPEGGRATYRGTAFGSDDAGG |
| | KLTYTIDFAAKQGNGKIEHLKSPELNVDLAAADIKPDGKRHAVISGSVLYNQAEKGSYSLG |
| | IFGGKAQEVAGSAEVKTVNGIRHIGLAAKQ (SEQ ID NO: 149) |
| serogroup B | >tr\|X5F9B9\|X5F9B9_NEIME Quinolinate synthase A OS = Neisseria |
| Neisseria | meningitidis OX = 487 GN = nadA2 PE = 3 SV = 1 |
| meningitidis | MQTAARRSFDYDMPLIQTPTSACQIRQAWAKVADTPDRETAGRLKDEIKALLKETNAVLVA |
| (MenB) | HYYVDPLIQDLALETGGCVGDSLEMARFGAEHEAGTLVVAGVRFMGESAKILCPEKTVLMP |
| NadA | DLEAECSLDLGCPEEAFSAFCDQHPDRTVVVYANTSAAVKARADWVVTSSVALEIVSYLKS |
| | RGEKLIWGPDRHLGDYIRRETGADMLLWQGSCIVHNEFKGQELAALKAEHPDAVVLVHPES |
| | PQSVIELGDVVGSTSKLLKAAVSRPEKKFIVATDLGILHEMQKQAPDKQFIAAPTAGNGGS |
| | CKSCAFCPWMAMNSLGGIKYALTSGHNEILLDRKLGEAAKLPLQRMLDFAAGLKRGDVENG |
| | MGPA (SEQ ID NO: 150) |
| serogroup B | >tr\|Q9JPH1\|Q9JPH1_NEIME Gna2132 OS = Neisseria meningitidis |
| Neisseria | OX = 487 GN = gna2132 PE = 4 SV = 1 |
| meningitidis | MFKRSVIAMACIFALSACGGGGGGSPDVKSADTLSKPAAPVVSEKETEAKEDAPQAGSQGQ |
| (MenB) | GAPSAQGGQDMAAVSEENTGNGGAAATDKPKNEDEGAQNDMPQNAADTDSLTPNHTPASNM |
| NHBA | PAGNMENQAPDAGESEQPANQPDMANTADGMQGDDPSAGGENAGNTAAQGTNQAENNQTAG |
| | SQNPASSTNPSATNSGGDFGRTNVGNSVVIDGPSQNITLTHCKGDSCSGNNFLDEEVQLKS |
| | EFEKLSDADKISNYKKDGKNDGKNDKFVGLVADSVQMKGINQYIIFYKPKPTSFARFRRSA |
| | RSRRSLPAEMPLIPVNQADTLIVDGEAVSLTGHSGNIFAPEGNYRYLTYGAEKLPGGSYAL |
| | RVQGEPSKGEMLAGTAVYNGEVLHFHTENGRPSPSRGRFAAKVDFGSKSVDGIIDSGDGLH |
| | MGTQKFKAAIDGNGFKGTWTENGGGDVSGKFYGPAGEEVAGKYSYRPTDAEKGGFGVFAGK |
| | KEQD (SEQ ID NO: 151) |

The disclosure also provides nanoparticles, comprising:
(a) a plurality of first assemblies, each first assembly comprising a plurality of identical first proteins comprising the amino acid sequence selected from the group consisting of SEQ ID NO: 10, 13, 23, and 59-60; and,
(b) a plurality of second assemblies, each second assembly comprising a plurality of second proteins comprising the amino acid sequence selected from the group consisting of SEQ ID NO:1-3, 5, 8-9, and 49-55;
wherein the plurality of first assemblies non-covalently interact with the plurality of second assemblies to form the nanoparticle.

A plurality (2, 3, 4, 5, 6, or more) of first proteins self-assemble to form a first assembly, and a plurality (2, 3, 4, 5, 6, or more) of second proteins self-assemble to form a second assembly. A plurality of these first and second assemblies then self-assemble non-covalently via the designed interfaces to produce the nanoparticles (i.e., particles having a dimension on the nanometer scale).

The number of first proteins in the first assemblies may be the same or different than the number of second proteins in the second assemblies. In one exemplary embodiment, the first assembly comprises trimers of the first proteins, and the second assembly comprises pentamers of the second proteins.

In one embodiment, each first assembly comprises a plurality of identical first proteins comprising an amino acid sequence selected from the group consisting of SEQ ID NO: 10, 13, and 59-60. In another embodiment, each first assembly comprises a plurality of identical first proteins comprising an amino acid sequence selected from the group consisting of SEQ ID NO: 59-60.

In one embodiment, each second assembly comprising a plurality of second proteins comprising an amino acid sequence selected from the group consisting of SEQ ID NO: 1-3, 5, 8-9, 26-28, 31-32, 34-38, 40, 42-46, 48-55, and 67-74. In another embodiment, each second assembly comprising a plurality of second proteins comprising an amino acid sequence selected from the group consisting of SEQ ID NO: 49-55, and 67-74. In a further embodiment, each second assembly comprising a plurality of second proteins comprising an amino acid sequence selected from the group consisting of SEQ ID NO: 55, 67, and 73.

In another embodiment, the disclosure provides nanoparticles, comprising:
(a) a plurality of first assemblies, each first assembly comprising a plurality of identical first proteins comprising the amino acid sequence selected of SEQ ID NO:152 or 153; and,
(b) a plurality of second assemblies, each second assembly comprising a plurality of second proteins comprising an amino acid sequence selected from the group consisting of SEQ ID NO: 26-48 and 61-77;
wherein the plurality of first assemblies non-covalently interact with the plurality of second assemblies to form the nanoparticle.

I53-50B-residues in parentheses optional, and may
be present or absent
(SEQ ID NO: 152)
(M)NQHSHKDYETVRIAVVRARWHAEIVDACVSAFEAAMADIGGDRFAVD
VFDVPGAYEIPLHARTLAETGRYGAVLGTAFVVNGGIYRHEFVASAVIDG
MMNVQLSTGVPVLSAVLTPHRYRDSDAHTLLFLALFAVKGMEAARACVEI
LAAREKIAA
SEQ ID NO: 153
(M)NQHSHKDHETVRIAVVRARWHAEIVDACVSAFEAAMRDIGGDRFAVD
VFDVPGAYEIPLHARTLAETGRYGAVLGTAFVVNGGIYRHEFVASAVING
MMNVQLNTGVPVLSAVLTPHNYDKSKAHTLLFLALFAVKGMEAARACVEI
LAAREKIAA(GSLEHHHHHH)

In various embodiments, the plurality of second proteins comprise an amino acid sequence selected from the group consisting of SEQ ID NO: 26-28, 31-32, 34-38, 40, 42-46, 48, and 67-77; or the plurality of second proteins comprise an amino acid sequence selected from the group consisting of SEQ ID NO: 67-74; or the plurality of second proteins comprise an amino acid sequence selected from the group consisting of SEQ ID NO: 67 and 73.

In one embodiment of all nanoparticles of the disclosure, some (at least 1%, 5%, 10%, 20%, 25%, 30%, 40%, 50%, 60%, 70%, 75%, 80%, 85%, 90%, 95%) of the second proteins comprise a fusion protein of any embodiment or combination of embodiments herein. In one embodiment, all of the second proteins comprise a fusion protein. In one embodiment, the fusion protein comprises an antigen according to any embodiment disclosed herein, and the nanoparticle displays the antigen(s) on an exterior of the nanoparticle. As used herein, “on an exterior of the nanoparticle” means that an antigenic portion of the one or more antigens or antigenic fragments thereof, are accessible for binding by B cell receptors, antibodies, or antibody fragments and not buried within the nanoparticle. In some embodiments, each second protein of the nanostructure bears an antigen as a genetic fusion; these nanoparticles display antigen at full (100%) valency. In other embodiments, the nanoparticles of the disclosure comprise one or more second proteins bearing antigens as genetic fusions as well as one or more second proteins that do not bear antigens as genetic fusions; these nanoparticles display the antigens at partial valency. In other embodiments, the nanoparticles of the disclosure comprise two or more distinct second proteins bearing different antigens as genetic fusions.

In various embodiments, the nanoparticles are between about 20 nanometers (nm) to about 40 nm in diameter, with interior lumens between about 15 nm to about 32 nm across and pore sizes in the protein shells between about 1 nm to about 14 nm in their longest dimensions.

In another aspect the disclosure provides nucleic acids encoding the polypeptide or fusion protein of any embodiment or combination of embodiments of the disclosure. The nucleic acid sequence may comprise single stranded or double stranded RNA or DNA in genomic or cDNA form, or DNA-RNA hybrids, each of which may include chemically or biochemically modified, non-natural, or derivatized nucleotide bases. Such nucleic acid sequences may comprise additional sequences useful for promoting expression and/or purification of the encoded peptide or chimeric molecular construct, including but not limited to polyA sequences, modified Kozak sequences, and sequences encoding epitope tags, export signals, and secretory signals, nuclear localization signals, and plasma membrane localization signals. It will be apparent to those of skill in the art, based on the teachings herein, what nucleic acid sequences will encode the peptide or chimeric molecular construct of the disclosure.

In a further aspect, the disclosure provides expression vectors comprising the nucleic acid of any aspect of the disclosure operatively linked to a suitable control sequence. “Expression vector” includes vectors that operatively link a nucleic acid coding region or gene to any control sequences capable of effecting expression of the gene product. “Control sequences” operably linked to the nucleic acid sequences of the disclosure are nucleic acid sequences capable of effecting the expression of the nucleic acid molecules. The control sequences need not be contiguous with the nucleic acid sequences, so long as they function to direct the expression thereof. Thus, for example, intervening untranslated yet transcribed sequences can be present between a promoter sequence and the nucleic acid sequences and the promoter sequence can still be considered “operably linked” to the coding sequence. Other such control sequences include, but are not limited to, polyadenylation signals, termination signals, and ribosome binding sites. Such expression vectors can be of any type, including but not limited plasmid and viral-based expression vectors. The control sequence used to drive expression of the disclosed nucleic acid sequences in a mammalian system may be constitutive (driven by any of a variety of promoters, including but not limited to, CMV, SV40, RSV, actin, EF) or inducible (driven by any of a number of inducible promoters including, but not limited to, tetracycline, ecdysone, steroid-responsive). The expression vector must be replicable in the host organisms either as an episome or by integration into host chromosomal DNA. In various embodiments, the expression vector may comprise a plasmid, viral-based vector, or any other suitable expression vector.

In another aspect, the disclosure provides host cells that comprise the polypeptide, fusion protein, nanoparticle, nucleic acid or expression vector (i.e.: episomal or chromosomally integrated) disclosed herein, wherein the host cells can be either prokaryotic or eukaryotic. The cells can be transiently or stably engineered to incorporate the expression vector of the disclosure, using techniques including but not limited to bacterial transformations, calcium phosphate co-precipitation, electroporation, or liposome mediated-, DEAE dextran mediated-, polycationic mediated-, or viral mediated transfection.

In a further aspect, the disclosure provides a composition comprising a plurality of the proteins, fusion proteins, nucleic acids, expression vectors, and/or nanoparticles of the disclosure. In one embodiment, the composition comprises a pharmaceutical composition or an immunogenic composition (such as a vaccine) comprising an effective amount of the proteins, fusion proteins, nucleic acids, expression vectors, and/or nanoparticles of any embodiment or combination of embodiments of the disclosure that incorporates an antigen; and a pharmaceutically acceptable carrier. The composition may comprise (a) a lyoprotectant; (b) a surfactant; (c) a bulking agent; (d) a tonicity adjusting agent; (e) a stabilizer; (f) a preservative and/or (g) a buffer.

In some embodiments, the buffer in the pharmaceutical composition is a Tris buffer, a histidine buffer, a phosphate buffer, a citrate buffer or an acetate buffer. The composition may also include a lyoprotectant, e.g. sucrose, sorbitol or trehalose. In certain embodiments, the composition includes a preservative e.g. benzalkonium chloride, benzethonium, chlorohexidine, phenol, m-cresol, benzyl alcohol, methylparaben, propylparaben, chlorobutanol, o-cresol, p-cresol, chlorocresol, phenylmercuric nitrate, thimerosal, benzoic acid, and various mixtures thereof. In other embodiments, the composition includes a bulking agent, like glycine. In yet other embodiments, the composition includes a surfactant e.g., polysorbate-20, polysorbate-40, polysorbate-60, polysorbate-65, polysorbate-80 polysorbate-85, poloxamer-188, sorbitan monolaurate, sorbitan monopalmitate, sorbitan monostearate, sorbitan monooleate, sorbitan trilaurate, sorbitan tristearate, sorbitan trioleaste, or a combination thereof. The composition may also include a tonicity adjusting agent, e.g., a compound that renders the formulation substantially isotonic or isoosmotic with human blood. Exemplary tonicity adjusting agents include sucrose, sorbitol, glycine, methionine, mannitol, dextrose, inositol, sodium chloride, arginine and arginine hydrochloride. In other embodiments, the composition additionally includes a stabilizer, e.g., a molecule which substantially prevents or reduces chemical and/or physical instability of the nanostructure, in lyophilized or liquid form. Exemplary stabilizers include sucrose, sorbitol, glycine, inositol, sodium chloride, methionine, arginine, and arginine hydrochloride.

The nanoparticle may be the sole active agent in the composition, or the composition may further comprise one or more other agents suitable for an intended use, including but not limited to adjuvants to stimulate the immune system generally and improve immune responses overall. Any suitable adjuvant can be used. The term “adjuvant” refers to a compound or mixture that enhances the immune response to an antigen. Exemplary adjuvants include, but are not limited to, Adju-Phos™, Adjumer™, albumin-heparin microparticles, Algal Glucan, Algammulin, Alum, Antigen Formulation, AS-2 adjuvant, autologous dendritic cells, autologous PBMC, Avridine™, B7-2, BAK, BAY R1005, Bupivacaine, Bupivacaine-HCl, BWZL, Calcitriol, Calcium Phosphate Gel, CCR5 peptides, CFA, Cholera holotoxin (CT) and Cholera toxin B subunit (CTB), Cholera toxin A1-subunit-Protein A D-fragment fusion protein, CpG, CRL1005, Cytokine-containing Liposomes, D-Murapalmitine, DDA, DHEA, Diphtheria toxoid, DL-PGL, DMPC, DMPG, DOC/Alum Complex, Fowlpox, Freund's Complete Adjuvant, Gamma Inulin, Gerbu Adjuvant, GM-CSF, GMDP, hGM-CSF, hIL-12 (N222L), hTNF-alpha, IFA, IFN-gamma in pcDNA3, IL-12 DNA, IL-12 plasmid, IL-12/GMCSF plasmid (Sykes), IL-2 in pcDNA3, IL-2/Ig plasmid, IL-2/Ig protein, IL-4, IL-4 in pcDNA3, Imiquimod™, ImmTher™ Immunoliposomes Containing Antibodies to Costimulatory Molecules, Interferon-gamma, Interleukin-1 beta, Interleukin-12, Interleukin-2, Interleukin-7, ISCOM(s)™, Iscoprep 7.0.3™, Keyhole Limpet Hemocyanin, Lipid-based Adjuvant, Liposomes, Loxoribine, LT(R192G), LT-OA or LT Oral Adjuvant, LT-R192G, LTK63, LTK72, MF59, MONTANIDE ISA 51, MONTANIDE ISA 720, MPL.TM., MPL-SE, MTP-PE, MTP-PE Liposomes, Murametide, Murapalmitine, NAGO, nCT native Cholera Toxin, Non-Ionic Surfactant Vesicles, non-toxic mutant E112K of Cholera Toxin mCT-E112K, p-Hydroxybenzoique acid methyl ester, pCIL-10, pCIL12, pCMVmCAT1, pCMVN, Peptomer-NP, Pleuran, PLG, PLGA, PGA, and PLA, Pluronic L121, PMMA, PODDS™, Poly rA: Poly rU, Polysorbate 80, Protein Cochleates, QS-21, Quadri A saponin, Quil-A, Rehydragel HPA, Rehydragel LV, RIBI, Ribilike adjuvant system (MPL, TMD, CWS), S-28463, SAF-1, Sclavo peptide, Sendai Proteoliposomes, Sendai-containing Lipid Matrices, Span 85, Specol, Squalane 1, Squalene 2, Stearyl Tyrosine, Tetanus toxoid (TT), Theramide™, Threonyl muramyl dipeptide (TMDP), Ty Particles, and Walter Reed Liposomes. Selection of an adjuvant depends on the subject to be treated. Preferably, a pharmaceutically acceptable adjuvant is used.

In another aspect, the disclosure provides methods for generating an immune response to an antigen in a subject, comprising administering to the subject an effective amount of the immunogenic composition of any embodiment or combination of embodiments of the disclosure to generate the immune response. In a further aspect, the disclosure provides methods for treating or preventing an infection in a subject, comprising administering to the subject an effective amount of the immunogenic composition of any embodiment or combination of embodiments of the disclosure that comprises an antigen, or antigenic fragment thereof, from the infectious agent to be treated or prevented, thereby treating or preventing infection in the subject. Exemplary antigens and infectious agents are disclosed herein.

As used herein, “treat” or “treating” includes, but is not limited to accomplishing one or more of the following (depending on the infectious agent): (a) reducing viral titer in the subject; (b) limiting any increase of viral titer in the subject; (c) reducing the severity of infectious agent symptoms; (d) limiting or preventing development of infectious agent symptoms after infection; (e) inhibiting worsening of infectious agent symptoms; (f) limiting or preventing recurrence of infectious agent symptoms in subjects that were previously symptomatic; and/or promoting maternal transmission of infectious agent antibodies to infants (after maternal immunization).

When the method comprises limiting an infection, the immunogenic compositions are administered prophylactically to a subject that is not known to be infected, but may be at risk of exposure to the infectious agent of interest. As used herein, “limiting” means to limit infection in subjects at risk of infection.

As used herein, an “effective amount” refers to an amount of the immunogenic composition that is effective for treating and/or limiting infection. The immunogenic compositions are typically formulated as a pharmaceutical composition, such as those disclosed above, and can be administered via any suitable route, including orally, parentally, by inhalation spray, rectally, or topically in dosage unit formulations containing conventional pharmaceutically acceptable carriers, adjuvants, and vehicles. The term parenteral as used herein includes, subcutaneous, intravenous, intra-arterial, intramuscular, intrasternal, intratendinous, intraspinal, intracranial, intrathoracic, infusion techniques or intraperitoneally. Polypeptide compositions may also be administered via microspheres, liposomes, immune-stimulating complexes (ISCOMs), or other microparticulate delivery systems or sustained release formulations introduced into suitable tissues (such as blood). Dosage regimens can be adjusted to provide the optimum desired response (e.g., a therapeutic or prophylactic response). A suitable dosage range may, for instance, be 0.1 ug/kg-100 mg/kg body weight of the antigen or antigenic fragment thereof. The composition can be delivered in a single bolus, or may be administered more than once (e.g., 2, 3, 4, 5, or more times) as determined by attending medical personnel.

### EXAMPLES

The rules for how immune responses specific to nanoparticle scaffolds affect the immunogenicity of displayed antigens have not been established. Here we define relationships between anti-scaffold and antigen-specific antibody responses elicited by protein nanoparticle immunogens. We found that dampening anti-scaffold responses by physical masking did not enhance antigen-specific antibody responses. In a series of immunogens that all used the same nanoparticle scaffold but displayed four different antigens, only HIV-1 envelope glycoprotein (Env) was subdominant to the scaffold. Yet we also demonstrated that scaffold-specific antibody responses can competitively inhibit antigen-specific responses when the scaffold is provided in excess. Overall, our results suggest that anti-scaffold antibody responses are unlikely to suppress antigen-specific antibody responses for protein nanoparticle immunogens in which the antigen is immunodominant over the scaffold.

Here, we address questions about the role of anti-scaffold responses in shaping the immunogenicity of protein nanoparticle immunogens through (1) physically masking the nanoparticle scaffold using three different approaches, (2) studying how antigen immunodominance impacts anti-scaffold responses, and (3) assessing immunogenic competition between the displayed antigen and nanoparticle scaffold. We found that scaffold masking reduced scaffold-specific antibody responses, which may be desirable in a vaccine intended to elicit protective antibody responses against a displayed antigen. Reducing anti-scaffold responses would be particularly useful in instances where the same animal or human receives different vaccines displaying distinct antigens but using the same underlying nanoparticle scaffold.

### Results

Design and Characterization of HA-I53_Dn5 Nanoparticle Immunogens with a Glycosylated, PEGylated, or PASylated Nanoparticle Scaffold

To test the impact of masking the nanoparticle scaffold on antigen-specific antibody responses, we selected as our model scaffold the I53_dn5 protein nanoparticle (Ueda et al., 2020) due to its robust self-assembly and stability and its use as the scaffold for a mosaic nanoparticle influenza vaccine in clinical testing (Boyoglu-Barnum et al., 2021; NCT04896086). I53_dn5 is a 25 nm, two-component nanoparticle with icosahedral symmetry constructed from 12 pentameric and 20 trimeric building blocks. We compared three different approaches to masking I53_dn5 surfaces: glycosylation, PEGylation, and genetic fusion of unstructured polypeptides rich in Pro, Ala, and Ser (i.e., PASylation (Schlapschy et al., 2013)).

To introduce NxT/S potential N-linked glycosylation sites (PNGS) into the exposed surfaces of the I53_dn5A pentamer and the I53_dn5B trimer, we used a custom “sugarcoat” protocol that we recently developed (Adolf-Bryfogle et al., 2021) as part of the Rosetta™ macromolecular modeling and design software (Fleishman et al., 2011; Leman et al., 2020).

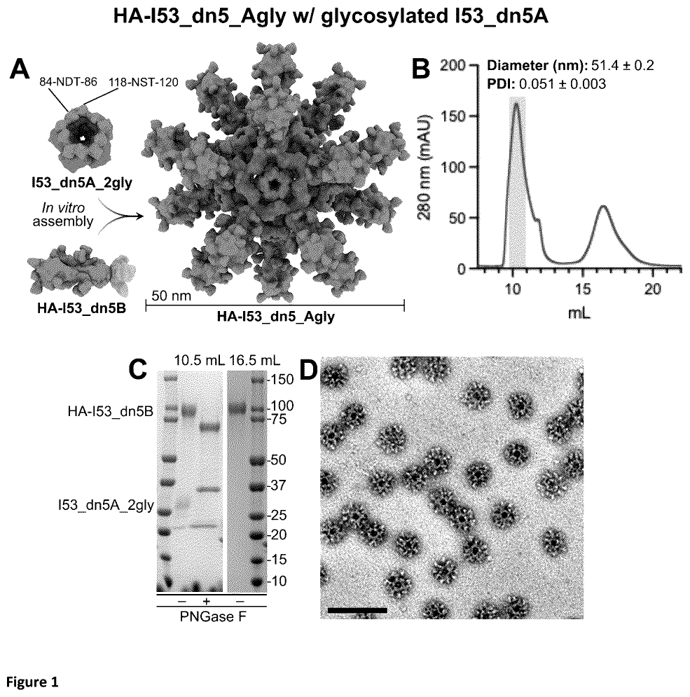

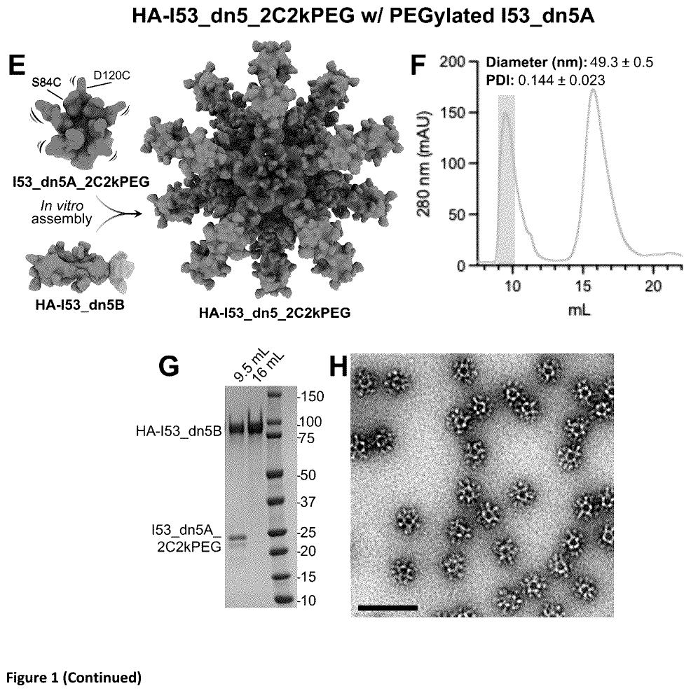

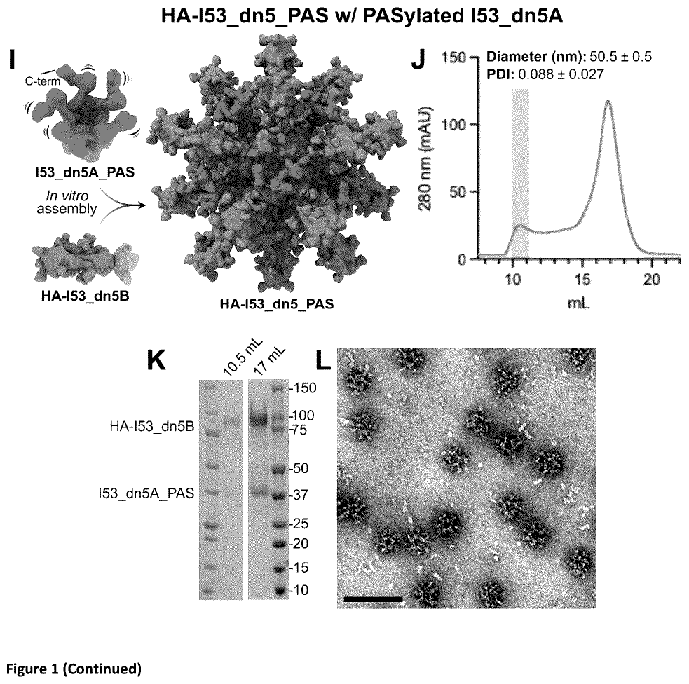

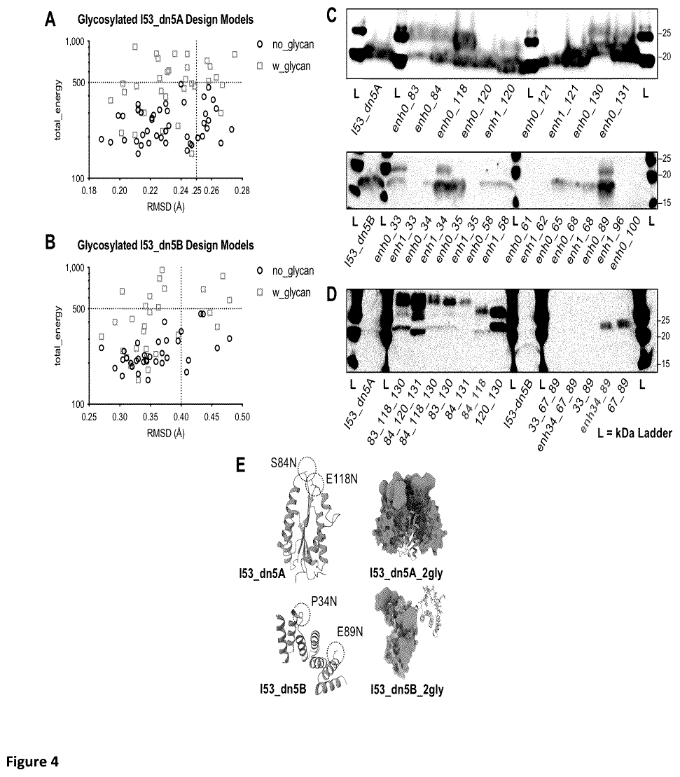

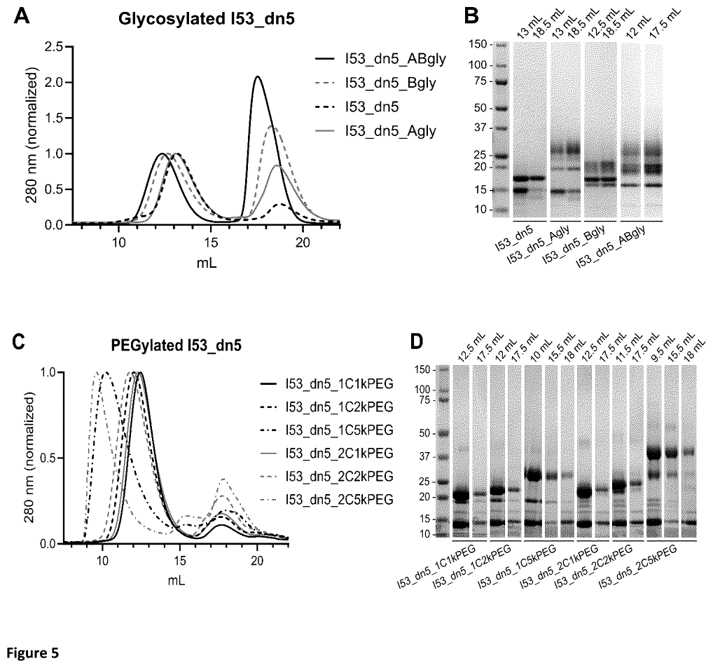

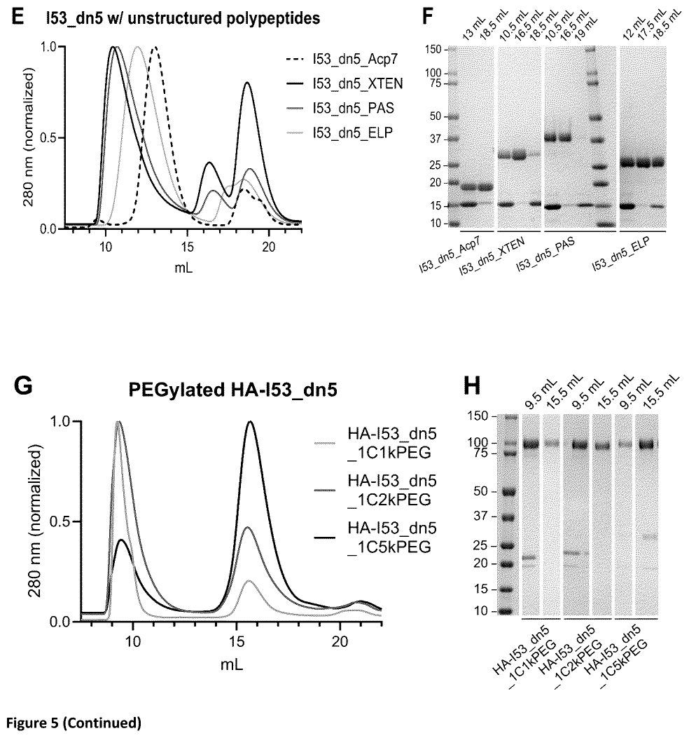

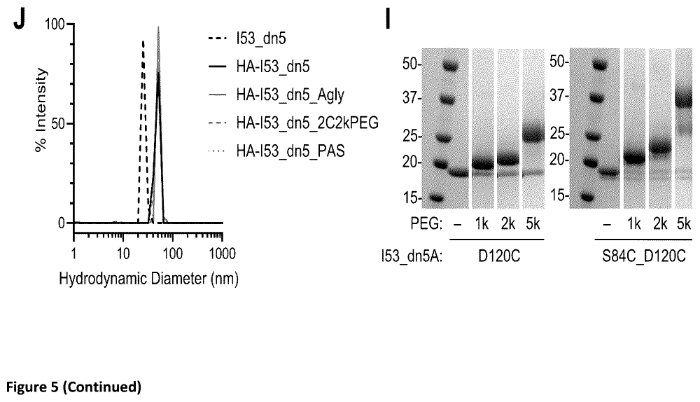

Sequences corresponding to design models containing a single inserted NxT/S sequon, modeled with and without a Man9 glycan tree present, that had a Rosetta™ “total_energy”<500, as well as <0.25 Å and <0.40 Å backbone (Ca) root mean square deviation (RMSD) compared to the parent I53_dn5A and I53_dn5B design models, respectively, were tested for protein expression and glycosylation (FIGS. 4A-B). Reducing western blot analysis of cell culture supernatants showed variable expression and glycosylation for nine I53_dn5A and sixteen I53_dn5B variants that contained a single NxT/S sequon (FIG. 4C). For I53_dn5A, six variants that expressed better than the parent sequence and/or exhibited migration indicating glycosylation were considered further. For I53_dn5B, three variants exhibited partial glycosylation. Next, variants that contained combinations of these validated single NxT/S PNGS were tested for expression, glycosylation, and nanoparticle assembly competency. A single variant of I53_dn5A (I53_dn5A_2gly; 84-NDT-86, 118-NST-120) and I53_dn5B (I53_dn5B_2gly; 32-YDNLT-36, 89-NAT-91), each with two glycans per protomer, exhibited the most efficient expression, glycosylation, and nanoparticle assembly based on reducing western blot analysis of cell culture supernatants and size exclusion chromatography (SEC) (FIGS. 4D, 5A). These two glycosylated variants also assembled with each other to form glycosylated I53_dn5 particles (I53_dn5_ABgly) bearing 240 glycans on the exterior surface, although with somewhat reduced efficiency (FIGS. 5A-B). To produce hemagglutinin (HA)-bearing nanoparticle immunogens with a glycosylated scaffold, I53_dn5A_2gly was mixed with a genetic fusion of an H1 HA (H1/A/Michigan/45/2015, “MI15”) and the I53_dn5B trimer (HA-I53_dn5B) in vitro with a slight molar excess of the trimeric components (FIG. 1A), and purified via SEC (FIG. 1B).

PNGase F digestion of N-linked glycans followed by reducing SDS-PAGE analysis verified glycosylation of the I53_dn5A_2gly component of the SEC-purified HA-I53_dn5_Agly particles (FIG. 1C). Dynamic light scattering (DLS) (FIGS. 1B, 5J) and negative stain transmission electron microscopy (nsTEM) (FIG. 1D) analysis verified assembly into monodisperse nanoparticle immunogens with the intended icosahedral architecture.

To specifically couple PEG to precise locations on the I53_dn5 nanoparticle surface, we designed I53_dn5A pentamer variants with surface-exposed cysteines to enable PEG-maleimide conjugation (Goodson and Katre, 1990). We did not design I53_dn5B trimer cysteine knock-ins due to the potential for coupled PEG to occlude membrane-proximal epitopes on fused antigens (e.g., the conserved HA stem region). Seven I53_dn5A cysteine knock-ins were designed with either one or two surface-exposed cysteines per protomer. Two designs (I53_dn5A_D120C, I53_dn5A_S84C_D120C) had acceptable expression (>100 mg/L of bacterial expression media); did not aggregate during 4° C. storage; coupled efficiently to 1, 2, and 5 kDa PEG; and assembled into PEGylated I53_dn5 nanoparticles based on SEC purification (FIGS. 5C, 5I). However, I53_dn5A_D120C pentamers with a single conjugated 5 kDa PEG per subunit did not efficiently assemble with HA-bearing I53_dn5B trimers (FIG. 5G). By contrast, PEGylated HA-I53_dn5 immunogens with 1 or 2 kDa PEG coupled to the ten thiol groups on each I53_dn5A_S84C_D120C pentamer (FIG. 1E) were found to form monodisperse particles based on SEC, DLS, and nsTEM (FIGS. 1F, 1H, 5J), and these were carried forward for in vivo testing.

An alternative physical masking approach to PEGylation is the genetic fusion of hydrophilic unstructured polypeptides, such as XTENylation and PASylation (Schellenberger et al., 2009; Schlapschy et al., 2013; Zaman et al., 2019); these have been expressed on ferritin to extend its circulation time in vivo (Falvo et al., 2016; Lee et al., 2017). To express XTEN and PAS polypeptides on the outer surface of the I53_dn5A pentamer, we first designed a circularly permuted variant of I53_dn5A, called I53_dn5Acp7, with the N and C termini both facing outward. I53_dn5 nanoparticle formation was observed via SEC when XTEN, PAS, and another unstructured polypeptide known as ELP (Luginbuhl et al., 2017) were fused to the C terminus of the I53_dn5Acp7 pentamer (FIG. 5E). PASylated I53_dn5Acp7 (I53_dn5A_PAS) assembled, albeit inefficiently, with HA-I53_dn5B trimers (FIG. 11) to form monodisperse nanoparticle immunogens based on SEC, DLS, and nsTEM, with nsTEM revealing some presence of unassembled components in the nanoparticle sample (FIGS. 1J, 1K, 1L, 5J), and were used for in vivo studies.

Overall, based on SEC and SDS-PAGE analysis, the larger PAS polypeptide and PEG molecules impeded efficient nanoparticle assembly (FIGS. 1J, 1K, 5C-H) more than smaller PEG and glycans did (FIGS. 1B, 1F, 1C, 1G, 5A-D, 5G-H). This trend of less efficient nanoparticle assembly with bulkier masking groups is consistent with the estimated molecular weight of each masking moiety on the I53_dn5A pentamer (and hydrodynamic diameter of assembled I53_dn5 nanoparticles with the respective masked I53_dn5A): 9-mannose N-linked glycan, 1,884 Da (31 nm); 2 kDa PEG, 2,000 Da (31 nm); 63 amino acid PAS polypeptide, 5,187 Da (38 nm), suggesting that the presence of larger, flexible masking agents interferes with nanoparticle assembly. However, we note that very large glycoprotein antigens such as HIV-1 Env and SARS-CoV-2 Spike (approximately 120 and 170 kDa per monomer including glycans, respectively) are able to efficiently assemble into I53_dn5 and similarly sized I53-50 nanoparticles (Brouwer et al., 2019, 2021a), presumably because they are not quite so dynamic.

Masking the I53_Dn5 Nanoparticle Scaffold does not Enhance Anti-HA Antibody Responses

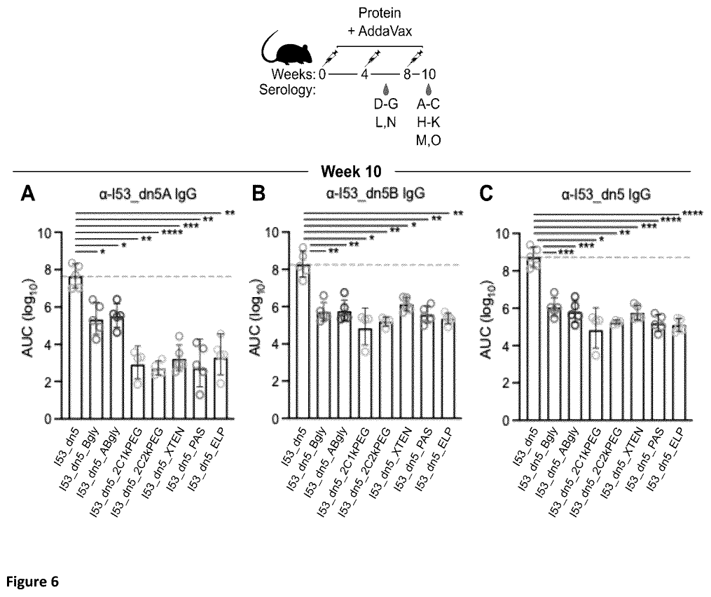

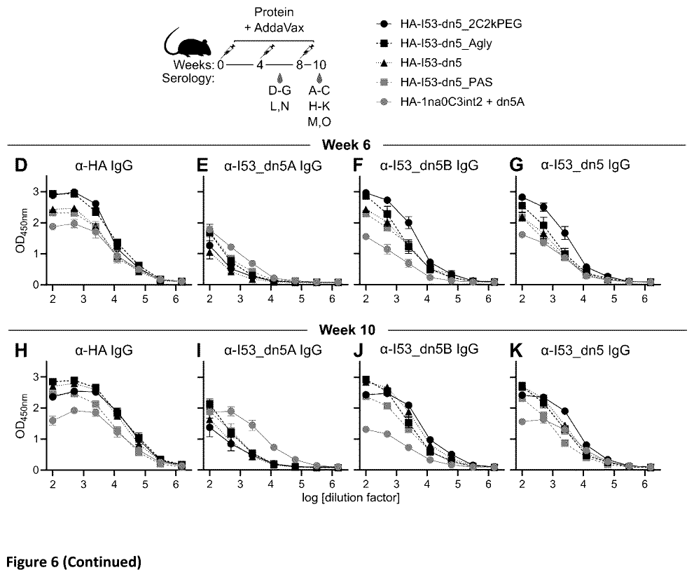

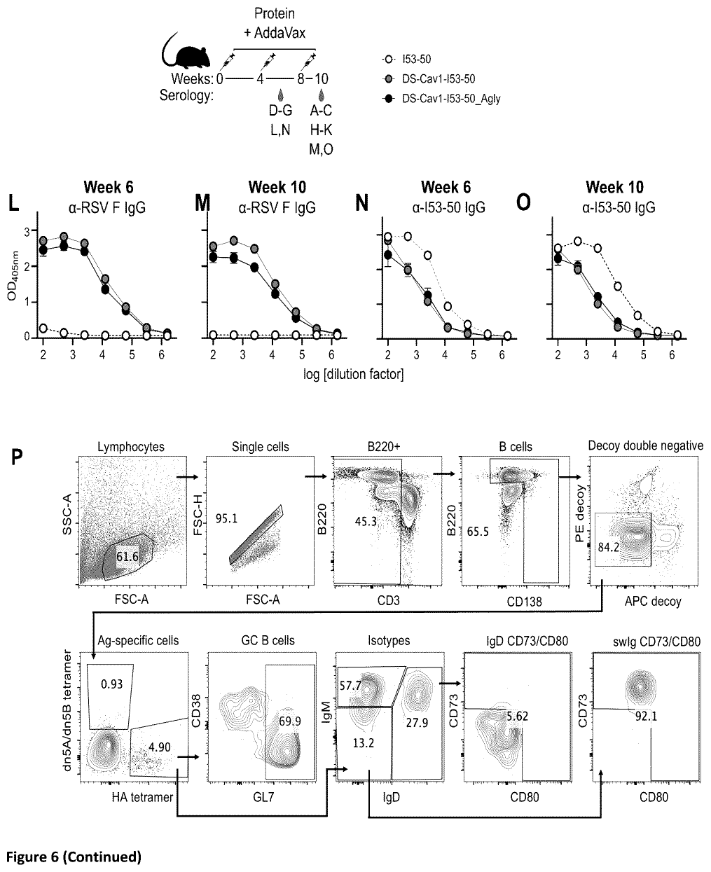

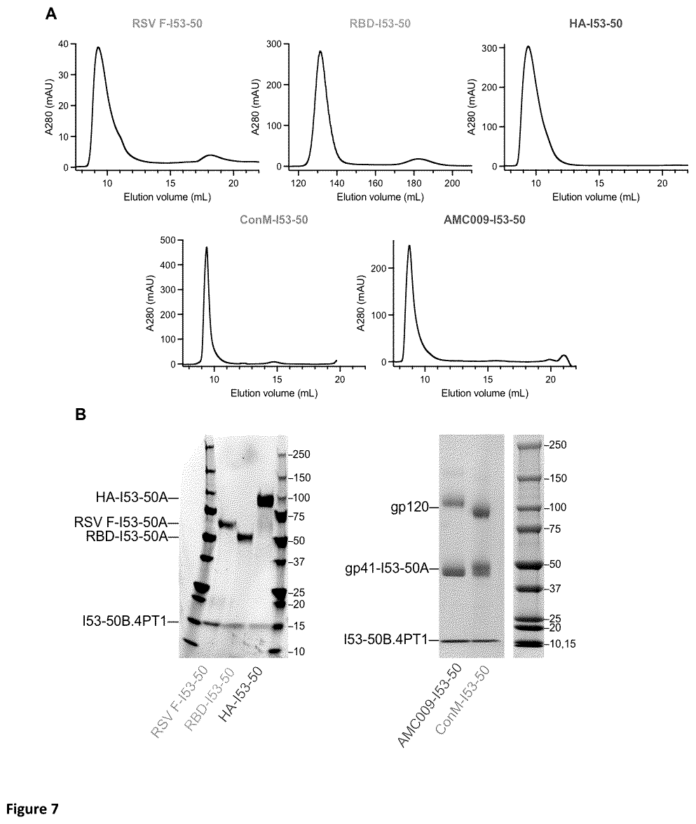

We first tested how effectively these three different surface masking strategies dampen antibody responses against the I53_dn5 nanoparticle without any viral glycoprotein antigen present. After three immunizations of 0.6 μg protein adjuvanted with AddaVax, the presence of glycans on either the I53_dn5B trimer (I53_dn5_Bgly) or both the I53_dn5A pentamer and I53_dn5B trimer (I53_dn5_ABgly) significantly reduced anti-I53_dn5A pentamer antibody responses compared to immunization with unmodified I53_dn5 nanoparticle (FIG. 6A). Anti-I53_dn5A pentamer antibody titers were even further reduced when 10 chains of 1 or 2 kDa PEG (I53_dn5_2C1kPEG, I53_dn5_2C2kPEG) or 5 unstructured polypeptides (I53_dn5_XTEN, I53_dn5_PAS, I53_dn5_ELP) masked each I53_dn5A pentamer in the nanoparticle immunogen (FIG. 6A). All scaffold masking approaches significantly reduced anti-I53_dn5B trimer and anti-I53_dn5 nanoparticle IgG titers compared to those elicited by unmodified I53_dn5 particles (FIGS. 6B-C). In summary, all three masking strategies reduced antibody responses against the I53_dn5B trimer and assembled I53_dn5 nanoparticle, with PEG and unstructured polypeptides masking the I53_dn5A pentamer more efficiently than glycans.

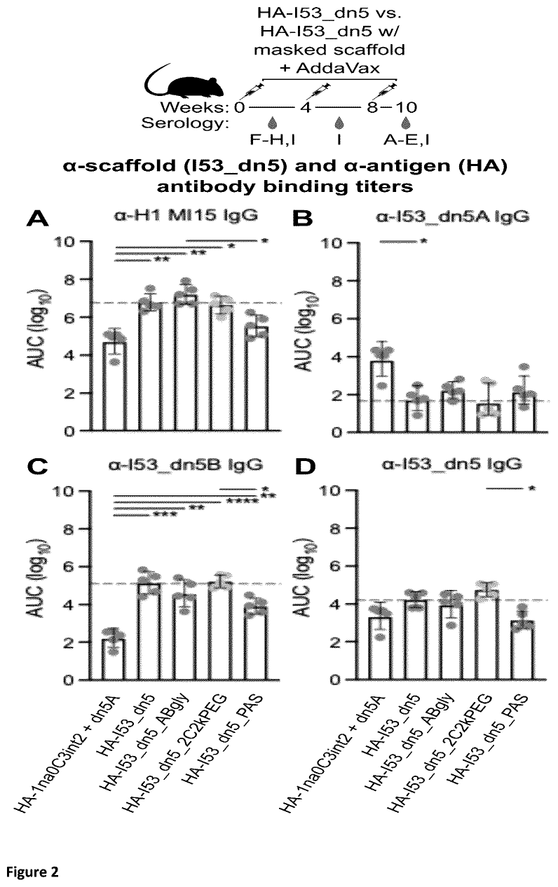

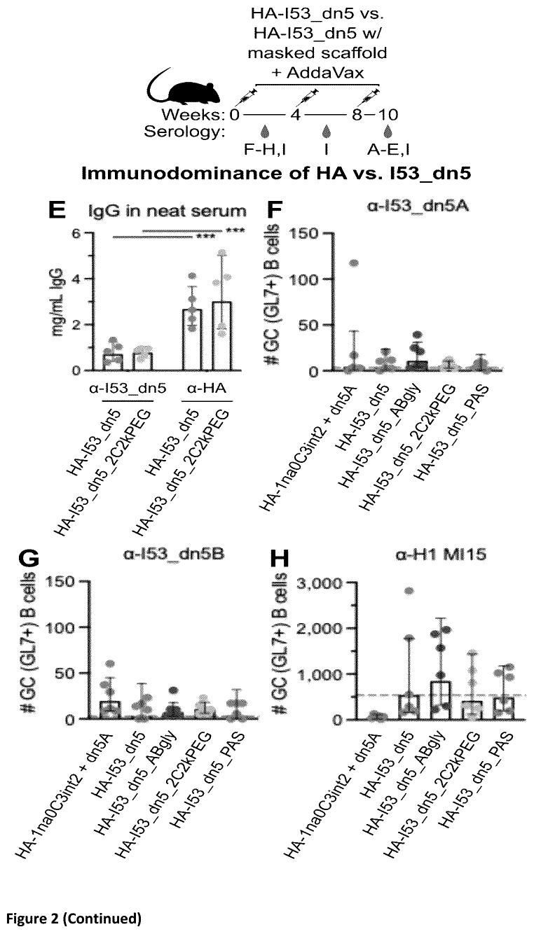

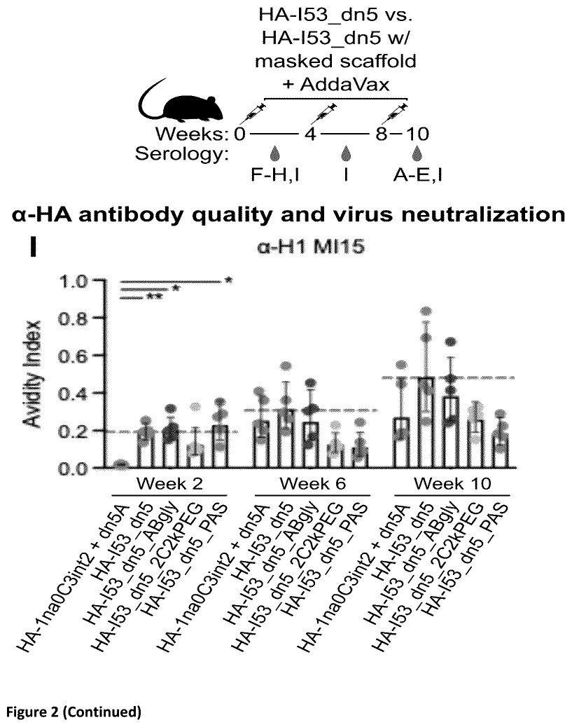

We next assessed the immunological impact of masking the nanoparticle scaffold when the HA antigen was presented on the nanoparticle. Scaffold-masked HA-I53_dn5 nanoparticle immunogens were formed by assembling HA-I53_dn5B with I53_dn5A pentamers bearing either 10 glycans (HA-I53_dn5_Agly), 10 linear 2 kDa PEG chains (HA-I53_dn5_2C2kPEG), or 5 unstructured PAS polypeptides (HA-I53_dn5_PAS) (FIGS. 1A-C). Based on our finding that scaffold masking reduced anti-particle responses when no viral glycoprotein antigen was displayed (FIG. 6A-C), we hypothesized that shielding surface-exposed epitopes on the nanoparticle scaffold could potentially focus and enhance the immune response to HA by reducing competition in germinal centers from scaffold-specific B cells (Mesin et al., 2016). However, following three immunizations of 0.9 μg HA (1.5 μg total protein) with AddaVax, anti-HA IgG titers were not enhanced for any of the scaffold-masked immunogens compared to the HA-I53_dn5 immunogen (FIGS. 2A, 6H). At the same time, when HA was present on the particle, scaffold masking had a much smaller—in some cases indiscernible-effect on reducing anti-scaffold IgG titers (FIGS. 2B-D, 6I-K), suggesting immunodominance of HA over the underlying I53_dn5 scaffold. Similarly, for a different icosahedral nanoparticle immunogen in which half of the 20 trimers displayed prefusion RSV F antigen and the other half bore 12 glycans per trimer (120 glycans per particle), the presence of the glycans did not dampen the anti-I53-50 scaffold IgG responses and also did not enhance anti-F responses relative to a corresponding non-glycosylated immunogen (FIGS. 6L-O). However, the presence of prefusion F on the nanoparticle immunogens significantly reduced antibody responses against the I53-50 nanoparticle compared to immunization with unmodified I53-50 (FIGS. 6N-O), again suggesting immunodominance of the displayed antigen.

Interestingly, a non-assembling control immunogen in which the trimeric component lacked the computationally designed interface that drives nanoparticle assembly (HA-1na0C3int2+I53_dn5A; (Ueda et al., 2020)) elicited significantly higher anti-I53_dn5A pentamer titers (FIGS. 2B, 6E, 6I) and significantly lower anti-HA (FIGS. 2A, 6D, 6H) and anti-I53_dn5B (FIGS. 2C, 6F, 6J) titers than HA-I53_dn5 nanoparticles. The fact that the immunogenicity of I53_dn5A was enhanced when the pentamer was physically separated from HA-I53_dn5B further suggested that in the nanoparticle context HA (i) is immunodominant over and outcompetes responses to the I53_dn5A pentamer or (ii) sterically occludes BCR access to I53_dn5A. This immunodominance of HA and suppression of antibody responses to other proteins in close proximity to HA is consistent with influenza neuraminidase (NA) being more immunogenic when not co-delivered with HA (Johansson et al., 1989).

To further characterize the magnitude of the response to various parts of the HA-bearing nanoparticle immunogens, we quantified antigen- and scaffold-specific IgG concentrations in neat serum as well as antigen- and scaffold-specific B cells in lymph node germinal centers (GCs). We found the amount of anti-HA IgG in undiluted serum (˜3 mg/mL) was ˜3-fold higher than the amount of anti-I53_dn5 IgG (˜1 mg/mL), for both the HA-I53_dn5 and HA-I53_dn5_2C2kPEG immunogens (FIG. 2E). In lymph nodes, the numbers of HA-specific GC (GL7⁺) B cells were ˜50-fold higher than I53_dn5A- or I53_dn5B-specific GC B cells (FIGS. 2F-H, 6P). These data are consistent with our ELISA data, again indicating the displayed HA antigen is immunodominant over the underlying I53_dn5 scaffold.

We also assessed anti-HA IgG quality and binding affinity in a chaotropic ELISA that challenged serum IgG binding with 2 M NaSCN, which showed a non-significant trend of diminished antibody avidity in sera after the second and third immunizations for the non-assembling immunogen and the nanoparticle immunogens with PEG and PAS masking (FIG. 2I). Instability of the HA-I53_dn5_PAS nanoparticle immunogen may be a factor here, since these nanoparticles did not fully assemble in vitro (FIGS. 1J, 1K, 1L) and anti-HA and anti-I53_dn5 IgG titers trended towards those elicited by the non-assembling control (FIGS. 2A-D). However, in an immunodepletion experiment that eliminated antibodies against the I53_dn5 nanoparticle exterior, residual antibody binding against epitopes on the interior surface of the nanoparticle or buried upon nanoparticle assembly-which could become exposed upon nanoparticle disassembly in vivo-were nearly completely removed from the sera of mice that received the HA-I53_dn5 and HA-I53_dn5_PAS immunogens, but not the non-assembling control immunogen. This result indicates that both the HA-I53_dn5 and HA-I53_dn5_PAS immunogens are stable in vivo and remain intact long enough to prevent the elicitation of substantial antibody responses against epitopes on nanoparticle interior surfaces.

In summary, these three scaffold masking strategies reduced antibody responses against the I53_dn5 particle when no viral glycoprotein antigen was displayed. However, when HA and RSV F were presented on the I53_dn5 and I53-50 scaffolds, respectively, scaffold masking did not dampen anti-scaffold antibody responses and did not enhance, but in some cases diminished (e.g., PEG and PAS), antigen-specific antibody responses.

In a Series of Nanoparticle Immunogens that all Used the Same I53-50 Scaffold, Only HIV-1 Env was Subdominant to the Nanoparticle Scaffold

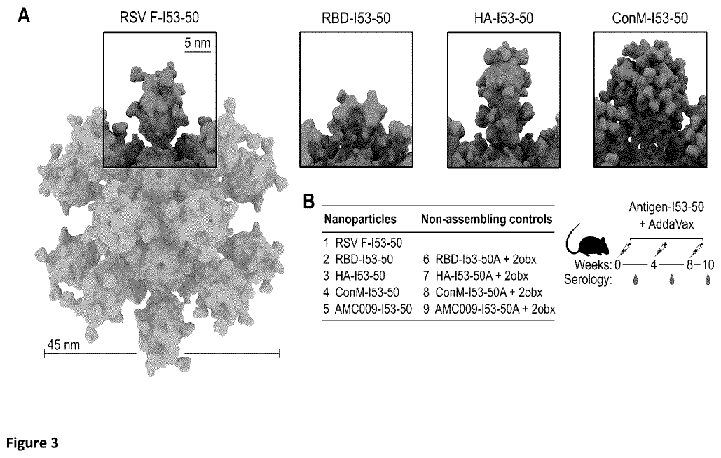

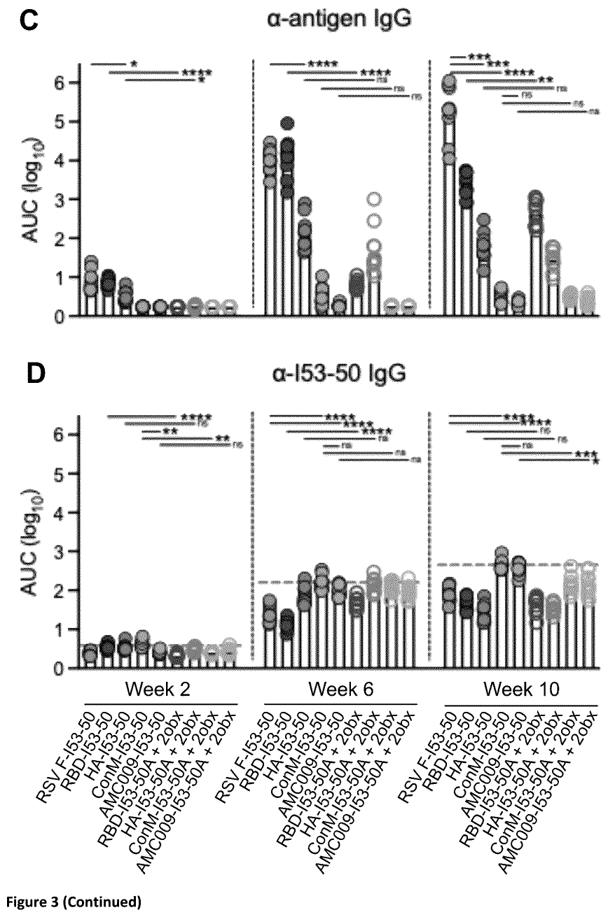

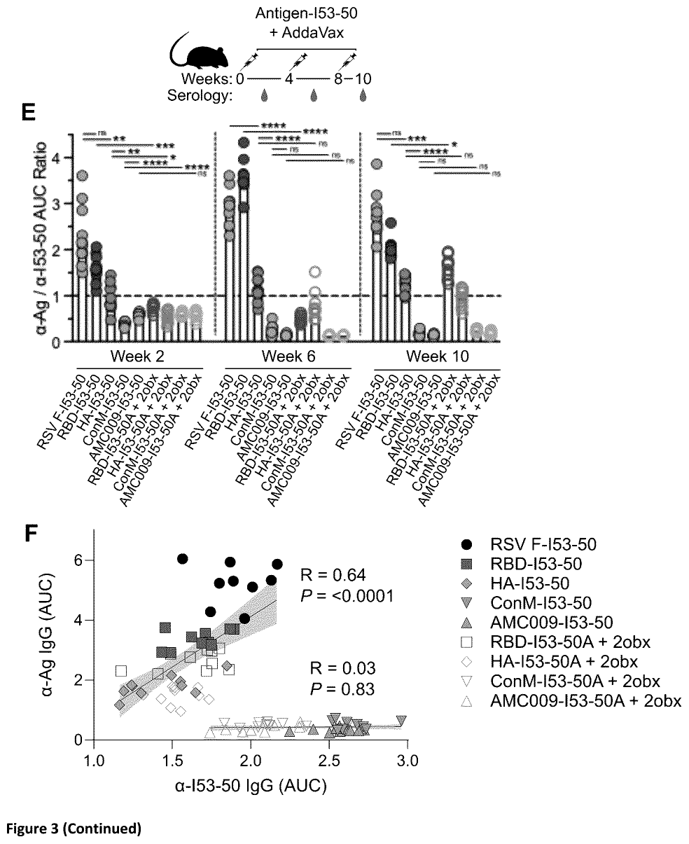

To our knowledge, there is no reported head-to-head immunogenicity study of different viral glycoprotein antigens on the same protein nanoparticle scaffold (Klasse et al., 2020). To comparatively evaluate the immunogenicity of a range of different viral glycoprotein antigens displayed on the same protein nanoparticle scaffold and the level of anti-scaffold antibody responses elicited by each, we displayed five different viral glycoprotein antigens (prefusion RSV F, SARS-CoV-2 RBD, influenza HA, and two different native-like HIV-1 Env trimers: ConM and AMC009) separately on the two-component nanoparticle I53-50 (FIG. 3A, 7). We used I53-50 as the nanoparticle scaffold for these experiments because we and others have used I53-50 to display a wide variety of antigens, including RSV F, SARS-CoV-2 RBD, and HIV-1 Env (Brouwer et al., 2019; Marcandalli et al., 2019; Walls et al., 2020). Two different native-like HIV-1 Env trimers were used because of their different immunogenicities: ConM is more immunogenic than AMC009, as the latter trimer has a denser glycan shield (Schorcht et al., 2020; Sliepen et al., 2019). To specifically explore the role of nanoparticle formation in the relative immunogenicity of antigen and scaffold, we also prepared non-assembling control immunogens for RBD, HA, and the two Env trimers comprising a version of the I53-50B pentamer (“2obx”) lacking the computationally designed interface that drives nanoparticle assembly.

Following one, two, and three immunizations with 72.4 pmol antigen (equal to 5 μg HIV-1 Env ConM and 3.0 μg I53-50 for each nanoparticle immunogen; FIG. 3B), RSV F and SARS-CoV-2 RBD on I53-50 elicited the highest antigen-specific antibody titers; HA on I53-50 elicited intermediate antigen-specific titers; and both HIV-1 Env trimers (ConM and AMC009) on I53-50 elicited the lowest antigen-specific titers (FIG. 3C). Thus, the antigen immunogenicity hierarchy for I53-50-scaffolded nanoparticle immunogens was: RSVF>SARS-CoV-2 RBD>HA>HIV-1 Env ConM>HIV-1 Env AMC009. Comparison of the antigen-specific titers elicited by assembled versus non-assembled nanoparticles showed that significant improvement in antigen-specific titers was only observed for RBD-I53-50, but not for HA-I53-50, ConM-I53-50, or AMC009-I53-50 (FIG. 3C). Antigen-specific titers from the non-assembling controls increased after booster immunizations for the RBD and HA immunogens, but the antigen-specific titers for the HIV-1 Env non-assembling controls remained near baseline levels (FIG. 3C). Conversely, anti-I53-50 titers for the ConM-I53-50 group were the highest among all immunogens at all time points (FIG. 3D). This suggests that despite its relatively large size, the poorly immunogenic HIV-1 Env may impart less antigenic competition with the nanoparticle scaffold than the other more immunogenic antigens. Alternatively, more efficient trafficking of HIV-1 Env immunogens to lymph nodes due to the high oligomannose glycan density on Env (Read et al., 2022; Struwe et al., 2018; Tokatlian et al., 2019) may be another mechanism by which the immunogenicity of the underlying I53-50 scaffold is increased. Furthermore, the ratio of antigen-specific over anti-I53-50 titers for the HIV-1 Env groups was consistently less than 1 at all time points, while the other nanoparticle immunogen groups exhibited ratios equal to or greater than 1 (FIG. 3E). Therefore, for this series of nanoparticle immunogens, only HIV-1 Env was immunosubdominant to the nanoparticle scaffold.

The antigen-specific and scaffold-specific antibody titers did not correlate with the physical size of the antigen, measured by either antigen height or molecular weight. Furthermore, for the most part there are not substantial differences between the anti-scaffold responses in corresponding assembling and non-assembling groups (the exception being the RBD immunogens at Week 6) (FIG. 3D). These data suggest that the anti-scaffold response is not primarily determined by sterics/physical access to the scaffold surfaces. However, we cannot rule out that particle disassembly in vivo could be a factor here. Another potential factor driving the antigen immunogenicity hierarchy could be glycan density on the antigens; however, this failed to correlate with antigen-specific or scaffold-specific antibody titers. Moreover, each immunogen failed to show a negative correlation between antigen-specific and scaffold-specific responses, suggesting that anti-scaffold responses do not interfere with antigen-specific responses for any of these immunogens. Instead, when all immunogens (except Env immunogens) were grouped together, antigen-specific and scaffold-specific responses were highly positively correlated (P<0.0001), while the Env immunogens grouped together exhibited no correlation (P=0.83) (FIG. 3F). Taken together, these data indicate that within a nanoparticle immunogen there is not zero-sum antigen competition between antigen-specific and scaffold-specific antibody responses, since an increase in one does not result in a proportional decrease in the other. Instead, there is a significant positive correlation between antigen-specific and anti-scaffold responses across this set of non-Env antigens (FIG. 3F).

Competition from Excess I53-50 Nanoparticle Scaffold Suppresses Antigen-Specific Antibody Responses

To better understand the antigen vs. scaffold immunodominance hierarchies observed above and the potential role of antigenic competition between the displayed antigen and nanoparticle scaffold, we compared antigen-specific and scaffold-specific antibody responses elicited by a 10,000-fold dose range of RBD-I53-50 co-administered with a constant dose of excess I53-50 protein. Although we were unable to observe clear evidence of antigenic competition in the experiments presented above, we hypothesized that the addition of excess I53-50—to artificially inflate the scaffold to antigen ratio-might allow us to observe suppression of antigen-specific antibody responses due to antigenic competition, similar to how excess carrier protein outcompeted and suppressed hapten-specific antibody responses (Woodruff et al., 2018). We used the RBD nanoparticle immunogen in this experiment based on our finding above that the RBD is strongly immunodominant to the I53-50 scaffold (FIGS. 3C-E).

We immunized mice with RBD-I53-50 comprising 1.7, 0.1, 0.01, 0.001, or 0.0001 μg RBD with or without co-administration of excess I53-50 to a total of 3 μg of the nanoparticle scaffold. After a single immunization, we observed a typical dose-response effect in both the RBD-specific and anti-scaffold antibody responses, with loss of detectable antibodies at 0.0001 g RBD and 0.002 μg I53-50, respectively. The effect of co-administering excess I53-50 was already apparent post-prime. RBD-specific antibodies were 8.1- and 4.4-fold lower at the 0.1 and 0.01 μg RBD doses relative to the conditions without excess scaffold, respectively, while anti-scaffold responses, in the presence of excess I53-50, were roughly constant over the entire dose range. The average post-prime antigen-specific to scaffold-specific AUC ratio was greater than 1 for all groups post-prime except for the 0.01, 0.001, and 0.0001 μg RBD doses with excess I53-50. Post-boost, these trends were amplified, with the exception that there was no diminution in the RBD-specific antibody responses when decreasing the RBD dose from 1.7 to 0.01 μg, although further decreases in dose led to lower anti-RBD responses. Suppression of the RBD-specific antibodies by excess I53-50 was more pronounced post-boost, with decreases of 113-, 266-, and 147-fold at doses of 0.1, 0.01, and 0.001 μg RBD relative to the conditions without excess scaffold, respectively. There was also a trend of reduced post-boost pseudovirus neutralization (IC₅₀) in the presence of excess I53-50, with decreases of 23-, 32-, and 13-fold at doses of 0.1, 0.01, and 0.001 μg RBD, respectively. There was still a clear dose-response effect in the anti-scaffold responses in the absence of co-administered I53-50. Interestingly, the anti-scaffold responses with co-administered I53-50 trended higher than the 1.7 μg dose of RBD-I53-50, despite containing the same total amount of I53-50 scaffold. The average post-boost antigen-specific to scaffold-specific AUC ratio was less than 1 for only the 0.001 and 0.0001 μg RBD doses with excess I53-50, whereas this AUC ratio progressively increased for 1.7, 0.1, 0.01, and 0.001 μg RBD when no excess I53-50 was present. We also tried a similar competition experiment with ConM (but with a smaller dose range), but the anti-ConM antibody responses were so weak that no suppression of anti-ConM titers was detected when excess I53-50 was co-delivered. In addition, we tested if excess heterologous nanoparticle scaffold suppressed antigen-specific antibody responses by immunizing mice with RBD-I53-50 in the presence of excess I53_dn5 nanoparticles. Interestingly, we found that excess heterologous I53_dn5 scaffold did not suppress RBD-specific antibodies. Taken together, these data confirm that in the context of protein nanoparticle immunogens that display viral glycoprotein antigens, excess homologous nanoparticle scaffold, but not heterologous nanoparticle scaffold, can compete with and suppress antigen-specific binding and pseudovirus neutralizing antibody responses.

### DISCUSSION

To better understand the role of anti-scaffold immune responses, we masked the underlying I53_dn5 nanoparticle in the HA-I53_dn5 immunogen using three different approaches: glycosylation, PEGylation, and PASylation. All three approaches successfully yielded nanoparticle immunogens co-displaying a large glycoprotein antigen and the masking moieties, showcasing the robustness and versatility of computationally designed two-component nanoparticles as a multivalent display platform. However, there are limits to what can be displayed on the nanoparticle exterior: the efficiency of in vitro assembly was substantially reduced for the PASylated particle. We then examined how shielding the scaffold impacted anti-HA antibody responses. To our knowledge, this is the first report of immune responses to a protein nanoparticle immunogen comprising a masked scaffold displaying an oligomeric viral glycoprotein antigen. Overall, scaffold masking did not increase anti-HA antibody titers and in some instances (i.e., PEGylation and PASylation) appeared to occlude cross-reactive epitopes in the HA stem. The partial disassembly of HA-I53_dn5_PAS nanoparticles due to their instability may also have contributed to the reduced HA stem responses. Overall, these observations suggest that masking the scaffolds of other nanoparticle immunogens that display an immunodominant antigen may be ineffective at improving the magnitude of the antigen-specific antibody response, although there are other beneficial effects that could derive from scaffold masking, particularly with glycans. For example, protein nanoparticle immunogens bearing high-mannose N-linked glycans can traffic more efficiently to draining lymph nodes and B cell follicles in vivo, resulting in enhanced germinal center formation and antibody responses against the displayed antigen or nanoparticle immunogen.

Here, we confirmed the immunodominance of prefusion RSV F, SARS-CoV-2 RBD, and influenza HA on the I53-50 scaffold, and showed the subdominance of two variants of HIV-1 Env (ConM and AMC009) on I53-50. To our knowledge, this is the first head-to-head comparison of the immunogenicity of different viral glycoprotein nanoparticle immunogens that all use the same scaffold. This study design allowed for direct comparison of the antigen-specific and scaffold-specific immune responses, and we showed that anti-scaffold antibody responses are not negatively correlated with antigen-specific responses for this set of immunogens. The subdominance of HIV-1 Env suggests that, in contrast to the other immunodominant antigens, masking the underlying scaffold may enhance anti-Env antibody responses.

Antigenic competition determines immunodominance patterns for complex immunogens (Brody and Siskind, 1969; Johansson et al., 1987). Subdominant antibody responses arise when BCR access is occluded and/or low frequency B cells or those with low-affinity BCRs cannot compete for expansion within germinal centers (Abbott and Crotty, 2020; Abbott et al., 2018; Dosenovic et al., 2018). Here, we showed that co-delivery of excess I53-50 scaffold with RBD-I53-50 immunogens suppressed immunodominant antigen-specific antibody responses, but co-delivery of excess heterologous I53_dn5 scaffold with RBD-I53-50 immunogens did not suppress antigen-specific antibody responses. These data suggest that immunodominant antibody responses (e.g., RBD-specific) are suppressed when subdominant (e.g., scaffold) epitopes are increased in abundance, are no longer physically linked to immunodominant epitopes, and/or are more accessible to BCRs. Therefore, for nanoparticle immunogens in which scaffold- and antigen-specific responses are on a roughly equal footing, anti-scaffold responses may impede antigen-specific responses. Our data also imply that protein nanoparticle immunogens with reduced antigen valency, in which some of the potential antigen-bearing sites are left vacant, could suffer from anti-scaffold responses suppressing antigen-specific responses. Overall, we have shown that protein nanoparticle scaffolds are a potential source of antigenic competition, which is an important consideration when designing complex immunogens.

In summary, our results inform the design of protein nanoparticle immunogens. Physically masking protein nanoparticle scaffolds reduces antibody responses against the scaffolds themselves, which is desirable since these antibodies will not contribute to protection upon subsequent infection. Scaffold masking using N-linked glycans in particular can have the additional benefit of enhancing vaccine trafficking and uptake in vivo.

### Experimental Model and Subject Details

### Cell Lines

Expi293F cells are derived from the HEK293F cell line (Life Technologies). Expi293F cells were grown in Expi293 Expression Medium (Life Technologies), cultured at 36.5° C. with 8% C02 and shaking at 150 rpm. HEK293T/17 is a female human embryonic kidney cell line (ATCC). VeroE6-TMPRSS2 cells are an African Green monkey Kidney cell line expressing TMPRSS2 (Lempp et al., 2021). Adherent cells were cultured at 37° C. with 5% C02 in flasks with DMEM+10% FBS (Hyclone)+1% penicillin-streptomycin. Adherent cells were not tested for mycoplasma contamination nor authenticated.

### Mice

Female BALB/c mice (Stock #000651, BALB/c cByJ mice) four weeks old were obtained from Jackson Laboratory, Bar Harbor, Maine, and maintained at the Comparative Medicine Facility at the University of Washington, Seattle, WA, accredited by the American Association for the Accreditation of Laboratory Animal Care International (AAALAC). Animal procedures were performed under the approvals of the Institutional Animal Care and Use Committee (IACUC) of the University of Washington, Seattle, WA.

### Method Details

### Computational Design of Glycosylated Proteins

Detailed methods and code are reported elsewhere (Adolf-Bryfogle et al., 2021). Briefly, all possible residues on the outward facing surfaces of the I53_dn5A pentamer and I53_dn5B trimer when assembled into I53_dn5 nanoparticles were manually selected as candidate locations for designing in an NxT/S PNGS. Next, the CreateGlycanSequonMover in Rosetta™ was used to sequentially knock-in a single NxT/S sequon at these selected locations and obtain calculated energies of the new protein structure using the Rosetta™ score function. Both typical and enhanced sequons, which include an aromatic amino acid in the N-2 position to potentially increase glycosylation efficiency (Huang et al., 2017; Murray et al., 2015), were attempted at each position. Protein structures were first scored by Rosetta™ without a model glycan tree present to eliminate any potential interference of the glycan atoms. To filter out bad designs, outputs with a “total_energy” of >500 and an RMSD>0.25 Å and >0.40 Å compared to the original I53_dn5A and I53_dn5B scaffolds, respectively, were discarded. The re-designed protein structures that passed this filtering step were then glycosylated using the SimpleGlycosylateMover with a model tri-antennary Man9 N-linked glycan, modeled using the GlycanTreeModeler, and scored by Rosetta™. A second round of filtering was performed using the same criteria as above. After proteins with a single PNGS were experimentally screened for expression and glycan occupancy (see below), combinations of PNGS were designed using the same computational pipeline. The XML file for this combinatorial selection is provided as Supplementary Material. The recently reported I53-50A_4gly subunit (Read et al., 2022) was used to generate the glycosylated RSV F-I53-50 immunogen shown in FIG. 5L-O.

### Gene Synthesis and Vector Construction

For each design that resulted from the above computational pipeline, the final construct contained an N-terminal signal peptide derived from bovine prolactin (MDSKGSSQKGSRLLLLLVVSNLLLPQGVLA; SEQ ID NO:97) and C-terminal myc and hexa-histidine tags (LEEQKLISEEDLHI-HHHHH; SEQ ID NO:98). These constructs and others used in this study were cloned by GenScript into the pCMV/R (VRC 8400) mammalian expression vector using the restriction sites Xbal and AvrII. Preparation of plasmids for expression of the following proteins have been previously described: I53_dn5A pentamer and I53_dn5B trimer (Ueda et al., 2020), I53-50B.4PT1 pentamer (Bale et al., 2016), influenza HIMI15 fusion to I53_dn5B trimer (Boyoglu-Barnum et al., 2021), HIV-1 ConM Env fusion to I53-50A trimer (Brouwer et al., 2019), HIV-1 AMC009 Env trimer (Sliepen et al., 2019), RSV DS-Cav1 fusion to I53-50A trimer (Marcandalli et al., 2019), SARS-CoV-2 RBD fusion to I53-50A trimer (Walls et al., 2020), and SARS-CoV-2 Spike HexaPro trimer (Hsieh et al., 2020). HIV-1 AMC009 Env trimer was fused to I53-50A trimers as described in (Brouwer et al., 2019). The amino acid sequences for all proteins used in this study are provided in Supplementary Table 1.

### Microbial Protein Expression and Purification

The nanoparticle components I53-50A and I53-50B.4.PT1 (Bale et al., 2016), and I53_dn5A and I53_dn5B (Ueda et al., 2020), were expressed in Lemo21 (DE3) (NEB) in LB (10 g Tryptone, 5 g Yeast Extract, 10 g NaCl) and grown in 2 L baffled shake flasks. Cells were grown at 37° C. to an OD600 ˜0.8, and then induced with 1 mM IPTG. Expression temperature was reduced to 18° C. and the cells were shaken for ˜16 h. The cells were harvested and lysed by microfluidization using a Microfluidics M110P at 18,000 psi in 50 mM Tris, 500 mM NaCl, 30 mM imidazole, 1 mM PMSF, (with 0.75% CHAPS only for I53-50 proteins). Lysates were clarified by centrifugation at 24,000 g for 30 min and applied to a 2.6×10 cm Ni Sepharose™ 6 FF column (Cytiva) for purification by IMAC on an AKTA Avant150 FPLC system (Cytiva). Protein of interest was eluted over a linear gradient of 30 mM to 500 mM imidazole in a background of 50 mM Tris pH 8, 500 mM NaCl, (with 0.75% CHAPS only for I53-50 proteins) buffer. Peak fractions were pooled, concentrated in 10K MWCO centrifugal filters (Millipore), sterile filtered (0.22 m) and applied to a Superdex™ 200 Increase 10/300 SEC column (Cytiva) using 50 mM Tris pH 8, 500 mM NaCl, (with 0.75% CHAPS only for I53-50 proteins) buffer. After sizing, bacterial-derived components were tested to confirm low levels of endotoxin before using for nanoparticle assembly.

### Mammalian Protein Expression and Purification

Small-scale 2.0 mL cultures of Expi293F cells were grown in suspension to a density of 3.0×10⁶ cells per mL and transiently transfected using PEI-MAX (Polyscience) and cultivated for 5 days in Expi293F expression medium (Life Technologies) at 37° C., 70% humidity, 8% C02, and rotating at 150 rpm. Supernatants were clarified by centrifugation (5 min at 4000 ref), PDADMAC solution was added to a final concentration of 0.0375% (Sigma Aldrich, #409014), and a final spin was performed (5 min at 4000 rcf). Supernatants were concentrated using a 5 kDa MWCO spin filter (Sartorius) to a final volume of ˜50 μL. These concentrated supernatants were then assessed for protein expression by Western blot using an anti-myc mouse primary antibody and an anti-mouse HRP-conjugated goat secondary antibody. Glycan occupancy for each protein design was assessed by increased molecular weight gel shifts on the Western blots compared to the unglycosylated parent protein.

For large-scale protein expression, 800 mL cultures of Expi293F cells were transiently transfected and cultivated for 5 days as described above. Proteins were purified from clarified supernatants via a batch bind method where Talon cobalt affinity resin (Takara) was added to supernatants and allowed to incubate for 15 min with gentle shaking. Resin was isolated using 0.2 μm vacuum filtration and transferred to a gravity column, where it was washed with 20 mM Tris pH 8.0, 300 mM NaCl, and protein was eluted with 3 column volumes of 20 mM Tris pH 8.0, 300 mM NaCl, 300 mM imidazole. This batch bind process was repeated a second time on the supernatant flow-through from the filtration step. Eluate with protein was concentrated to ˜2 mL using a 30 kDa MWCO Amicon concentrator (Millipore Sigma). The concentrated sample was sterile filtered (0.2 μm) and applied to a Superdex™ 200 Increase 10/300 SEC column (Cytiva) using 25 mM Tris pH 8.0, 150 mM NaCl, 0.75% CHAPS, 5% glycerol buffer.

Env-I53-50A constructs together with a plasmid expressing furin were transfected into Expi293F cells using PEI-MAX and cultured for 6 days. Furin was added to ensure optimal furin-mediated cleavage of Env (ConM-I53-50A:furin ratio was 3:1, AMC009-I53-50A:furin ratio was 2:1). Cells were spun down and supernatants filtered through a 0.2 μm Steritop™ filter. Env-I53-50A proteins were purified by running the clarified supernatant over a PGT145 bNAb-affinity chromatography column. Eluted proteins were concentrated using vivaspin 100 kDa spin columns. Concentrated proteins were subsequently applied to a Superose™ 6 increase 10/300 GL column (Cytiva) to remove aggregated proteins using a buffer of 25 mM Tris pH 8.0, 125 mM NaCl, 5% glycerol. I53-50B.4PT1 was added in a 1:1 ratio and incubated at 4° C. overnight. Assembled particles were again applied to a Superose™ 6 increase 10/300 GL column (GE healthcare) to remove unassembled components. Particles were buffer-exchanged into PBS with 250 mM sucrose by dialysis at 4° C. overnight, followed by a second dialysis step of 4 h, using a Slide-A-Lyzer™ MINI dialysis device (20 kDa cutoff, ThermoFisher Scientific). The 250 mM sucrose was added to increase recovery after freeze-thawing.

### PEG-Maleimide to HS-Protein Coupling

Protein with reduced unpaired cysteines was first purified by size exclusion chromatography (SEC) using buffer that contained 1 mM TCEP, and then SEC-purified again to exchange buffer with HEPES coupling buffer (pH 7.4, 20 mM HEPES, 150 mM NaCl, 1 mM EDTA, 0.75% CHAPS). Using a freshly prepared 10 mM PEG-maleimide solution in HEPES coupling buffer, a 1.0 mL maleimide-thiol coupling reaction was prepared at 5:1 PEG:Cys (mol/mol) and a 50 μM final protein concentration. This reaction was incubated with rocking at ambient temperature for 3 h, then overnight at 4° C. The reaction was quenched by adding reduced glutathione (GSH) to 2 mM. Unreacted PEG was removed using SEC.

### In Vitro Nanoparticle Assembly and Purification

The protein concentration of individual nanoparticle components (e.g., I53_dn5A pentamer and I53_dn5B trimer, or I53-50A trimer and I53-50B.4PT1 pentamer) was determined by measuring 280 nm absorbance using a UV/vis spectrophotometer (Agilent Cary 8454) and estimated extinction coefficients (Marcandalli et al., 2019). Particle assembly was performed by adding equimolar amounts of trimer and pentamer components to reach a final protein concentration of 20 μM (10 μM for each individual component) and resting on ice for at least 30 min. Assembled particles were sterile filtered (0.2 μm) immediately before SEC purification using a Superose™ 6 Increase 10/300 GL column or a HiLoad™ 26/600 Superdex™ 200 pg column for the RBD-I53-50 nanoparticle immunogen.

### Negative-Stain Electron Microscopy

A sample volume of 3 μL at a concentration of 70 μg/mL protein in 50 mM Tris pH 8, 150 mM NaCl, 5% v/v glycerol was applied to a freshly glow-discharged 300-mesh copper grid (Ted Pella) and incubated on the grid for 1 minute. The grid was then dipped in a 40 μL droplet of water, excess liquid was blotted away with filter paper (Whatman), the grid was dipped into 3 μL of 0.75% w/v uranyl formate stain, stain was immediately blotted off with filter paper, then the grid was dipped again into another 3 μL of stain and incubated for ˜30 seconds. Finally, the stain was blotted away and the grids were allowed to dry for 1 minute prior to storage or imaging. Prepared grids were imaged in a Talos model L120C transmission electron microscope using a Gatan camera at 57,000×.

### Antigenic Characterization

ELISA was used to measure binding of HA-foldon, HA-1na0C3int2, HA-ferritin, HA-I53_dn5, HA-I53-dn5_ABgly, and HA-I53_dn5_2C2kPEG to monoclonal antibodies CR9114 and 5J8 using the ELISA method described below. Monoclonal antibodies were serially diluted from 300 to 0.5 ng/mL.

### Dynamic Light Scattering

Dynamic light scattering (DLS) was used to measure the hydrodynamic diameter of nanoparticle immunogens on a DynaPro™ NanoStar instrument (Wyatt Technologies). 2 μL of 0.1 mg/mL protein was applied to a quartz cuvette to obtain intensity measurements from 10 acquisitions of 10 s each. Increased viscosity due to 5% glycerol in the buffer was accounted for by the software.

### Endotoxin Measurements

Endotoxin levels in immunogen samples were measured using the EndoSafe Nexgen-MCS System (Charles River). Samples were diluted 1:100 in Endotoxin-free LAL reagent water, and applied into wells of an EndoSafe LAL reagent cartridge. Endotoxin content was analyzed using Charles River EndoScan-V software, which automatically back-calculates for the 1:100 dilution factor. Endotoxin values reported as EU/mL were converted to EU/mg based on protein concentration obtained by UV-Vis measurements. All endotoxin values were <100 EU/mg.

### Mouse Immunizations and Sera Collection

Mice were inoculated with 0.9 μg HA and/or 0.6 μg I53_dn5 scaffold (1.2 μg I53_dn5 scaffold for the HA-I53_dn5_ABgly group due to 50% HA valency) for the scaffold masking experiments (FIGS. 2, 5); 7.24×10⁻⁵ μmol antigen (equal to 5 μg HIV-1 Env) and 1.21×10⁻⁶ mol (3 μg) I53-50 scaffold for the antigen immunodominance experiment (FIGS. 3, 4, 6). Prior to inoculation, immunogen suspensions were gently mixed 1:1 (vol/vol) with AddaVax adjuvant (Invivogen, San Diego, CA). Mice were injected intramuscularly into the gastrocnemius muscle of each hind leg using a 27-gauge needle with 50 μL per injection site (100 μL total) of immunogen under isoflurane anesthesia. For sera collection, mice were bled via submental venous puncture 2 weeks following each inoculation. Serum was isolated from hematocrit via centrifugation at 2,000 g for 10 min, and stored at −80° C. until use.

### Serum Antibody ELISA

The protocol was adapted from Tiller et al. (Tiller et al., 2008). First, protein or goat anti-mouse IgG+IgM (Jackson ImmunoResearch, 115-005-068) was incubated for 1 h on 96-well Nunc MaxiSorp plates (Thermo Scientific) (2.0 μg/mL, 50 μL per well). Then, 200 μL of Tris Buffered Saline Tween (TBST: 25 mM Tris pH 8.0, 150 mM NaCl, 0.05% (v/v) Tween20) with 2% (w/v) BSA was added to each well and incubated for 1 h. Plates were washed 3× in TBST using a robotic plate washer (BioTek). Then, 50 μL of serum dilutions starting at 1:100 and serially diluting 5-fold seven times using TBST with 2% (w/v) BSA (8 total dilutions) were added to each well and incubated for 1 h. In wells with anti-mouse IgG capture antibody, mouse IgG lambda control (BD Pharminogen, 553485) was serially diluted from 500 to 0.5 ng/mL in TBST in triplicate and 50 μL of each dilution incubated for 1 h. After washing plates 3× with TBST, 50 μL of anti-mouse HRP-conjugated goat secondary antibody (CellSignaling Technology) diluted 1:2,000 in TBST with 2% (w/v) BSA was incubated in each well for 1 h. Following a final 3× TBST plate wash, 100 μL of ABTS (2,2′-Azinobis [3-ethylbenzothiazoline-6-sulfonic acid]-diammonium salt, Thermo Scientific) or TMB (3,3′,5′,5-tetramethylbenzidine, SeraCare) was added to each well and rested for 30 or 2 min, respectively. TMB was quenched with 100 μL of 1 N HCl. Absorbance at 405 or 450 nm, respectively, was immediately collected for each well on a SpectraMax™ M5 plate reader (Molecular Devices). All steps were performed at ambient temperature. Data were plotted in Prism (GraphPad) to determine AUC values. A logarithmic equation fit to the linear portion of the sigmoidal curve of the mouse IgG control was used to calculate concentration (mg/mL) of IgG in mouse sera for anti-I53_dn5 and anti-HA titers.

### Serum Antibody Avidity/Chaotropic ELISA

The protocol was adapted from Langowski et al. (Langowski et al., 2020). First, recombinant I53_dn5 nanoparticle or H1 MI15-foldon protein was incubated for 1 h on 96-well Nunc MaxiSorp™ plates (Thermo Scientific) (2.0 μg/mL, 50 μL per well). Then, 200 μL of Tris Buffered Saline Tween (TBST: 25 mM Tris pH 8.0, 150 mM NaCl, 0.05% (v/v) Tween20) with 2% (w/v) BSA was added to each well and incubated for 1 hr. Plates were washed 3× in TBST using a robotic plate washer (BioTek). Then, 50 μL of a 1:2,500 serum dilution in TBST with 2% (w/v) BSA was added to each well and incubated for 1 hr. To test for avidity, 50 μL of 2 M sodium thiocyanate (NaSCN) or PBS (control) was added to wells for 15 min. After washing plates 3× with TBST, 50 μL of anti-mouse HRP-conjugated goat secondary antibody (CellSignaling Technology) diluted 1:2,000 in TBST with 2% (w/v) BSA was incubated in each well for 1 hr. Following a final 3× TBST plate wash, 100 μL of TMB (SeraCare) was added to each well and rested for 2 min before quenching with 100 μL of 1 N HCl. Absorbance at 450 nm was immediately collected for each well on a SpectraMax M5 plate reader (Molecular Devices). All steps were performed at ambient temperature. Percentage OD450 in the corresponding NaSCN/PBS wells were used to determine the avidity index.

### Ni-NTA-Capture ELISA

The protocol was adapted from Brouwer et al. (Brouwer et al., 2019). First, 50 μL of 6.5 nM His-tagged protein per well was incubated for 1 h in 96-well Ni-NTA plates (Qiagen). Then, 200 μL of Tris Buffered Saline Tween (TBST: 25 mM Tris pH 8.0, 150 mM NaCl, 0.05% v/v Tween20) with 2% (w/v) BSA was added to each well and incubated for 1 h. Plates were washed 3× in TBST using a robotic plate washer (BioTek). Then, 50 μL of serum dilutions starting at 1:100 and serially diluting 5-fold seven times using TBST with 2% (w/v) BSA (8 total dilutions) were added to each well and incubated for 1 h. After washing plates 3× with TBST, 50 μL of anti-mouse HRP-conjugated goat secondary antibody (CellSignaling Technology) diluted 1:2,000 in TBST with 2% (w/v) BSA was incubated in each well for 1 h. Following a final 3× TBST plate wash, 100 μL of TMB (SeraCare) was added to each well and rested for 2 min, then 100 μL of 1 N HCl was added to each well to quench the reaction. Absorbance at 450 nm was immediately collected for each well on a SpectraMax™ M5 plate reader (Molecular Devices). Data were plotted in Prism (GraphPad) to determine AUC values. All steps were performed at ambient temperature.

### Sera Immunodepletion

Depletion antigen (I53_dn5) was added to reach a final concentration of 0.3 mg/mL in the starting 1:100 serum dilution used in ELISA and incubated for 15 min at room temperature. Then, serial dilutions and the ELISA procedure was performed as described above.

### Reporter-Based Microneutralization Assay

Reporter viruses were prepared as previously described (Creanga et al., 2021). In brief, H1N1 virus was made with a modified PB1 segment expressing the TdKatushka reporter gene (R3ΔPB1) and propagated in MDCK-SIAT-PB1 cells, while H5N1 reporter virus was made with a modified HA segment expressing the reporter (R3ΔHA) and produced in cells stably expressing H5 HA. Virus stocks were stored at −80° C. Mouse sera were treated with receptor destroying enzyme (RDE II; Denka Seiken) and heat-inactivated before use in neutralization assays. Immune sera was serially diluted and incubated for 1 h at 37° C. with pre-titrated virus. Serum-virus mixtures were then transferred to 96-well plates (PerkinElmer), and 1.0×10⁴ MDCK-SIAT1-PB1 cells (Bloom et al., 2010; Creanga et al., 2021) were added into each well. After overnight incubation at 37° C., the number of fluorescent cells in each well was counted automatically using a Celigo image cytometer (Nexcelom Biosciences). IC50 values, defined as the serum dilution or antibody concentration that gives 50% reduction in virus-infected cells, were calculated from neutralization curves using a four-parameter nonlinear regression model and plotted with GraphPad Prism.

### Pseudovirus Production

D614G SARS-CoV-2 S (Crawford et al., 2020) pseudotyped vesicular stomatitis viruses (VSVs) were prepared as described previously (McCallum et al., 2021; Sauer et al., 2021). Briefly, 293T cells in DMEM supplemented with 10% FBS, 1% PenStrep seeded in 10-cm dishes were transfected with the plasmid encoding for the S glycoprotein using lipofectamine 2000 (Life Technologies) following manufacturer's indications. One day post-transfection, cells were infected with VSV(G*ΔG-luciferase) and after 2 h were washed five times with DMEM before adding medium supplemented with anti-VSV-G antibody (I1-mouse hybridoma supernatant, CRL-2700, ATCC). Virus pseudotypes were harvested 18-24 h post-inoculation, clarified by centrifugation at 2,500×g for 5 min, filtered through a 0.45 m cut off membrane, concentrated 10 times with a 30 kDa cut off membrane, aliquoted and stored at −80° C.

### Pseudovirus Neutralization

VeroE6-TMPRSS2 cells (Lempp et al., 2021) were cultured in DMEM with 10% FBS (Hyclone), 1% PenStrep, and 8 μg/mL puromycin with 5% CO2 in a 37° C. incubator (Caron-VWR). One day prior to infection, 96-well plates were plated with 20,000 cells. The following day, cells were checked to be at 80% confluence. In an empty half-area 96-well plate a 1:3 serial dilution of sera was made in DMEM and then diluted pseudovirus was added to the serial dilution and incubated at room temperature for 30-60 min. After incubation, the sera-virus mixture was added to the cells at 37° C. and 2 hours post-infection, 40 μL 20% FBS-2% PenStrep DMEM was added. After 17-20 hours 40 μL/well of One-Glo-EX™ substrate (Promega) was added to the cells and incubated in the dark for 5-10 min prior to reading on a BioTek plate reader. Measurements were done in at least duplicate. Relative luciferase units were plotted and normalized in Prism (GraphPad). Nonlinear regression of log(inhibitor) versus normalized response was used to determine IC50 values from curve fits.

### Tetramer Production

Recombinant I53_dn5A pentamer, I53_dn5B trimer, and H1 MI15 hemagglutinin trimer were biotinylated using the EZ-Link™ Sulfo-NHS-LC Biotinylation Kit (ThermoFisher). Biotinylated protein was then incubated with differing amounts of streptavidin-PE (Prozyme) and probed with SA-AF680 (Invitrogen) to determine the ratio of biotin to streptavidin at which there was excess biotin available for SA-AF680 to bind. This ratio was used to determine the concentration of biotinylated protein, allowing for calculation of the amount of SA-PE required to create a 6:1 molar ratio of protein protomer to SA-PE. Biotinylated HA was incubated with SA-APC for 30 min at room temperature and purified on a Superose™ 6 Increase 10/300 GL size exclusion column (Cytiva), and the tetramer fraction was centrifuged in a 100 kDa molecular weight cutoff Amicon Ultra filter (Millipore). The tetramer concentration was determined by measuring the absorbance of APC at 650 nm. I53_dn5A and I53_dn5B proteins were biotinylated and tetramerized with SA-PE in the same manner, and the concentration was determined by measuring the absorbance of PE at 565 nm. The APC decoy reagent was generated by conjugating SA-APC to Dylight 755 using a DyLight™ 755 antibody labeling kit (ThermoFisher), washing and removing unbound DyLight 755, and incubating with excess of an irrelevant biotinylated His-tagged protein. The PE decoy was generated in the same manner, by conjugating SA-PE to Alexa Fluor 647 with an AF647 antibody labeling kit (ThermoFisher).

### Mouse Immunization, Cell Enrichment, and Flow Cytometry

For phenotyping B cells, 6-week old female BALB/c mice, three per dosing group, were immunized intramuscularly with 50 μL per injection site of immunogen formulations mixed 1:1 (vol/vol) with AddaVax™ adjuvant on day 0. All experimental mice were euthanized for harvesting of inguinal and popliteal lymph nodes on day 11. The experiment was repeated twice. Popliteal and inguinal lymph nodes were collected and pooled for individual mice. Cell suspensions were prepared by mashing lymph nodes and filtering through 100 μm Nitex™ mesh. Cells were resuspended in PBS containing 2% FBS and Fc block (2.4G2), and were incubated with 10 nM decoy tetramers at room temperature for 20 min. I53_dn5A-PE tetramer and HA-APC tetramer, or I53_dn5B-PE tetramer and HA-APC tetramer, were added at a concentration of 10 nM and incubated on ice for 20 min. Cells were washed, incubated with anti-PE and anti-APC magnetic beads on ice for 30 min, then passed over magnetized LS columns (Miltenyi Biotec). Bound B cells were stained with anti-mouse B220 (BUV737), CD3 (PerCP-Cy5.5), CD138 (BV650), CD38 (Alexa™ Fluor 700), GL7 (eFluor 450), IgM (BV786), IgD (BUV395), CD73 (PE-Cy7), and CD80 (BV605) on ice for 20 min. Cells were run on a Cytek Aurora and analyzed using FlowJo software (Treestar). Cell counts were determined using Accucheck cell counting beads.

### Statistical Analysis

Multi-group comparisons were performed using the Brown-Forsythe one-way ANOVA test and Dunnett's T3 post hoc analysis in Prism 9 (GraphPad) unless mentioned otherwise. All correlations were two-tailed Spearman's correlations based on ranks. Differences were considered significant when P values were less than 0.05.

TABLE SQ

| SEQ ID NO | Amino acid sequences for proteins used in this study. |
|---|---|
| | Non-antigen-bearing nanoparticle components |
| 154 | >I53_dn5A pentamer |
| | MGKYDGSKLRIGILHARWNAEIILALVLGALKRLQEFGVKRENIIIETVPGSFELPYGSKL |
| | FVEKQKRLGKPLDAIIPIGVLIKGSTMHFEYICDSTTHQLMKLNFELGIPVIFGVLTCLTD |
| | EQAEARAGLIEGKMHNHGEDWGAAAVEMATKFNLEHHHHHH (SEQ ID NO: 154) |
| 155 | >I53_dn5A 1cys pentamer |
| | MGKYDGSKLRIGILHARGNAEIILALVLGALKRLQEFGVKRENIIIETVPGSFELPYGSKL |
| | FVEKQKRLGKPLDAIIPIGVLIRGSTPHFDYIADSTTHQLMKLNFELGIPVIFGVITADTC |
| | EQAEARAGLIEGKMHNHGEDWGAAAVEMATKFNGGWELQLEGSHHHHHH (SEQ ID |
| | NO: 155) |
| 156 | >I53_dn5A 2cys pentamer |
| | MGKYDGSKLRIGILHARGNAEIILALVLGALKRLQEFGVKRENIIIETVPGSFELPYGSKL |
| | FVEKQKRLGKPLDAIIPIGVLIRGCTPHFDYIADSTTHQLMKLNFELGIPVIFGVITADTC |
| | EQAEARAGLIEGKMHNHGEDWGAAAVEMATKFNGGWELQLEGSHHHHHH (SEQ ID |
| | NO: 156) |
| 157 | >I53_dn5Acp7 ELP1 pentamer |
| | MGSHHHHHHGSDEQAEERAGTKAGNHGEDWGAAAVEMATKFNGSGGSGKYDGSKLRIGILH |
| | ARGNAEIILELVLGALKRLQEFGVKRENIIIETVPGSFELPYGSKLFVEKQKRLGKPLDAI |
| | IPIGVLIRGSTAHFDYIADSTTHQLMKLNFELGIPVIFGVLTTESGGSVPGAGVPGVGVPG |
| | VGVPGAGVPGVGVPGAGVPGVGVPGAGVPGVGVPGVGVPGAGVPGVG (SEQ ID |
| | NO: 157) |
| 158 | >I53_dn5Acp7 ELP2 pentamer |
| | MGSHHHHHHGSDEQAEERAGTKAGNHGEDWGAAAVEMATKFNGSGGSGKYDGSKLRIGILH |
| | ARGNAEIILELVLGALKRLQEFGVKRENIIIETVPGSFELPYGSKLFVEKQKRLGKPLDAI |
| | IPIGVLIRGSTAHFDYIADSTTHQLMKLNFELGIPVIFGVLTTESGGSVPGAGVPGAGVPG |
| | AGVPGAGVPGAGVPGAGVPGAGVPGAGVPGAGVPGAGVPGAGVPGAG (SEQ ID |
| | NO: 158) |
| 159 | >I53_dn5Acp7 PASpentamer |
| | MGSHHHHHHGSDEQAEERAGTKAGNHGEDWGAAAVEMATKFNGSGGSGKYDGSKLRIGILH |
| | ARGNAEIILELVLGALKRLQEFGVKRENIIIETVPGSFELPYGSKLFVEKQKRLGKPLDAI |
| | IPIGVLIRGSTAHFDYIADSTTHQLMKLNFELGIPVIFGVLTTESGGSASPAAPAPASPAA |
| | PAPSAPAAASPAAPAPASPAAPAPSAPAAASPAAPAPASPAAPAPSAPAA (SEQ ID |
| | NO: 159) |
| 160 | >I53_dn5A pentamer (I53_dn5A.2, optimized for mammalian cell |
| | secretion) |
| | MDSKGSSQKGSRLLLLLVVSNLLLPQGVLAKYDGSKLRIGILHARGNAEIILELVLGALKR |
| | LQEFGVKRENIIIETVPGSFELPYGSKLFVEKQKRLGKPLDAIIPIGVLIRGSTAHFDYIA |
| | DSTTHQLMKLNFELGIPVIFGVLTTESDEQAEERAGTKAGNHGEDWGAAAVEMATKFNLEE |
| | QKLISEEDLHHHHHH (SEQ ID NO: 160) |
| 161 | >I53_dn5A_2gly pentamer |
| | MDSKGSSQKGSRLLLLLVVSNLLLPQGVLAKYDGSKLRIGILHARGNAEIILELVLGALKR |
| | LQEFGVKRENIIIETVPGSFELPYGSKLFVEKQKRLGKPLDAIIPIGVLIRGNDTHFDYIA |
| | DSTTHQLMKLNFELGIPVIFGVLTTNSTEQAEERAGTKAGNHGEDWGAAAVEMATKFNLEE |
| | QKLISEEDLHHHHHH (SEQ ID NO: 161) |
| 162 | >I53_dn5B trimer |
| | MEEAELAYLLGELAYKLGEYRIAIRAYRIALKRDPNNAEAWYNLGNAYYKQGRYREAIEYY |
| | QKALELDPNNAEAWYNLGNAYYERGEYEEAIEYYRKALRLDPNNADAMQNLLNAKMREE |
| | GGWELQGSLEHHHHHH (SEQ ID NO: 162) |
| 163 | >I53_dn5B_2gly trimer |
| | MDSKGSSQKGSRLLLLLVVSNLLLPQGVLAEEAELAYLLGELAYKLGEYRIAIRAYRIALK |
| | YDNLTAEAWYNLGNAYYKQGRYREAIEYYQKALELDPNNAEAWYNLGNAYYERGEYENATE |
| | YYRKALRLDPNNADAMQNLLNAKMREELEEQKLISEEDLHHHHHH (SEQ ID NO: 163) |
| 164 | >I53-50A trimer |
| | MKMEELFKKHKIVAVLRANSVEEAIEKAVAVFAGGVHLIEITFTVPDADTVIKALSVLKEK |
| | GAIIGAGTVTSVEQCRKAVESGAEFIVSPHLDEEISQFCKEKGVFYMPGVMTPTELVKAMK |
| | LGHTILKLFPGEVVGPQFVKAMKGPFPNVKFVPTGGVNLDNVCEWFKAGVLAVGVGSALVK |
| | GTPDEVREKAKAFVEKIRGCLEEQKLISEEDLHHHHHH (SEQ ID NO: 164) |
| 165 | >I53-50A_4gly trimer |
| | MDSKGSSQKGSRLLLLLVVSNLLLPQGVLAEELFKKHKIVAVLRANSVEEAIEKAVAVFAG |
| | GVHLIEITFTVPNATTVIKALSVLKEKGAIIGAGTVTSVEYANETVESGAEFIVSPHLDEE |
| | ISNFTKEKGVFYMPGVMTPTELVKAMKLGHTILKLFPGEVVGPQFVKAMKGPFHNATFVPT |
| | GGVNLDNVCEWFKAGVLAVGVGSALVKGTPDEVREKAKAFVEKIRGCLEEQKLISEEDLHH |
| | HHHH (SEQ ID NO: 165) |
| 166 | >I53-50B.4PT1 pentamer |
| | MNQHSHKDHETVRIAVVRARWHAEIVDACVSAFEAAMRDIGGDRFAVDVEDVPGAYEIPLH |
| | ARTLAETGRYGAVLGTAFVVNGGIYRHEFVASAVINGMMNVQLNTGVPVLSAVLTPHNYDK |
| | SKAHTLLFLALFAVKGMEAARACVEILAAREKIAAGSLEHHHHHH (SEQ ID NO: 166) |
| 167 | >2obx pentamer |
| | MNQHSHKDYETVRIAVVRARWHADIVDQCVSAFEAEMADIGGDRFAVDVEDVPGAYEIPLH |
| | ARTLAETGRYGAVLGTAFVVNGGIYRHEFVASAVIDGMMNVQLSTGVPVLSAVLTPHNYHD |
| | SAEHHRFFFEHFTVKGKEAARACVEILAAREKIAAGSLEHHHHHH (SEQ ID NO: 167) |
| | Antigen-bearing nanoparticle components and ELISA antigens |
| 168 | >H1MI15-I53_dn5B trimer (A/Michigan/45/2015 HA 1-676 Y98F no |
| | 1kr dn5B.SA.WELQ-H) |
| | MKAILVVLLYTFTTANADTLCIGYHANNSTDTVDTVLEKNVTVTHSVNLLEDKHNGKLCKL |
| | RGVAPLHLGKCNIAGWILGNPECESLSTASSWSYIVETSNSDNGTCFPGDFINYEELREQL |
| | SSVSSFERFEIFPKTSSWPNHDSNKGVTAACPHAGAKSFYKNLIWLVKKGNSYPKLNQSYI |
| | NDKGKEVLVLWGIHHPSTTADQQSLYQNADAYVFVGTSRYSKKFKPEIATRPKVRDQEGRM |
| | NYYWTLVEPGDKITFEATGNLVVPRYAFTMERNAGSGIIISDTPVHDCNTTCQTPEGAINT |
| | SLPFQNIHPITIGKCPKYVKSTKLRLATGLRNVPSIQSRGLFGAIAGFIEGGWTGMVDGWY |
| | GYHHQNEQGSGYAADLKSTQNAIDKITNKVNSVIEKMNTQFTAVGKEFNHLEKRIENLNKK |
| | VDDGFLDIWTYNAELLVLLENERTLDYHDSNVKNLYEKVRNQLKNNAKEIGNGCFEFYHKC |
| | DNTCMESVKNGTYDYPKYSEEAKLNREKIDGVSAEEAELAYLLGELAYKLGEYRIAIRAYR |
| | IALKRDPNNAEAWYNLGNAYYKQGRYREAIEYYQKALELDPNNAEAWYNLGNAYYERGEYE |
| | EAIEYYRKALRLDPNNADAMQNLLNAKMREEGGWELQHHHHHH (SEQ ID NO: 168) |
| 169 | >H1MI15-I53-50A trimer (A/Michigan/45/2015 HA 1-676 Y98F) |
| | MDSKGSSQKGSRLLLLLVVSNLLLPQGVLADTLCIGYHANNSTDTVDTVLEKNVTVTHSVN |
| | LLEDKHNGKLCKLRGVAPLHLGKCNIAGWILGNPECESLSTASSWSYIVETSNSDNGTCFP |
| | GDFINYEELREQLSSVSSFERFEIFPKTSSWPNHDSNKGVTAACPHAGAKSFYKNLIWLVK |
| | KGNSYPKLNQSYINDKGKEVLVLWGIHHPSTTADQQSLYQNADAYVFVGTSRYSKKFKPEI |
| | ATRPKVRDQEGRMNYYWTLVEPGDKITFEATGNLVVPRYAFTMERNAGSGIIISDTPVHDC |
| | NTTCQTPEGAINTSLPFQNIHPITIGKCPKYVKSTKLRLATGLRNVPSIQSRGLFGAIAGF |
| | IEGGWTGMVDGWYGYHHQNEQGSGYAADLKSTQNAIDKITNKVNSVIEKMNTQFTAVGKEF |
| | NHLEKRIENLNKKVDDGFLDIWTYNAELLVLLENERTLDYHDSNVKNLYEKVRNQLKNNAK |
| | EIGNGCFEFYHKCDNTCMESVKNGTYDYPKYSEEAKLNREKIDGVSAGSGGSGGSGGSGGS |
| | EKAAKAEEAARKMEELFKKHKIVAVLRANSVEEAIEKAVAVFAGGVHLIEITFTVPDADTV |
| | IKALSVLKEKGAIIGAGTVTSVEQCRKAVESGAEFIVSPHLDEEISQFCKEKGVFYMPGVM |
| | TPTELVKAMKLGHTILKLFPGEVVGPQFVKAMKGPFPNVKFVPTGGVNLDNVCEWFKAGVL |
| | AVGVGSALVKGTPDEVREKAKAFVEKIRGCTELEEQKLISEEDLHHHHHH (SEQ ID |
| | NO: 169) |
| 170 | >H1MI15-foldon trimer (A/Michigan/45/2015 HA 1-676 Y98F FAH) |
| | MDSKGSSQKGSRLLLLLVVSNLLLPQGVLADTLCIGYHANNSTDTVDTVLEKNVTVTHSVN |
| | LLEDKHNGKLCKLRGVAPLHLGKCNIAGWILGNPECESLSTASSWSYIVETSNSDNGTCFP |
| | GDFINYEELREQLSSVSSFERFEIFPKTSSWPNHDSNKGVTAACPHAGAKSFYKNLIWLVK |
| | KGNSYPKLNQSYINDKGKEVLVLWGIHHPSTTADQQSLYQNADAYVFVGTSRYSKKFKPEI |
| | ATRPKVRDQEGRMNYYWTLVEPGDKITFEATGNLVVPRYAFTMERNAGSGIIISDTPVHDC |
| | NTTCQTPEGAINTSLPFQNIHPITIGKCPKYVKSTKLRLATGLRNVPSIQSRGLFGAIAGE |
| | IEGGWTGMVDGWYGYHHQNEQGSGYAADLKSTQNAIDKITNKVNSVIEKMNTQFTAVGKEF |
| | NHLEKRIENLNKKVDDGFLDIWTYNAELLVLLENERTLDYHDSNVKNLYEKVRNQLKNNAK |
| | EIGNGCFEFYHKCDNTCMESVKNGTYDYPKYSEEAKLNREKIDGVGSGYIPEAPRDGQAYV |
| | RKDGEWVLLSTFLGSGLNDIFEAQKIEWHEGHHHHHH (SEQ ID NO: 170) |
| 171 | >H1MI15-1na0C3_int2 trimer (A/Michigan/45/2015 HA 1-676 Y98F |
| | no lkr 1na0C3_int2.SA. WELQ-H) |
| | MKAILVVLLYTFTTANADTLCIGYHANNSTDTVDTVLEKNVTVTHSVNLLEDKHNGKLCKL |
| | RGVAPLHLGKCNIAGWILGNPECESLSTASSWSYIVETSNSDNGTCFPGDFINYEELREQL |
| | SSVSSFERFEIFPKTSSWPNHDSNKGVTAACPHAGAKSFYKNLIWLVKKGNSYPKLNQSYI |
| | NDKGKEVLVLWGIHHPSTTADQQSLYQNADAYVFVGTSRYSKKFKPEIATRPKVRDQEGRM |
| | NYYWTLVEPGDKITFEATGNLVVPRYAFTMERNAGSGIIISDTPVHDCNTTCQTPEGAINT |
| | SLPFQNIHPITIGKCPKYVKSTKLRLATGLRNVPSIQSRGLFGAIAGFIEGGWTGMVDGWY |
| | GYHHQNEQGSGYAADLKSTQNAIDKITNKVNSVIEKMNTQFTAVGKEFNHLEKRIENLNKK |
| | VDDGFLDIWTYNAELLVLLENERTLDYHDSNVKNLYEKVRNQLKNNAKEIGNGCFEFYHKC |
| | DNTCMESVKNGTYDYPKYSEEAKLNREKIDGVSAEEAELAYLLGELAYKLGEYRIAIRAYR |
| | IALKRDPNNAEAWYNLGNAYYKQGDYDEAIEYYQKALELDPNNAEAWYNLGNAYYKQGDYD |
| | EAIEYYQKALELDPNNAEAKQNLGNAKQKQGGGWELQHHHHHH (SEQ ID NO: 171) |
| 172 | >SARS-COV-2_RBD-I53-50A trimer (16GSlinker, using wild type |
| | RBD from Wuhan-Hu-1) |
| | MGILPSPGMPALLSLVSLLSVLLMGCVAETGTRFPNITNLCPFGEVFNATRFASVYAWNRK |
| | RISNCVADYSVLYNSASFSTFKCYGVSPTKLNDLCFTNVYADSFVIRGDEVRQIAPGQTGK |
| | IADYNYKLPDDFTGCVIAWNSNNLDSKVGGNYNYLYRLERKSNLKPFERDISTEIYQAGST |
| | PCNGVEGFNCYFPLQSYGFQPTNGVGYQPYRVVVLSFELLHAPATVCGPKKSTGGSGGSGS |
| | GGSGGSGSEKAAKAEEAARKMEELFKKHKIVAVLRANSVEEAIEKAVAVFAGGVHLIEITE |
| | TVPDADTVIKALSVLKEKGAIIGAGTVTSVEQARKAVESGAEFIVSPHLDEEISQFAKEKG |
| | VFYMPGVMTPTELVKAMKLGHTILKLFPGEVVGPQFVKAMKGPFPNVKFVPTGGVNLDNVA |
| | EWFKAGVLAVGVGSALVKGTPDEVREKAKAFVEKIRGATEGGSHHHHHHHH (SEQ ID |
| | NO: 172) |
| 173 | >SARS-COV-2_SpikeHexaPro-foldon trimer |
| | MFVFLVLLPLVSSQCVNLTTRTQLPPAYTNSFTRGVYYPDKVERSSVLHSTQDLFLPFFSN |
| | VTWFHAIHVSGTNGTKRFDNPVLPFNDGVYFASTEKSNIIRGWIFGTTLDSKTQSLLIVNN |
| | ATNVVIKVCEFQFCNDPFLGVYYHKNNKSWMESEFRVYSSANNCTFEYVSQPFLMDLEGKQ |
| | GNFKNLREFVFKNIDGYFKIYSKHTPINLVRDLPQGESALEPLVDLPIGINITRFQTLLAL |
| | HRSYLTPGDSSSGWTAGAAAYYVGYLQPRTELLKYNENGTITDAVDCALDPLSETKCTLKS |
| | FTVEKGIYQTSNERVQPTESIVRFPNITNLCPFGEVFNATRFASVYAWNRKRISNCVADYS |
| | VLYNSASFSTFKCYGVSPTKLNDLCFTNVYADSFVIRGDEVRQIAPGQTGKIADYNYKLPD |
| | DETGCVIAWNSNNLDSKVGGNYNYLYRLERKSNLKPFERDISTEIYQAGSTPCNGVEGENC |
| | YFPLQSYGFQPTNGVGYQPYRVVVLSFELLHAPATVCGPKKSTNLVKNKCVNFNENGLTGT |
| | GVLTESNKKELPFQQFGRDIADTTDAVRDPQTLEILDITPCSFGGVSVITPGTNTSNQVAV |
| | LYQDVNCTEVPVAIHADQLTPTWRVYSTGSNVFQTRAGCLIGAEHVNNSYECDIPIGAGIC |
| | ASYQTQTNSPGSASSVASQSIIAYTMSLGAENSVAYSNNSIAIPTNFTISVTTEILPVSMT |
| | KTSVDCTMYICGDSTECSNLLLQYGSFCTQLNRALTGIAVEQDKNTQEVFAQVKQIYKTPP |
| | IKDFGGFNFSQILPDPSKPSKRSPIEDLLENKVTLADAGFIKQYGDCLGDIAARDLICAQK |
| | FNGLTVLPPLLTDEMIAQYTSALLAGTITSGWTFGAGPALQIPFPMQMAYRFNGIGVTQNV |
| | LYENQKLIANQFNSAIGKIQDSLSSTPSALGKLQDVVNQNAQALNTLVKQLSSNFGAISSV |
| | LNDILSRLDPPEAEVQIDRLITGRLQSLQTYVTQQLIRAAEIRASANLAATKMSECVLGQS |
| | KRVDFCGKGYHLMSFPQSAPHGVVFLHVTYVPAQEKNFTTAPAICHDGKAHFPREGVEVSN |
| | GTHWFVTQRNFYEPQIITTDNTFVSGNCDVVIGIVNNTVYDPLQPELDSFKEELDKYFKNH |
| | TSPDVDLGDISGINASVVNIQKEIDRLNEVAKNLNESLIDLQELGKYEQGSGYIPEAPRDG |
| | QAYVRKDGEWVLLSTFLGRSLEVLFQGPGHHHHHHHHSAWSHPQFEKGGGSGGGGSGGSAW |
| | SHPQFEK (SEQ ID NO: 173) |
| 174 | >RSV-F_DS-Cav1-I53-50A trimer |
| | MELLILKANAITTILTAVTFCFASGQNITEEFYQSTCSAVSKGYLSALRTGWYTSVITIEL |
| | SNIKFNKCNGTDAKVKLIKQELDKYKNAVTELQLLMQSTPATNNRARRELPREMNYTLNNA |
| | KKTNVTLSKKRKRRFLGFLLGVGSAIASGVAVCKVLHLEGEVNKIKSALLSTNKAVVSLSN |
| | GVSVLTFKVLDLKNYIDKQLLPILNKQSCSISNIETVIEFQQKNNRLLEITREFSVNAGVT |
| | TPVSTYMLTNSELLSLINDMPITNDQKKLMSNNVQIVRQQSYSIMCIIKEEVLAYVVQLPL |
| | YGVIDTPCWKLHTSPLCTTNTKEGSNICLTRTDRGWYCDNAGSVSFFPQAETCKVQSNRVF |
| | CDTMNSLTLPSEVNLCNVDIFNPKYDCKIMTSKTDVSSSVITSLGAIVSCYGKTKCTASNK |
| | NRGIIKTFSNGCDYVSNKGVDTVSVGNTLYYVNKQEGKSLYVKGEPIINFYDPLVFPSDEF |
| | DASISQVNEKINQSLAFIRKSDELLGSGGSGSGSGGSEKAAKAEEAARKMEELFKKHKIVA |
| | VLRANSVEEAIEKAVAVFAGGVHLIEITFTVPDADTVIKALSVLKEKGAIIGAGTVTSVEQ |
| | CRKAVESGAEFIVSPHLDEEISQFCKEKGVFYMPGVMTPTELVKAMKLGHTILKLFPGEVV |
| | GPQFVKAMKGPFPNVKFVPTGGVNLDNVCEWFKAGVLAVGVGSALVKGTPDEVREKAKAFV |
| | EKIRGCTELEHHHHHH (SEQ ID NO: 174) |
| 175 | >RSV-F_DS-Cav1-foldon trimer |
| | MELLILKANAITTILTAVTFCFASGQNITEEFYQSTCSAVSKGYLSALRTGWYTSVITIEL |
| | SNIKFNKCNGTDAKVKLIKQELDKYKNAVTELQLLMQSTPATNNRARRELPREMNYTLNNA |
| | KKTNVTLSKKRKRRFLGFLLGVGSAIASGVAVCKVLHLEGEVNKIKSALLSTNKAVVSLSN |
| | GVSVLTFKVLDLKNYIDKQLLPILNKQSCSISNIETVIEFQQKNNRLLEITREFSVNAGVT |
| | TPVSTYMLTNSELLSLINDMPITNDQKKLMSNNVQIVRQQSYSIMCIIKEEVLAYVVQLPL |
| | YGVIDTPCWKLHTSPLCTTNTKEGSNICLTRTDRGWYCDNAGSVSFFPQAETCKVQSNRVF |
| | CDTMNSLTLPSEVNLCNVDIFNPKYDCKIMTSKTDVSSSVITSLGAIVSCYGKTKCTASNK |
| | NRGIIKTFSNGCDYVSNKGVDTVSVGNTLYYVNKQEGKSLYVKGEPIINFYDPLVFPSDEF |
| | DASISQVNEKINQSLAFIRKSDELLSAIGGYIPEAPRDGQAYVRKDGEWVLLSTFLENLYF |
| | QSSAWSHPQFEKGGGSGGGSGGSAWSHPQFEKGSGSGSGLNDIFEAQKIEWHEGSGSGSHH |
| | HHHHHH (SEQ ID NO: 175) |
| 176 | >HIVenv(AMC009)-I53-50A trimer |
| | ADKLWVTVYYGVPVWKDACTTLFCASDAKAYDTEKRNVWATHCCVPTDPNPQEVVLENVTE |
| | NFNMWKNDMVEQMHEDIISLWDQSLKPCVKLTPLCVTLNCTDYVGNATNASTTNATGGIGG |
| | TVERGEIKNCSFNITTSLRDKVQKEYALFYKLDIVPIDNDNTNNTYRLINCNTTVIKQACP |
| | KVSFEPIPIHYCAPAGFAILKCNDKKFNGTGPCTNVSTVQCTHGIRPVVSTQLLINGSLAE |
| | KEVIIRSQNFTNNAKVIIVQLNESVVINCTRPNNNTVKSIHIAPGQWFYYTGAIIGDIRQA |
| | HCNISRVKWNNTLKQIATKLREQFKNKTIAFNQSSGGDPEIVMHSFNCGGEFFYCNTTQLF |
| | NSTWNDTEVSNYTDITHITLPCRIKQIINMWQRVGQAMYAPPIRGQIRCSSNITGLLLTRD |
| | GGSNENKTSETETFRPAGGDMRDNWRSELYKYKVVKIEPLGVAPTRCKRRVVQRRRRRRAV |
| | GAIGAVSLGFLGAAGSTMGAASMTLTVQARQLLSGIVQQQNNCLRAPECQQHMLKDTHWGI |
| | KQLQARVLAVEHYLRDQQLLGIWGCSGKLICCTAVPWNNTWSNRSLDMIWNNMTWIEWERE |
| | IDNYTGLIYNLLEESQNQQEKNEQELLELDGSGGSGGSGGSGGSEKAAKAEEAARKMEELE |
| | KKHKIVAVLRANSVEEAIEKAVAVFAGGVHLIEITFTVPDADTVIKALSVLKEKGAIIGAG |
| | TVTSVEQCRKAVESGAEFIVSPHLDEEISQFCKEKGVFYMPGVMTPTELVKAMKLGHTILK |
| | LFPGEVVGPQFVKAMKGPFPNVKFVPTGGVNLDNVCEWFKAGVLAVGVGSALVKGTPDEVR |
| | EKAKAFVEKIRGCTE (SEQ ID NO: 176) |
| 177 | >HIVenv(ConM)-I53-50A trimer |
| | AENLWVTVYYGVPVWKDAETTLFCASDAKAYDTEKRNVWATHCCVPTDPNPQEIVLENVTE |
| | NFNMWKNNMVEQMHTDIISLWDQSLKPCVKLTPLCVTLNCTDVNATNNTTNNEEIKNCSEN |
| | ITTELRDKKKKVYALFYKLDVVPIDDNNSYRLINCNTSAITQACPKVSFEPIPIHYCAPAG |
| | FAILKCNDKKFNGTGPCKNVSTVQCTHGIKPVVSTQLLLNGSLAEEEIIIRSENITNNAKT |
| | IIVQLNESVEINCTRPNNNTRKSIRIGPGQWFYATGDIIGDIRQAHCNISRTKWNKTLQQV |
| | AKKLREHENKTIIFNPSSGGDLEITTHSFNCGGEFFYCNTSELENSTWNGTNNTITLPCRI |
| | KQIINMWQRVGQAMYAPPIEGKIRCTSNITGLLLTRDGGNNNTETFRPGGGDMRDNWRSEL |
| | YKYKVVKIEPLGVAPTRCKRRVVERRRRRRAVGIGAVELGELGAAGSTMGAASMTLTVQAR |
| | NLLSGIVQQQSNLLRAPECQQHLLQLTVWGIKQLQARVLAVERYLKDQQLLGIWGCSGKLI |
| | CCTNVPWNSSWSNKSQDEIWDNMTWMEWDKEINNYTDIIYSLIEESQNQQEKNEQELLALD |
| | GSGGSGGSGGSGGSEKAAKAEEAARKMEELFKKHKIVAVLRANSVEEAIEKAVAVFAGGVH |
| | LIEITFTVPDADTVIKALSVLKEKGAIIGAGTVTSVEQCRKAVESGAEFIVSPHLDEEISQ |
| | FCKEKGVFYMPGVMTPTELVKAMKLGHTILKLFPGEVVGPQFVKAMKGPFPNVKFVPTGGV |
| | NLDNVCEWFKAGVLAVGVGSALVKGTPDEVREKAKAFVEKIRGCTE (SEQ ID |
| | NO: 177) |
| 178 | >HIVenv(AMC009)-8xHis trimer |
| | ADKLWVTVYYGVPVWKDACTTLFCASDAKAYDTEKRNVWATHCCVPTDPNPQEVVLENVTE |
| | NFNMWKNDMVEQMHEDIISLWDQSLKPCVKLTPLCVTLNCTDYVGNATNASTTNATGGIGG |
| | TVERGEIKNCSFNITTSLRDKVQKEYALFYKLDIVPIDNDNTNNTYRLINCNTTVIKQACP |
| | KVSFEPIPIHYCAPAGFAILKCNDKKFNGTGPCTNVSTVQCTHGIRPVVSTQLLLNGSLAE |
| | KEVIIRSQNFTNNAKVIIVQLNESVVINCTRPNNNTVKSIHIAPGQWFYYTGAIIGDIRQA |
| | HCNISRVKWNNTLKQIATKLREQFKNKTIAFNQSSGGDPEIVMHSFNCGGEFFYCNTTQLE |
| | NSTWNDTEVSNYTDITHITLPCRIKQIINMWQRVGQAMYAPPIRGQIRCSSNITGLLLTRD |
| | GGSNENKTSETETFRPAGGDMRDNWRSELYKYKVVKIEPLGVAPTRCKRRVVQRRRRRRAV |
| | GAIGAVSLGFLGAAGSTMGAASMTLTVQARQLLSGIVQQQNNCLRAPECQQHMLKDTHWGI |
| | KQLQARVLAVEHYLRDQQLLGIWGCSGKLICCTAVPWNNTWSNRSLDMIWNNMTWIEWERE |
| | IDNYTGLIYNLLEESQNQQEKNEQELLELDGSGSGGSGHHHHHHHH (SEQ ID |
| | NO: 178) |
| 179 | >HIVenv(ConM)-8xHis trimer |
| | AENLWVTVYYGVPVWKDAETTLFCASDAKAYDTEKRNVWATHCCVPTDPNPQEIVLENVTE |
| | NENMWKNNMVEQMHTDIISLWDQSLKPCVKLTPLCVTLNCTDVNATNNTTNNEEIKNCSEN |
| | ITTELRDKKKKVYALFYKLDVVPIDDNNSYRLINCNTSAITQACPKVSFEPIPIHYCAPAG |
| | FAILKCNDKKFNGTGPCKNVSTVQCTHGIKPVVSTQLLINGSLAEEEIIIRSENITNNAKT |
| | IIVQLNESVEINCTRPNNNTRKSIRIGPGQWFYATGDIIGDIRQAHCNISRTKWNKTLQQV |
| | AKKLREHENKTIIFNPSSGGDLEITTHSENCGGEFFYCNTSELENSTWNGTNNTITLPCRI |
| | KQIINMWQRVGQAMYAPPIEGKIRCTSNITGLLLTRDGGNNNTETFRPGGGDMRDNWRSEL |
| | YKYKVVKIEPLGVAPTRCKRRVVERRRRRRAVGIGAVELGELGAAGSTMGAASMTLTVQAR |
| | NLLSGIVQQQSNLLRAPECQQHLLQLTVWGIKQLQARVLAVERYLKDQQLLGIWGCSGKLI |
| | CCTNVPWNSSWSNKSQDEIWDNMTWMEWDKEINNYTDIIYSLIEESQNQQEKNEQELLALD |
| | GSGSGGSGHHHHHHHH (SEQ ID NO: 179) |

### REFERENCES

Abbott, R. K., and Crotty, S. (2020). Factors in B cell competition and immunodominance. Immunol. Rev. 296, 120-131.doi.org/10.1111/imr.12861.
Abbott, R. K., Lee, J. H., Menis, S., Skog, P., Rossi, M., Ota, T., Kulp, D. W., Bhullar, D., Kalyuzhniy, O., Havenar-Daughton, C., et al. (2018). Precursor Frequency and Affinity Determine B Cell Competitive Fitness in Germinal Centers, Tested with Germline-Targeting HIV Vaccine Immunogens. Immunity 48, 133-146.e6.doi.org/10.1016/j.immuni.2017.11.023.
Adolf-Bryfogle, J., Labonte, J. W., Kraft, J. C., Shapovalov, M., Raemisch, S., Lütteke, T., DiMaio, F., Bahl, C. D., Pallesen, J., King, N. P., et al. (2021). Growing Glycans in Rosetta: Accurate de novo glycan modeling, density fitting, and rational sequon design. bioRxiv 2021.09.27.462000.doi.org/10.1101/2021.09.27.462000.
Aide, P., Dobaño, C., Sacarlal, J., Aponte, J. J., Mandomando, I., Guinovart, C., Bassat, Q., Renom, M., Puyol, L., Macete, E., et al. (2011). Four year immunogenicity of the RTS,S/AS02(A) malaria vaccine in Mozambican children during a phase IIb trial. Vaccine 29, 6059-6067.doi.org/10.1016/j.vaccine.2011.03.041.
Arunachalam, P. S., Walls, A. C., Golden, N., Atyeo, C., Fischinger, S., Li, C., Aye, P., Navarro, M. J., Lai, L., Edara, V. V., et al. (2021). Adjuvanting a subunit COVID-19 vaccine to induce protective immunity. Nature 594, 253-258.doi.org/10.1038/s41586-021-03530-2.
Baden, L. R., El Sahly, H. M., Essink, B., Kotloff, K., Frey, S., Novak, R., Diemert, D., Spector, S. A., Rouphael, N., Creech, C. B., et al. (2021). Efficacy and Safety of the mRNA-1273 SARS-CoV-2 Vaccine. N. Engl. J. Med. 384, 403-416.doi.org/10.1056/NEJMoa2035389.
Bajic, G., Maron, M. J., Adachi, Y., Onodera, T., McCarthy, K. R., McGee, C. E., Sempowski, G. D., Takahashi, Y., Kelsoe, G., Kuraoka, M., et al. (2019). Influenza Antigen Engineering Focuses Immune Responses to a Subdominant but Broadly Protective Viral Epitope. Cell Host Microbe 25, 827-835.e6.doi.org/10.1016/j.chom.2019.04.003.
Bale, J. B., Park, R. U., Liu, Y., Gonen, S., Gonen, T., Cascio, D., King, N. P., Yeates, T. O., and Baker, D. (2015). Structure of a designed tetrahedral protein assembly variant engineered to have improved soluble expression. Protein Sci. 24, 1695-1701.doi.org/10.1002/pro.2748.
Bale, J. B., Gonen, S., Liu, Y., Sheffler, W., Ellis, D., Thomas, C., Cascio, D., Yeates, T. O., Gonen, T., King, N. P., et al. (2016). Accurate design of megadalton-scale two-component icosahedral protein complexes. Science 353, 389-394.doi.org/10.1126/science.aaM18.
Bloom, J. D., Gong, L. I., and Baltimore, D. (2010). Permissive secondary mutations enable the evolution of influenza oseltamivir resistance. Science 328, 1272-1275. doi.org/10.1126/science.1187816.
Boyoglu-Barnum, S., Hutchinson, G. B., Boyington, J. C., Moin, S. M., Gillespie, R. A., Tsybovsky, Y., Stephens, T., Vaile, J. R., Lederhofer, J., Corbett, K. S., et al. (2020). Glycan repositioning of influenza hemagglutinin stem facilitates the elicitation of protective cross-group antibody responses. Nat. Commun. 11, 791.doi.org/10.1038/s41467-020-14579-4.
Boyoglu-Barnum, S., Ellis, D., Gillespie, R. A., Hutchinson, G. B., Park, Y. J., Moin, S. M., Acton, O. J., Ravichandran, R., Murphy, M., Pettie, D., et al. (2021). Quadrivalent influenza nanoparticle vaccines induce broad protection. Nature 592, 623-628.doi.org/10.1038/s41586-021-03365-x.
Brody, N. I., and Siskind, G. W. (1969). Studies on antigenic competition. J. Exp. Med. 130, 821-832.doi.org/10.1084/jem.130.4.821.
Brouwer, P. J. M., Antanasijevic, A., Berndsen, Z., Yasmeen, A., Fiala, B., Bijl, T. P. L., Bontjer, I., Bale, J. B., Sheffler, W., Allen, J. D., et al. (2019). Enhancing and shaping the immunogenicity of native-like HIV-1 envelope trimers with a two-component protein nanoparticle. Nat. Commun. 10, 4272.doi.org/10.1038/s41467-019-12080-1.
Brouwer, P. J. M., Brinkkemper, M., Maisonnasse, P., Dereuddre-Bosquet, N., Grobben, M., Claireaux, M., de Gast, M., Marlin, R., Chesnais, V., Diry, S., et al. (2021a). Two-component spike nanoparticle vaccine protects macaques from SARS-CoV-2 infection. Cell 184, 1188-1200.e19.doi.org/10.1016/j.ce11.2021.01.035.
Brouwer, P. J. M., Antanasijevic, A., de Gast, M., Allen, J. D., Bijl, T. P. L., Yasmeen, A., Ravichandran, R., Burger, J. A., Ozorowski, G., Tones, J. L., et al. (2021b). Immunofocusing and enhancing autologous Tier-2 HIV-1 neutralization by displaying Env trimers on two-component protein nanoparticles. NPJ Vaccines 6, 24.doi.org/10.1038/s41541-021-00285-9.
Bruun, T. U. J., Andersson, A. M. C., Draper, S. J., and Howarth, M. (2018). Engineering a Rugged Nanoscaffold To Enhance Plug-and-Display Vaccination. ACS Nano 12, 8855-8866. doi.org/10.1021/acsnano.8b02805.
Burton, D. R. (2002). Antibodies, viruses and vaccines. Nat. Rev. Immunol. 2, 706-713. doi.org/10.1038/nri891.
Chackerian, B. (2007). Virus-like particles: flexible platforms for vaccine development. Expert Rev. Vaccines 6, 381-390.doi.org/10.1586/14760584.6.3.381.
Cohen, A. A., Gnanapragasam, P. N. P., Lee, Y. E., Hoffman, P. R., Ou, S., Kakutani, L. M., Keeffe, J. R., Wu, H. J., Howarth, M., West, A. P., et al. (2021). Mosaic nanoparticles elicit cross-reactive immune responses to zoonotic coronaviruses in mice. Science 371, 735-741. doi.org/10.1126/science.abf6840.
Collins, K. A., Snaith, R., Cottingham, M. G., Gilbert, S. C., and Hill, A. V. S. (2017). Enhancing protective immunity to malaria with a highly immunogenic virus-like particle vaccine. Sci. Rep. 7, 46621.doi.org/10.1038/srep46621.
Crawford, K. H. D., Eguia, R., Dingens, A. S., Loes, A. N., Malone, K. D., Wolf, C. R., Chu, H. Y., Tortorici, M. A., Veesler, D., Murphy, M., et al. (2020). Protocol and Reagents for Pseudotyping Lentiviral Particles with SARS-CoV-2 Spike Protein for Neutralization Assays. Viruses 12. doi.org/10.3390/v12050513.
Creanga, A., Gillespie, R. A., Fisher, B. E., Andrews, S. F., Lederhofer, J., Yap, C., Hatch, L., Stephens, T., Tsybovsky, Y., Crank, M. C., et al. (2021). A comprehensive influenza reporter virus panel for high-throughput deep profiling of neutralizing antibodies. Nat. Commun. 12, 1722.doi.org/10.1038/s41467-021-21954-2.
Crooke, S. N., Zheng, J., Ganewatta, M. S., Guldberg, S. M., Reineke, T. M., and Finn, M. G. (2019). Immunological Properties of Protein-Polymer Nanoparticles. ACS Appl. Bio Mater. 2, 93-103.doi.org/10.1021/acsabm.8b00418.
Datoo, M. S., Natama, M. H., Somé, A., Traoré, O., Rouamba, T., Bellamy, D., Yameogo, P., Valia, D., Tegneri, M., Ouedraogo, F., et al. (2021). Efficacy of a low-dose candidate malaria vaccine, R21 in adjuvant Matrix-M, with seasonal administration to children in Burkina Faso: a randomised controlled trial. Lancet 397, 1809-1818.doi.org/10.1016/S0140-6736(21)00943-0.
Dosenovic, P., Kara, E. E., Pettersson, A. K., McGuire, A. T., Gray, M., Hartweger, H., Thientosapol, E. S., Stamatatos, L., and Nussenzweig, M. C. (2018). Anti-HIV-1 B cell responses are dependent on B cell precursor frequency and antigen-binding affinity. Proc. Natl. Acad. Sci. U. S. A 115, 4743-4748.doi.org/10.1073/pnas.1803457115.
Duan, H., Chen, X., Boyington, J. C., Cheng, C., Zhang, Y., Jafari, A. J., Stephens, T., Tsybovsky, Y., Kalyuzhniy, O., Zhao, P., et al. (2018). Glycan Masking Focuses Immune Responses to the HIV-1 CD4-Binding Site and Enhances Elicitation of VRC01-Class Precursor Antibodies. Immunity 49, 301-311.e5. doi.org/10.1016/j.immuni.2018.07.005.
Eggink, D., Goff, P. H., and Palese, P. (2014). Guiding the immune response against influenza virus hemagglutinin toward the conserved stalk domain by hyperglycosylation of the globular head domain. J. Virol. 88, 699-704.doi.org/10.1128/JVI.02608-13.
Ellis, D., Brunette, N., Crawford, K. H. D., Walls, A. C., Pham, M. N., Chen, C., Herpoldt, K. L., Fiala, B., Murphy, M., Pettie, D., et al. (2021). Stabilization of the SARS-CoV-2 Spike Receptor-Binding Domain Using Deep Mutational Scanning and Structure-Based Design. Front. Immunol. 12, 710263.doi.org/10.3389/fimmu.2021.710263.
Eto, Y., Yoshioka, Y., Ishida, T., Yao, X., Morishige, T., Narimatsu, S., Mizuguchi, H., Mukai, Y., Okada, N., Kiwada, H., et al. (2010). Optimized PEGylated adenovirus vector reduces the anti-vector humoral immune response against adenovirus and induces a therapeutic effect against metastatic lung cancer. Biol. Pharm. Bull. 33, 1540-1544.doi.org/10.1248/bpb.33.1540.
Falvo, E., Tremante, E., Arcovito, A., Papi, M., Elad, N., Boffi, A., Morea, V., Conti, G., Toffoli, G., Fracasso, G., et al. (2016). Improved Doxorubicin Encapsulation and Pharmacokinetics of Ferritin-Fusion Protein Nanocarriers Bearing Proline, Serine, and Alanine Elements. Biomacromolecules 17, 514-522.doi.org/10.1021/acs.biomac.5b01446.
Fleishman, S. J., Leaver-Fay, A., Corn, J. E., Strauch, E. M., Khare, S. D., Koga, N., Ashworth, J., Murphy, P., Richter, F., Lemmon, G., et al. (2011). RosettaScripts: a scripting language interface to the Rosetta macromolecular modeling suite. PLoS One 6, e20161. doi.org/10.1371/journal.pone.0020161.
Garrity, R. R., Rimmelzwaan, G., Minassian, A., Tsai, W. P., Lin, G., de Jong, J. J., Goudsmit, J., and Nara, P. L. (1997). Refocusing neutralizing antibody response by targeted dampening of an immunodominant epitope. J. Immunol. 159, 279-289.
Goddard, T. D., Huang, C. C., Meng, E. C., Pettersen, E. F., Couch, G. S., Morris, J. H., and Ferrin, T. E. (2018). UCSF ChimeraX: Meeting modern challenges in visualization and analysis. Protein Sci. 27, 14-25.doi.org/10.1002/pro.3235.
Goodson, R. J., and Katre, N. V. (1990). Site-directed pegylation of recombinant interleukin-2 at its glycosylation site. Biotechnology 8, 343-346.doi.org/10.1038/nbt0490-343.
Gordon, D. M., McGovern, T. W., Krzych, U., Cohen, J. C., Schneider, I., LaChance, R., Heppner, D. G., Yuan, G., Hollingdale, M., and Slaoui, M. (1995). Safety, immunogenicity, and efficacy of a recombinantly produced Plasmodium falciparum circumsporozoite protein-hepatitis B surface antigen subunit vaccine. J. Infect. Dis. 171, 1576-1585.doi.org/10.1093/infdis/171.6.1576.
Herzenberg, L. A., and Tokuhisa, T. (1982). Epitope-specific regulation. I. Carrier-specific induction of suppression for IgG anti-hapten antibody responses. J. Exp. Med. 155, 1730-1740. doi.org/10.1084/jem.155.6.1730.
Houser, K. V., Chen, G. L., Carter, C., Crank, M. C., Nguyen, T. A., Burgos Florez, M. C., Berkowitz, N. M., Mendoza, F., Hendel, C. S., Gordon, I. J., et al. (2022). Safety and immunogenicity of a ferritin nanoparticle H2 influenza vaccine in healthy adults: a phase 1 trial. Nat. Med. in press.doi.org/10.1038/s41591-021-01660-8.
Hsieh, C. L., Goldsmith, J. A., Schaub, J. M., DiVenere, A. M., Kuo, H. C., Javanmardi, K., Le, K. C., Wrapp, D., Lee, A. G., Liu, Y., et al. (2020). Structure-based design of prefusion-stabilized SARS-CoV-2 spikes. Science 369, 1501-1505.doi.org/10.1126/science.abd0826.
Huang, Y. W., Yang, H. I., Wu, Y. T., Hsu, T. L., Lin, T. W., Kelly, J. W., and Wong, C. H. (2017). Residues Comprising the Enhanced Aromatic Sequon Influence Protein N-Glycosylation Efficiency. J. Am. Chem. Soc. 139, 12947-12955.doi.org/10.1021/jacs.7b03868.
Irvine, D. J., and Read, B. J. (2020). Shaping humoral immunity to vaccines through antigen-displaying nanoparticles. Curr. Opin. Immunol. 65, 1-6.doi.org/10.1016/j.coi.2020.01.007.
Jardine, J., Julien, J. P., Menis, S., Ota, T., Kalyuzhniy, O., McGuire, A., Sok, D., Huang, P. S., MacPherson, S., Jones, M., et al. (2013). Rational HIV immunogen design to target specific germline B cell receptors. Science 340, 711-716.doi.org/10.1126/science.1234150.
Jardine, J. G., Ota, T., Sok, D., Pauthner, M., Kulp, D. W., Kalyuzhniy, O., Skog, P. D., Thinnes, T. C., Bhullar, D., Briney, B., et al. (2015). HIV-1 VACCINES. Priming a broadly neutralizing antibody response to HIV-1 using a germline-targeting immunogen. Science 349, 156-161. doi.org/10.1126/science.aac5894.
Jardine, J. G., Kulp, D. W., Havenar-Daughton, C., Sarkar, A., Briney, B., Sok, D., Sesterhenn, F., Ereño-Orbea, J., Kalyuzhniy, O., Deresa, I., et al. (2016). HIV-1 broadly neutralizing antibody precursor B cells revealed by germline-targeting immunogen. Science 351, 1458-1463. doi.org/10.1126/science.aad9195.
Jegerlehner, A., Wiesel, M., Dietmeier, K., Zabel, F., Gatto, D., Saudan, P., and Bachmann, M. F. (2010). Carrier induced epitopic suppression of antibody responses induced by virus-like particles is a dynamic phenomenon caused by carrier-specific antibodies. Vaccine 28, 5503-5512.doi.org/10.1016/j.vaccine.2010.02.103.
Johansson, B. E., Moran, T. M., and Kilbourne, E. D. (1987). Antigen-presenting B cells and helper T cells cooperatively mediate intravirionic antigenic competition between influenza A virus surface glycoproteins. Proc. Natl. Acad. Sci. U. S. A 84, 6869-6873. doi.org/10.1073/pnas.84.19.6869.
Johansson, B. E., Bucher, D. J., and Kilbourne, E. D. (1989). Purified influenza virus hemagglutinin and neuraminidase are equivalent in stimulation of antibody response but induce contrasting types of immunity to infection. J. Virol. 63, 1239-1246. doi.org/10.1128/JVI.63.3.1239-1246.1989.
Joyce, M. G., King, H. A. D., Naouar, I. E., Ahmed, A., Peachman, K. K., Cincotta, C. M., Subra, C., Chen, R. E., Thomas, P. V., Chen, W. H., et al. (2021). Efficacy of a Broadly Neutralizing SARS-CoV-2 Ferritin Nanoparticle Vaccine in Nonhuman Primates. bioRxiv doi.org/10.1101/2021.03.24.436523.
Joyce, M. G., King, H. A. D., Elakhal-Naouar, I., Ahmed, A., Peachman, K. K., Macedo Cincotta, C., Subra, C., Chen, R. E., Thomas, P. V., Chen, W. H., et al. (2022). A SARS-CoV-2 ferritin nanoparticle vaccine elicits protective immune responses in nonhuman primates. Sci. Transl. Med. 14, eabi5735.doi.org/10.1126/scitranslmed.abi5735.
Kanekiyo, M., Wei, C. J., Yassine, H. M., McTamney, P. M., Boyington, J. C., Whittle, J. R. R., Rao, S. S., Kong, W. P., Wang, L., and Nabel, G. J. (2013). Self-assembling influenza nanoparticle vaccines elicit broadly neutralizing H1N1 antibodies. Nature 499, 102-106. doi.org/10.1038/nature12202.
Kanekiyo, M., Bu, W., Joyce, M. G., Meng, G., Whittle, J. R. R., Baxa, U., Yamamoto, T., Narpala, S., Todd, J. P., Rao, S. S., et al. (2015). Rational Design of an Epstein-Barr Virus Vaccine Targeting the Receptor-Binding Site. Cell 162, 1090-1100. doi.org/10.1016/j.ce11.2015.07.043.
Kanekiyo, M., Joyce, M. G., Gillespie, R. A., Gallagher, J. R., Andrews, S. F., Yassine, H. M., Wheatley, A. K., Fisher, B. E., Ambrozak, D. R., Creanga, A., et al. (2019a). Mosaic nanoparticle display of diverse influenza virus hemagglutinins elicits broad B cell responses. Nat. Immunol. 362-372.doi.org/10.1038/s41590-018-0305-x.
Kanekiyo, M., Ellis, D., and King, N. P. (2019b). New Vaccine Design and Delivery Technologies. J. Infect. Dis. 219, S88-S96.doi.org/10.1093/infdis/jiy745.
Kato, Y., Abbott, R. K., Freeman, B. L., Haupt, S., Groschel, B., Silva, M., Menis, S., Irvine, D. J., Schief, W. R., and Crotty, S. (2020). Multifaceted Effects of Antigen Valency on B Cell Response Composition and Differentiation In Vivo. Immunity 53, 548-563.e8. doi.org/10.1016/j.immuni.2020.08.001.
Katz, D. H., Paul, W. E., Goidl, E. A., and Benacerraf, B. (1970). Carrier function in anti-hapten immune responses. I. Enhancement of primary and secondary anti-hapten antibody responses by carrier preimmunization. J. Exp. Med. 132, 261-282.doi.org/10.1084/jem.132.2.261.
Keech, C., Albert, G., Cho, I., Robertson, A., Reed, P., Neal, S., Plested, J. S., Zhu, M., Cloney-Clark, S., Zhou, H., et al. (2020). Phase 1-2 Trial of a SARS-CoV-2 Recombinant Spike Protein Nanoparticle Vaccine. N. Engl. J. Med. 383, 2320-2332.doi.org/10.1056/NEJMoa2026920.
Kelly, H. G., Tan, H. X., Juno, J. A., Esterbauer, R., Ju, Y., Jiang, W., Wimmer, V. C., Duckworth, B. C., Groom, J. R., Caruso, F., et al. (2020). Self-assembling influenza nanoparticle vaccines drive extended germinal center activity and memory B cell maturation. JCI Insight 5. doi.org/10.1172/jci.insight.136653.
Kim, P. H., Sohn, J. H., Choi, J. W., Jung, Y., Kim, S. W., Haam, S., and Yun, C. O. (2011). Active targeting and safety profile of PEG-modified adenovirus conjugated with herceptin. Biomaterials 32, 2314-2326.doi.org/10.1016/j.biomaterials.2010.10.031.
King, N. P., Bale, J. B., Sheffler, W., McNamara, D. E., Gonen, S., Gonen, T., Yeates, T. O., and Baker, D. (2014). Accurate design of co-assembling multi-component protein nanomaterials. Nature 510, 103-108.doi.org/10.1038/nature13404.
Klasse, P. J., Ozorowski, G., Sanders, R. W., and Moore, J. P. (2020). Env Exceptionalism: Why Are HIV-1 Env Glycoproteins Atypical Immunogens? Cell Host Microbe 27, 507-518. doi.org/10.1016/j.chom.2020.03.018.
Klinke, S., Zylberman, V., Bonomi, H. R., Haase, I., Guimarães, B. G., Braden, B. C., Bacher, A., Fischer, M., and Goldbaum, F. A. (2007). Structural and kinetic properties of lumazine synthase isoenzymes in the order Rhizobiales. J. Mol. Biol. 373, 664-680. doi.org/10.1016/j.jmb0.2007.08.021.
Lainšček, D., Fink, T., Forstnerič, V., Hafner-Bratkovič, I., Orehek, S., Strmšek, Ž., Manček-Keber, M., Pečan, P., Esih, H., Malenšek, Š., et al. (2021). A Nanoscaffolded Spike-RBD Vaccine Provides Protection against SARS-CoV-2 with Minimal Anti-Scaffold Response. Vaccines (Basel) 9.doi.org/10.3390/vaccines9050431.
Langowski, M. D., Khan, F. A., Bitzer, A. A., Genito, C. J., Schrader, A. J., Martin, M. L., Soto, K., Zou, X., Hadiwidjojo, S., Beck, Z., et al. (2020). Optimization of a Plasmodium falciparum circumsporozoite protein repeat vaccine using the tobacco mosaic virus platform. Proc. Natl. Acad. Sci. U.S.A 117, 3114-3122.doi.org/10.1073/pnas.1911792117.
Lavinder, J. J., Hoi, K. H., Reddy, S. T., Wine, Y., and Georgiou, G. (2014). Systematic characterization and comparative analysis of the rabbit immunoglobulin repertoire. PLoS One 9, e101322.doi.org/10.1371/journal.pone.0101322.
Lee, N. K., Lee, E. J., Kim, S., Nam, G. H., Kih, M., Hong, Y., Jeong, C., Yang, Y., Byun, Y., and Kim, I. S. (2017). Ferritin nanocage with intrinsically disordered proteins and affibody: A platform for tumor targeting with extended pharmacokinetics. J. Control. Release 267, 172-180. doi.org/10.1016/j.jconre1.2017.08.014.
Lell, B., Agnandji, S., von Glasenapp, I., Haertle, S., Oyakhiromen, S., Issifou, S., Vekemans, J., Leach, A., Lievens, M., Dubois, M. C., et al. (2009). A randomized trial assessing the safety and immunogenicity of ASO1 and AS02 adjuvanted RTS,S malaria vaccine candidates in children in Gabon. PLoS One 4, e7611.doi.org/10.1371/journal.pone.0007611.
Leman, J. K., Weitzner, B. D., Lewis, S. M., Adolf-Bryfogle, J., Alam, N., Alford, R. F., Aprahamian, M., Baker, D., Barlow, K. A., Barth, P., et al. (2020). Macromolecular modeling and design in Rosetta: recent methods and frameworks. Nat. Methods 17, 665-680. doi.org/10.1038/s41592-020-0848-2.
Lempp, F. A., Soriaga, L. B., Montiel-Ruiz, M., Benigni, F., Noack, J., Park, Y. J., Bianchi, S., Walls, A. C., Bowen, J. E., Zhou, J., et al. (2021). Lectins enhance SARS-CoV-2 infection and influence neutralizing antibodies. Nature 598, 342-347.doi.org/10.1038/s41586-021-03925-1.
López-Sagaseta, J., Malito, E., Rappuoli, R., and Bottomley, M. J. (2016). Self-assembling protein nanoparticles in the design of vaccines. Comput. Struct. Biotechnol. J. 14, 58-68. doi.org/10.1016/j.csbj.2015.11.001.
Luginbuhl, K. M., Schaal, J. L., Umstead, B., Mastria, E. M., Li, X., Banskota, S., Arnold, S., Feinglos, M., D'Alessio, D., and Chilkoti, A. (2017). One-week glucose control via zero-order release kinetics from an injectable depot of glucagon-like peptide-1 fused to a thermosensitive biopolymer. Nat Biomed Eng 1.doi.org/10.1038/s41551-017-0078.
Marcandalli, J., Fiala, B., Ols, S., Perotti, M., de van der Schueren, W., Snijder, J., Hodge, E., Benhaim, M., Ravichandran, R., Carter, L., et al. (2019). Induction of Potent Neutralizing Antibody Responses by a Designed Protein Nanoparticle Vaccine for Respiratory Syncytial Virus. Cell 176, 1420-1431.e17.doi.org/10.1016/j.ce11.2019.01.046.
McCallum, M., De Marco, A., Lempp, F. A., Tortorici, M. A., Pinto, D., Walls, A. C., Beltramello, M., Chen, A., Liu, Z., Zatta, F., et al. (2021). N-terminal domain antigenic mapping reveals a site of vulnerability for SARS-CoV-2. Cell 184, 2332-2347.e16.doi.org/10.1016/j.ce11.2021.03.028.
McCluskie, M. J., Evans, D. M., Zhang, N., Benoit, M., McElhiney, S. P., Unnithan, M., DeMarco, S. C., Clay, B., Huber, C., Deora, A., et al. (2016). The effect of preexisting anti-carrier immunity on subsequent responses to CRM197 or Qb-VLP conjugate vaccines. Immunopharmacol. Immunotoxicol. 38, 184-196.doi.org/10.3109/08923973.2016.1165246.
McCoy, L. E., van Gils, M. J., Ozorowski, G., Messmer, T., Briney, B., Voss, J. E., Kulp, D. W., Macauley, M. S., Sok, D., Pauthner, M., et al. (2016). Holes in the Glycan Shield of the Native HIV Envelope Are a Target of Trimer-Elicited Neutralizing Antibodies. Cell Rep. 16, 2327-2338.doi.org/10.1016/j.celrep.2016.07.074.
McLellan, J. S., Chen, M., Joyce, M. G., Sastry, M., Stewart-Jones, G. B. E., Yang, Y., Zhang, B., Chen, L., Srivatsan, S., Zheng, A., et al. (2013). Structure-based design of a fusion glycoprotein vaccine for respiratory syncytial virus. Science 342, 592-598.doi.org/10.1126/science.1243283.
Mesin, L., Ersching, J., and Victora, G. D. (2016). Germinal Center B Cell Dynamics. Immunity 471-482.doi.org/10.1016/j.immuni.2016.09.001.
Mitchison, N. A. (1971a). The carrier effect in the secondary response to hapten-protein conjugates. I. Measurement of the effect with transferred cells and objections to the local environment hypothesis. Eur. J. Immunol. 1, 10-17.doi.org/10.1002/eji.1830010103.
Mitchison, N. A. (1971b). The carrier effect in the secondary response to hapten-protein conjugates. II. Cellular cooperation. Eur. J. Immunol. 1, 18-27.doi.org/10.1002/eji.1830010104.
Molino, N. M., Bilotkach, K., Fraser, D. A., Ren, D., and Wang, S. W. (2012). Complement activation and cell uptake responses toward polymer-functionalized protein nanocapsules. Biomacromolecules 13, 974-981.doi.org/10.1021/bm300083e.
Murray, A. N., Chen, W., Antonopoulos, A., Hanson, S. R., Wiseman, R. L., Dell, A., Haslam, S. M., Powers, D. L., Powers, E. T., and Kelly, J. W. (2015). Enhanced Aromatic Sequons Increase Oligosaccharyltransferase Glycosylation Efficiency and Glycan Homogeneity. Chem. Biol. 22, 1052-1062.doi.org/10.1016/j.chembio1.2015.06.017.
NCT03186781 Influenza HA Ferritin Vaccine, Alone or in Prime-Boost Regimens With an Influenza DNA Vaccine in Healthy Adults (clinicaltrials.govict2/show/NCT03186781).
NCT03547245 A Phase I Trial to Evaluate the Safety and Immunogenicity of eOD-GT8 60mer Vaccine, Adjuvanted (clinicaltrials.govict2/show/NCT03547245).
NCT03814720 Dose, Safety, Tolerability and Immunogenicity of an Influenza H1 Stabilized Stem Ferritin Vaccine, VRCFLUNPF099-00-VP, in Healthy Adults (clinicaltrials.gov/ct2/show/NCT03814720).
NCT04645147 Safety and Immunogenicity of an Epstein-Barr Virus (EBV) gp350-Ferritin Nanoparticle Vaccine in Healthy Adults With or Without EBV Infection (clinicaltrials.gov/ct2/show/NCT04645147).
NCT04784767 SARS-COV-2-Spike-Ferritin-Nanoparticle (SpFN) Vaccine With ALFQ Adjuvant for Prevention of COVID-19 in Healthy Adults (clinicaltrials.gov/ct2/show/NCT04784767).
NCT04896086 First-in-Human Clinical Trial of a Mosaic Quadrivalent Influenza Vaccine Compared With a Licensed Inactivated Seasonal QIV, in Healthy Adults (clinicaltrials.gov/ct2/show/NCT04896086).
NCT05001373 A Phase 1 Study to Evaluate the Safety and Immunogenicity of eOD-GT8 60mer mRNA Vaccine (mRNA-1644) and Core-g28v2 60mer mRNA Vaccine (mRNA-1644v2-Core) (clinicaltrials.govict2/show/NCT05001373).
NCT05007951 A Phase III Study to Assess the Immunogenicity and Safety of SK SARS-CoV-2 Recombinant Nanoparticle Vaccine Adjuvanted With AS03 (GBP510) in Adults Aged 18 Years and Older (www.clinicaltrials.govict2/show/NCT05007951).
O'Riordan, C. R., Lachapelle, A., Delgado, C., Parkes, V., Wadsworth, S. C., Smith, A. E., and Francis, G. E. (1999). PEGylation of adenovirus with retention of infectivity and protection from neutralizing antibody in vitro and in vivo. Hum. Gene Ther. 10, 1349-1358. doi.org/10.1089/10430349950018021.
Pallesen, J., Wang, N., Corbett, K. S., Wrapp, D., Kirchdoerfer, R. N., Turner, H. L., Cottrell, C. A., Becker, M. M., Wang, L., Shi, W., et al. (2017). Immunogenicity and structures of a rationally designed prefusion MERS-CoV spike antigen. Proc. Natl. Acad. Sci. U.S.A 114, E7348-E7357.doi.org/10.1073/pnas.1707304114.
Pantophlet, R., and Burton, D. R. (2006). GP120: target for neutralizing HIV-1 antibodies. Annu. Rev. Immunol. 24, 739-769.doi.org/10.1146/annurev.immuno1.24.021605.090557.
Pantophlet, R., Wilson, I. A., and Burton, D. R. (2003). Hyperglycosylated mutants of human immunodeficiency virus (HIV) type 1 monomeric gp120 as novel antigens for HIV vaccine design. J. Virol. 77, 5889-5901.doi.org/10.1128/jvi.77.10.5889-5901.2003.
Pardi, N., Hogan, M. J., Porter, F. W., and Weissman, D. (2018). mRNA vaccines—a new era in vaccinology. Nat. Rev. Drug Discov. 17, 261-279.doi.org/10.1038/nrd.2017.243.
Polack, F. P., Thomas, S. J., Kitchin, N., Absalon, J., Gurtman, A., Lockhart, S., Perez, J. L., Perez Marc, G., Moreira, E. D., Zerbini, C., et al. (2020). Safety and Efficacy of the BNT162b2 mRNA Covid-19 Vaccine. N. Engl. J. Med. 383, 2603-2615.doi.org/10.1056/NEJMoa2034577.
Rahikainen, R., Rijal, P., Tan, T. K., Wu, H. J., Andersson, A. M. C., Barrett, J. R., Bowden, T. A., Draper, S. J., Townsend, A. R., and Howarth, M. (2021). Overcoming Symmetry Mismatch in Vaccine Nanoassembly through Spontaneous Amidation. Angew. Chem. Int. Ed Engl. 60, 321-330.doi.org/10.1002/anie.202009663.
Raja, K. S., Wang, Q., Gonzalez, M. J., Manchester, M., Johnson, J. E., and Finn, M. G. (2003). Hybrid virus-polymer materials. 1. Synthesis and properties of PEG-decorated cowpea mosaic virus. Biomacromolecules 4, 472-476.doi.org/10.1021/bm025740+.
Rappuoli, R., Bottomley, M. J., D'Oro, U., Finco, O., and De Gregorio, E. (2016). Reverse vaccinology 2.0: Human immunology instructs vaccine antigen design. J. Exp. Med. 213, 469-481.doi.org/10.1084/jem.20151960.
Read, B. J., Won, L., Kraft, J. C., Sappington, I., Aung, A., Wu, S., Bals, J., Chen, C., Lee, K. K., Lingwood, D., et al. (2022). Mannose-binding lectin and complement mediate follicular localization and enhanced immunogenicity of diverse protein nanoparticle immunogens. Cell Rep. 38, 110217.doi.org/10.1016/j.celrep.2021.110217.
Ringe, R. P., Ozorowski, G., Rantalainen, K., Struwe, W. B., Matthews, K., Tones, J. L., Yasmeen, A., Cottrell, C. A., Ketas, T. J., LaBranche, C. C., et al. (2017). Reducing V3 Antigenicity and Immunogenicity on Soluble, Native-Like HIV-1 Env SOSIP Trimers. J. Virol. 91.doi.org/10.1128/JVI.00677-17.
Ringe, R. P., Pugach, P., Cottrell, C. A., LaBranche, C. C., Seabright, G. E., Ketas, T. J., Ozorowski, G., Kumar, S., Schorcht, A., van Gils, M. J., et al. (2019). Closing and Opening Holes in the Glycan Shield of HIV-1 Envelope Glycoprotein SOSIP Trimers Can Redirect the Neutralizing Antibody Response to the Newly Unmasked Epitopes. J. Virol. 93. doi.org/10.1128/JVI.01656-18.
Sanders, R. W., Vesanen, M., Schuelke, N., Master, A., Schiffner, L., Kalyanaraman, R., Paluch, M., Berkhout, B., Maddon, P. J., Olson, W. C., et al. (2002). Stabilization of the soluble, cleaved, trimeric form of the envelope glycoprotein complex of human immunodeficiency virus type 1. J. Virol. 76, 8875-8889.doi.org/10.1128/jvi.76.17.8875-8889.2002.
Sauer, M. M., Tortorici, M. A., Park, Y. J., Walls, A. C., Homad, L., Acton, O. J., Bowen, J. E., Wang, C., Xiong, X., de van der Schueren, W., et al. (2021). Structural basis for broad coronavirus neutralization. Nat. Struct. Mol. Biol. 28, 478-486.doi.org/10.1038/s41594-021-00596-4.
Saunders, K. O., Lee, E., Parks, R., Martinez, D. R., Li, D., Chen, H., Edwards, R. J., Gobeil, S., Barr, M., Mansouri, K., et al. (2021). Neutralizing antibody vaccine for pandemic and pre-emergent coronaviruses. Nature 594, 553-559.doi.org/10.1038/s41586-021-03594-0.
Schellenberger, V., Wang, C. W., Geething, N.C., Spink, B. J., Campbell, A., To, W., Scholle, M. D., Yin, Y., Yao, Y., Bogin, O., et al. (2009). A recombinant polypeptide extends the in vivo half-life of peptides and proteins in a tunable manner. Nat. Biotechnol. 27, 1186-1190. doi.org/10.1038/nbt.1588.
Schlapschy, M., Binder, U., Börger, C., Theobald, I., Wachinger, K., Kisling, S., Haller, D., and Skerra, A. (2013). PASylation: a biological alternative to PEGylation for extending the plasma half-life of pharmaceutically active proteins. Protein Eng. Des. Sel. 26, 489-501. doi.org/10.1093/protein/gzt023.
Schödel, F., Wirtz, R., Peterson, D., Hughes, J., Warren, R., Sadoff, J., and Milich, D. (1994). Immunity to malaria elicited by hybrid hepatitis B virus core particles carrying circumsporozoite protein epitopes. J. Exp. Med. 180, 1037-1046.doi.org/10.1084/jem.180.3.1037.
Schödel, F., Peterson, D., Hughes, J., Wirtz, R., and Milich, D. (1996). Hybrid hepatitis B virus core antigen as a vaccine carrier moiety: I. presentation of foreign epitopes. J. Biotechnol. 44, 91-96.doi.org/10.1016/0168-1656(95)00118-2.
Schorcht, A., van den Kerkhof, T. L. G. M., Cottrell, C. A., Allen, J. D., Torres, J. L., Behrens, A. J., Schermer, E. E., Burger, J. A., de Taeye, S. W., Torrents de la Peña, A., et al. (2020). Neutralizing Antibody Responses Induced by HIV-1 Envelope Glycoprotein SOSIP Trimers Derived from Elite Neutralizers. J. Virol. 94.doi.org/10.1128/JVI.01214-20.
Selvarajah, S., Puffer, B., Pantophlet, R., Law, M., Doms, R. W., and Burton, D. R. (2005). Comparing antigenicity and immunogenicity of engineered gp120. J. Virol. 79, 12148-12163. doi.org/10.1128/JVI.79.19.12148-12163.2005.
Sliepen, K., Ozorowski, G., Burger, J. A., van Montfort, T., Stunnenberg, M., LaBranche, C., Montefiori, D. C., Moore, J. P., Ward, A. B., and Sanders, R. W. (2015a). Presenting native-like HIV-1 envelope trimers on ferritin nanoparticles improves their immunogenicity. Retrovirology 12, 82.doi.org/10.1186/s12977-015-0210-4.
Sliepen, K., van Montfort, T., Melchers, M., Isik, G., and Sanders, R. W. (2015b). Immunosilencing a highly immunogenic protein trimerization domain. J. Biol. Chem. 290, 7436-7442.doi.org/10.1074/jbc.M114.620534.
Sliepen, K., Han, B. W., Bontjer, I., Mooij, P., Garces, F., Behrens, A. J., Rantalainen, K., Kumar, S., Sarkar, A., Brouwer, P. J. M., et al. (2019). Structure and immunogenicity of a stabilized HIV-1 envelope trimer based on a group-M consensus sequence. Nat. Commun. 10, 2355.doi.org/10.1038/s41467-019-10262-5.
Sliepen, K., Schermer, E., Bontjer, I., Burger, J. A., Lévai, R. F., Mundsperger, P., Brouwer, P. J. M., Tolazzi, M., Farsang, A., Katinger, D., et al. (2021). Interplay of diverse adjuvants and nanoparticle presentation of native-like HIV-1 envelope trimers. NPJ Vaccines 6, 103. doi.org/10.1038/s41541-021-00364-x.
Sok, D., Briney, B., Jardine, J. G., Kulp, D. W., Menis, S., Pauthner, M., Wood, A., Lee, E. C., Le, K. M., Jones, M., et al. (2016). Priming HIV-1 broadly neutralizing antibody precursors in human Ig loci transgenic mice. Science 353, 1557-1560.doi.org/10.1126/science.aah3945.
Song, J. Y., Choi, W. S., Heo, J. Y., Lee, J. S., Jung, D. S., Kim, S. W., Park, K. H., Eom, J. S., Jeong, S. J., Lee, J., et al. (2022). Safety and immunogenicity of a SARS-CoV-2 recombinant protein nanoparticle vaccine (GBP510) adjuvanted with AS03: a phase 1/2, randomized, placebo-controlled, observer-blinded trial.
Steinmetz, N. F., and Manchester, M. (2009). PEGylated viral nanoparticles for biomedicine: the impact of PEG chain length on VNP cell interactions in vitro and ex vivo. Biomacromolecules 784-792.doi.org/10.1021/bm8012742.
Struwe, W. B., Chertova, E., Allen, J. D., Seabright, G. E., Watanabe, Y., Harvey, D. J., Medina-Ramirez, M., Roser, J. D., Smith, R., Westcott, D., et al. (2018). Site-Specific Glycosylation of Virion-Derived HIV-1 Env Is Mimicked by a Soluble Trimeric Immunogen. Cell Rep. 24, 1958-1966.e5.doi.org/10.1016/j.celrep.2018.07.080.
Swanson, K. A., Rainho-Tomko, J. N., Williams, Z. P., Lanza, L., Peredelchuk, M., Kishko, M., Pavot, V., Alamares-Sapuay, J., Adhikarla, H., Gupta, S., et al. (2020). A respiratory syncytial virus (RSV) F protein nanoparticle vaccine focuses antibody responses to a conserved neutralization domain. Sci Immunol 5.doi.org/10.1126/sciimmunol.aba6466.
Thornlow, D. N., Macintyre, A. N., Oguin, T. H., Karlsson, A. B., Stover, E. L., Lynch, H. E., Sempowski, G. D., and Schmidt, A. G. (2021). Altering the Immunogenicity of Hemagglutinin Immunogens by Hyperglycosylation and Disulfide Stabilization. Front. Immunol. 12, 737973. doi.org/10.3389/fimmu.2021.737973.
Tiller, T., Meffre, E., Yurasov, S., Tsuiji, M., Nussenzweig, M. C., and Wardemann, H. (2008). Efficient generation of monoclonal antibodies from single human B cells by single cell RT-PCR and expression vector cloning. J. Immunol. Methods 329, 112-124. doi.org/10.1016/j.jim.2007.09.017.
Tokatlian, T., Read, B. J., Jones, C. A., Kulp, D. W., Menis, S., Chang, J. Y. H., Steichen, J. M., Kumari, S., Allen, J. D., Dane, E. L., et al. (2019). Innate immune recognition of glycans targets HIV nanoparticle immunogens to germinal centers. Science 363, 649-654. doi.org/10.1126/science.aat9120.
Tytgat, H. L. P., Lin, C. W., Levasseur, M. D., Tomek, M. B., Rutschmann, C., Mock, J., Liebscher, N., Terasaka, N., Azuma, Y., Wetter, M., et al. (2019). Cytoplasmic glycoengineering enables biosynthesis of nanoscale glycoprotein assemblies. Nat. Commun. 10, 5403. doi.org/10.1038/s41467-019-13283-2.
Ueda, G., Antanasijevic, A., Fallas, J. A., Sheffler, W., Copps, J., Ellis, D., Hutchinson, G. B., Moyer, A., Yasmeen, A., Tsybovsky, Y., et al. (2020). Tailored design of protein nanoparticle scaffolds for multivalent presentation of viral glycoprotein antigens. Elife 9. doi.org/10.7554/eLife.57659.
Vannucci, L., Falvo, E., Fornara, M., Di Micco, P., Benada, O., Krizan, J., Svoboda, J., Hulikova-Capkova, K., Morea, V., Boffi, A., et al. (2012). Selective targeting of melanoma by PEG-masked protein-based multifunctional nanoparticles. Int. J. Nanomedicine 7, 1489-1509. doi org/10.2147/IJN. S28242.
Walls, A. C., Fiala, B., Schäfer, A., Wrenn, S., Pham, M. N., Murphy, M., Tse, L. V., Shehata, L., O'Connor, M. A., Chen, C., et al. (2020). Elicitation of Potent Neutralizing Antibody Responses by Designed Protein Nanoparticle Vaccines for SARS-CoV-2. Cell 183, 1367-1382.e17. doi.org/10.1016/j.ce11.2020.10.043.
Walls, A. C., Miranda, M. C., Schäfer, A., Pham, M. N., Greaney, A., Arunachalam, P. S., Navarro, M. J., Tortorici, M. A., Rogers, K., O'Connor, M. A., et al. (2021). Elicitation of broadly protective sarbecovirus immunity by receptor-binding domain nanoparticle vaccines. Cell 184, 5432-5447.e16.doi.org/10.1016/j.ce11.2021.09.015.
Weidenbacher, P. A., and Kim, P. S. (2019). Protect, modify, deprotect (PMD): A strategy for creating vaccines to elicit antibodies targeting a specific epitope. Proc. Natl. Acad. Sci. U.S.A 116, 9947-9952.doi.org/10.1073/pnas.1822062116.
Weissman, D., Ni, H., Scales, D., Dude, A., Capodici, J., McGibney, K., Abdool, A., Isaacs, S. N., Cannon, G., and Karikó, K. (2000). HIV gag mRNA transfection of dendritic cells (DC) delivers encoded antigen to WIC class I and II molecules, causes DC maturation, and induces a potent human in vitro primary immune response. J. Immunol. 165, 4710-4717. doi.org/10.4049/jimmuno1.165.8.4710.
Woodruff, M. C., Kim, E. H., Luo, W., and Pulendran, B. (2018). B Cell Competition for Restricted T Cell Help Suppresses Rare-Epitope Responses. Cell Rep. 25, 321-327.e3. doi.org/10.1016/j.celrep.2018.09.029.
Wu, X., Yang, Z. Y., Li, Y., Hogerkorp, C. M., Schief, W. R., Seaman, M. S., Zhou, T., Schmidt, S. D., Wu, L., Xu, L., et al. (2010). Rational design of envelope identifies broadly neutralizing human monoclonal antibodies to HIV-1. Science 329, 856-861. doi.org/10.1126/science.1187659.
Yassine, H. M., Boyington, J. C., McTamney, P. M., Wei, C. J., Kanekiyo, M., Kong, W. P., Gallagher, J. R., Wang, L., Zhang, Y., Joyce, M. G., et al. (2015). Hemagglutinin-stem nanoparticles generate heterosubtypic influenza protection. Nat. Med. 21, 1065-1070. doi.org/10.1038/nm.3927.
Zakeri, B., Fierer, J. O., Celik, E., Chittock, E. C., Schwarz-Linek, U., Moy, V. T., and Howarth, M. (2012). Peptide tag forming a rapid covalent bond to a protein, through engineering a bacterial adhesin. Proc. Natl. Acad. Sci. U.S.A 109, E690-E697. doi.org/10.1073/pnas.1115485109.
Zaman, R., Islam, R. A., Ibnat, N., Othman, I., Zaini, A., Lee, C. Y., and Chowdhury, E. H. (2019). Current strategies in extending half-lives of therapeutic proteins. J. Control. Release 301, 176-189.doi.org/10.1016/j.jconre1.2019.02.016.

We claim:

1. A polypeptide comprising the amino acid sequence of SEQ ID NO:78-80, substituted with one or more sequon, wherein the N-terminal residue may be present or may be absent

2. The polypeptide of claim 1, wherein each sequon independently consists of the amino acid sequence selected from the group consisting of NET, NDS, NST, FSNES (SEQ ID NO:81), NES, FENES (SEQ ID NO:82), NAS, NGS, NHT, FFNHT (SEQ ID NO:83), NLS, FDNLS (SEQ ID NO:84), NNS, WHNNS (SEQ ID NO:85), NYS, FINYS (SEQ ID NO:86), NIS, FLNAT (SEQ ID NO:87), NAT, FLNAS (SEQ ID NO:88), WVNNS (SEQ ID NO:89), NKS, YLNKS (SEQ ID NO:90), FSNET (SEQ ID NO:91), YVNVT (SEQ ID NO:92), NRS, YANRS (SEQ ID NO:93), WANAS (SEQ ID NO:94), NFT, WANFT (SEQ ID NO:95), NVS, NGT, NVT, WLNHT (SEQ ID NO:96), and NTS.

3. The polypeptide of claim 1, wherein the polypeptide comprises the amino acid sequence selected from the group consisting of SEQ ID NO: 1-3, 5, 8-10, 13, 23, 26-28, 31-32, 34-38, 40, 42-46, 48-55, 59-60, and 67-74, wherein:
(a) each sequon may independently be substituted with any other sequon;
(b) X1 may be present or absent, and when present comprises a signal peptide; and
(c) X2 may be present or absent, and when present comprises a purification tag.

4. The polypeptide of claim 1, wherein the polypeptide comprises the amino acid sequence selection from the group consisting of SEQ ID NO: 1-3, 5, 8-10, 13, 23, 26-28, 31-32, 34-38, 40, 42-46, 48-55, 59-60, and 67-74.

5. The polypeptide of claim 1, wherein the polypeptide comprises the amino acid sequence selection from the group consisting of SEQ ID NO: 55, 59, 67, and 73.

6. A fusion protein, comprising
(a) the polypeptide of claim 1; and
(b) a functional domain linked to the polypeptide, either directly or via an optional amino acid linker.

7. The fusion protein of claim 8, wherein the functional domain comprises a bacterial antigen, a viral antigen, a fungal antigen, or a cancer antigen.

8. The fusion protein of claim 6, wherein the polypeptide comprises the amino acid sequence selection from the group consisting of SEQ ID NO: 59, 67, and 73.

9. A nanoparticle, comprising:
(a) a plurality of first assemblies, each first assembly comprising a plurality of identical first proteins comprising an amino acid sequence selected from the group consisting of SEQ ID NO: 10, 13, 23, and 59-60; and,
(b) a plurality of second assemblies, each second assembly comprising a plurality of second proteins comprising an amino acid sequence selected from the group consisting of SEQ ID NO: 1-3, 5, 8-9, and 49-55;
wherein the plurality of first assemblies non-covalently interact with the plurality of second assemblies to form the nanoparticle; and wherein:
(a) each sequon may independently be substituted with any other sequon;
(b) X1 may be present or absent, and when present comprises a signal peptide;
(c) X2 may be present or absent, and when present comprises a purification tag.

10. The nanoparticle of claim 9, wherein each first assembly comprises a plurality of identical first proteins comprising an amino acid sequence selected from the group consisting of SEQ ID NO: 10, 13, and 59-60, wherein:
(a) each sequon may independently be substituted with any other sequon;
(b) X1 may be present or absent, and when present comprises a signal peptide;
(c) X2 may be present or absent, and when present comprises a purification tag.

11. The nanoparticle of claim 9, wherein each second assembly comprising a plurality of second proteins comprising an amino acid sequence selected from the group consisting of SEQ ID NO: 55, 67, and 73, wherein:
(a) each sequon may independently be substituted with any other sequon;
(b) X1 may be present or absent, and when present comprises a signal peptide;
(c) X2 may be present or absent, and when present comprises a purification tag.

12. A nanoparticle, comprising:
(a) a plurality of first assemblies, each first assembly comprising a plurality of identical first proteins comprising the amino acid sequence selected of SEQ ID NO:152 or 153; and,
(b) a plurality of second assemblies, each second assembly comprising a plurality of second proteins comprising an amino acid sequence selected from the group consisting of SEQ ID NO: 26-48 and 61-77, wherein:
(i) each sequon may independently be substituted with any other sequon;
(ii) X1 may be present or absent, and when present comprises a signal peptide; and
(iii) X2 may be present or absent, and when present comprises a purification tag;
wherein the plurality of first assemblies non-covalently interact with the plurality of second assemblies to form the nanoparticle.

13. The nanoparticle of claim 12, wherein the plurality of second proteins comprise an amino acid sequence selected from the group consisting of SEQ ID NO: 67 and 73; wherein:
(i) each sequon may independently be substituted with any other sequon;
(ii) X1 may be present or absent, and when present comprises a signal peptide; and
(iii) X2 may be present or absent, and when present comprises a purification tag.

14. The nanoparticle of claim 9, wherein some or all of the second proteins comprise a fusion protein comprising an antigen.

15. A composition, comprising a plurality of nanoparticles of claim 9.

16. A nucleic acid molecule encoding the polypeptide of claim 1.

17. An expression vector comprising the nucleic acid molecule of claim 16 operatively linked to a suitable control sequence.

18. A cell comprising the expression vector of claim 17.

19. A pharmaceutical composition comprising
(a) a plurality of nanoparticles according to claim 9; and
(b) a pharmaceutically acceptable carrier.

20. A method to induce an immune response, comprising administering to a subject in need thereof an amount effective to induce an immune response of the nanoparticle of claim 9.
